(19) United States
(12) Patent Application Publication
(10) Pub. No.: US 2024/0390506 A1
(43) Pub. Date: Nov. 28, 2024

(54) IMMUNOCONJUGATES CONTAINING TNF-ALPHA AND RELATED METHODS AND COMPOSITIONS THEREOF

(71) Applicant: SUZHOU FUSE BIOSCIENCES LIMITED, Jiangsu (CN)

(72) Inventors: Qufei LI, Jiangsu (CN); Lucas BAILEY, Jiangsu (CN)

(21) Appl. No.: 18/696,943

(22) PCT Filed: May 13, 2022
(86) PCT No.: PCT/CN2022/092786

§ 371 Date: Mar. 28, 2024

(30) Foreign Application Priority Data
Sep. 28, 2021 (WO) PCT/CN2021/121318

(51) Int. Cl. A61K 47/62; A61K 47/68

(52) U.S. Cl. CPC A61K 47/62; A61K 47/6845; A61K 47/6851; A61K 47/6871

(57) ABSTRACT

Provided herein are immunoconjugate molecules containing a TNF-α polypeptide and a masking moiety capable of inhibiting and activating the TNF-α activity under suitable conditions. Also provided herein are methods for producing the immunoconjugate molecules. Finally, provided herein are the therapeutic uses of the immunoconjugate molecules due to their modulating effects on the immune system for treating diseases such as cancer and other chronic infectious diseases.

### CROSS-REFERENCE TO RELATED APPLICATIONS

This application claims the benefit of priority of PCT/CN2021/121318 filed on Sep. 28, 2021, the content of which is herein incorporated by reference in its entirety.

### REFERENCE TO SEQUENCE LISTING SUBMITTED ELECTRONICALLY

This application contains a sequence listing, which is submitted electronically as an ASCII formatted sequence listing with a file “14625-007-228_SEQLIST.txt” and a creation date of May 9, 2022 and having a size of 21,664 bytes. The sequence listing submitted electronically is part of the specification and is herein incorporated by reference in its entirety.

### 1. FIELD

The present disclosure generally relates to tumor necrosis factor alpha (TNF-α) containing immunoconjugate molecules. More particularly, the present disclosure concerns immunoconjugate molecules exhibiting improved properties for use as immunotherapeutic agents due to the ability of modulating the immune system. The present disclosure further relates to therapeutic uses and pharmaceutical compositions of the immunoconjugate molecules for treating diseases such as cancer and other chronic infectious diseases.

### 2. BACKGROUND

Tumor necrosis factor alpha (TNF-α) is a 17-kDa protein consisting of 157 amino acids that is a homotrimer in solution. TNF-α is a potent, pleiotropic cytokine capable of triggering apoptosis of tumor endothelial cells, and consequently tumor necrosis. TNF-α has gained approval in Europe for use in the treatment of soft tissue sarcoma in combination with chemotherapy. However, due to its severe systemic toxicities, TNF-α has a low maximum tolerated total systemic dose of ˜0.4 mg. The systemic toxicities include respiratory failure, coagulopathies, hypotension, thrombocytopenia, leukopenia, neurotoxicity, fever, headache, nausea/vomiting, hepatopathy, as well as general symptoms of malaise and weakness. As a result, authorized use of TNF-α in soft tissue sarcoma is limited to isolated limb perfusion to reduce systemic exposure. Therefore, the full potential of TNF-α has not been realized, and there remains a need in the art to further enhance the therapeutic usefulness of TNF-α. The present disclosure meets this need.

### 3. SUMMARY

The present disclosure provides immunoconjugate molecules comprising a TNF-α polypeptide. The present disclosure also provides, in certain embodiments, polynucleotides and vectors comprising sequences encoding such immunoconjugate molecules, and compositions, reagents, and kits comprising such immunoconjugate molecules. In related aspect, provided herein are also methods for delivery and/or activation of a TNF-α activity at a target site, or reduced toxicity and/or other side-effects associated with systemic exposure to the TNF-α activity in a subject through the use of the immunoconjugate molecules according to the present disclosure.

In one aspect, provided herein are immunoconjugate molecules. In some embodiments, an immunoconjugate molecule according to the present disclosure comprises (a) a cytokine moiety comprising a cytokine polypeptide having a cytokine activity; and (b) a masking moiety; wherein the masking moiety comprises a bispecific antibody or antigen binding fragment thereof capable of binding to the cytokine polypeptide and a first target antigen; wherein when binding to the cytokine polypeptide, the masking moiety reduces or inhibits the cytokine activity; wherein when binding to the first target antigen, the masking moiety disassociates from the cytokine polypeptide, thereby activating the cytokine activity; and wherein the cytokine polypeptide is wild-type or mutant TNF-α.

In some embodiments, the masking moiety comprises an intact antibody, a Fab, a scFab, a Fab′, a F(ab′)₂, a Fv, a scFv, a dsFv, a diabody, a triabody, a tetrabody, or a VHH formed from antibody fragments. In some embodiments, the bispecific antibody is a two-in-one antibody.

In some embodiments, the first target antigen is not the cytokine polypeptide. In some embodiments, the first target antigen is expressed on a first cell surface. In some embodiments, the first cell does not express a TNF-α receptor. In some embodiments, the first cell also expresses a TNF-α receptor. In some embodiments, the first cell is a cancer cell or a cell in a tumor microenvironment. In some embodiments, the first target antigen is soluble.

In some embodiments, the first target antigen is a tumor associated antigen. In some embodiments, the first target antigen is fibroblast activation protein (FAP). In some embodiments, the first target antigen is Tumor-associated calcium signal transducer 2 (TROP2).

In some embodiments, the TNF-α is wild-type or mutant human TNF-α. In some embodiments, the mutant human TNF-α comprises at least one point mutation selected from (a) A33T, N34A, N34H, Y87A, Y87F, S95A, A145G, A145V, S147A, and S147G in SEQ ID NO:1; (b) P08C, L55C, Q102C, E104C, S95C, G148C, I97C, Y115C, H73C, and P113C of SEQ ID NO:1; or (c) (i) double mutations of P08C and L55C; (ii) double mutations of Q102C and E104C, (iii) double mutations of S95C and G148C, (iv) double mutations of I97C and Y115C; (v) double mutations of H73C and P113C; or (vi) any combinations of (i) to (v), of SEQ ID NO:1.

In some embodiments, the TNF-α is wild-type or mutant TNF-α from a non-human species. In some embodiments, the mutant TNF-α comprises at least one point mutation corresponding to (a) A33T, N34A, N34H, Y87A, Y87F, S95A, A145G, A145V, S147A, and S147G in SEQ ID NO:1; (b) P08C, L55C, Q102C, E104C, S95C, G148C, I97C, Y115C, H73C, and P113C of SEQ ID NO:1; or (c) (i) double mutations of P08C and L55C; (ii) double mutations of Q102C and E104C, (iii) double mutations of S95C and G148C, (iv) double mutations of 197C and Y115C; (v) double mutations of H73C and P113C; or (vi) any combinations of (i) to (v), of SEQ ID NO:1.

In some embodiments, the immunoconjugate molecule further comprises (c) an anchoring moiety comprising an antibody or antigen binding fragment thereof that specifically binds to a second target antigen.

In some embodiments, the second target antigen is expressed on a second cell surface. In some embodiments, the second cell is a cancer cell or a cell in a tumor microenvironment. In some embodiments, the second target antigen is soluble. In some embodiments, the second target antigen is a tumor associated antigen. In some embodiments, the first and second target antigens are the same.

In some embodiments, the bispecific masking moiety and the anchoring moiety bind to the same epitope of the first or second target antigen. In some embodiments, the bispecific masking moiety and the anchoring moiety bind to different epitopes of the first or second target antigen. In some embodiments, the second target antigen is fibroblast activation protein (FAP) or Tumor-associated calcium signal transducer 2 (TROP2). In some embodiments, the first and second target antigens are different. In some embodiments, the first and second cells are the same or different.

In some embodiments, the anchoring moiety comprises an intact antibody, a Fab, a scFab, a Fab′, a F(ab′)₂, a Fv, a scFv, a dsFv, a diabody, a triabody, a tetrabody, or a VHH formed from antibody fragments. In some embodiments, the bispecific antibody or antigen binding fragment of the masking moiety is a Fab, scFv or VHH, and wherein the antibody or antigen binding fragment thereof of the anchoring moiety is a Fab, scFv or VHH. In some embodiments, the bispecific antibody or antigen binding fragment of the masking moiety is a Fab, and wherein the antibody or antigen binding fragment thereof of the anchoring moiety is a scFv.

In some embodiments, the masking moiety is fused directly to the cytokine moiety. In some embodiments, the masking moiety is fused to the cytokine moiety via a peptidic linker. In some embodiments, the masking moiety is fused to the C-terminus the TNF-α polypeptide. In some embodiments, the masking moiety is fused to the N-terminus the TNF-α polypeptide. In some embodiments, the anchoring moiety is fused directly to the cytokine moiety or is fused directly to the masking moiety. In some embodiments, the anchoring moiety is fused to the cytokine moiety or the masking moiety via a peptidic linker.

In some embodiments, the masking moiety comprises a Fab having a heavy chain subunit and a light chain subunit, wherein the N terminus of the heavy chain subunit is fused via a linker to the TNF-α polypeptide, and wherein the N terminus of light chain subunit is fused via a linker to the anchoring moiety. In some embodiments, the anchoring moiety is a scFv.

In some embodiments, the immunoconjugate molecule further comprises (d) a conjugating moiety, wherein the conjugating moiety operably connects two or more of the cytokine moiety, the masking moiety, and the anchoring moiety.

In some embodiments, the conjugating moiety comprises an immunoglobulin Fc domain or a mutant thereof. In some embodiments, the Fc domain comprises a first subunit and a second subunit that are two non-identical polypeptidic chains; and wherein the Fc domain comprises a first modification promoting hetero-dimerization of the two non-identical polypeptidic chains. In some embodiments, the first modification is a knob-into-hole modification comprising a knob modification in the first subunit and a hole modification in the second subunit.

In some embodiments, the Fc domain comprises a second modification, wherein the Fc domain has reduced binding affinity to an Fc receptor compared to a native Fc domain without said second modification. In some embodiments, the Fc domain has reduced binding affinity to a Fcγ receptor as compared to the native Fc domain without said second modification. In some embodiments, the Fcγ receptor is an FcγRIIIα, FcγRI or FcγRIIα receptor.

In some embodiments, the Fc domain has reduced binding affinity to a complement component as compared to the native Fc domain without said second modification. In some embodiments, the complement component is C1q.

In some embodiments, the Fc domain has reduced Fc effector function as compared to an Fc domain without said second modification. In some embodiments, the reduced Fc effector function is selected from complement dependent cytotoxicity (CDC), antibody-dependent cell-mediated cytotoxicity (ADCC), antibody-dependent cellular phagocytosis (ADCP), cytokine secretion, downregulation of cell surface receptors, and B cell activation.

In some embodiments, the second modification comprises one or more mutations selected from S228P, E233P, L234V, L234A, L235A, L235E, ΔG236, D265G, N297A, N297D, P329E, P329S, P329A, P329G, A330S, or P331S, wherein the numbering is that of the EU index as in Kabat. In some embodiments, the second modification comprises one or more mutations selected from E233P, L234V, L234A, L235A, ΔG236, D265G, P327E, A328S, P329E, A330S, or P331S, wherein the numbering is that of the EU index as in Kabat.

In some embodiments, the cytokine moiety is connected to the C-terminus of one of the first and second subunits of the Fc domain, and the masking moiety is connected to the C-terminus of the other of the first and second subunits of the Fc domain. In some embodiments, the anchoring moiety is connected to the N-terminus of one of the first and second subunits of the Fc domain. In some embodiments, the anchoring moiety and the cytokine moiety are connected to the same subunit of the Fc domain. In some embodiments, the anchoring moiety and the masking moiety are connected to the same subunit of the Fc domain. In some embodiments, the masking moiety is connected to the C-terminus of one of the first and second subunits of the Fc domain; and wherein the cytokine moiety is connected to the masking moiety. In some embodiments, the anchoring moiety is connected to the N-terminus of one of the first and second subunits of the Fc domain. In some embodiments, the anchoring moiety and the masking moiety are connected to the same subunit of the Fc domain; or wherein the anchoring moiety and the masking moiety are connected to different subunits of the Fc domain. In some embodiments, the masking moiety is connected to the N-terminus of one of the first and second subunits of the Fc domain, and the cytokine moiety is connected to the masking moiety. In some embodiments, the masking moiety is connected to the N-terminus of one of the first and second subunits of the Fc domain, and wherein the anchoring moiety is connected to the N-terminus of the other one of the first and second subunits of the Fc domain. In some embodiments, the cytokine moiety is connected to the masking moiety. In some embodiments, the cytokine moiety is connected to the anchoring moiety. In some embodiments, the connection between two or more of the cytokine moiety, the masking moiety, the anchoring moiety and the conjugating moiety is via a peptidic linker.

In one aspect, provided herein are also protein complexes comprising one or more of the immunoconjugate molecules according to the present disclosure. In some embodiments, provided herein is a complex comprising a homotrimer of the immunoconjugate molecule disclosed herein, and wherein the trimerization is through the TNF-α polypeptide in the cytokine moiety of the immunoconjugate molecule.

In one aspect, provided herein are compositions comprising the immunoconjugate molecules according to the present disclosure. In some embodiments, the composition comprises the immunoconjugate molecule and/or complex of the present disclosure, and a pharmaceutical acceptable carrier.

In one aspect, provided herein are polynucleotides encoding the immunoconjugate molecule according to the present disclosure. In some embodiments, the polynucleotide is operably linked to a promoter. In one aspect, provided herein are vectors encoding the immunoconjugate molecule according to the present disclosure. In some embodiments, the vector comprises the polynucleotide of the present disclosure.

In one aspect, provided herein are cells comprising the polynucleotide according to the present disclosure. In some embodiments, the cell comprises the vector according to the present disclosure. In some embodiments, provided herein are an isolated cell producing the immunoconjugate molecule or the complex according to the present disclosure.

In one aspect, provided herein is a kit comprising the immunoconjugate molecule or the complex according to the present disclosure.

In one aspect, provided herein is a method of making an immunoconjugate molecule according to the present disclosure. In some embodiments, the method comprises culturing the cell according to the present disclosure to express the immunoconjugate molecule, wherein the immunoconjugate molecule is in a monomeric, dimeric, or trimeric form. In some embodiments, the method comprises expressing the polynucleotide according to the present disclosure, wherein the immunoconjugate molecule is in a monomeric, dimeric, or trimeric form.

In one aspect, provided herein is a method for activating a TNF-α mediated effect at a target site, the method comprising delivering to the target site an immunoconjugate molecule comprising the TNF-α polypeptide and a masking moiety; wherein the masking moiety comprises a two-in-one antibody or antigen binding fragment thereof that binds to TNF-α through intramolecular interaction and inhibits the TNF-α mediated effect; wherein the two-in-one antibody or antigen binding fragment is capable of binding to a first target antigen in the target site; wherein when the immunoconjugate molecule is at the target site, the two-in-one antibody binds to the first target antigen and disassociate from TNF-α; and wherein the TNF-α mediated effect is activated at the target site.

In some embodiments, the immunoconjugate molecule further comprises an anchoring moiety; wherein the anchoring moiety comprises an antibody or antigen binding fragment thereof capable of binding to a second target antigen in the target site. In some embodiments, when the immunoconjugate molecule is at the target site, the antibody or antigen binding fragment of the anchoring moiety binds to the second target antigen; and wherein the immunoconjugate molecule is immobilized at the target site.

In some embodiments, delivering the immunoconjugate molecule to the target site comprises administering the immunoconjugate molecule to a subject. In some embodiments, the TNF-α mediated effect is at least about 10%, 20%, 30%, 40%, 45%, 50%, 55%, 60%, 65%, 70%, 75%, 80%, 85%, 90%, 95%, or 98% lower at a non-target site as compared to the TNF-α mediated effect at the target site after administering the immunoconjugate molecule to the subject. In some embodiments, the TNF-α mediated effect is cell apoptosis.

In one aspect, provided herein are a method for enriching a TNF-α polypeptide at a target site, the method comprising delivering to the target site an immunoconjugate molecule comprising a TNF-α polypeptide and an anchoring moiety; wherein the anchoring moiety comprises an antibody or antigen binding fragment thereof capable of binding to a second target antigen in the target site; wherein when the immunoconjugate molecule is at the target site, the anchoring moiety binds to the second target antigen; and wherein the TNF-α polypeptide is distributed at a higher concentration at the target site compared to a non-target site.

In some embodiments, delivering the immunoconjugate molecule to the target site comprises administering the immunoconjugate molecule to a subject. In some embodiments, the concentration of the TNF-α polypeptide is at least about 10%, 20%, 30%, 40%, 45%, 50%, 55%, 60%, 65%, 70%, 75%, 80%, 85%, 90%, 95%, or 98% lower at a non-target site as compared to the concentration of the TNF-α polypeptide at the target site after administering the immunoconjugate molecule to the subject.

In some embodiments of the present methods, a toxicity or side-effect associated with TNF-α in the subject is reduced. In some embodiments, cytokine toxicity or side-effect is reduced at least about 10%, 20%, 30%, 40%, 45%, 50%, 55%, 60%, 65%, 70%, 75%, 80%, 85%, 90%, 95%, or 98% as compared to administering to the subject an equivalent amount of TNF-α in an unconjugated form.

In some embodiments, the reduction in toxicity or side-effect is measured as the elongation of life span of the administered subject. In some embodiments, the reduction in toxicity or side-effect is measured as reduction in loss of body weight of the administered subject. In some embodiments, the reduction in toxicity or side-effect is measured as change in the level of an immune response in the administered subject. In some embodiments, the reduction in toxicity or side-effect is measured as a change in an inflammatory response in the administered subject.

In some embodiments, the immunoconjugate molecule further comprises a masking moiety; wherein the masking moiety comprises a two-in-one antibody or antigen binding fragment thereof that binds to the TNF-α polypeptide through intramolecular interaction and inhibits an TNF-α mediated effect; wherein the two-in-one antibody or antigen binding fragment thereof is capable of binding to a first target antigen in the target site; wherein when the immunoconjugate molecule is at the target site, the two-in-one antibody binds to the first target antigen and disassociate from the TNF-α polypeptide; and wherein the TNF-α mediated effect is activated at the target site.

In some embodiments, the first antigen and second antigen are the same antigen or different antigens. In some embodiments, the target site is a tumor microenvironment. In some embodiments, the target site is a cancerous cell. In some embodiments, the first and/or second antigen is expressed on the surface of cancer cells. In some embodiments, the first and/or second antigen is expressed by cells in the tumor microenvironment.

In some embodiments, the cell expressing the first and/or second antigen also expresses a TNF-α receptor. In some embodiments, the cell expressing the first and/or second antigen does not express a TNF-α receptor. In some embodiments, the first and/or second antigen is fibroblast activation protein (FAP) or Tumor-associated calcium signal transducer 2 (Trop-2). In some embodiments, the immunoconjugate molecule is any of the immunoconjugate molecule according to the present disclosure. In some embodiments, the immunoconjugate molecule is in a monomeric, dimeric, or trimeric form.

In one aspect, provided herein is a method for inhibiting growth or induce apoptosis of a cancer cell comprising contacting the cancer cell with an effective amount of the immunoconjugate molecule comprising an TNF-α polypeptide as described herein.

In one aspect, provided herein is a method of treating cancer in a subject in need thereof, comprising administering to the subject an effective amount of the immunoconjugate molecule comprising an TNF-α polypeptide as described herein. In some embodiments, the method further comprises co-administration of a second therapy. In some embodiments, the second therapy comprises a chemotherapeutic agent. In some embodiments, the second therapy comprises Actinomycin D.

### 4. BRIEF DESCRIPTION OF THE FIGURES

FIG. 1A is a schematic illustration of a soluble cytokine polypeptide in the form of homotrimer.

FIGS. 1B to 1L are schematic illustrations of complexes containing multimerized antibody-cytokine immunoconjugates having different molecular configurations according to the present disclosure. The multimerization between the immunoconjugate molecules can be via covalent (e.g., disulfide bond or peptidic linker) or non-covalent interactions. Although FIGS. 1B to 1L illustrate the configurations in the timer form, immunoconjugates in the monomer, dimer, or higher-order multimer (e.g., greater than 3) forms are also within the scope of the present disclosure.

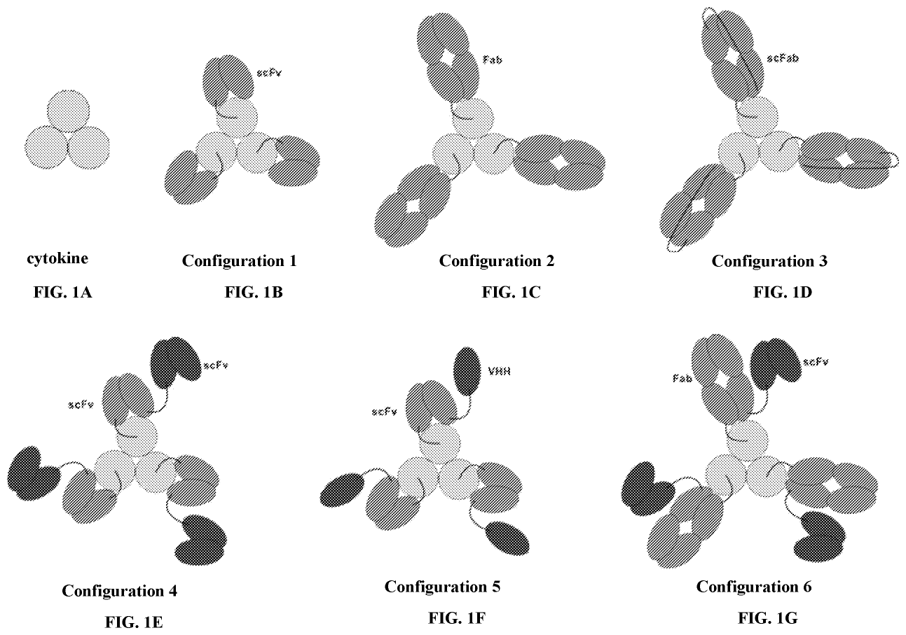

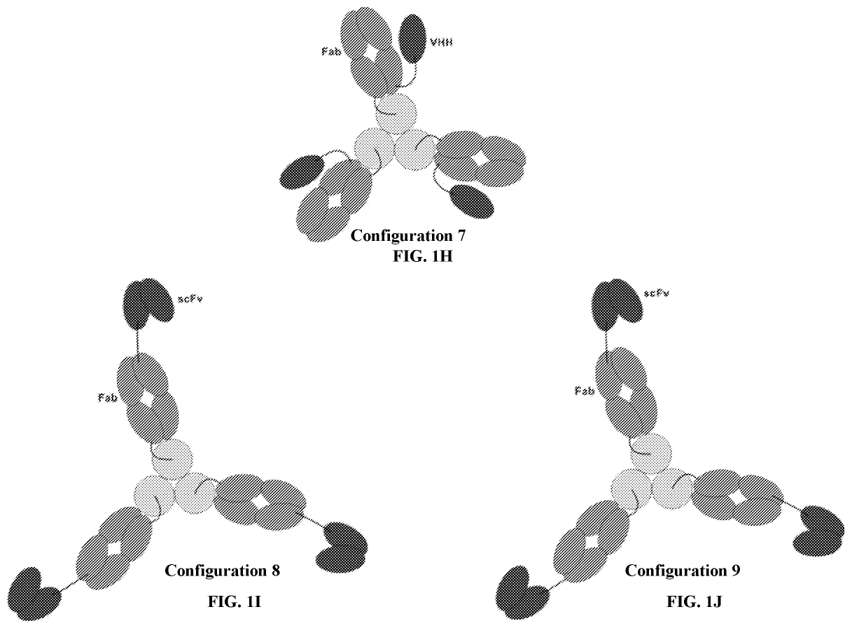

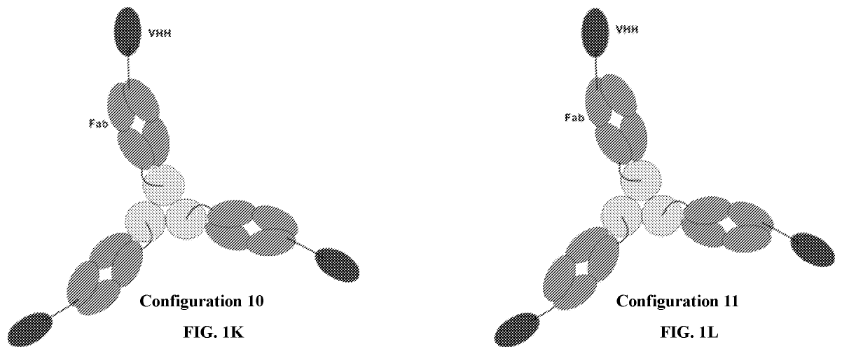

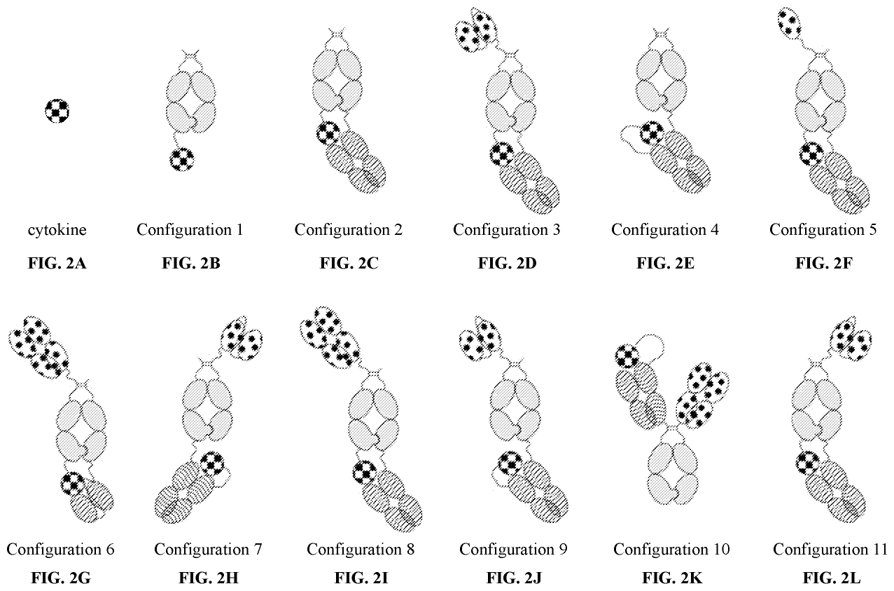

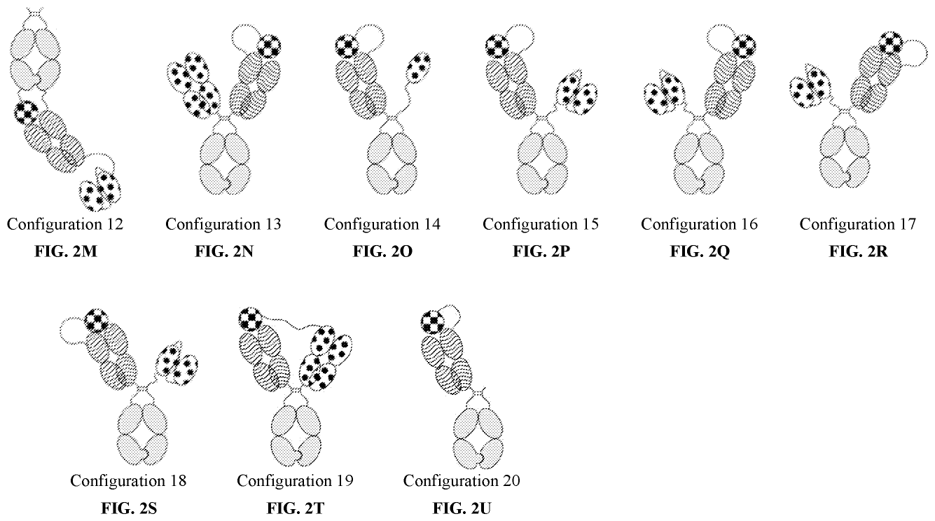

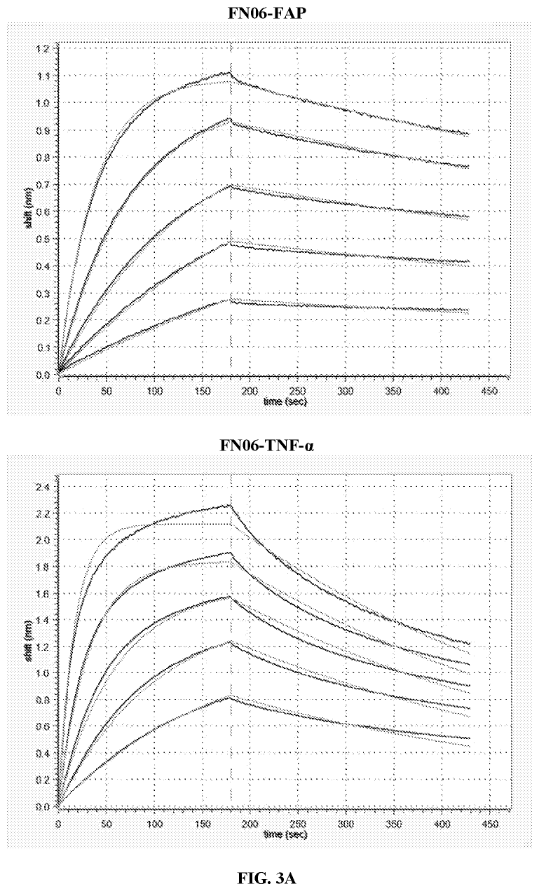

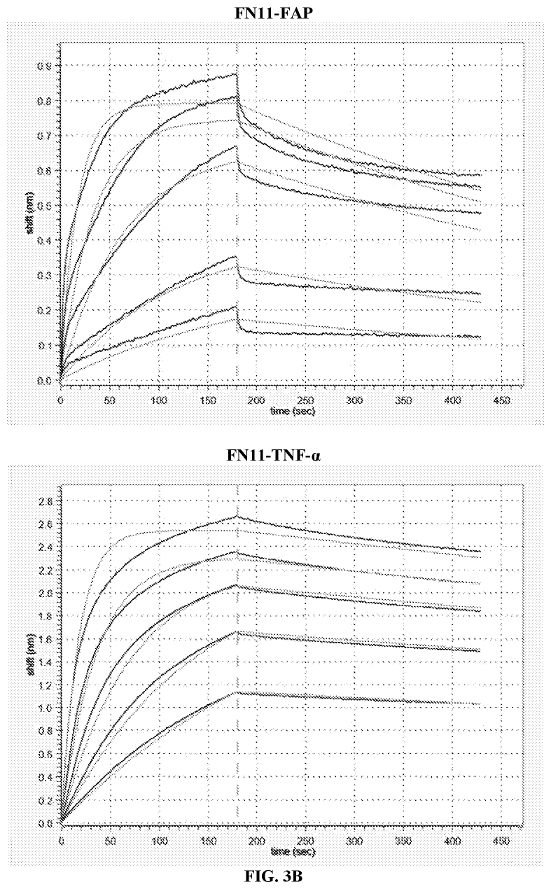

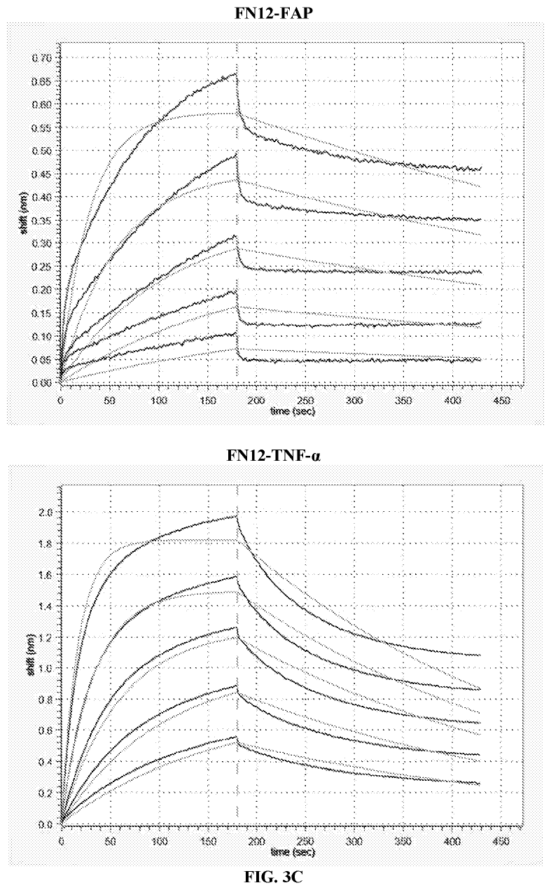

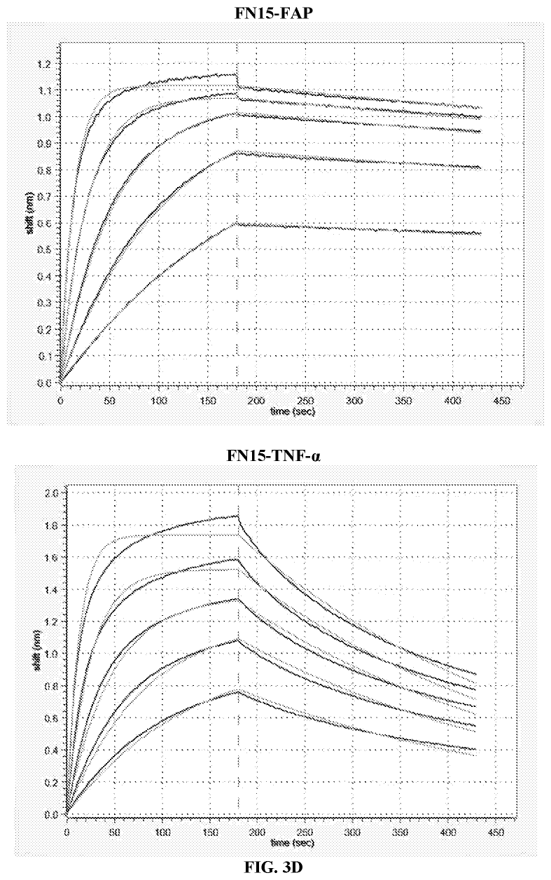

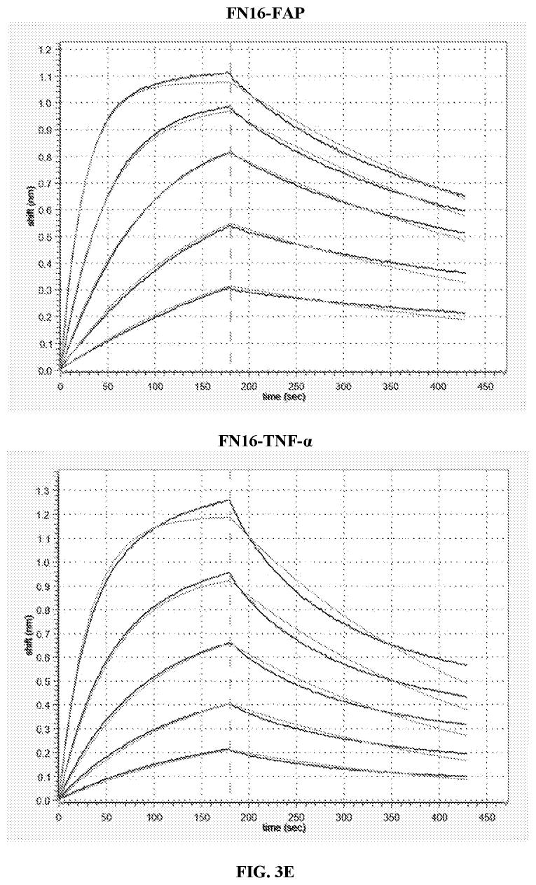

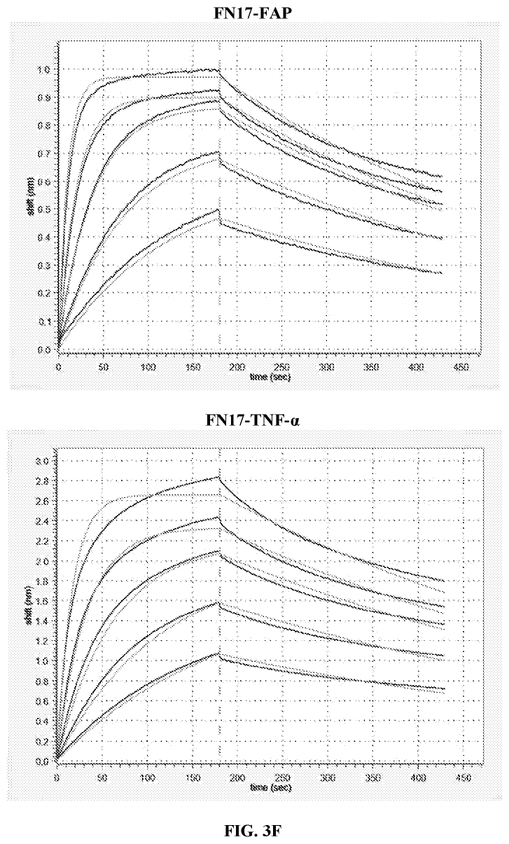

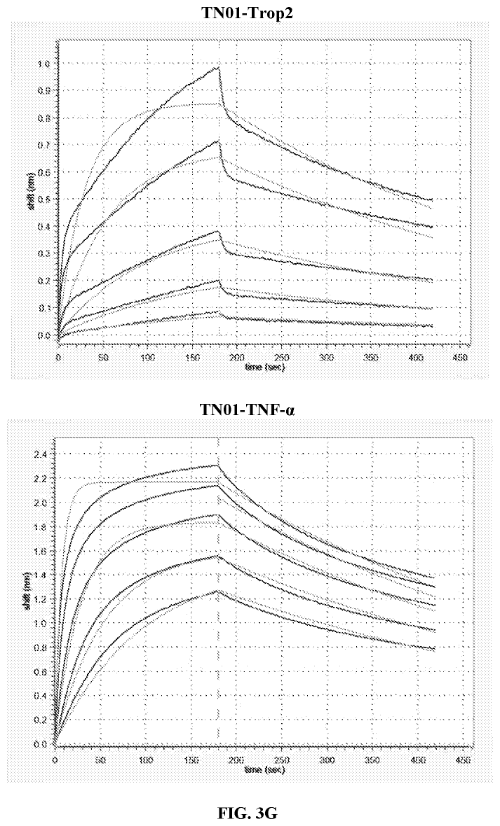

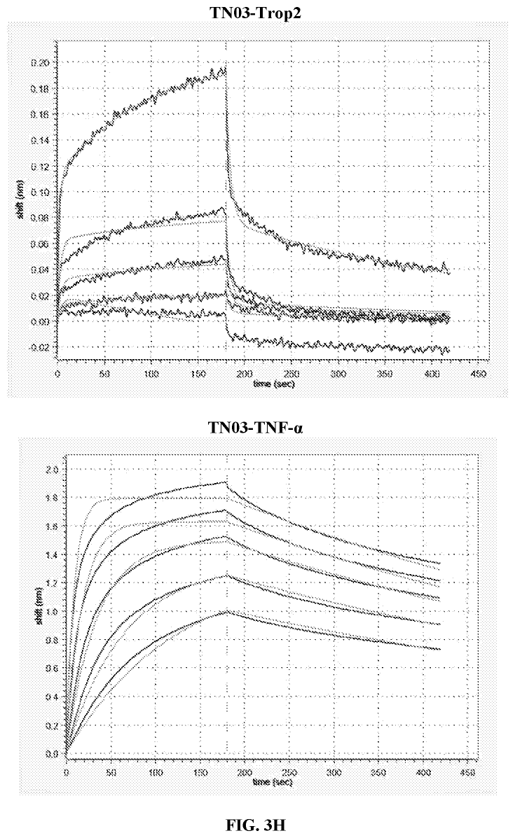

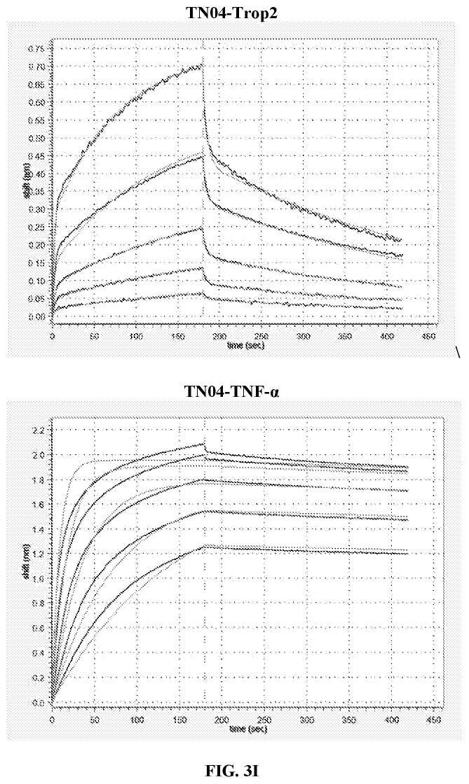

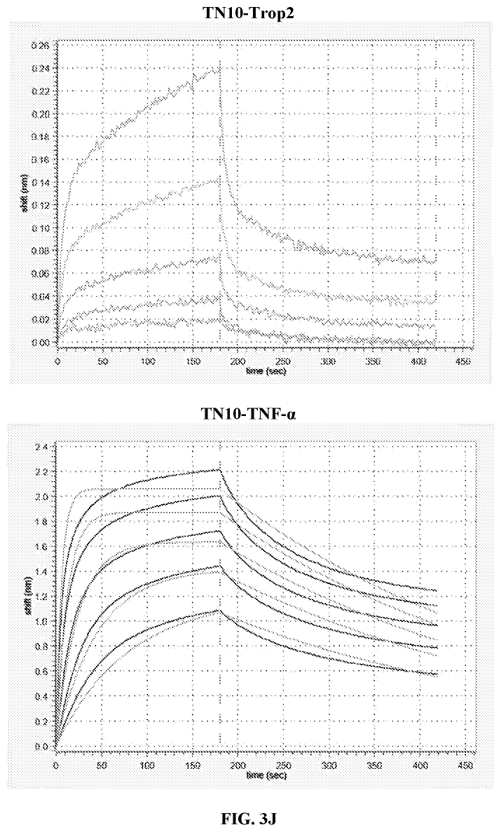

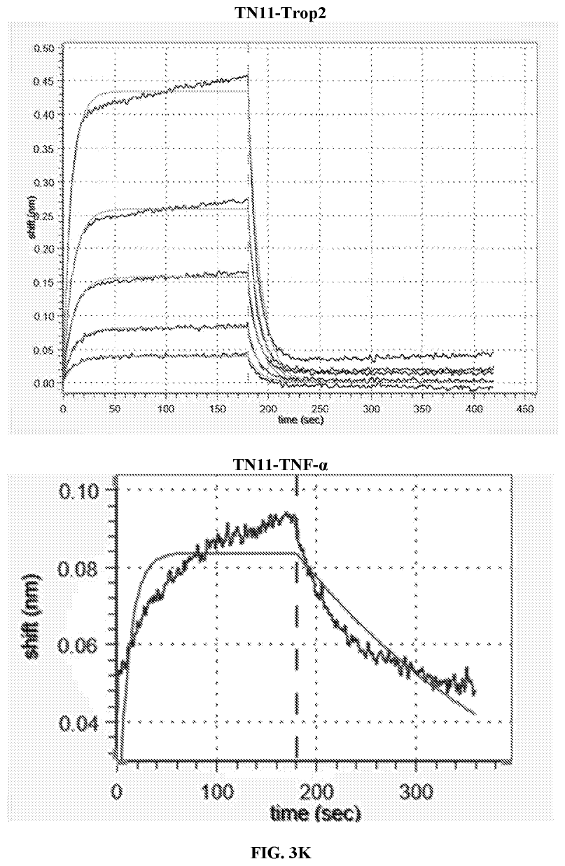

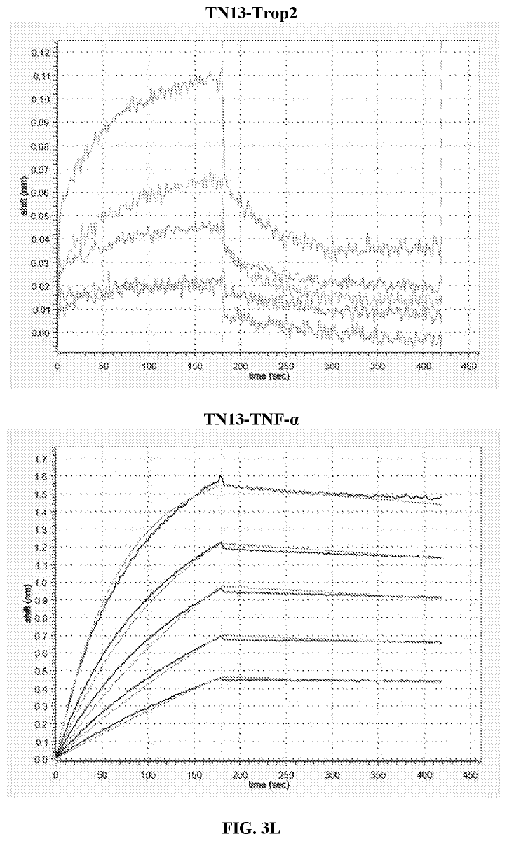

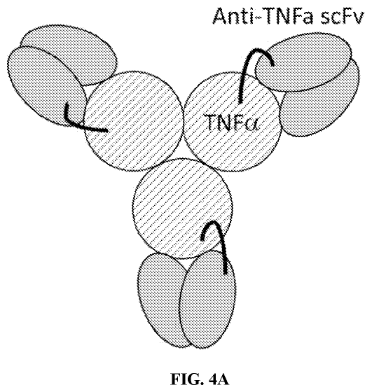

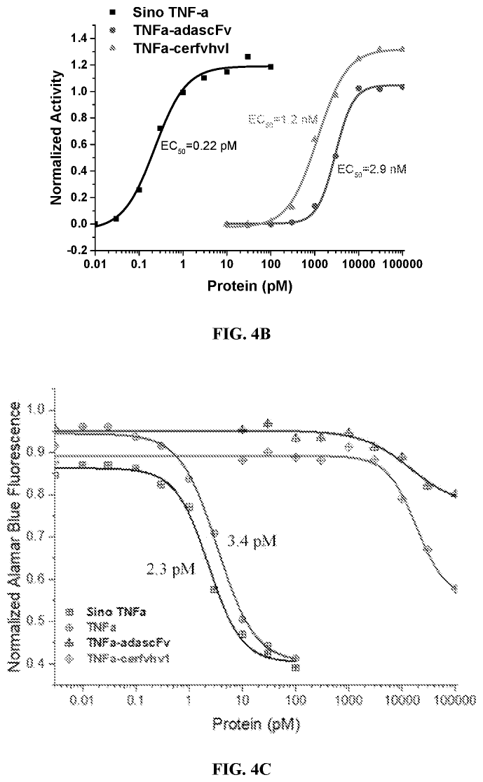

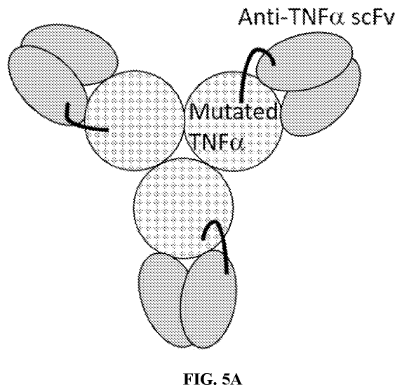

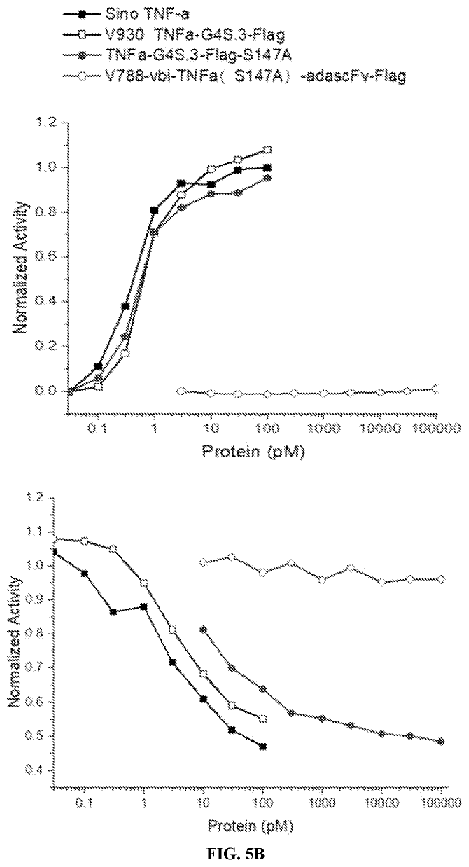

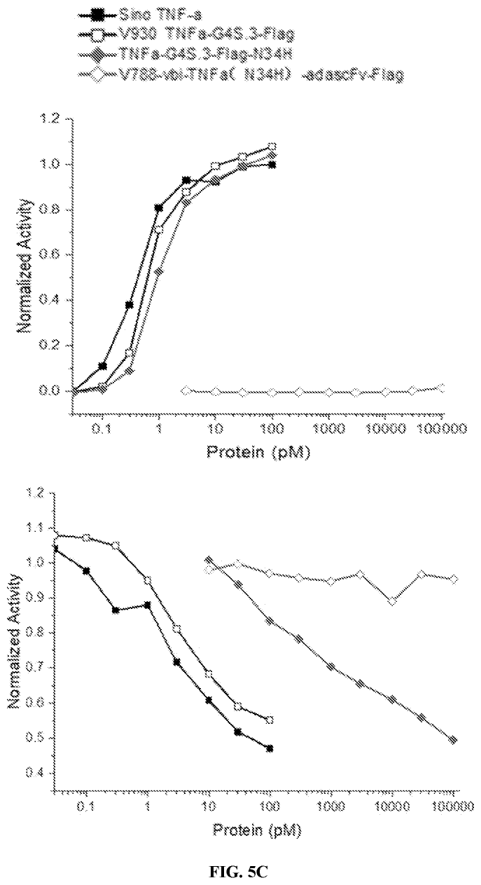

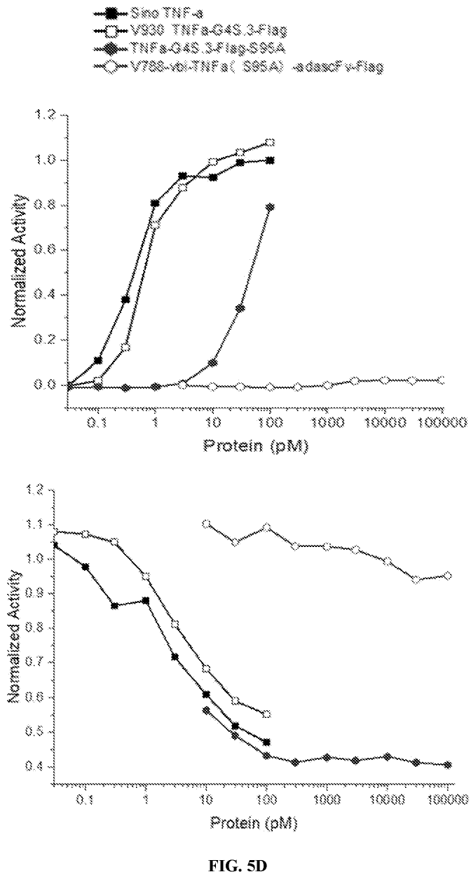

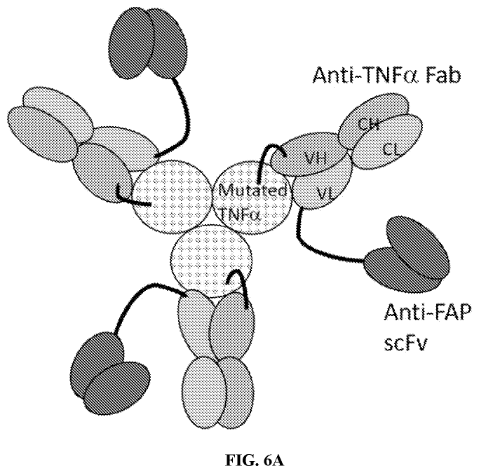

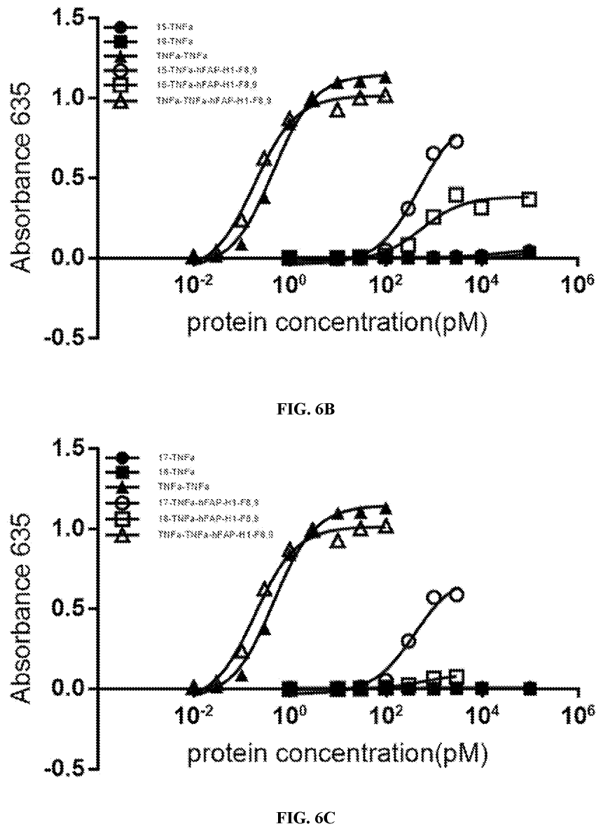

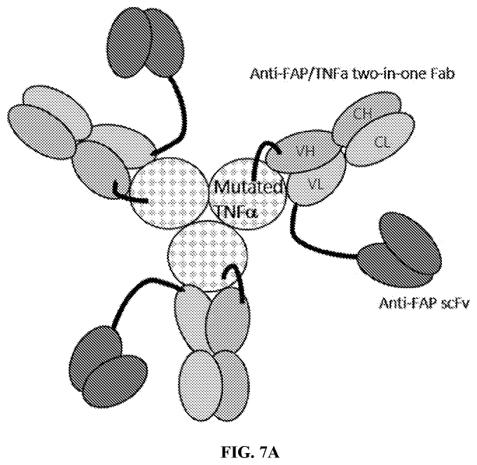

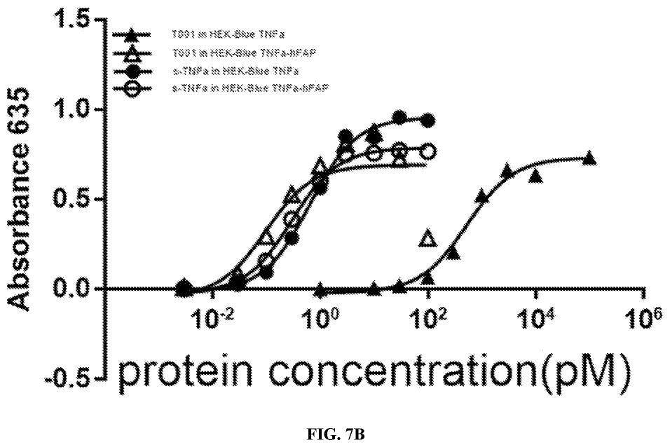

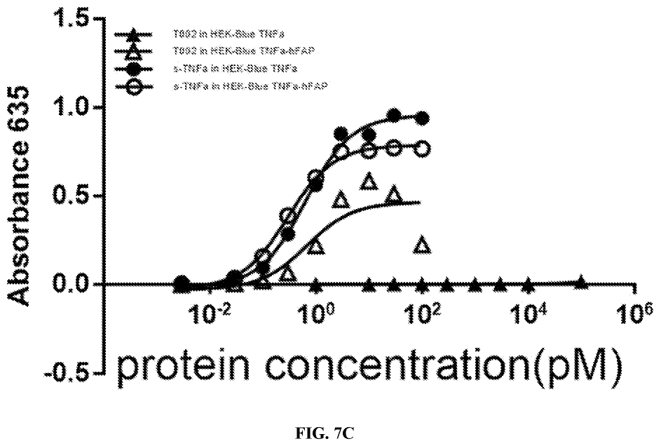

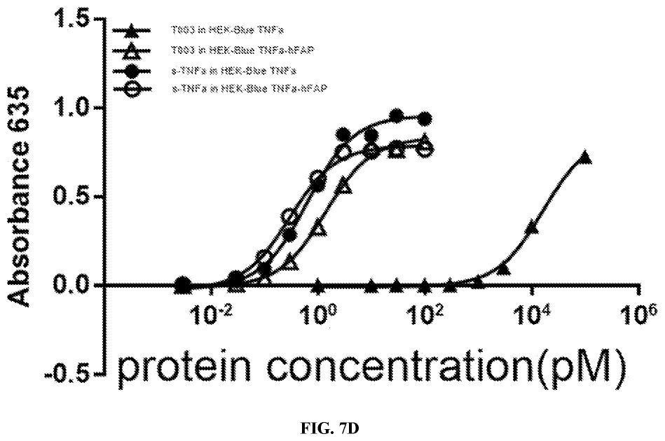

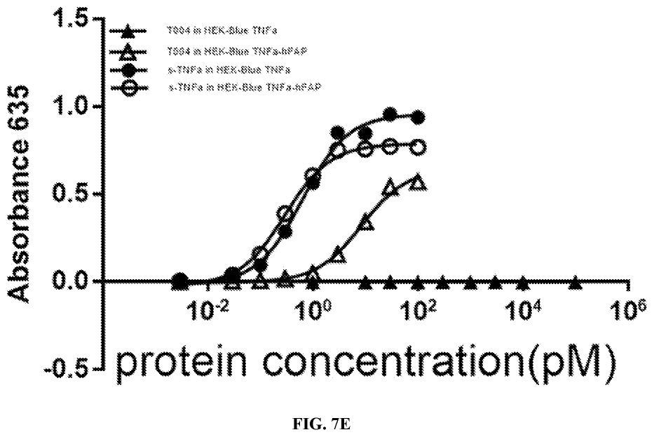

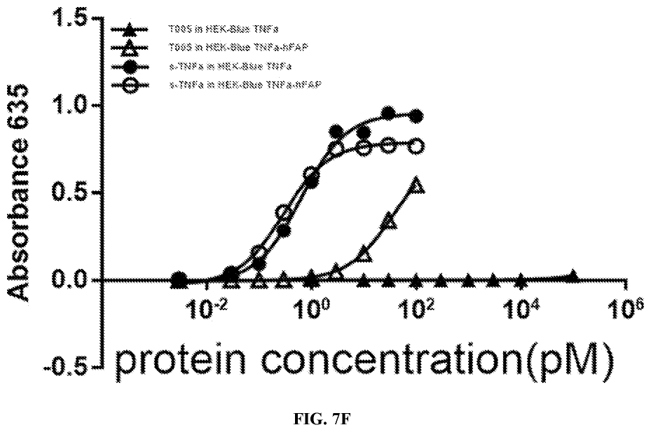

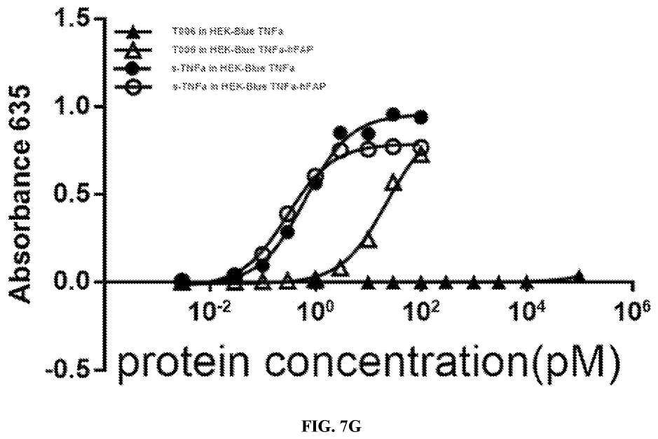

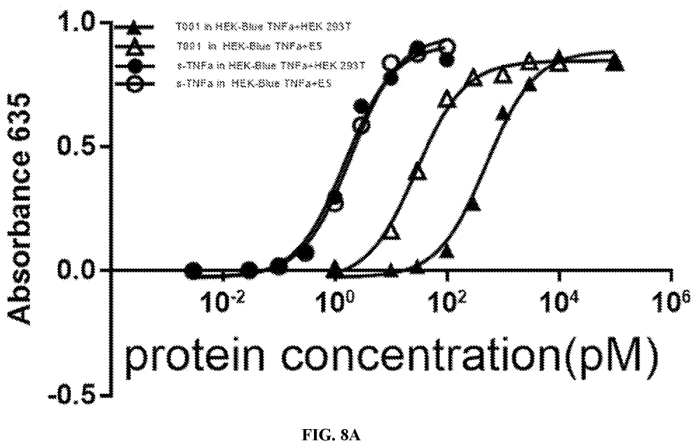

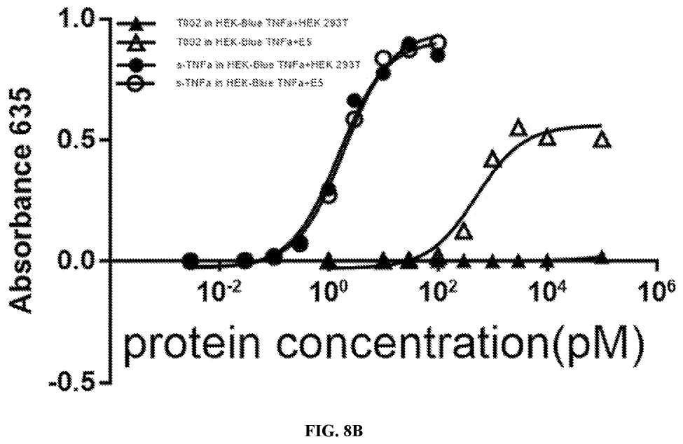

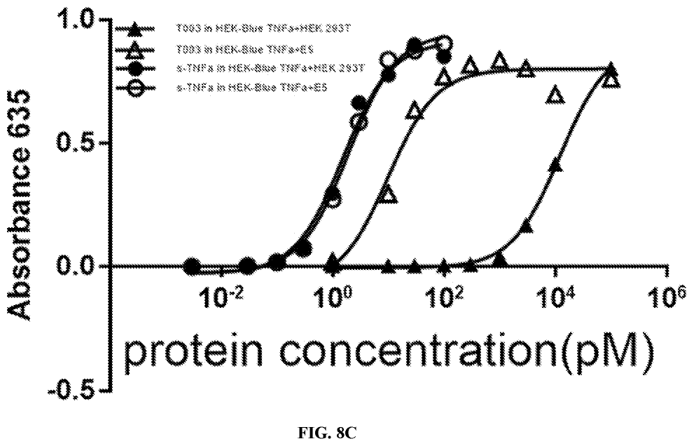

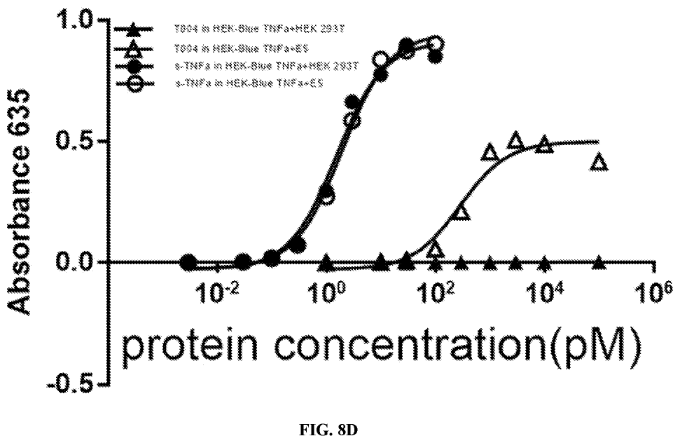

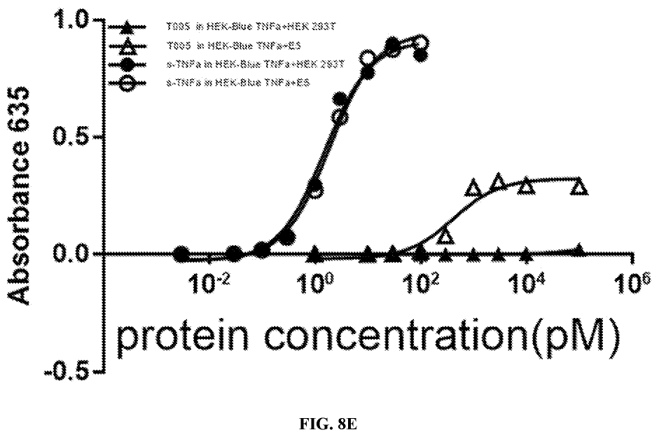

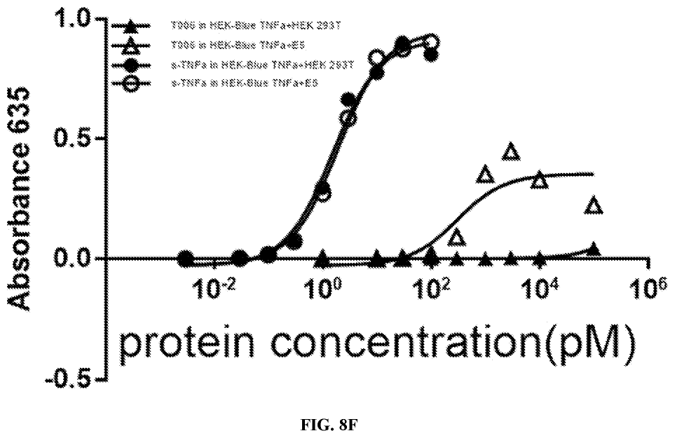

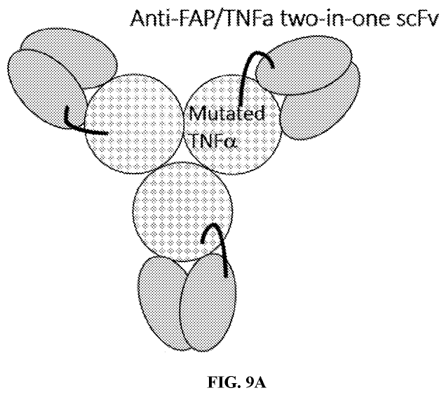

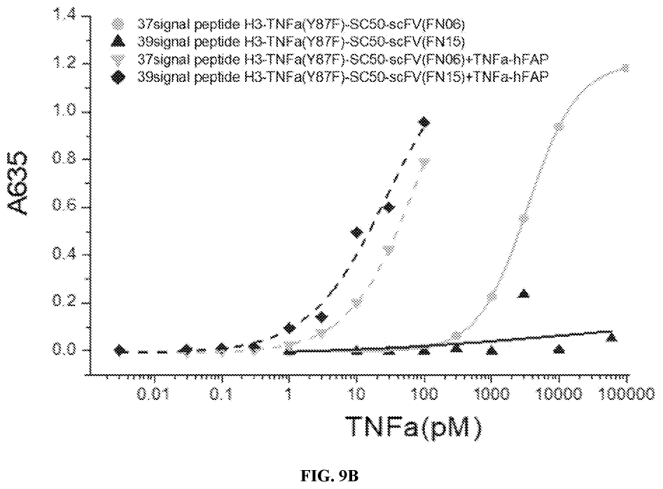

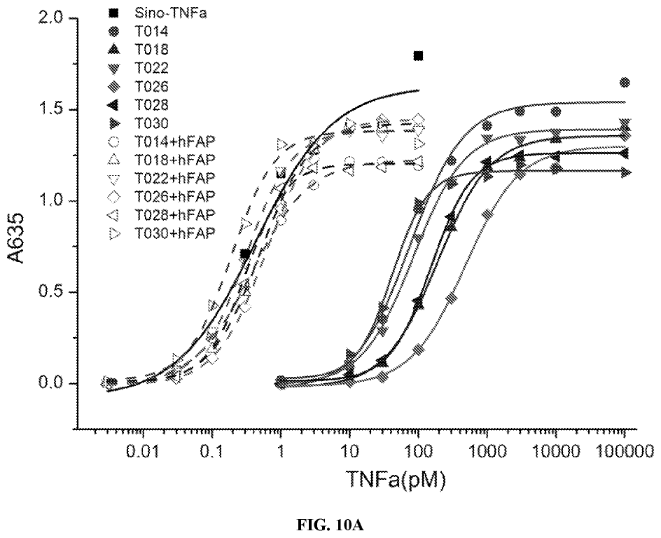

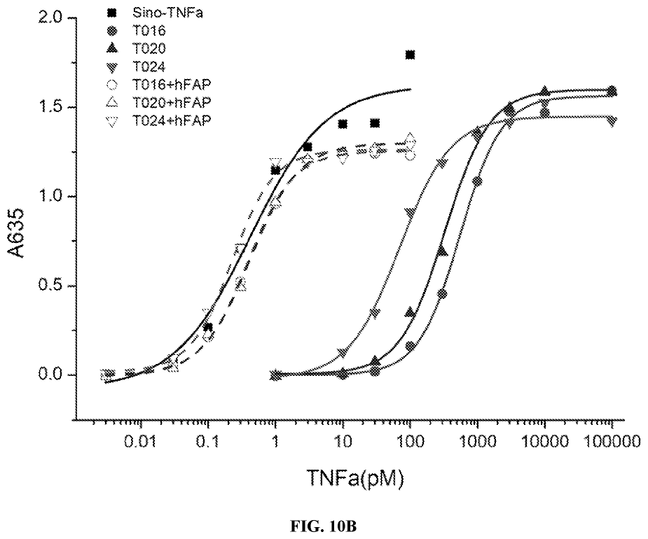

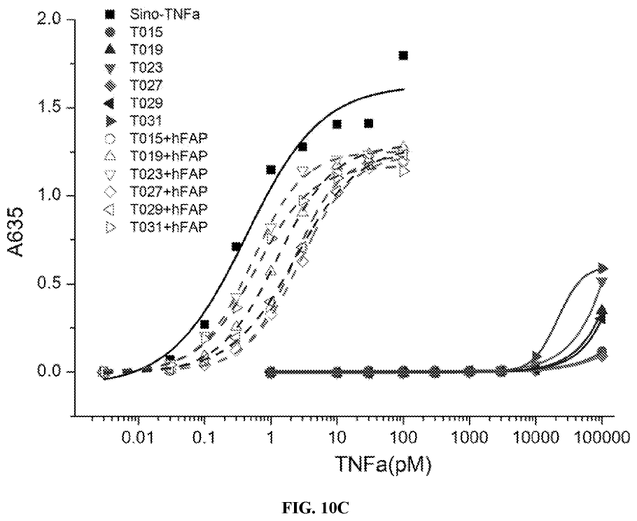

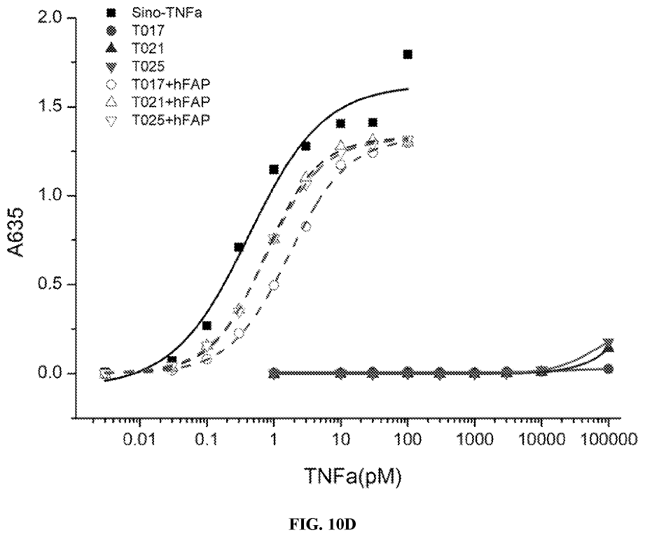

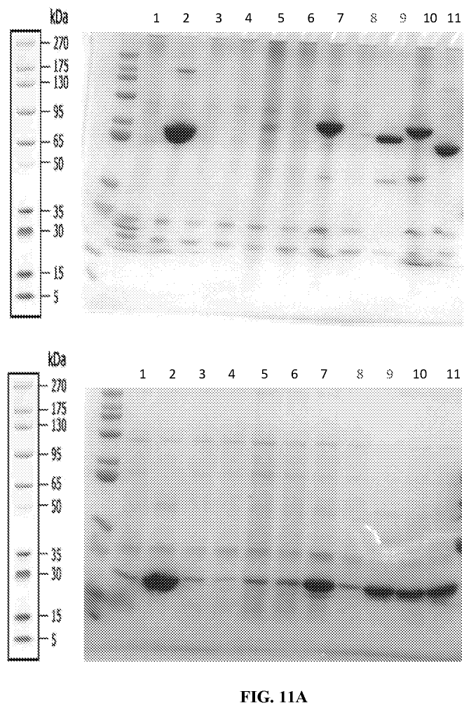

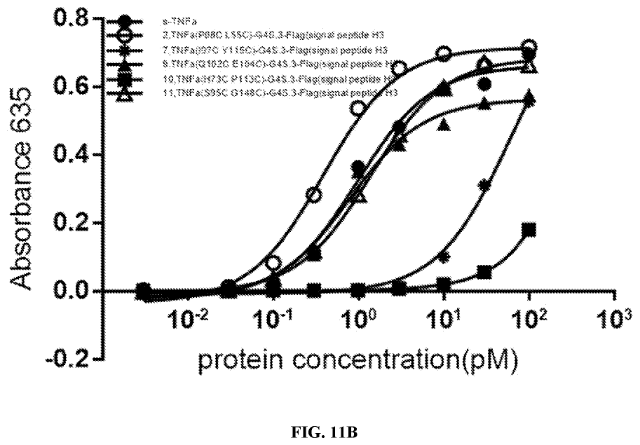

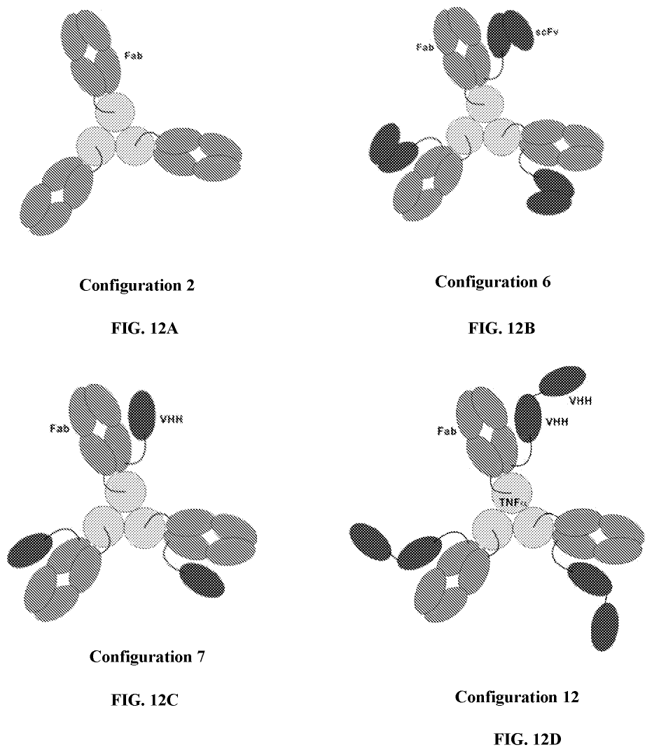

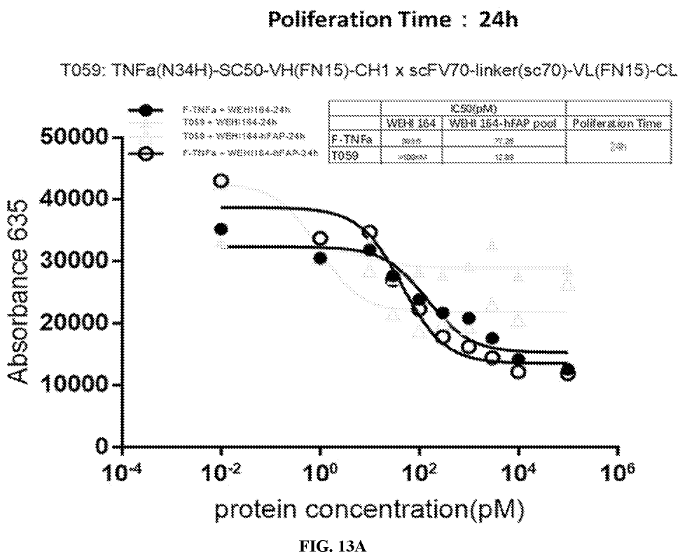

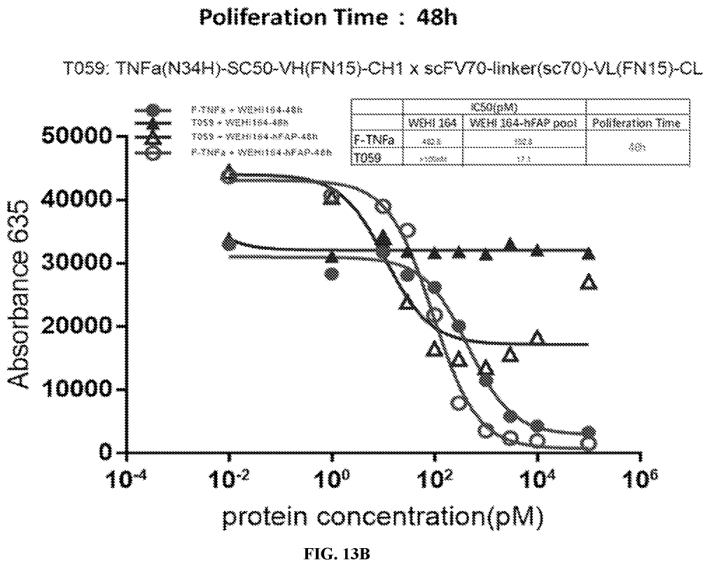

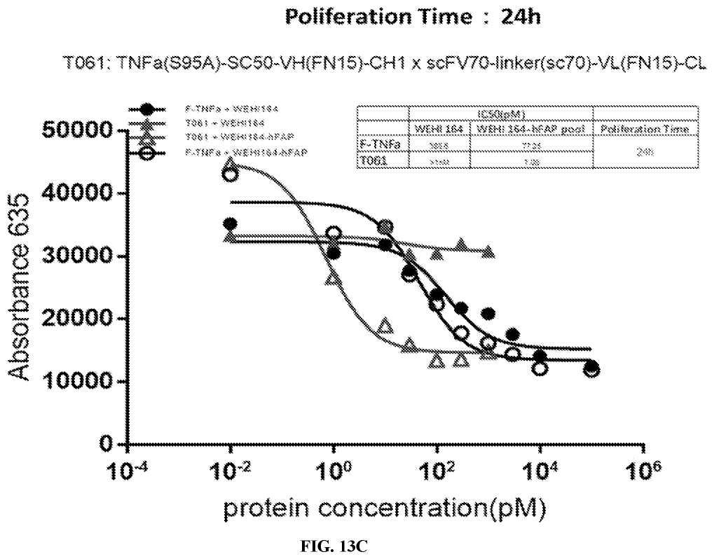

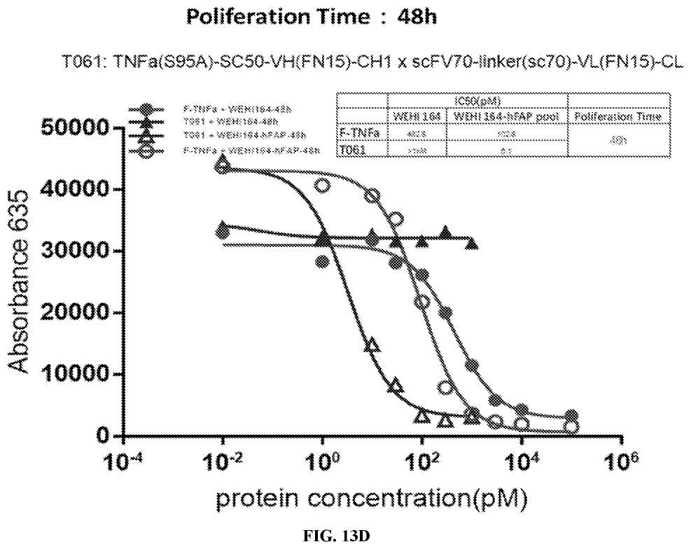

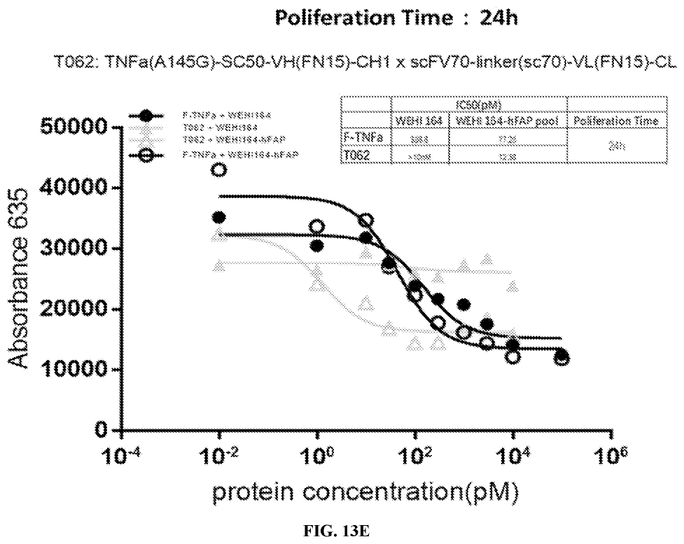

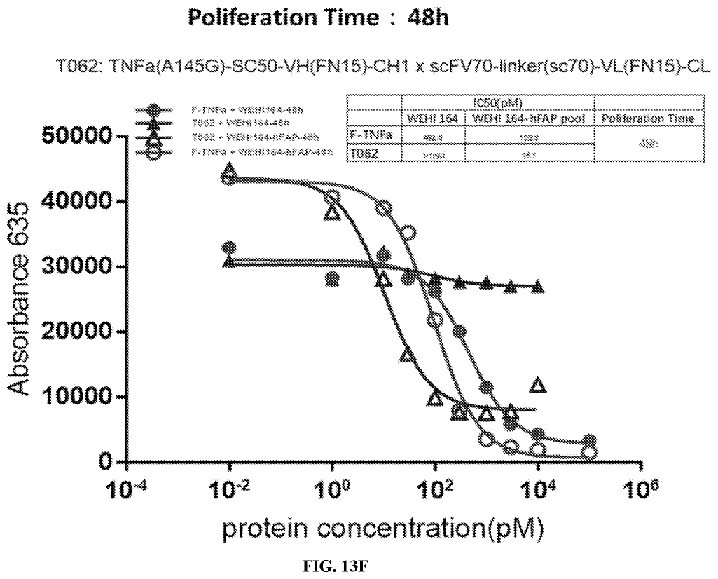

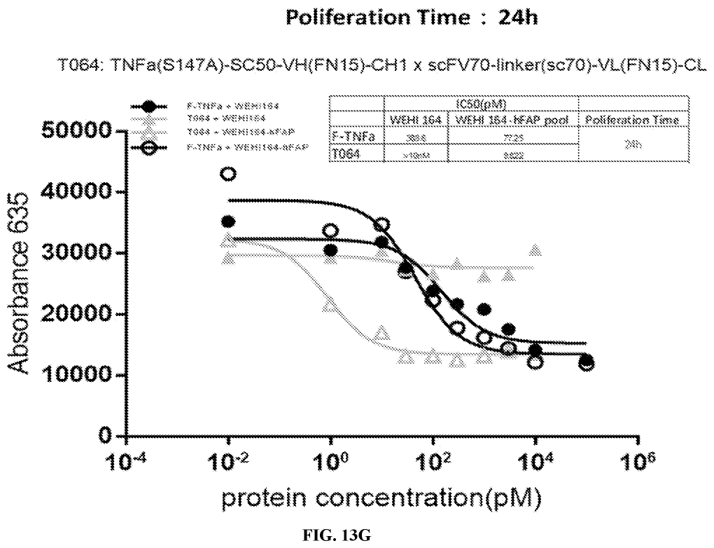

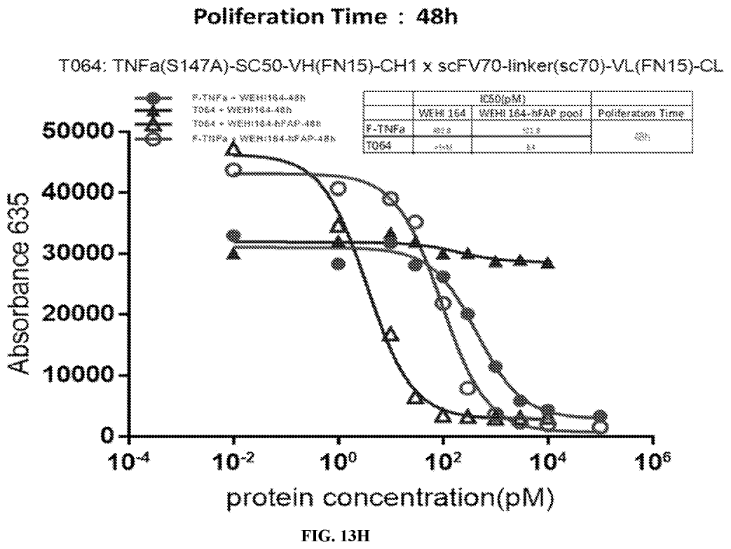

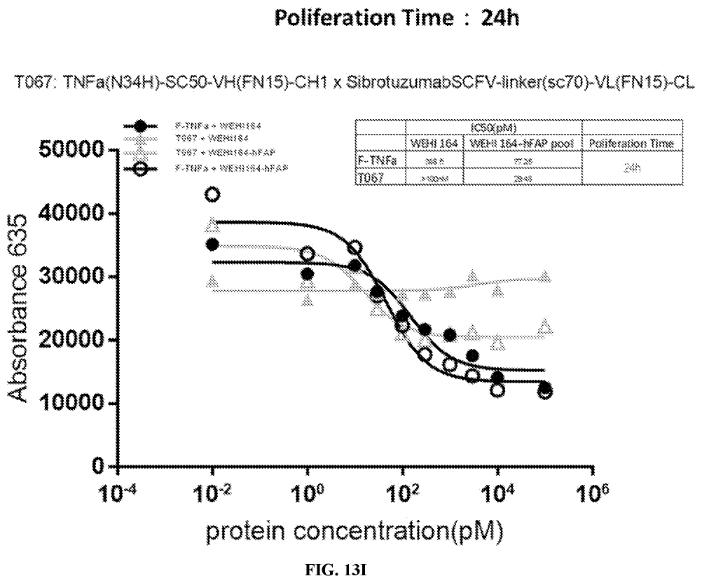

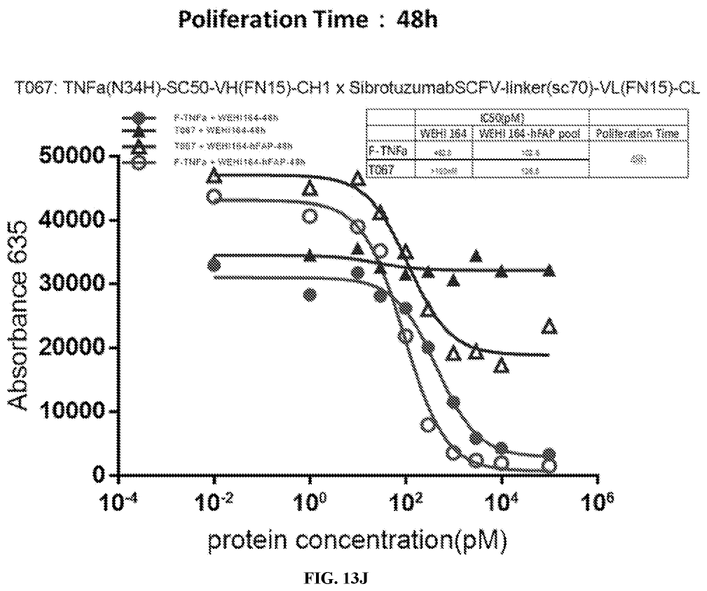

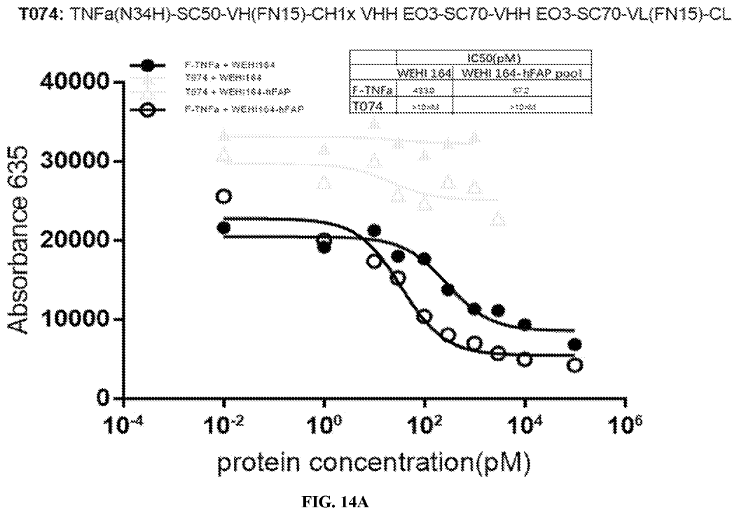

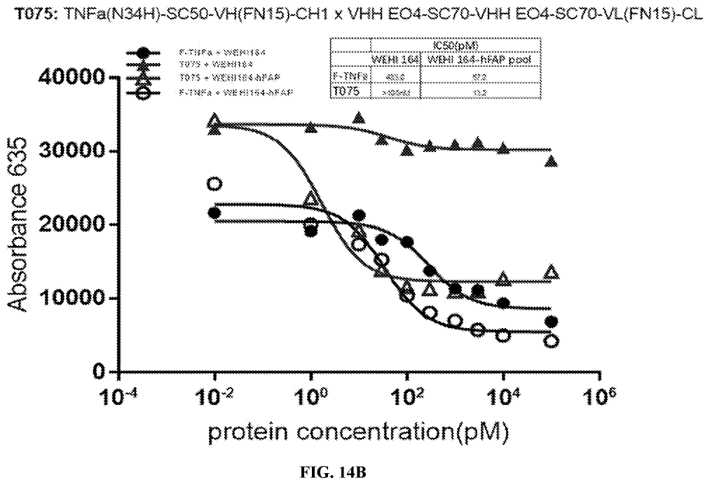

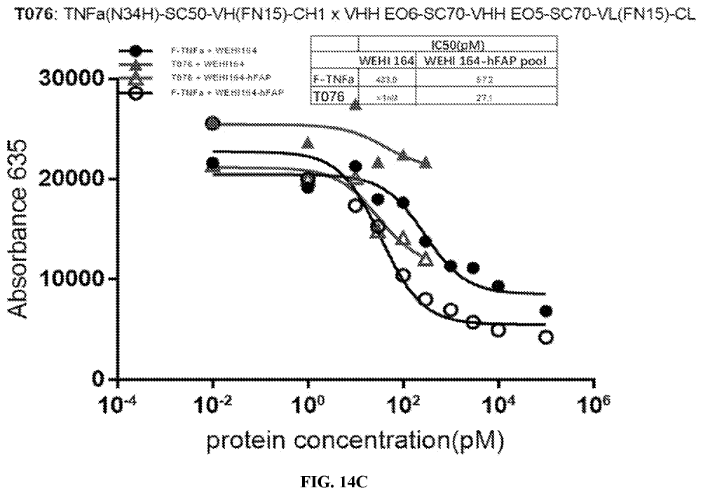

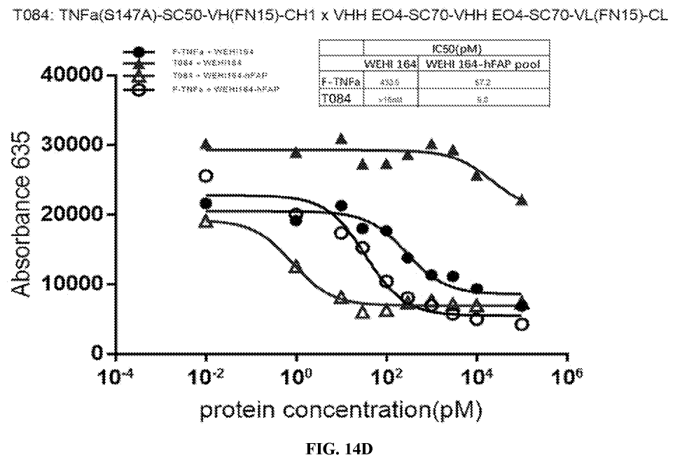

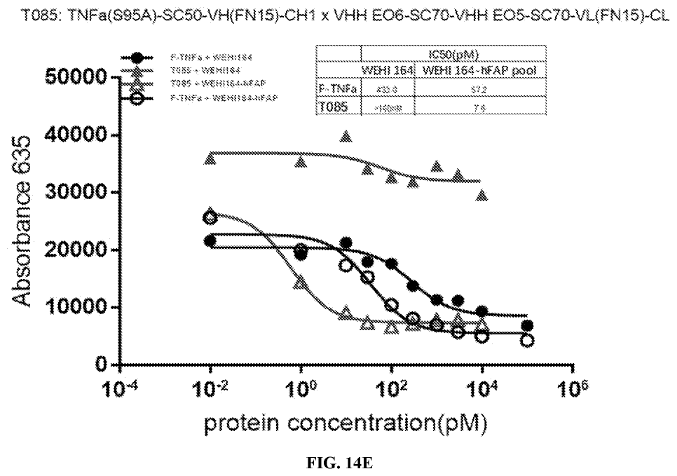

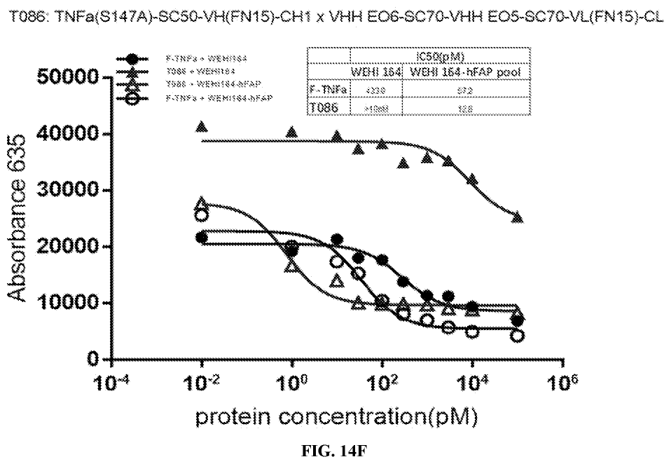

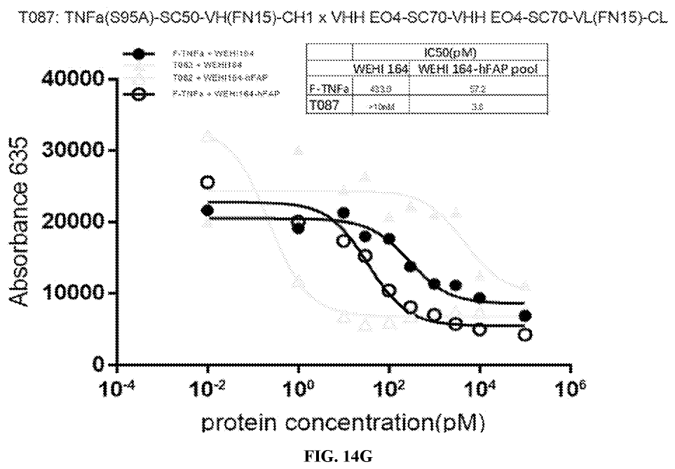

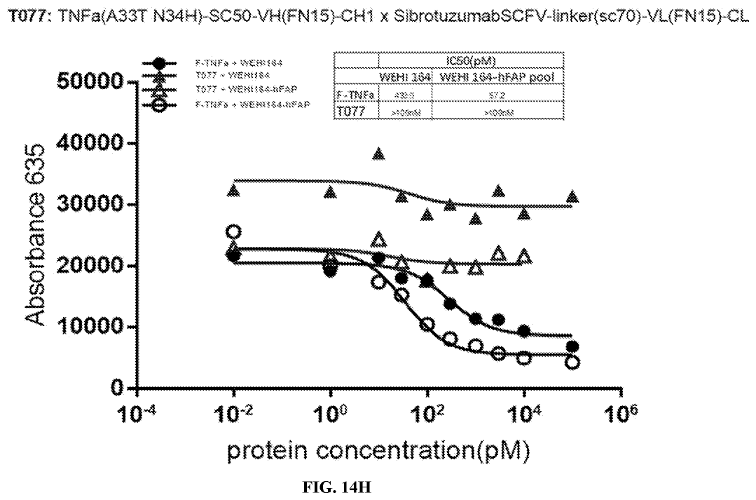

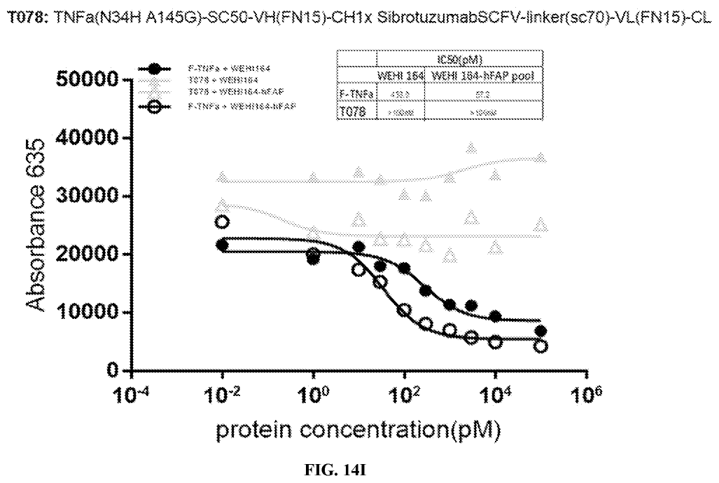

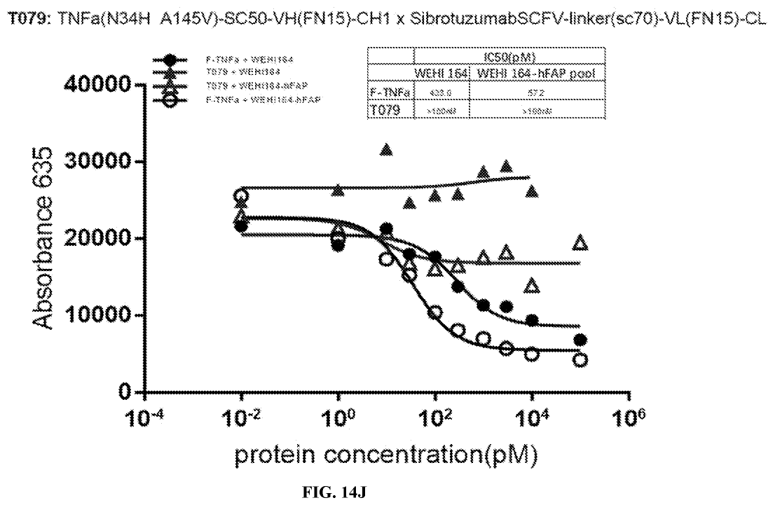

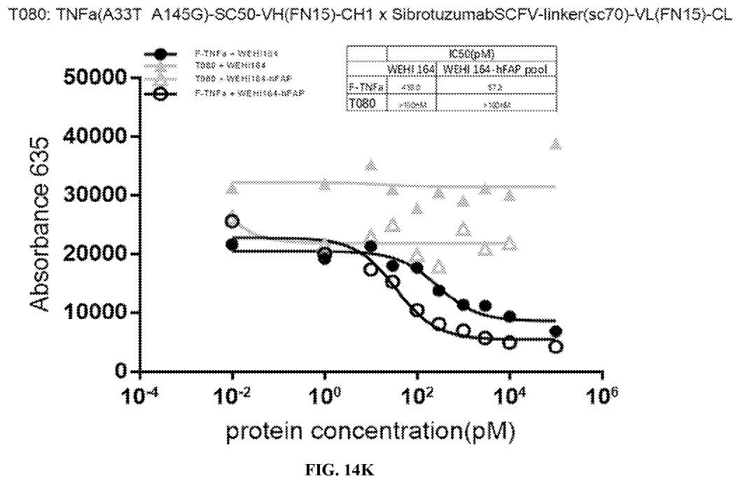

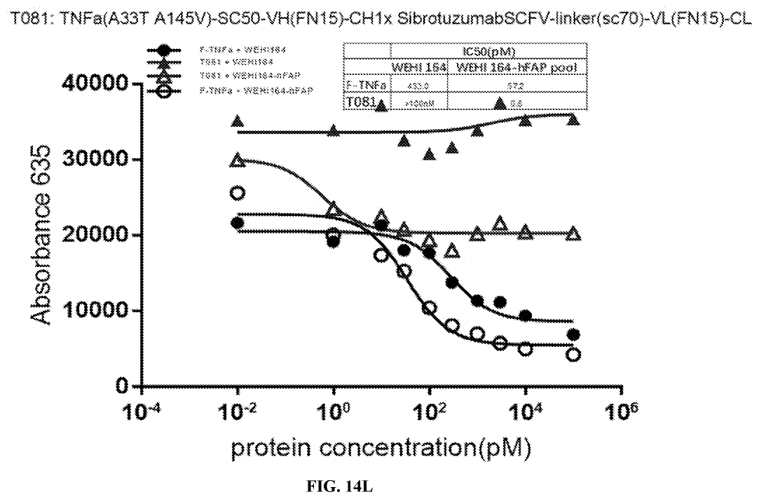

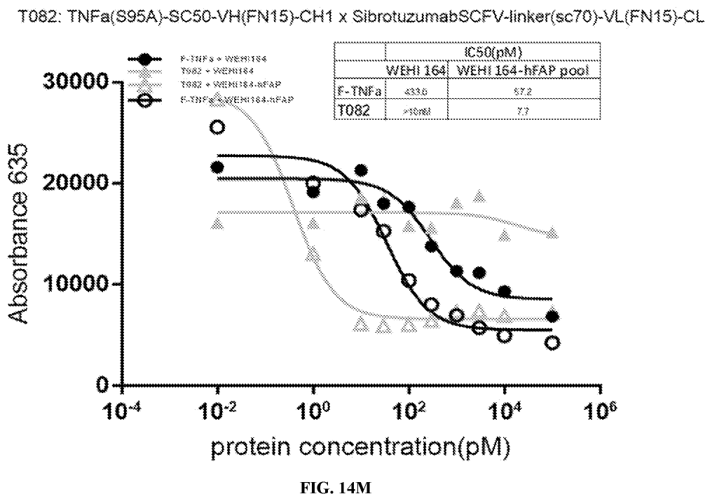

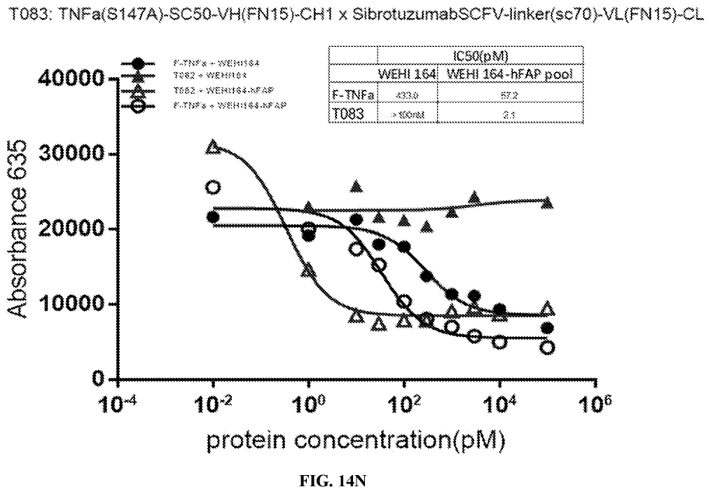

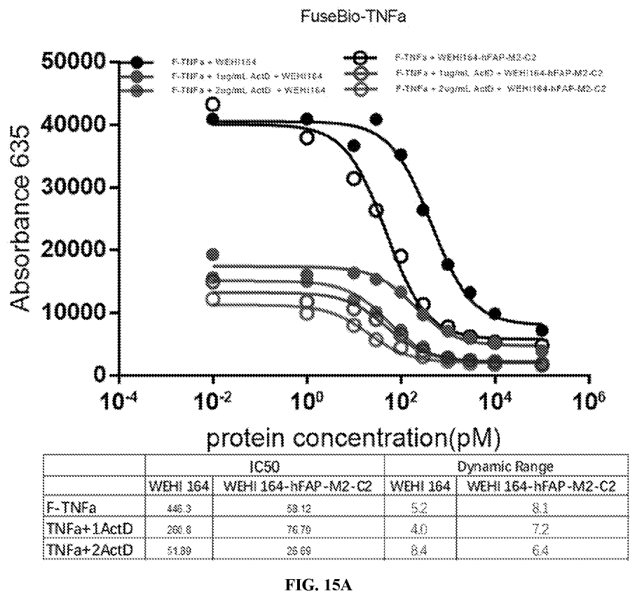

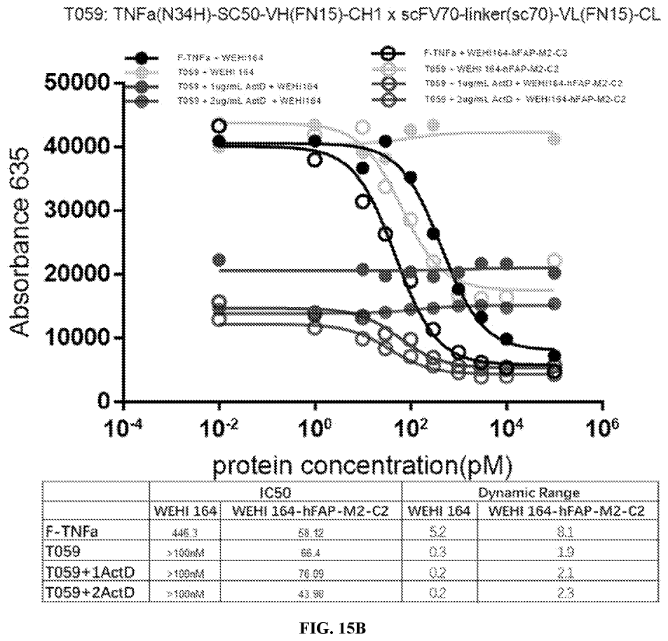

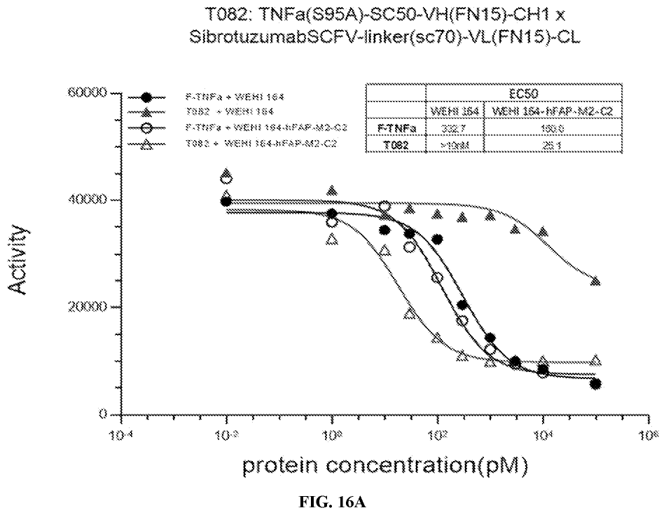

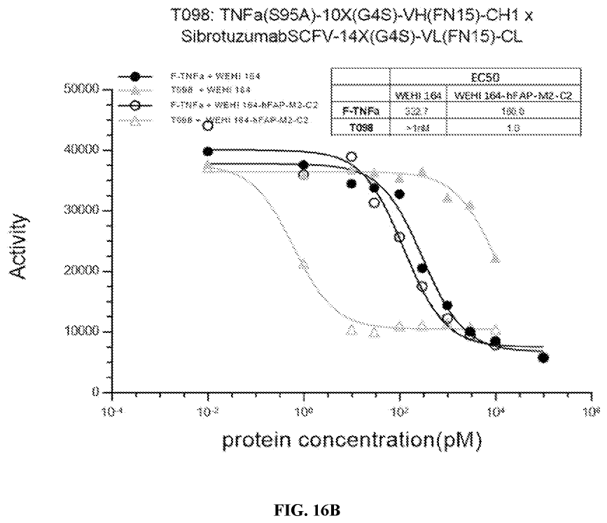

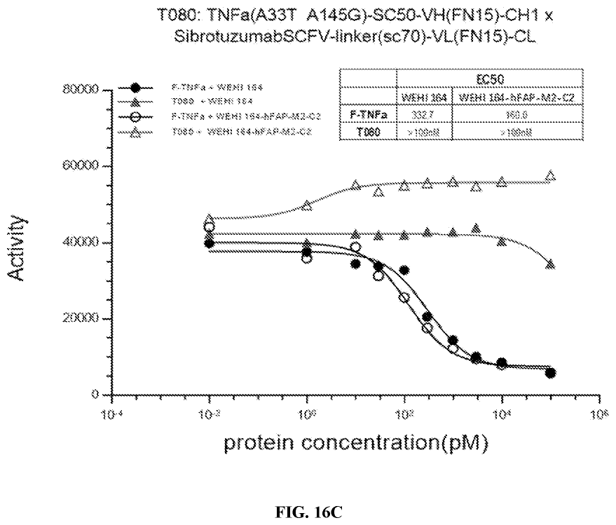

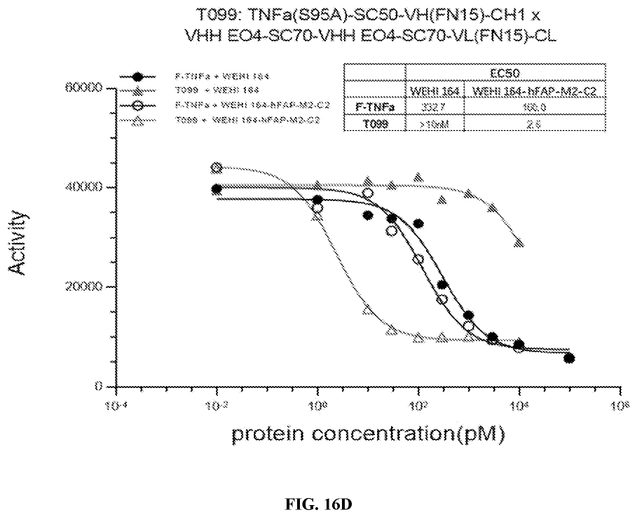

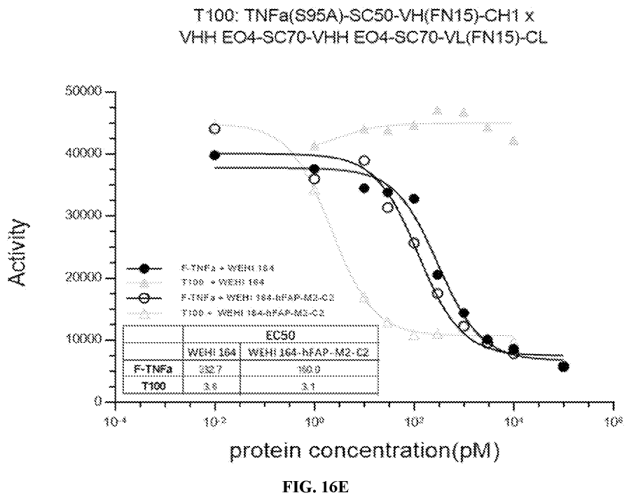

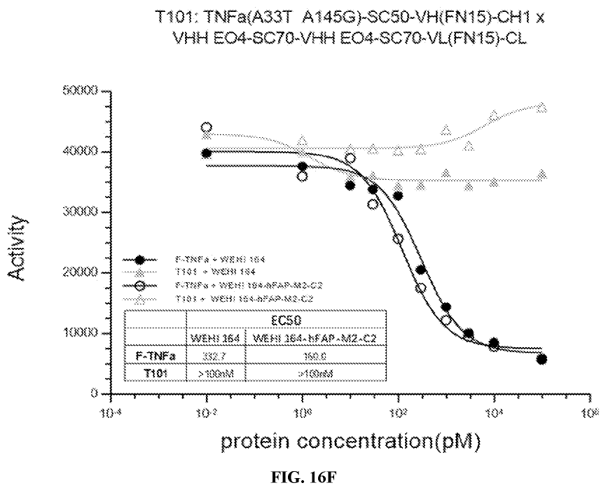

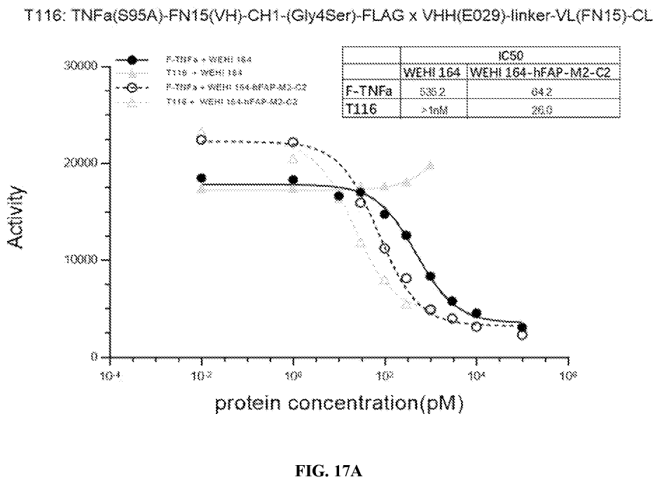

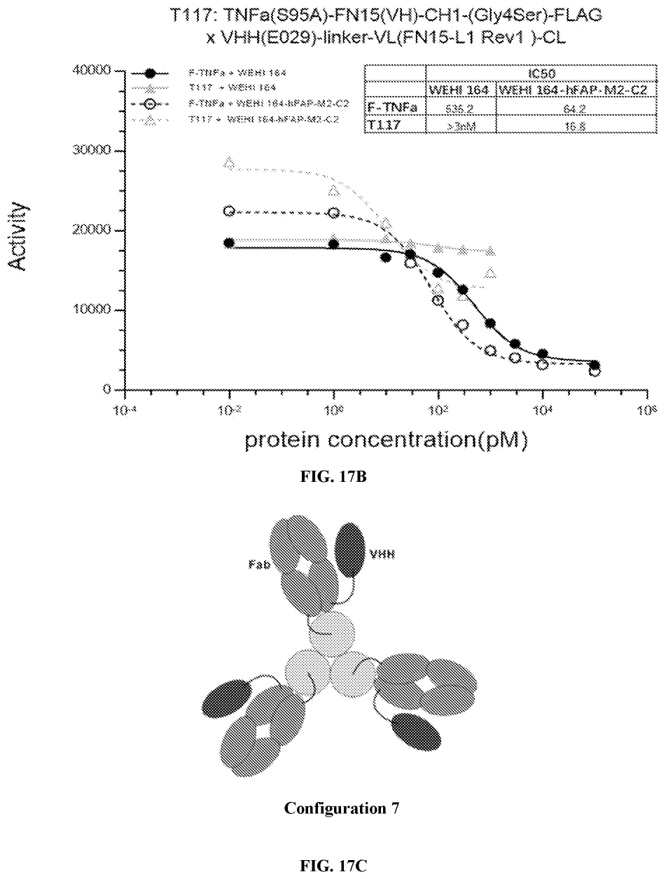

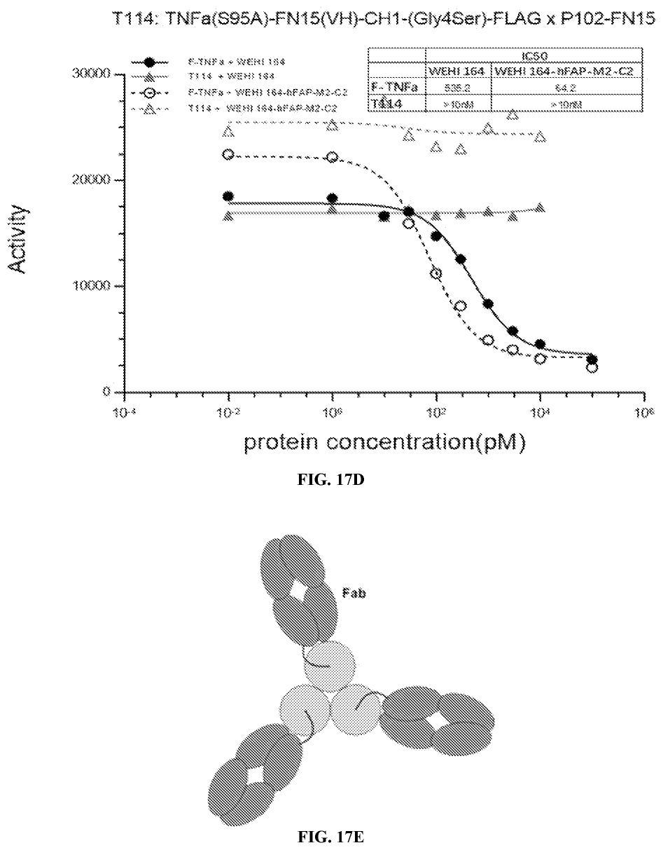

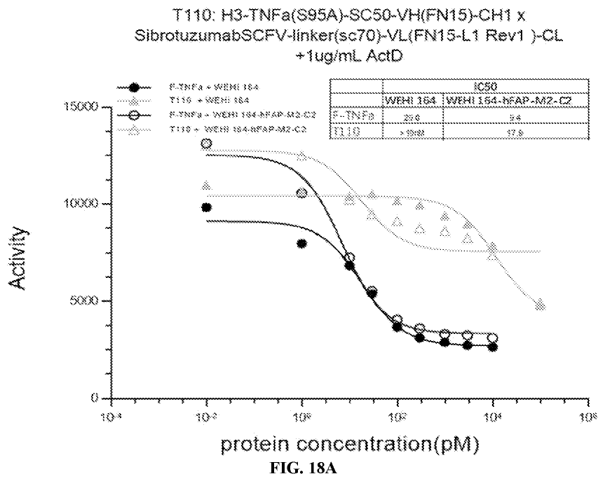

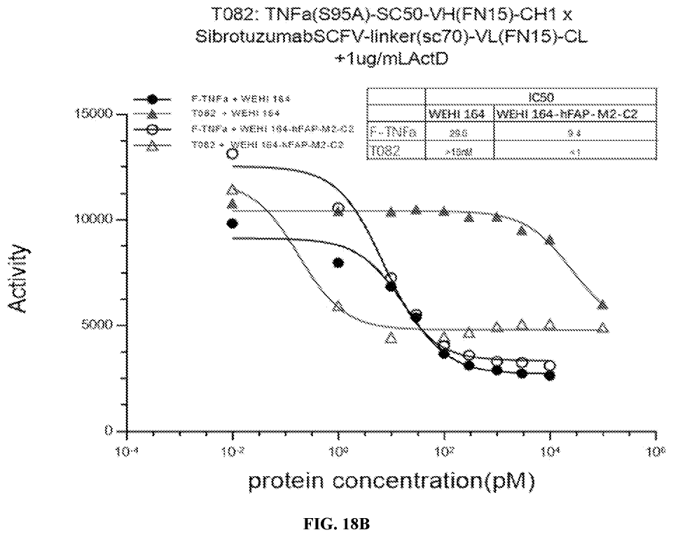

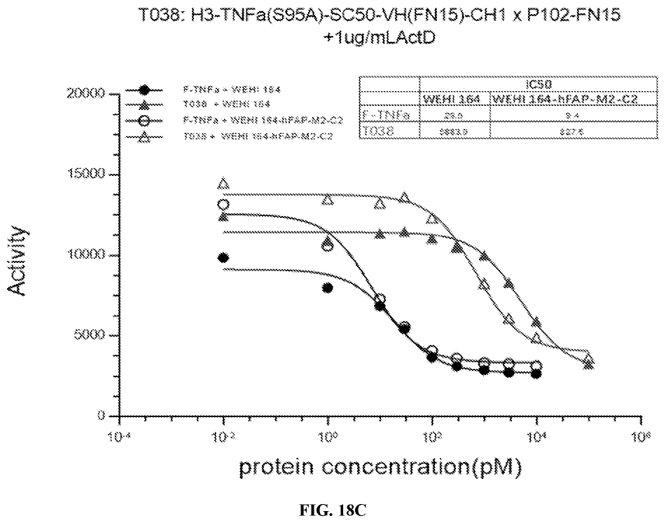

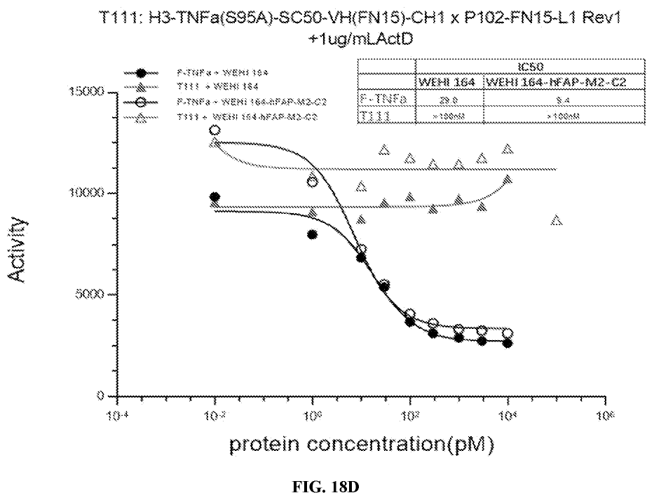

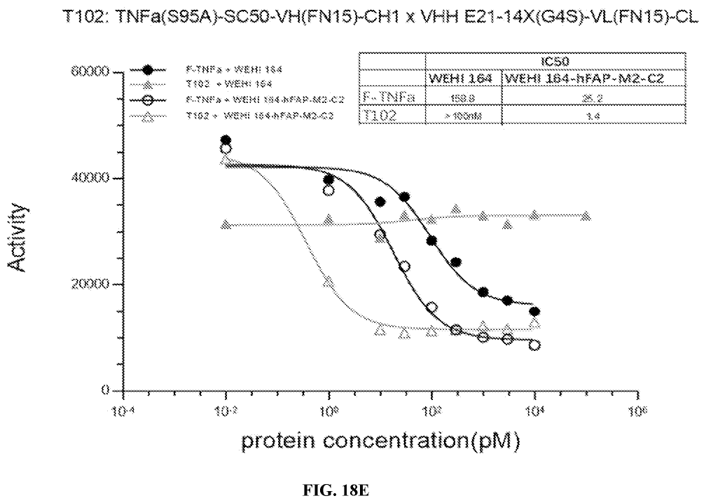

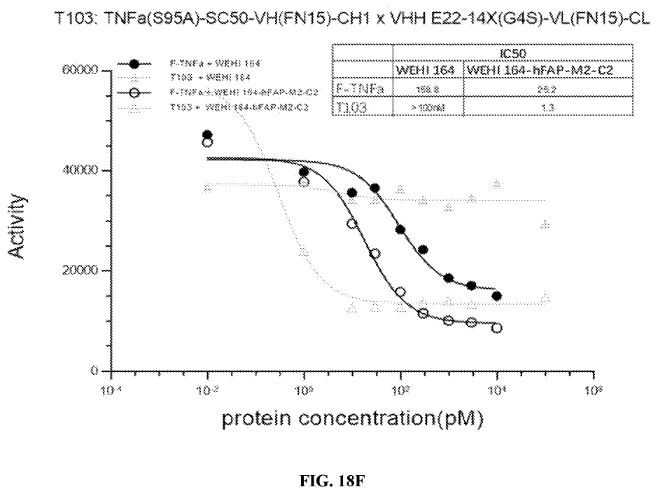

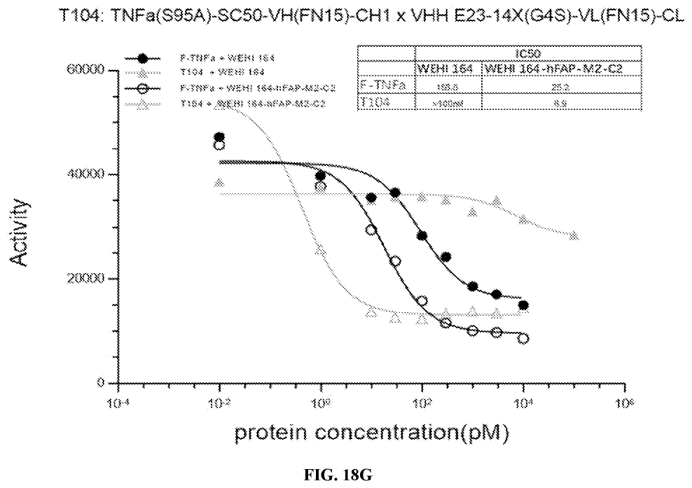

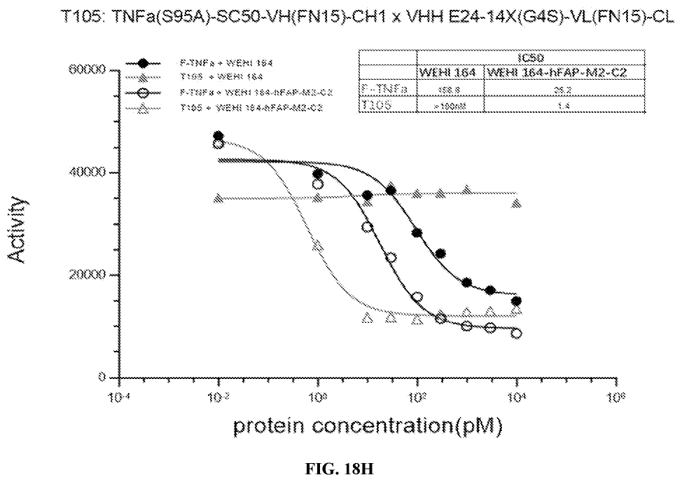

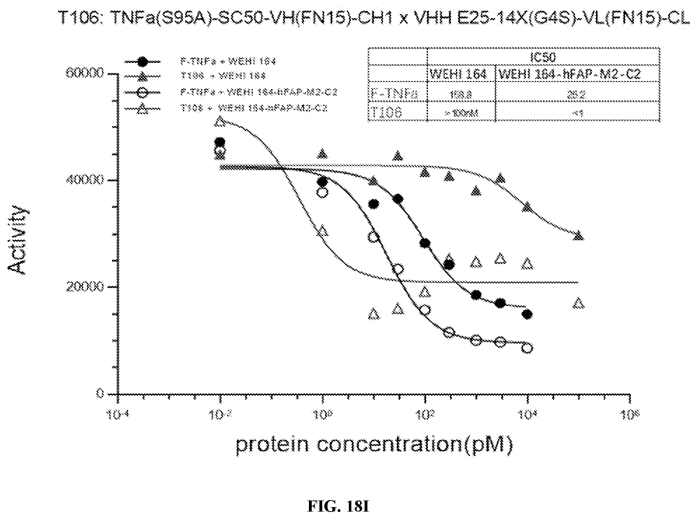

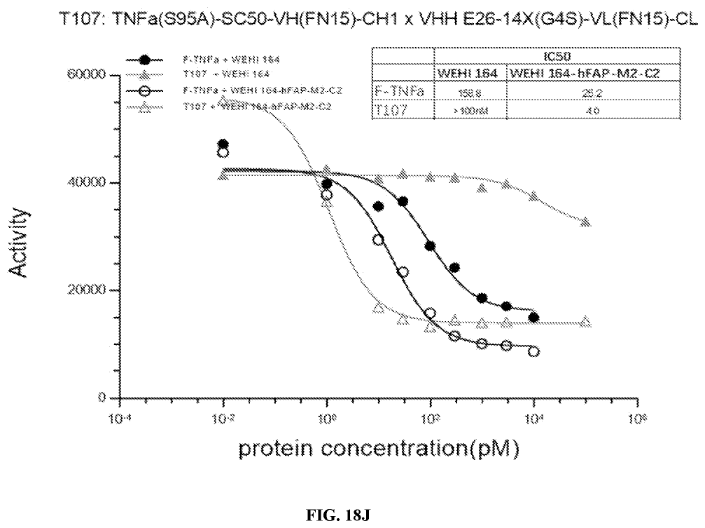

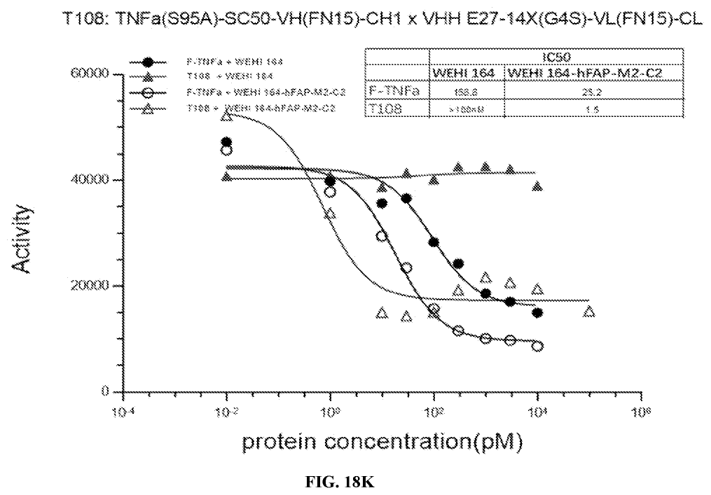

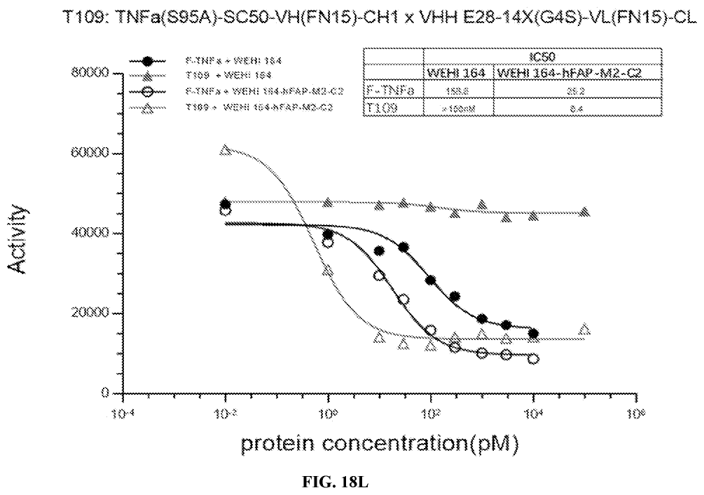

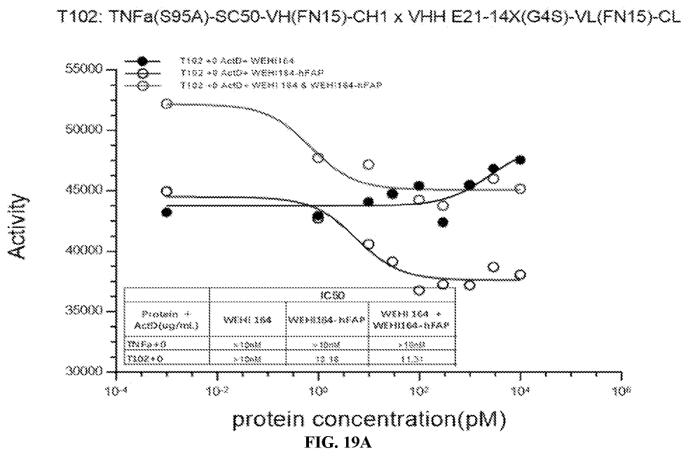

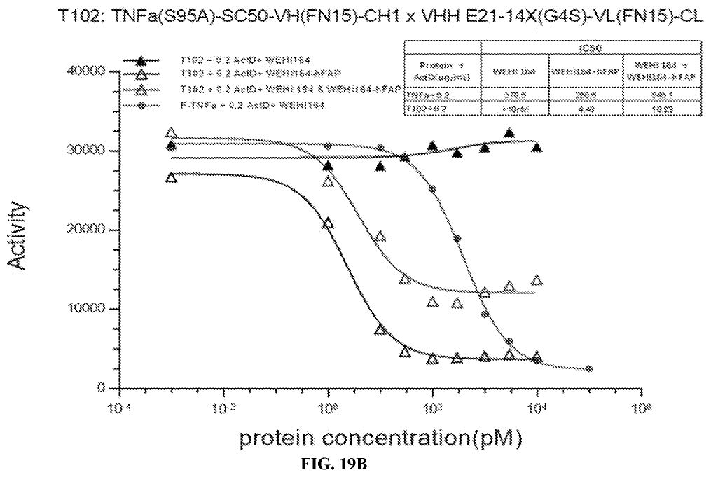

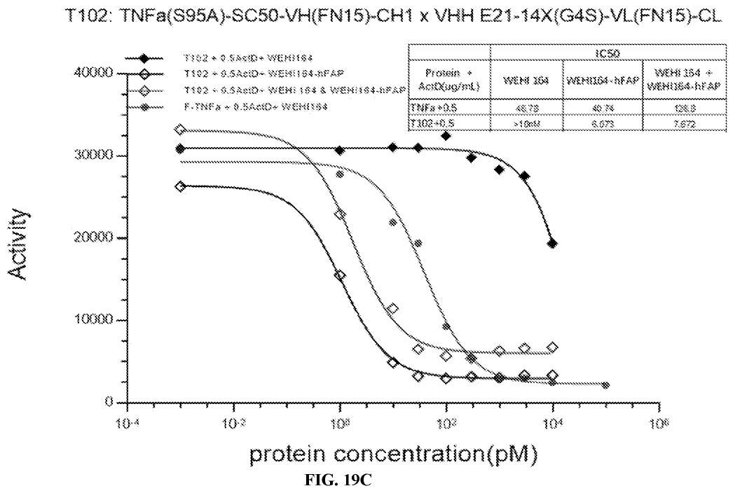

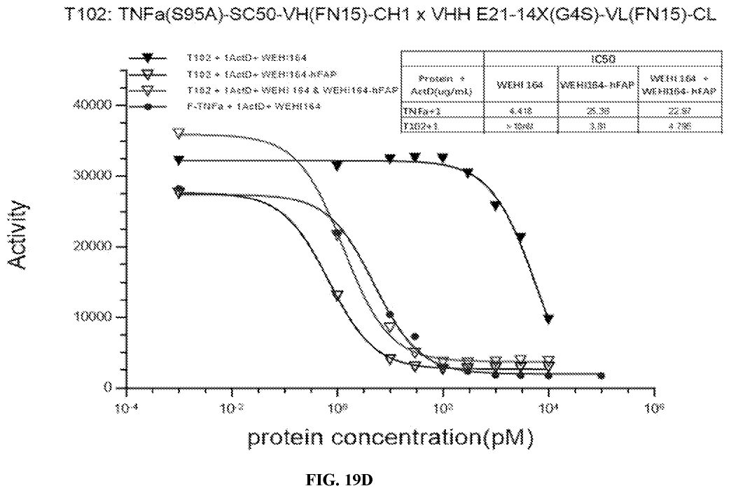

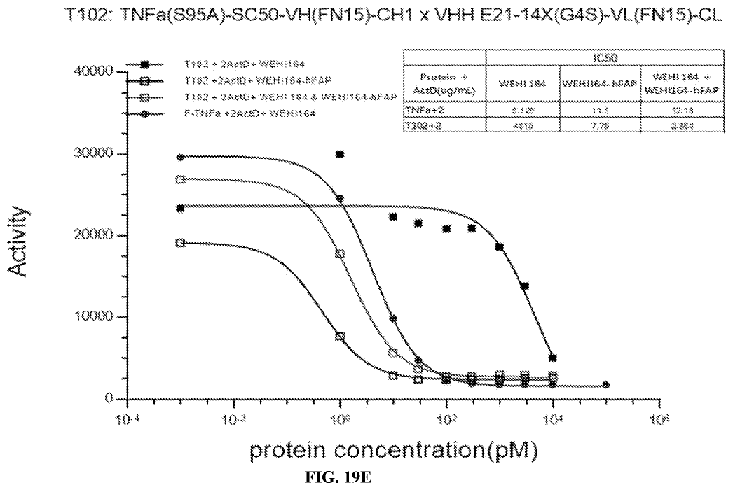

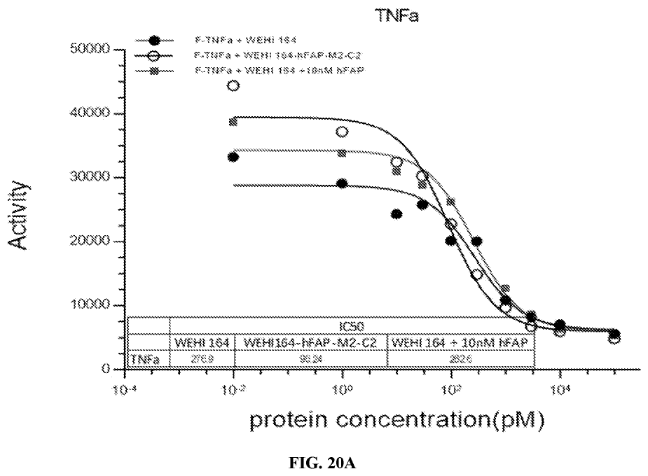

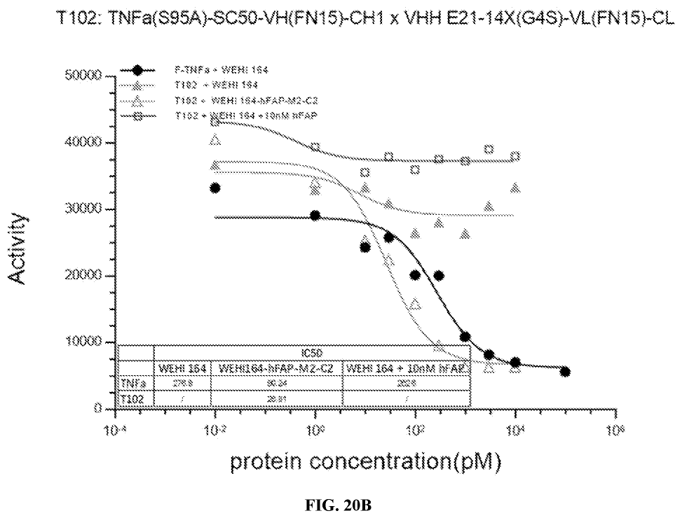

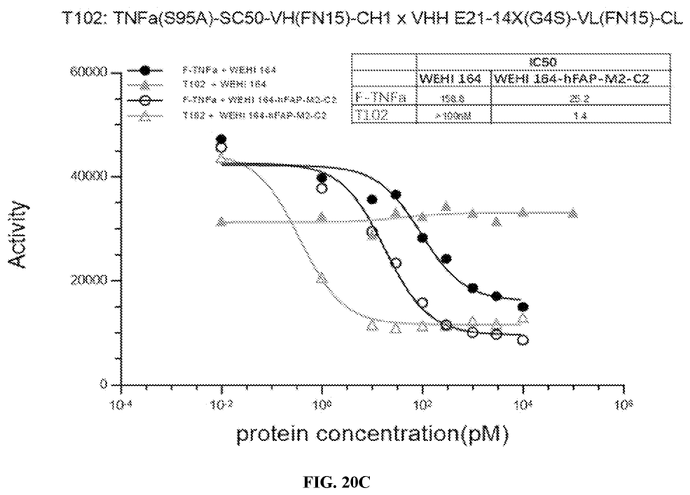

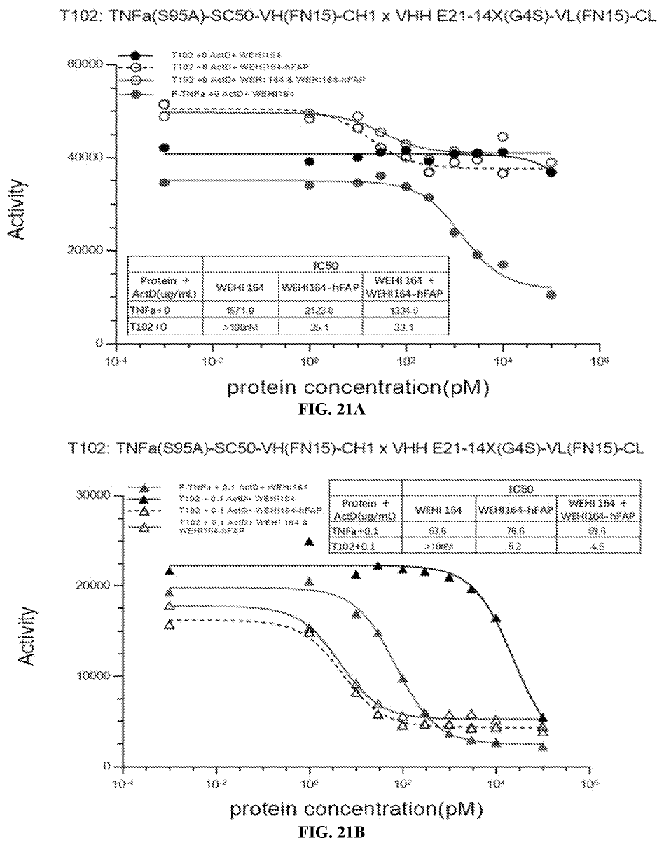

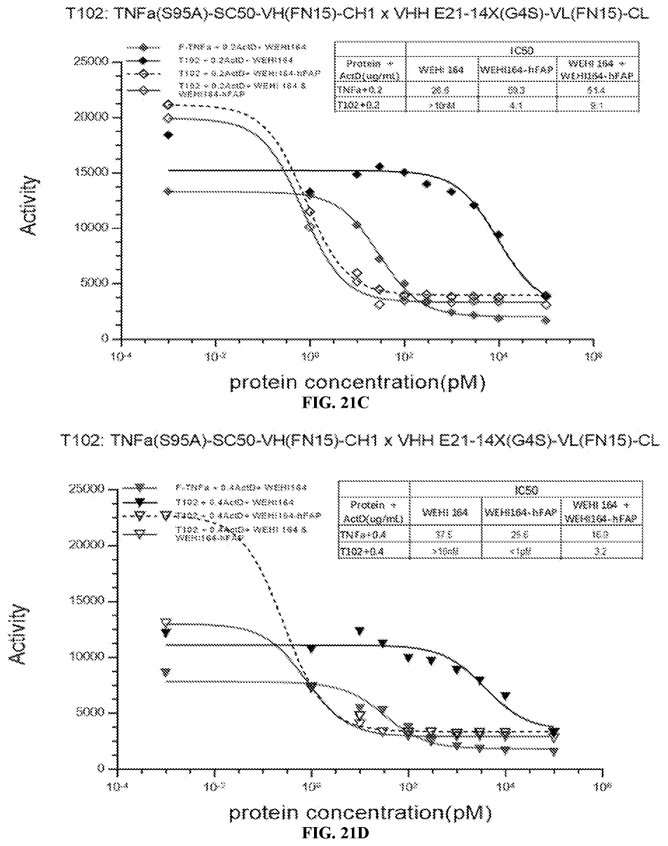

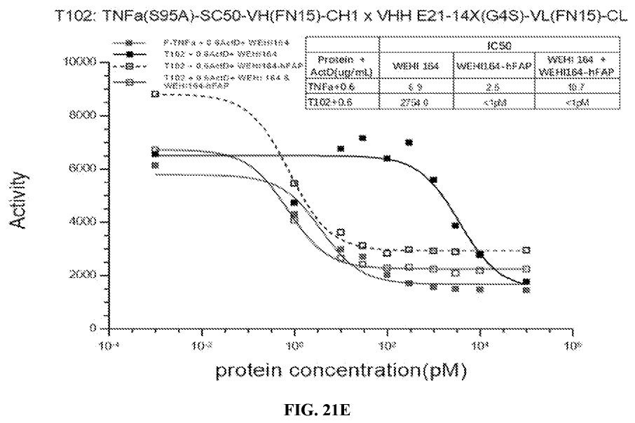

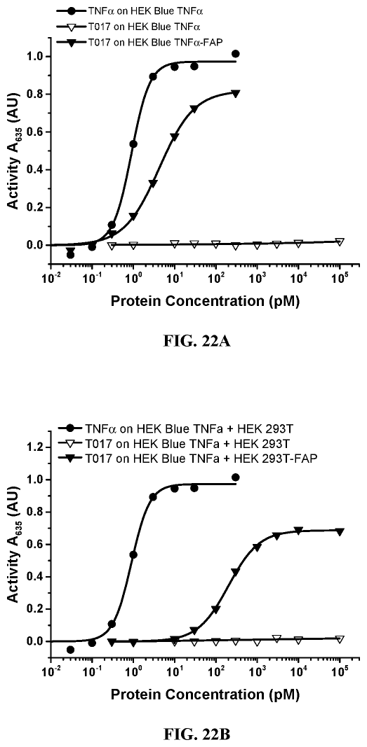

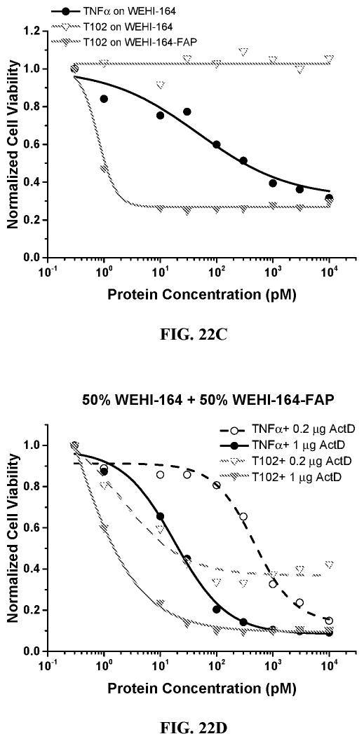

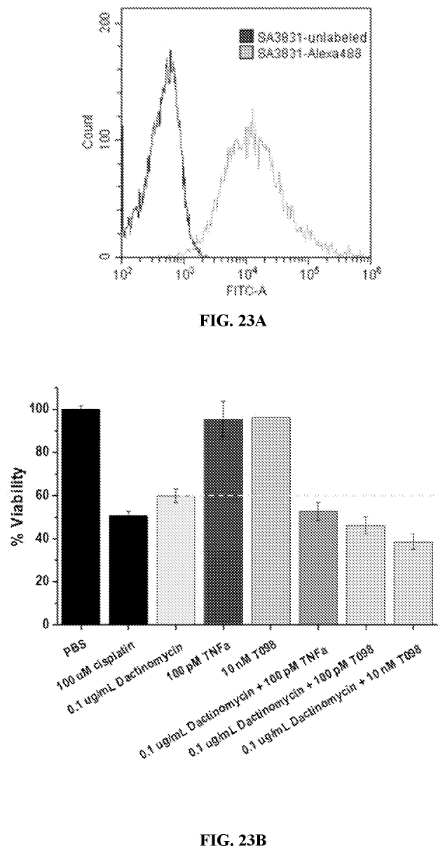

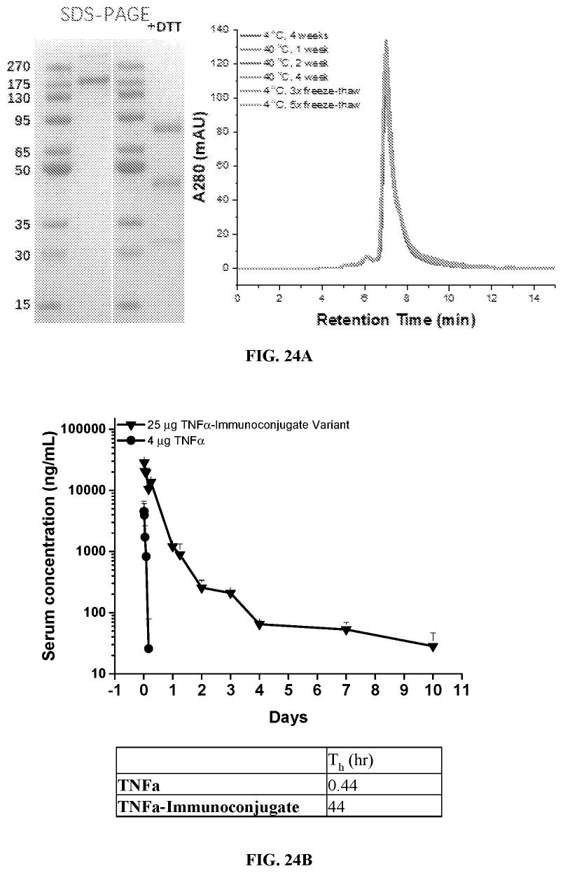

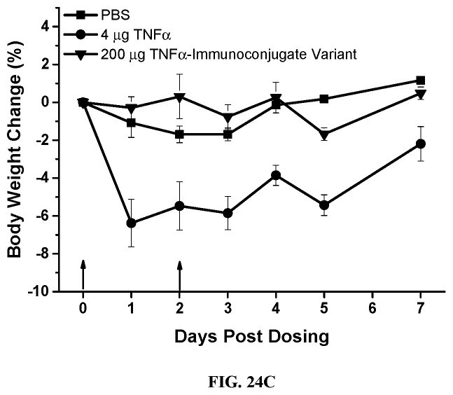

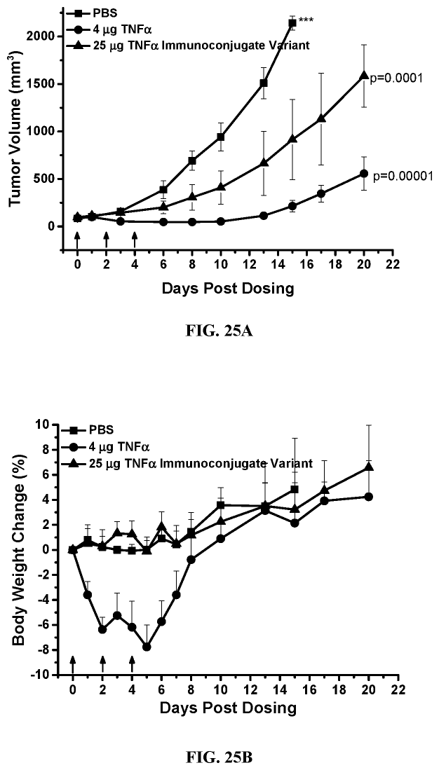

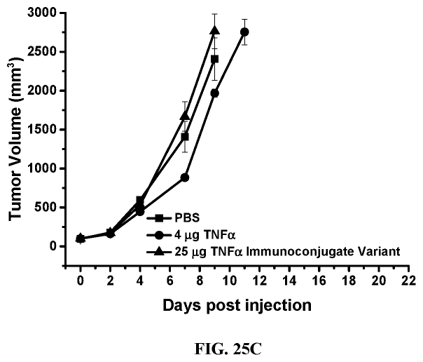

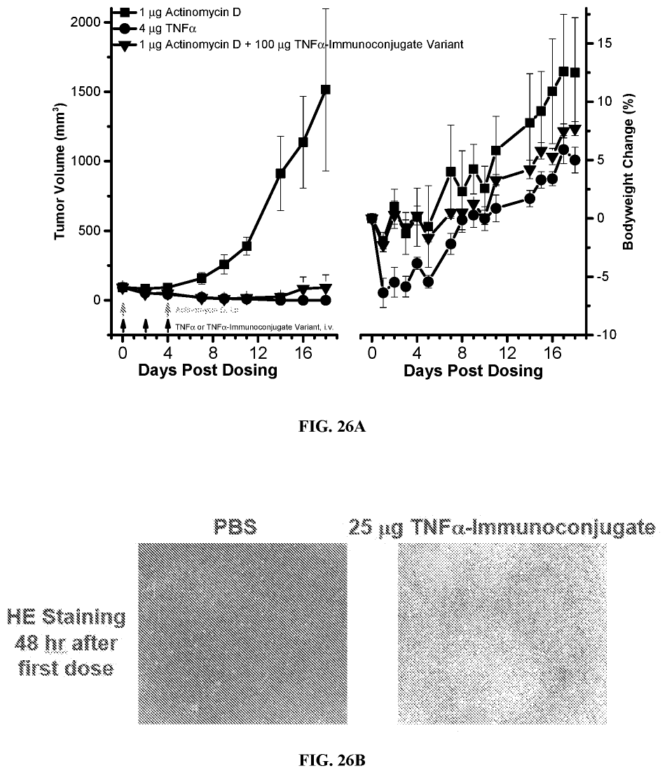

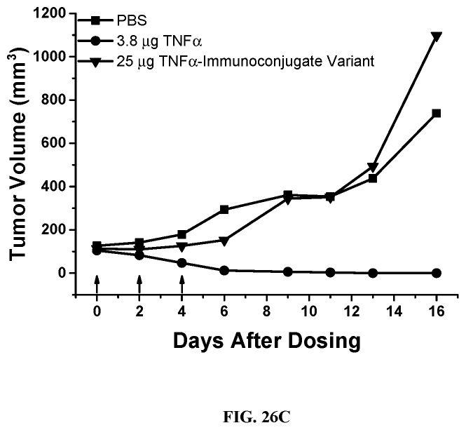

Particularly, FIG. 1B shows an immunoconjugate containing an anti-cytokine/anti-tumor associated antigen (TAA) two-in-one single chain Fv (scFv) antibody fused to a cytokine. There are four possible configurations: (i) cytokine-scFv(VH-VL), (ii) cytokine-scFv(VL-VH), (iii) scFv(VH-VL)-cytokine, and (iv) scFv(VL-VH)-cytokine, where “-” denotes a linker or direct fusion between different moieties, and the different moieties in a peptidic chain are listed according to the N to C orientation in the peptidic chain. The trimeric complex as shown in the figure can be a homotrimer containing three copies of the immunoconjugate molecules of the same configuration, or alternatively a heterotrimer containing three copies of the immunoconjugate molecules having two or three different configurations as described herein. In specific embodiments, the cytokine is wild-type or mutant TNF-α. In specific embodiments, the TAA is transmembrane glycoprotein encoded by the Tacstd2 gene (Trop-2) or fibroblast activation protein alpha (FAP).

FIG. 1C shows an immunoconjugate containing an anti-cytokine/anti-TAA two-in-one Fab antibody fused to a cytokine. As shown, there are four possible configurations: (i) cytokine-VH-CH1×VL-CL, (ii) cytokine-VL-CL×VH-CH1, (iii) VH-CH1-cytokine×VL-CL, and (iv) VL-CL-cytokine×VH-CH1, where “x” separates different peptidic chains forming part of an immunoconjugate molecule, “-” denotes a linker or direct fusion between different moieties, and the different moieties in a peptidic chain are listed according to the N to C orientation in the peptidic chain. The trimeric complex as shown in the figure can be a homotrimer containing three copies of the immunoconjugate molecules of the same configuration, or alternatively a heterotrimer containing three copies of the immunoconjugate molecules having two or three different configurations as described herein. In specific embodiments, the cytokine is wild-type or mutant TNF-α. In specific embodiments, the TAA is Trop-2 or FAP.

FIG. 1D shows an immunoconjugate containing an anti-cytokine/anti-TAA two-in-one single chain fragment antigen binding (scFab) antibody fused to a cytokine. There are four possible configurations: (i) cytokine-scFab(VH-CH1-VL-CL), (ii) cytokine-scFab(VL-CL-VH-CH1), (iii) scFab(VH-CH1-VL-CL)-cytokine, and (iv) scFab(VL-CL-VH-CH1)-cytokine, where “-” denotes a linker or direct fusion between different moieties, and the different moieties in a peptidic chain are listed according to the N to C orientation in the peptidic chain. The trimeric complex as shown in the figure can be a homotrimer containing three copies of the immunoconjugate molecules of the same configuration, or alternatively a heterotrimer containing three copies of the immunoconjugate molecules having two or three different configurations as described herein. In specific embodiments, the cytokine is wild-type or mutant TNF-α. In specific embodiments, the TAA is Trop-2 or FAP.

FIG. 1E shows an immunoconjugate containing (a) an anti-cytokine/anti-TAA two-in-one scFv antibody, (b) a cytokine polypeptide, and (c) an anti-TAA scFv antibody. There are eight possible configurations: (i) cytokine-scFv1(VH-VL)-scFv2(VH-VL), (ii) cytokine-scFv1(VL-VH)-scFv2(VH-VL), (iii) cytokine-scFv1(VH-VL)-scFv2(VL-VH), (iv) cytokine-scFv1(VL-VH)-scFv2(VL-VH), (v) scFv2(VH-VL)-scFv1(VH-VL)-cytokine, (vi) scFv2(VL-VH)-scFv1(VH-VL)-cytokine, (vii) scFv2(VH-VL)-scFv1(VL-VH)-cytokine, and (viii) scFv2(VL-VH)-scFv1(VL-VH)-cytokine, where scFv1 is the two-in-one antibody and scFv2 is the anti-TAA antibody, “-” denotes a linker or direct fusion between different moieties, and the different moieties in a peptidic chain are listed according to the N to C orientation in the peptidic chain. The trimeric complex as shown in the figure can be a homotrimer containing three copies of the immunoconjugate molecules of the same configuration, or alternatively a heterotrimer containing three copies of the immunoconjugate molecules having two or three different configurations as described herein. In specific embodiments, the cytokine is wild-type or mutant TNF-α. In specific embodiments, the TAA is Trop-2 or FAP.

FIG. 1F shows an immunoconjugate containing (a) an anti-cytokine/anti-TAA two-in-one scFv antibody, (b) a cytokine polypeptide, and (c) an anti-TAA VHH antibody. There are four possible configurations: (i) cytokine-scFv(VH-VL)-VHH, (ii) cytokine-scFv(VL-VH)-VHH, (iii) scFv(VH-VL)-VHH-cytokine, and (iv) scFv(VL-VH)-VHH-cytokine. The trimeric complex as shown in the figure can be a homotrimer containing three copies of the immunoconjugate molecules of the same configuration, or alternatively a heterotrimer containing three copies of the immunoconjugate molecules having two or three different configurations as described herein. In specific embodiments, the cytokine is wild-type or mutant TNF-α. In specific embodiments, the TAA is Trop-2 or FAP.

FIG. 1G shows an immunoconjugate containing (a) an anti-cytokine/anti-TAA two-in-one Fab antibody, (b) a cytokine polypeptide, and (c) an anti-TAA scFv antibody. There are eight possible configurations: (i) cytokine-VH-CH1×scFv(VH-VL)-VL-CL, (ii) cytokine-VL-CL×scFv(VH-VL)-VH-CH1, (iii) cytokine-VH-CH1×scFv(VL-VH)-VL-CL, (iv) cytokine-VL-CL×scFv(VL-VH)-VH-CH1, (v) VH-CH1-cytokine×scFv(VH-VL)-VL-CL, (vi) VL-CL-cytokine×scFv(VH-VL)-VH-CH1, (vii) VH-CH1-cytokine×scFv(VL-VH)-VL-CL, and (viii) VL-CL-cytokine×scFv(VL-VH)—VH-CH1, where “x” separates different peptidic chains forming part of a immunoconjugate molecule, “-” denotes a linker or direct fusion between different moieties, and the different moieties in a peptidic chain are listed according to the N to C orientation in the peptidic chain. The trimeric complex as shown in the figure can be a homotrimer containing three copies of the immunoconjugate molecules of the same configuration, or alternatively a heterotrimer containing three copies of the immunoconjugate molecules having two or three different configurations as described herein. In specific embodiments, the cytokine is wild-type or mutant TNF-α. In specific embodiments, the TAA is Trop-2 or FAP.

FIG. 1H shows an immunoconjugate containing (a) an anti-cytokine/anti-TAA two-in-one Fab antibody, (b) a cytokine polypeptide, and (c) an anti-TAA VHH antibody. There are four possible configurations: (i) cytokine-VH-CH1×VHH-VL-CL, (ii) cytokine-VL-CL×VHH-VH-CH1, (iii) VH-CH1-cytokine×VL-CL-VHH, (iv) VL-CL-cytokine×VH-CH1-VHH, where “x” separates different peptidic chains forming part of a immunoconjugate molecule, “-” denotes a linker or direct fusion between different moieties, and the different moieties in a peptidic chain are listed according to the N to C orientation in the peptidic chain. The trimeric complex as shown in the figure can be a homotrimer containing three copies of the immunoconjugate molecules of the same configuration, or alternatively a heterotrimer containing three copies of the immunoconjugate molecules having two or three different configurations as described herein. In specific embodiments, the cytokine is wild-type or mutant TNF-α. In specific embodiments, the TAA is Trop-2 or FAP.

FIG. 1I shows an immunoconjugate containing (a) an anti-cytokine/anti-TAA two-in-one Fab antibody, (b) a cytokine polypeptide, and (c) an anti-TAA scFv antibody. There are eight possible configurations: (i) cytokine-VH-CH1×VL-CL-scFv(VH-VL), (ii) cytokine-VL-CL×VH-CH1-scFv(VH-VL), (iii) cytokine-VH-CH1×VL-CL-scFv(VL-VH), (iv) cytokine-VL-CL×VH-CH1-scFv(VL-VH), (v) VH-CH1-cytokine×VL-CL-scFv(VH-VL), (vi) VL-CL-cytokine×VH-CH1-scFv(VH-VL), (vii) VH-CH1-cytokine×VL-CL-scFv(VL-VH), and (viii) VL-CL-cytokine×VH-CH1-scFv(VL-VH), where “x” separates different peptidic chains forming part of a immunoconjugate molecule, “-” denotes a linker or direct fusion between different moieties, and the different moieties in a peptidic chain are listed according to the N to C orientation in the peptidic chain. The trimeric complex as shown in the figure can be a homotrimer containing three copies of the immunoconjugate molecules of the same configuration, or alternatively a heterotrimer containing three copies of the immunoconjugate molecules having two or three different configurations as described herein. In specific embodiments, the cytokine is wild-type or mutant TNF-α. In specific embodiments, the TAA is Trop-2 or FAP.

FIG. 1J shows an immunoconjugate containing (a) an anti-cytokine/anti-TAA two-in-one Fab antibody, (b) a cytokine polypeptide, and (c) an anti-TAA scFv antibody. There are eight possible configurations: (i) cytokine-VH-CH1-scFv(VH-VL)×VL-CL, (ii) cytokine-VL-CL-scFv(VH-VL)×VH-CH1, (iii) cytokine-VH-CH1-scFv(VL-VH)×VL-CL, (iv) cytokine-VL-CL-scFv(VL-VH)×VH-CH1, (v) VH-CH1-scFv(VH-VL)-cytokine×VL-CL, (vi) VL-CL-scFv(VH-VL)-cytokine×VH-CH1, (vii) VH-CH1-scFv(VL-VH)-cytokine×VL-CL, and (viii) VL-CL-scFv(VL-VH)-cytokine×VH-CH1, where “x” separates different peptidic chains forming part of a immunoconjugate molecule, “-” denotes a linker or direct fusion between different moieties, and the different moieties in a peptidic chain are listed according to the N to C orientation in the peptidic chain. The trimeric complex as shown in the figure can be a homotrimer containing three copies of the immunoconjugate molecules of the same configuration, or alternatively a heterotrimer containing three copies of the immunoconjugate molecules having two or three different configurations as described herein. In specific embodiments, the cytokine is wild-type or mutant TNF-α. In specific embodiments, the TAA is Trop-2 or FAP.

FIG. 1K shows an immunoconjugate containing (a) an anti-cytokine/anti-TAA two-in-one Fab antibody, (b) a cytokine polypeptide, and (c) an anti-TAA VHH antibody. There are four possible configurations: (i) cytokine-VH-CH1×VL-CL-VHH, (ii) cytokine-VL-CL×VH-CH1-VHH, (iii) VH-CH1-cytokine×VHH-VL-CL, and (iv) VL-CL-cytokine×VHH-VH-CH1, where “x” separates different peptidic chains forming part of a immunoconjugate molecule, “-” denotes a linker or direct fusion between different moieties, and the different moieties in a peptidic chain are listed according to the N to C orientation in the peptidic chain. The trimeric complex as shown in the figure can be a homotrimer containing three copies of the immunoconjugate molecules of the same configuration, or alternatively a heterotrimer containing three copies of the immunoconjugate molecules having two or three different configurations as described herein. In specific embodiments, the cytokine is wild-type or mutant TNF-α. In specific embodiments, the TAA is Trop-2 or FAP.

FIG. 1L shows an immunoconjugate containing (a) an anti-cytokine/anti-TAA two-in-one Fab antibody, (b) a cytokine polypeptide, and (c) an anti-TAA VHH antibody. There are four possible configurations: (i) cytokine-VH-CH1-VHH×VL-CL, (ii) cytokine-VL-CL-VHH×VH-CH1, (iii) VHH-VH-CH1-cytokine×VL-CL, and (iv) VHH-VL-CL-cytokine×VH-CH1, where “x” separates different peptidic chains forming part of a immunoconjugate molecule, “-” denotes a linker or direct fusion between different moieties, and the different moieties in a peptidic chain are listed according to the N to C orientation in the peptidic chain. The trimeric complex as shown in the figure can be a homotrimer containing three copies of the immunoconjugate molecules of the same configuration, or alternatively a heterotrimer containing three copies of the immunoconjugate molecules having two or three different configurations as described herein. In specific embodiments, the cytokine is wild-type or mutant TNF-α. In specific embodiments, the TAA is Trop-2 or FAP.

FIG. 2A is a schematic illustration of a soluble cytokine polypeptide.

FIGS. 2B to 2U are schematic illustrations of antibody-cytokine immunoconjugates of different molecular configurations according to the present disclosure. Particularly, FIG. 2B shows an immunoconjugate containing a cytokine polypeptide fused to the C-terminus of one of the two heavy chain fragments in an immunoglobulin Fc domain (e.g., Fc-knob).

FIG. 2C shows an immunoconjugate containing (a) anti-cytokine/anti-TAA two-in-one Fab antibody fused at the N-terminus of its heavy chain fragment to the C-terminus of one of the heavy chain fragments of the immunoglobulin Fc domain (e.g., Fc-hole), and (b) a cytokine polypeptide fused to the C-terminus of the other heavy chain fragment in the immunoglobulin Fc domains (e.g., Fc-knob), and.

FIG. 2D shows an immunoconjugate containing (a) an anti-cytokine/anti-TAA two-in-one Fab antibody fused at the N-terminus of its heavy chain fragment to the C-terminus of one of the heavy chain fragments of the immunoglobulin Fc domain (e.g., the Fc-hole), (b) a cytokine polypeptide fused to the C-terminus of the other one of the two heavy chain fragments of an immunoglobulin Fc domain (e.g., the Fc-knob), and (c) an anti-TAA scFv antibody fused to the N terminus of one of the two heavy chain fragments in the immunoglobulin Fc domain (e.g., the Fc-knob).

FIG. 2E shows an immunoconjugate containing (a) an anti-cytokine/anti-TAA two-in-one Fab antibody fused at the N-terminus of its heavy chain fragment to the C-terminus of one of the two heavy chain fragments of the immunoglobulin Fc domain (e.g., Fc-hole), and (b) a cytokine polypeptide fused to the N terminus of the light chain fragment of the Fab antibody.

FIG. 2F shows an immunoconjugate containing (a) an anti-cytokine/anti-TAA two-in-one Fab antibody fused at the N-terminus of its heavy chain fragment to the C-terminus of one of the heavy chain fragments of an immunoglobulin Fc domain (e.g., Fc-knob); (b) a cytokine polypeptide fused to the C-terminus of the other heavy chain fragment of the immunoglobulin Fc domain (e.g., Fc-hole), and (c) an anti-TAA single domain antibody fused to the N-terminus of one of the two heavy chain fragments in the immunoglobulin Fc domain (e.g., Fc-knob).

FIG. 2G shows an immunoconjugate containing (a) an anti-cytokine/anti-TAA two-in-one scFv antibody fused at the C-terminus of its heavy chain fragment to the N-terminus of one of the heavy chain fragments of an immunoglobulin Fc domain (e.g., Fc-hole), (b) a cytokine polypeptide fused to the C-terminus of the other heavy chain fragment of the immunoglobulin Fc domain (e.g., Fc-knob), and (c) an anti-TAA Fab antibody fused to the N-terminus of one of the two heavy chain fragments in the immunoglobulin Fc domain (e.g., Fc-knob).

FIG. 2H shows an immunoconjugate containing (a) an anti-cytokine/anti-TAA two-in-one Fab antibody fused at the N-terminus of its heavy chain fragment to the C-terminus of one of the two heavy chain fragments of the immunoglobulin Fc domain (e.g., the Fc-knob), (b) a cytokine polypeptide fused to the N-terminus of the light chain fragment of the Fab antibody, and (c) an anti-TAA scFv antibody fused to the N-terminus of one of the two heavy chain fragments in the immunoglobulin Fc domain (e.g., Fc-hole).

FIG. 2I shows an immunoconjugate containing (a) an anti-cytokine/anti-TAA two-in-one Fab antibody fused at the N-terminus of its heavy chain fragment to the C-terminus of one of the two heavy chain fragments of the immunoglobulin Fc domain (e.g., Fc-hole), (b) a cytokine peptide fused to the C-terminus of the other heavy chain fragments in an immunoglobulin Fe domains (e.g., Fc-knob), and (c) an anti-TAA Fab fused to the N-terminus of one of the two heavy chain fragments in the immunoglobulin Fc domain (e.g., Fc-knob).

FIG. 2J shows an immunoconjugate containing (a) an anti-cytokine/anti-TAA two-in-one Fab antibody fused at the N-terminus of its heavy chain fragment to the C-terminus of one of the two heavy chain fragments of the immunoglobulin Fc domain (e.g., the Fc-hole), (b) a cytokine peptide fused to the N-terminus of the light chain fragment of the Fab antibody, and (c) an anti-TAA scFv antibody fused to the N-terminus of one of the two heavy chain fragments in the immunoglobulin Fc domain (e.g., Fc-knob).

FIG. 2K shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., the Fc-knob), (b) a cytokine peptide fused to the N-terminus of the heavy chain fragment of a Fab antibody, and (c) an anti-TAA Fab antibody fused at the C-terminus of its heavy chain fragment to the N-terminus of the other heavy chain fragment of the immunoglobulin Fc domain (e.g., the Fc-hole).

FIG. 2L shows an immunoconjugate containing (a) an anti-cytokine/anti-TAA two-in-one Fab antibody fused at the N-terminus of its heavy chain fragment to the C-terminus of one of the heavy chain fragments of the immunoglobulin Fc domain (e.g., the Fc-hole), (b) a cytokine polypeptide fused to the C-terminus of the other heavy chain fragment of the immunoglobulin Fc domain (e.g., the Fc-knob), and (c) an anti-TAA scFv antibody fused to the N-terminus of one of the two heavy chain fragments in the immunoglobulin Fc domain (e.g., the Fc-hole).

FIG. 2M shows an immunoconjugate containing (a) an anti-cytokine/anti-TAA two-in-one Fab antibody fused at the N-terminus of its heavy chain fragment to the C-terminus of one of the heavy chain fragments of the immunoglobulin Fc domain (e.g., the Fc-hole), (b) a cytokine peptide fused to the C-terminus of the other heavy chain fragment of the immunoglobulin Fc domain (Fc-knob), and (c) and anti-TAA scFv fused to the C-terminus of the heavy chain fragment of the anti-cytokine/anti-TAA two-in-one Fab antibody.

FIG. 2N shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., the Fc-hole), (b) a cytokine peptide fused to the N-terminus of the heavy chain fragment of a Fab antibody, and (c) an anti-TAA Fab antibody fused at the C-terminus of its heavy chain to the N-terminus of the other heavy chain fragment of the immunoglobulin Fc domain (e.g., the Fc-knob).

FIG. 2O shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., Fc-knob), (b) a cytokine peptide fused to the N-terminus of the heavy chain fragment of the Fab antibody, and (c) an anti-TAA single domain antibody fused to the N-terminus of the other one of the two heavy chain fragments in the immunoglobulin Fc domain (e.g., Fc-hole).

FIG. 2P shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., Fc-knob), (b) a cytokine peptide fused to the N-terminus of the heavy chain fragment of the Fab antibody, and (c) an anti-TAA scFv antibody fused to the N-terminus of the other one of the two heavy chain fragments in the immunoglobulin Fc domain (e.g., Fc-hole).

FIG. 2Q shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., Fc-hole), (b) a cytokine peptide fused to the N-terminus of the heavy chain fragment of the Fab antibody, and (c) an anti-TAA scFv antibody fused to the N-terminus of the other one of the two heavy chain fragments in the immunoglobulin Fc domain (e.g., Fc-knob).

FIG. 2R shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., Fc-hole), (b) a cytokine peptide fused to the N-terminus of the light chain fragment of the Fab antibody, and (c) an anti-TAA scFv antibody fused to the N-terminus of the other one of the two heavy chain fragments in the immunoglobulin Fc domain (e.g., Fc-knob).

FIG. 2S shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., Fc-knob), (b) a cytokine peptide fused to the N-terminus of the light chain fragment of the Fab antibody, and (c) an anti-TAA scFv antibody fused to the N-terminus of the other one of the two heavy chain fragments in the immunoglobulin Fc domain (e.g., Fc-hole).

FIG. 2T shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., Fc-knob), (b) an anti-TAA Fab antibody fused at the C-terminus of its heavy chain fragment to the N-terminus of the other heavy chain fragment in the immunoglobulin Fc domain (e.g., Fc-hole), and (c) a cytokine peptide fused to the N-terminus of the heavy chain fragment of the Fab antibody.

FIG. 2U shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., Fc-knob), and (b) a cytokine peptide fused to the N-terminus of the heavy chain fragment of the Fab antibody.

FIGS. 3A to 3L show binding affinities of two-in-one antibody variants to both TNF-α and tumor associated antigen (“TAA”) through biolayer interferometry analysis. Particularly, FIGS. 3A to 3F show binding affinities of six two-in-one Fab-Fc capable of binding to both TNF-α and FAP; FIGS. 3G to 3L show binding affinities of six two-in-one Fab-Fc capable of binding to both TNF-α and Trop2. The numeric values of the binding affinities are summarized in Table 3 and Table 4.

FIG. 4A is a schematic illustration of a trimer of TNF-α containing immunoconjugates according to one embodiment of the present disclosure. In this exemplary embodiment, each of the immunoconjugates comprises (i) a wild-type TNF-α polypeptide capable of mediating a cellular effect, (ii) a masking moiety capable of binding to TNF-α and inhibiting the cellular effect of TNF-α, and (iii) a peptidic linker connecting the portions described in (i) and (ii) of the immunoconjugate.

FIG. 4B shows TNF-α activities of immunoconjugates having the configuration in FIG. 4A measured using the HEK Blue TNF-α reporter cell line. X-axis shows the concentration (pM) of the TNF-α containing immunoconjugates; Y-axis shows the absorbance at 635 nm (A₆₃₅) determined using a TECAN plate reader, which reflects the level of secreted embryonic alkaline phosphatase (SEAP) and thus TNF-α activity. Two immunoconjugates were tested: a wild-type TNF-α polypeptide fused to a scFv derived from a commercially available anti-TNF-α antibody adalimumab (TNF-α-adascFv; solid circle) and a wild-type TNF-α polypeptide fused to a scFv derived from another commercially available anti-TNF-α antibody certolizumab (TNF-α-certvhvl; up triangle). A commercially available wild-type TNF-α polypeptide in the non-conjugated form was also included in the assay as a control (Sino Biological; square). As shown, TNF-α activities of both tested immunoconjugates were inhibited by anti-TNF-α scFvs in these immunoconjugates as compared to the unconjugated TNF-α control.

FIG. 4C shows TNF-α activities of immunoconjugates having the configuration in FIG. 4A measured using the murine L929 cell line. X-axis shows the concentration (pM) of the TNF-α containing immunoconjugates in the presence of 1 μg/mL actinomycin D; Y-axis shows the intensity of the Alamar blue fluorescence, which reflects the amount of live cells and thus TNF-α activity. Two immunoconjugates were tested: TNF-α-adascFv (up triangle) and TNF-α-certvhvl (diamond). Two versions of wild-type TNF-α polypeptide in the non-conjugated form were also included in the assay as controls: Sino TNF-α as used in FIG. 4B (square) and a TNF-α with a FLAG-tag at the C-terminus (circle). TNF-α activities of both tested immunoconjugates were inhibited by anti-TNF-α scFvs in these immunoconjugates as compared to the unconjugated TNF-α controls.

FIG. 5A is a schematic illustration of a trimer of immunoconjugates containing mutated TNF-α according to one embodiment of the present disclosure. In this exemplary embodiment, each of the immunoconjugates comprises (i) a mutated TNF-α polypeptide with further diminished activity under the shielded condition but capable of mediating a cellular effect under the de-shielded condition, (ii) a masking moiety capable of binding to TNF-α and inhibiting the cellular effect of TNF-α, and (iii) a peptidic linker connecting the portions described in (i) and (ii) of the immunoconjugate.

The top panels of FIGS. 5B to 5D show TNF-α activities of immunoconjugates having the configuration in FIG. 5A measured using the HEK Blue TNF-α reporter cell line. X-axis shows the concentration (pM) of the immunoconjugates containing the mutated TNF-α; Y-axis shows the absorbance at 635 nm (A₆₃₅) determined using a TECAN plate reader, which reflects the level of secreted embryonic alkaline phosphatase (SEAP) and thus TNF-α activity. The bottom panels of FIGS. 5B to 5D show TNF-α activities of immunoconjugates having the configuration in FIG. 5A measured using the murine L929 cell line. X-axis shows the concentration (pM) of the immunoconjugates containing the mutated TNF-α; Y-axis shows the intensity of the Alamar blue fluorescence, which reflects the amount of live cells and thus TNF-α activity. Three versions of mutant TNF-α were tested in both assays: TNF-α S147A in FIG. 5B, TNF-α N34H in FIG. 5C, and TNF-α S95A in FIG. 5D. TNF-α activities of the unconjugated mutant TNF-α (solid circle in FIG. 5B, solid diamond in FIG. 5C, solid circle in FIG. 5D) were comparable to that of the unconjugated wild-type TNF-α (solid square and open square). In contrast, under the shielded condition, TNF-α activities of immunoconjugates containing the mutant TNF-α polypeptide conjugated to a scFv derived from anti-TNF-α adalimumab (open circle in FIG. 5B, open diamond in FIG. 5C, open circle in FIG. 5D) were significantly inhibited.

FIG. 6A is a schematic illustration of a trimer of immunoconjugates containing mutated TNF-α according to one embodiment of the present disclosure. In this exemplary embodiment, each of the immunoconjugates comprises (i) a mutated TNF-α polypeptide with further diminished activity under the shielded condition but capable of mediating a cellular effect in the unconjugated form, (ii) a masking moiety capable of binding to TNF-α and inhibiting the cellular effect of TNF-α, (iii) an anchoring moiety capable of binding to a tumor associated antigen: fibroblast activation protein alpha (FAP), thereby immobilizing the immunoconjugate in an environment enriched of FAP, and (iv) peptidic linkers connecting the portions described in (i), (ii), and (iii) of the immunoconjugate.

FIGS. 6B and 6C show TNF-α activities of immunoconjugates having the configuration in FIG. 6A measured using the HEK Blue TNF-α reporter cell line. The assays were performed in the absence (solid circle and triangle) or presence (open circle and triangle) of FAP expressed on the surface of HEK Blue TNF-α reporter cells. X-axis shows the concentration (pM) of the immunoconjugates containing both the mutated TNF-α and the anchoring moiety; Y-axis shows the absorbance at 635 nm (A₆₃₅) determined using a TECAN plate reader, which reflects the level of secreted embryonic alkaline phosphatase (SEAP) and thus TNF-α activity. Two variants of mutated TNF-α in combination with two different linkers linking the anchoring moiety to the immunoconjugate were tested: TNF-α S147G plus linker (sc50) in Protein 15, TNF-α Y87F plus linker (sc50) in Protein 16, TNF-α S147G plus linker (sc70) in Protein 17, and TNF-α Y87F plus linker (sc70) in Protein 18. Unconjugated wild-type TNF-α polypeptides in the absence (solid circle) or presence of FAP-expressing cells (open circle) were included as controls. As shown, in the absence of FAP-expressing cells, TNF-α activities of the tested immunoconjugates (solid triangles) were significantly inhibited. In contrast, in the presence of FAP expression, TNF-α activities of the tested immunoconjugates (open triangles) were partially restored as compared to the control groups.

FIG. 7A is a schematic illustration of a trimer of immunoconjugates containing mutant TNF-α according to one embodiment of the present disclosure. In this exemplary embodiment, each of the immunoconjugates comprises (i) a mutant TNF-α polypeptide with further diminished activity under the shielded condition but capable of mediating a cellular effect under the naked condition; (ii) a two-in-one masking moiety capable of (a) binding to TNF-α and inhibiting the cellular effect of TNF-α, and (b) binding to FAP; (iii) an anchoring moiety capable of binding to FAP, thereby immobilizing the immunoconjugate in an environment enriched of FAP, and (iv) peptidic linkers connecting the portions described in (i), (ii), and (iii) of the immunoconjugate.

FIGS. 7B to 7G show TNF-α activities of immunoconjugates having the configuration in FIG. 7A measured using the HEK Blue TNF-α reporter cell line. The assays were performed in the absence (solid circle and triangle) or presence (open circle and triangle) of FAP expressed on the surface of HEK Blue TNF-α reporter cells. X-axis shows the concentration (pM) of the immunoconjugates; Y-axis shows the absorbance at 635 nm (A₆₃₅) determined using a TECAN plate reader, which reflects the level of secreted embryonic alkaline phosphatase (SEAP) and thus TNF-α activity. Each of the six tested immunoconjugates contained a Y87F or S147G point mutation of TNF-α, a different two-in-one masking moiety, and a different anchoring moiety. Unconjugated wild-type TNF-α polypeptides in the absence (solid circle) or presence of FAP-expressing cells (open circle) were included as controls. As shown, in the absence of FAP-expressing cells, TNF-α activities of the tested immunoconjugates (solid triangle) were significantly inhibited. In contrast, in the presence of FAP-expressing cells, TNF-α activities of the tested immunoconjugates (open triangle) were at least partially restored as compared to the control groups.

FIGS. 8A to 8F show TNF-α activities of immunoconjugates having the configuration in FIG. 7A measured using the HEK Blue TNF-α reporter cell line. The assays were performed in the absence (solid circle and triangle) or presence (open circle and triangle) of FAP-expressing cells co-cultured with HEK Blue TNF-α reporter cells. X-axis shows the concentration (pM) of the immunoconjugates; Y-axis shows the absorbance at 635 nm (A₆₃₅) determined using a TECAN plate reader, which reflects the level of secreted embryonic alkaline phosphatase (SEAP) and thus TNF-α activity. Each of the six immunoconjugates contained a Y87F or S147G point mutation of TNF-α, a different two-in-one masking moiety, and a different anchoring moiety. Unconjugated wild-type TNF-α polypeptides in the absence (solid circle) or presence of FAP-expressing cells (open circle) were included as controls. As shown, in the absence of FAP-expressing cells, TNF-α activities of the tested immunoconjugates (solid triangle) were significantly inhibited. In contrast, in the presence of FAP-expressing cells, TNF-α activities of the tested immunoconjugates (open triangle) were at least partially restored as compared to the control groups.

FIG. 9A is a schematic illustration of a trimer of immunoconjugates containing mutated TNF-α according to one embodiment of the present disclosure. In this exemplary embodiment, each of the immunoconjugates comprises (i) a mutated TNF-α polypeptide with further diminished activity under the shielded condition but capable of mediating a cellular effect under the unconjugated form; (ii) a two-in-one masking moiety capable of (a) binding to TNF-α and inhibiting the cellular effect of TNF-α, and (b) binding to FAP; and (iii) peptidic linkers connecting the portions described in (i) and (ii) of the immunoconjugate.

FIG. 9B shows TNF-α activities of immunoconjugates having the configuration in FIG. 9A measured using the HEK Blue TNF-α reporter cell line. The assays were performed in the absence (circle and up triangle) or presence (down triangle and diamond) of FAP expressed on the surface of HEK Blue TNF-α reporter cells. X-axis shows the concentration (pM) of the immunoconjugates; Y-axis shows the absorbance at 635 nm (A₆₃₅) determined using a TECAN plate reader, which reflects the level of secreted embryonic alkaline phosphatase (SEAP) and thus TNF-α activity. In both tested immunoconjugates, the mutated TNF-α has a Y87F point mutation. The two-in-one masking moiety is FN06 or FN15 in the two tested immunoconjugates. As shown, in the absence of FAP-expressing cells, TNF-α activities of the tested immunoconjugates (circle and up triangle) were significantly inhibited. In contrast, in the presence of FAP-expressing cells, TNF-α activities of the tested immunoconjugates (down triangle and diamond) were at least partially restored.

FIGS. 10A to 10D show TNF-α activities of immunoconjugates having the configuration in FIG. 7A and with different lengths of the linker between mutant TNF-α polypeptide and the two-in-one masking moiety (50, 70, or 160 amino acids long) or the linker between the anchoring moiety and the two-in-one masking moiety (70 or 160 amino acids long). TNF-α activities of the tested immunoconjugates were measured using the HEK Blue TNF-α reporter cell line. The assays were performed in the absence (solid circle and triangles) or presence (open circle and triangles) of FAP expressed on the surface of HEK Blue TNF-α reporter cells. X-axis shows the concentration (pM) of the immunoconjugates; Y-axis shows the absorbance at 635 nm (A₆₃₅) determined using a TECAN plate reader, which reflects the level of secreted embryonic alkaline phosphatase (SEAP) and thus TNF-α activity. Particularly, the immunoconjugates tested in FIG. 10A contained a S147G point mutation in the TNF-α polypeptide and FN06 as the two-in-one masking moiety; the immunoconjugates tested in FIG. 10B contained a S147G point mutation in the TNF-α polypeptide and FN15 as the two-in-one masking moiety; the immunoconjugates tested in FIG. 10C contained a Y87F point mutation in the TNF-α polypeptide and FN06 as the two-in-one masking moiety; the immunoconjugates tested in FIG. 10D contained a Y87F point mutation in the TNF-α polypeptide and FN15 as the two-in-one masking moiety; the anchoring moiety of all tested immunoconjugates is scFv70. In addition, the commercially available wild-type TNF-α polypeptide in the non-conjugated form was included in the assay as a control (Sino Biological; solid square). As shown, in the absence of FAP-expressing cells, TNF-α activities of the tested immunoconjugates (solid circle and triangles) were all significantly inhibited; in the presence of FAP-expressing cells, TNF-α activities of the tested immunoconjugates (open circle and triangles) were at least partially restored as compared to the control groups. Although there is no meaningful correlation between TNF-α activity and the length of the linker connecting the anchoring moiety and the masking moiety, there is a modest (˜10-fold) difference in TNF-α activity between the shortest and the longest length of the linker connecting TNF-α and the masking moiety.

FIG. 11A shows the SDS-PAGE results of purified protein samples from constructs designed to express covalent disulfide-linked TNF-α variants. Five of the eleven purified protein samples expressed at comparable level as wild type TNF-α, and appeared at expected size of a TNF-α trimer (51-kDa) (top panel of FIG. 11A). In addition, after being treated with the reducing agent dithiothreitol (DTT), the same five purified protein samples appeared at the size of a TNF-α monomer (17-kDa) (bottom panel of FIG. 11A).

FIG. 11B shows TNF-α activities of the five confirmed covalent disulfide-linked TNF-α variants (ID NOs: 2, 7, 9, 10, and 11), as well as the wild-type TNF-α. Three covalent disulfide-linked TNF-α variants (Protein ID NOs: 2, 9, and 11; open circle, open and solid triangles) maintained or exceeded the potency of wild-type non-covalent TNF-α (solid circle).

FIG. 12A to FIG. 12D show different configurations of TNF-α containing immunoconjugates screened for cell killing activity using the WEHI-164 co-culture assay.

FIGS. 13A to 13J show cell killing activities of different TNF-α containing immunoconjugate molecules screened using the WEHI-164 co-culture assay. Particularly, FIGS. 13A and 13B show TNF-α activities of an immunoconjugate having the configuration in FIG. 12B. The assays were performed in the absence (solid circles and up triangles) or presence (open circles and up triangles) of hFAP-M2C2 cells. The TNF-α activity was analyzed at a proliferation time of 24 hours (FIG. 21A) and 48 hours (FIG. 21B). The immunoconjugate contained a N34H point mutation of TNF-α, a scFV70 anchoring moiety, and an FN15 two-in-one masking moiety. Unconjugated wild-type TNF-α polypeptides were included as controls.

FIGS. 13C and 13D show TNF-α activities of an immunoconjugate having the configuration in FIG. 12B measuring using the WEHI-164 co-culture assay. The assays were performed in the absence (solid circles and up triangles) or presence (open circles and up triangles) of hFAP-M2C2 cells. The TNF-α activity was analyzed at a proliferation time of 24 hours (FIG. 21C) and 48 hours (FIG. 21D). The immunoconjugate contained a S95A point mutation of TNF-α, a scFV70 anchoring moiety, and an FN15 two-in-one masking moiety. Unconjugated wild-type TNF-α polypeptides were included as controls.

FIGS. 13E and 13F show TNF-α activities of an immunoconjugate having the configuration in FIG. 12B measuring using the WEHI-164 co-culture assay. The assays were performed in the absence (solid circles and up triangles) or presence (open circles and up triangles) of hFAP-M2C2 cells. The TNF-α activity was analyzed at a proliferation time of 24 hours (FIG. 21E) and 48 hours (FIG. 21F). The immunoconjugate contained a A145G point mutation of TNF-α, a scFV70 anchoring moiety, and an FN15 two-in-one masking moiety. Unconjugated wild-type TNF-α polypeptides were included as controls.

FIGS. 13G and 13H show TNF-α activities of an immunoconjugate having the configuration in FIG. 12B measuring using the WEHI-164 co-culture assay. The assays were performed in the absence (solid circles and up triangles) or presence (open circles and up triangles) of hFAP-M2C2 cells. The TNF-α activity was analyzed at a proliferation time of 24 hours (FIG. 21G) and 48 hours (FIG. 21H). The immunoconjugate contained a S147A point mutation of TNF-α, a scFV70 anchoring moiety, and an FN15 two-in-one masking moiety. Unconjugated wild-type TNF-α polypeptides were included as controls.

FIGS. 131 and 13J show TNF-α activities of an immunoconjugate having the configuration in FIG. 12B measuring using the WEHI-164 co-culture assay. The assays were performed in the absence (solid circles and up triangles) or presence (open circles and up triangles) of hFAP-M2C2 cells. The TNF-α activity was analyzed at a proliferation time of 24 hours (FIG. 21I) and 48 hours (FIG. 21J). The immunoconjugate contained a N34H point mutation of TNF-α, a SibrotuzumabSCFV anchoring moiety, and an FN15 two-in-one masking moiety. Unconjugated wild-type TNF-α polypeptides were included as controls.

FIGS. 14A to 14N show cell killing activities of different TNF-α containing immunoconjugate molecules screened using the WEHI-164 co-culture assay. Unconjugated wild-type TNF-α polypeptides were included as controls. Particularly, FIGS. 14A to 14G show TNF-α activities of an immunoconjugate having the configuration in FIG. 12D measured using the WEHI-164 co-culture assay. The assays were performed in the absence (solid circles and up triangles) or presence (open circles and up triangles) of hFAP-M2C2 cells. The immunoconjugates contained either a N34H, S147A, or S95A point mutation of TNF-α, a variant VHH anchoring moiety, and an FN15 two-in-one masking moiety.

FIGS. 14H to 14N show TNF-α activities of an immunoconjugate having the configuration in FIG. 12B measured using the WEHI-164 co-culture assay. The assays were performed in the absence (solid circles and up triangles) or presence (open circles and up triangles) of hFAP-M2C2 cells. The immunoconjugates contained either a N34H/A33T, N34H/A145G, N34H/A145V, A33T/A145G, A33T/A145V, S147A, or S95A point mutation of TNF-α, a Sibrotuzumab-SCFV anchoring moiety, and an FN15 two-in-one masking moiety.

FIG. 15A shows TNF-α activities of unconjugated wild-type TNF-α polypeptides measured using the WEHI-164 co-culture assay. The assays were performed in the absence (solid circles) or presence (open circles) of hFAP-M2C2 cells. The assays were also performed in the presence of 1.0 μg/mL ActD or 2.0 μg/mL ActD. X-axis shows the concentration (pM) of the immunoconjugate. Y-axis shows the absorbance of the immunoconjugate using a TECAN plate reader, which reflects the levels of secreted embryonic alkaline phosphatase (SEAP) and thus TNF-α activity.

FIG. 15B shows TNF-α activities of an immunoconjugate having the configuration in FIG. 12B measured using the WEHI-164 co-culture assay. The assays were performed in the absence (solid circles) or presence (open circles) of hFAP-M2C2 cells. The assays were also performed in the presence of 1.0 μg/mL ActD or 2.0 μg/mL ActD. The immunoconjugates contained a N34H point mutation of TNF-α, an scFV70 anchoring moiety, and an FN15 two-in-one masking moiety. Unconjugated wild-type TNF-α polypeptides were included as controls.

FIGS. 16A to 16C show TNF-α activities of immunoconjugates having the configuration in FIG. 12B measured using the WEHI-164 co-culture assay. The assays were performed in the absence (solid circles and up triangles) or presence (open circles and up triangles) of hFAP-M2C2 cells. X-axis shows the concentration (pM) of the immunoconjugate. Y-axis shows the activity of the immunoconjugate using a TECAN plate reader, which reflects the levels of secreted embryonic alkaline phosphatase (SEAP) and thus TNF-α activity. The immunoconjugates contained either a S95A or A33T point mutation of TNF-α, an SibrotuzumabSCFV anchoring moiety, and a variant FN15 two-in-one masking moiety. Unconjugated wild-type TNF-α polypeptides were included as controls.

FIGS. 16D to 16F show TNF-α activities of immunoconjugates having the configuration in FIG. 12D measured using the WEHI-164 co-culture assay. The assays were performed in the absence (solid circles and up triangles) or presence (open circles and up triangles) of hFAP-M2C2 cells. X-axis shows the concentration (pM) of the immunoconjugate. Y-axis shows the activity of the immunoconjugate using a TECAN plate reader, which reflects the levels of secreted embryonic alkaline phosphatase (SEAP) and thus TNF-α activity. The immunoconjugates contained either a S95A or A33T point mutation of TNF-α, a VHH anchoring moiety, and a variant FN15 two-in-one masking moiety. Unconjugated wild-type TNF-α polypeptides were included as controls.

FIGS. 17A to 17E show TNF-α activities of different immunoconjugates measured using the WEHI-164 co-culture assay. The immunoconjugate T116 (“full” immunoconjugate) shown in FIG. 17A has a configuration as shown in FIG. 7A. The immunoconjugate T117 (lacking the two-in-one masking moiety capable of binding to FAP) shown in FIG. 17B has a configuration as shown in FIG. 17C. The immunoconjugate T114 (lacking the anchoring moiety capable of binding to FAP) has a configuration shown in FIG. 17D has a configuration as shown in FIG. 17E. The assays were performed in the absence (solid circles and up triangles) or presence (open circles and up triangles) of FAP-expressing cell lines. Unconjugated wild-type TNF-α polypeptides were included as controls. These results demonstrates the impact of FAP-binding capability of an immunoconjugate molecule on the activation of cell killing activity of TNF-α containing immunoconjugate.

FIGS. 18A to 18L show screening of various TNF-α containing immunoconjugate molecules constructed with different anchoring moieties and/or two-in-one masking moiety derived from FAP/TNF-α two-in-one antibody FN15 using the WEHI-164 co-culture assay.

FIGS. 19A to 19E show TNF-α activities of the immunoconjugates having the configuration in FIG. 12C (T102 containing a S95A point mutation of TNF-α, a VHH anchoring moiety and the same FN15 two-in-one masking moiety). measured using the WEHI-164 co-culture assay. The assays were performed in the absence (solid circles, up triangles, diamonds, down triangles, and squares) or presence (open circles, up triangles, diamonds, down triangles, and squares) of FAP. The assays were performed with the following concentrations of Actinomycin D (ActD): 0.0 μg/mL ActD (FIG. 14A), 0.2 μg/mL ActD (FIG. 14B), 0.5 μg/mL ActD (FIG. 14C), 1.0 μg/mL ActD (FIG. 14D), and 2.0 μg/mL ActD (FIG. 14E). These data suggest that in certain instances (e.g., higher confluency cell assays), addition of Actinomycin D can increase cell killing activity.

FIGS. 20A to 20C show TNF-α activities of the T102 immunoconjugate measured using the WEHI-164 co-culture assay. The assays were performed in the absence (solid circles and up triangles) or presence (open circles, up triangles, and squares) of soluble FAP. Unconjugated wild-type TNF-α polypeptides were included as controls. As shown, 10 nM soluble FAP was unable to induce T102 to kill cells without FAP expression (FIG. 13B, open squares), suggesting this immunoconjugate molecule requires cell-surface anchorage (via expressed FAP) to unleash full activity.

FIGS. 21A to 21E show TNF-α activities of the T102 immunoconjugate in Tumor cell line WEHI-164 were co-cultured in the presence or absence of a WEHI-164 cell line ectopically expressing FAP (WEHI-164 Clone M2C2). Cell viability was determined with the alamarBlue assay (Invitrogen, Waltham, Massachusetts) monitoring fluorescence (540 nm excitation, 590 nm emission). Data were plotted as relative fluorescence (y-axis, relative fluorescence units) vs. immunoconjugate concentration (x-axis, in picomolar). The signal of the y-axis is proportional to the number of viable cells in the assay. The following concentrations of Actinomycin D (ActD): 0.0 μg/mL ActD (FIG. 12B), 0.1 μg/mL ActD (FIG. 12C), 0.2 μg/mL ActD (FIG. 12D), 0.4 μg/mL ActD (FIG. 12E), and 0.6 μg/mL ActD (FIG. 12F) were screened. Unconjugated wild-type TNF-α polypeptides were included as controls. These figures demonstrate that the capacity of TNF-α containing immunoconjugates as described herein in enabling bystander killing of cells without FAP expression when mixed with cells containing FAP (50:50 mix). In the presence of Actinomycin D, the extent of killing was similar with T102 when cells only contain FAP (black, open squares) and when only 50% of cells contain FAP (grey, open squares).

FIG. 22A shows TNF-α activities of measured using the HEK Blue TNF-α and HEK Blue TNF-α FAP reporter cell lines. The results show that the potency of TNF-α immunoconjugate T017 in HEK Blue TNF-α FAP assay (solid down triangle) in the presence of HEK Blue TNF-α with FAP in cis-presentation was comparable to unconjugated TNF-α (solid circle) in HEK Blue TNF-α assay. In the absence of surface FAP in HEK Blue TNF-α assay, the immunoconjugate T017 was almost silent (open down triangle).

FIG. 22B shows TNF-α activities of measured using the HEK Blue TNF-α reporter cell line co-cultured with either HEK 293T cells or HEK 293T-FAP cells. The results show that TNF-α immunoconjugate T017 was silent when co-cultured with HEK 293T cells (open down triangle). When co-cultured with HEK 293T-FAP cells in the presence of surface FAP at trans-presentation (solid down triangle), T017 was activated but its potency was ˜100 folds less than the TNF-α (solid circle).

FIG. 22C shows TNF-α activities of an immunoconjugate having the configuration in FIG. 12C measured using the WEHI-164 and WEHI-164-FAP apoptotic assays. The results show that TNF-α immunoconjugate T102 in WEHI-164-FAP assay (solid down triangle) in the presence of surface FAP at cis-presentation was more potent than unconjugated TNF-α (solid circle) in WEHI-164 assay. In the absence of surface FAP in WEHI-164 assay, the immunoconjugate T102 was silent (open down triangle).

FIG. 22D shows TNF-α activities of measured using the WEHI-164 and WEHI-164-FAP co-culture assays. The results show that increasing amount of Actinomycin D increased WEHI-164's apoptotic sensitivity to TNF-α (open and solid circles). In the presence of 1.0 μg Actinomycin D, T102 caused similar apoptotic response as TNF-α (solid down triangle) which indicates the 50% WEHI-164 cells were killed too likely due to the trans-presentation of FAP from the rest 50% WEHI-164-FAP cells. X-axis shows the concentration (pM) of the immunoconjugate. Y-axis shows normalized cell viability. The immunoconjugate contained a S95A point mutation of TNF-α, a VHH anchoring moiety, and an FN15 two-in-one masking moiety. Unconjugated wild-type TNF-α polypeptides were included as controls.

FIGS. 23A and 23B show the clinical potential of the TNF-α immunoconjugate molecules described herein. FIG. 23A shows that human soft tissue sarcoma cells (SA3831, CrownBio) express high levels of FAP ((˜1×10⁶ FAP/cell). FIG. 23B shows a TNF-α containing immunoconjugate molecule as described herein is synergistic with chemotherapeutic agent Actinomycin D on human primary tumor cells within therapeutically relevant concentrations.

FIG. 24A left panel shows the sizes of the purified immunoconjugate protein and components thereof confirmed via SDS-PAGE. Right panel shows accelerated stability of an TNF-α containing immunoconjugate as described herein measured using size-exclusion chromatography (SEC). As shown in the figure, the protein remained stable after storage at 40° C. for four weeks, or 5 rounds of freeze-thaw cycles.

FIG. 24B shows serum concentration (ng/mL) of the TNF-α containing immunoconjugate or a non-conjugated TNFα polypeptide days after administration to a test subject, and the measurement of half life (hour), which demonstrating that the immunoconjugate molecule had more than 100 folds increased half life compared to unconjugated TNF-α polypeptide.

FIG. 24C shows body weight change in mice administered PBS, or 4 pg unconjugated TNF-α polypeptide or 200 pg immunoconjugate molecule having the configuration in FIG. 12B (T098 containing a S95A point mutation of TNF-α, a scFV anchoring moiety derived from sibrotuzumab and FN15 two-in-one masking moiety). As shown T098 was less toxic compared to unconjugated TNF-α polypeptide as measured by body weight reduction following administration.

FIGS. 25A to 25C show tumor regression and efficacy in various animal models following administration of T098 immunoconjugate. FIG. 25A shows that the administration of the immunoconjugate was able to inhibit tumor growth in Balb/c mouse cancer model. FIG. 25B shows that the administration of the immunoconjugate did not change body weight in the mice. FIG. 25C shows that the administration of the immunoconjugate did not change tumor volume in M-NSG immunocompromised mice.

FIG. 26A shows tumor growth inhibition and body weight change in Balb/c mice following administration of 4 μg of unconjugated TNF-α polypeptide, 1 μg Actinomycin D, or 100 μg TNF-α containing immunoconjugate (T102) in combination with 1 μg Actinomycin D. The result shows that the administration of the combination treatment inhibited tumor growth comparable to unconjugated TNF-α polypeptide, but significantly reduced toxicity of the treatment as measured by body weight reduction following administration.

FIG. 26B shows Hematoxylin and Eosin (H&E) staining 48 hours after first dosing of PBS (control) or 25 μg TNF-α containing immunoconjugate as described herein, indicting the administered immunoconjugate molecule induced destruction and lymphocyte infiltration in tumor.

FIG. 26C shows the tumor inhibition efficacy of an example of a TNF-α containing immunoconjugates molecule as described herein. Two animal models were used: WEHI-164 WT and WEHI-164-FAP. The WEHI-164-FAP was engineered to express FAP, while the WEHI-164 WT model did not express FAP. This result demonstrates that tumor inhibition efficacy of this TNF-α containing immunoconjugates depends on the presence of FAP to unshield TNF-α activity.

### 5. DETAILED DESCRIPTION

The present disclosure provides immunoconjugate molecules comprising a cytokine polypeptide. The present disclosure also provides, in certain embodiments, polynucleotides and vectors comprising sequences encoding such immunoconjugate molecules, and compositions, reagents, and kits comprising such immunoconjugate molecules. In related aspect, provided herein are also methods for delivery and/or activation of a cytokine activity at a target site, or reduce toxicity and/or other side-effects associated with systemic exposure to the cytokine activity in a subject through the use of the immunoconjugate molecules according to the present disclosure.

### 5.1 General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); Phage Display in Biotechnology and Drug Discovery (Sidhu and Geyer eds., 2d ed. 2005); Phage Display: a Laboratory Manual (Barbas et al. eds., 2004); and Antibody Engineering Vols 1 and 2 (Kontermann and Dübel eds., 2d ed. 2010).

### 5.2 Terminology

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

As used herein, the singular terms “a,” “an,” and “the” include the plural reference unless the context clearly indicates otherwise.

Unless otherwise indicated, the terms “oligonucleotides” and “nucleic acids” are used interchangeably and are written left to right in 5′ to 3′ orientation; amino acid sequences are written left to right in amino to carboxy orientation, respectively. Therefore, in general, the codon at the 5′-terminus of an oligonucleotide will correspond to the N-terminal amino acid residue that is incorporated into a translated protein or peptide product. Similarly, in general, the codon at the 3′-terminus of an oligonucleotide will correspond to the C-terminal amino acid residue that is incorporated into a translated protein or peptide product. It is to be understood that this present disclosure is not limited to the particular methodology, protocols, and reagents described, as these may vary, depending upon the context they are used by those of skill in the art.

The term “soluble” when used in connection with a peptide or polypeptide (e.g., a cytokine or antigen) indicates that such peptide or polypeptide is not associated with solid surface or attached to the surface of a cell. In some embodiments, in contrast to a cell surface antigen, a soluble antigen is secreted to the extracellular space. According to the present disclosure, a tumor associated antigen can be expressed on the cell surface or soluble.

The term “TNF-α” or “tumor necrosis factor alpha” as used herein, refers to any native TNF-α from any vertebrate source, including mammals such as primates (e.g., humans) and rodents (e.g., mice and rats), unless otherwise indicated. The term encompasses unprocessed TNF-α as well as any form of TNF-α (e.g., protein complex containing multimerized TNF-α) that results from processing in the cell. The term also encompasses naturally occurring variants of TNF-α, e.g., splice variants or allelic variants. The amino acid sequence of an exemplary human TNF-α is VRSSSRTPSDKPVAHVVANPQAEGQLQWLNRRANALLANGVELRDNQLVVPSEGLYLIYS QVLFKGQGCPSTHVLLTHTISRIAVSYQTKVNLLSAIKSPCQRETPEGAEAKPWYEPIYLGGV FQLEKGDRLSAEINRPDYLDFAESGQVYFGIIAL (SEQ ID NO:1). In some embodiments, TNF-α polypeptides can form a homotrimer complex through the extensive trimer interface of the TNF-α polypeptides. Eck and Sprang, J Biol Chem. 1989 Oct. 15; 264(29):17595-605.

The term “TNF-α mutant” or “mutant TNF-α” as used herein is intended to encompass any mutant forms of various forms of the TNF-α molecule including full-length TNF-α, truncated forms of TNF-α and forms where TNF-α polypeptide containing one or more amino acid mutations in its sequence. “Full-length” when used in reference to TNF-α is intended to mean the mature, natural length TNF-α molecule. For example, full-length human TNF-α refers to a molecule that has 157 amino acids (see e.g., SEQ ID NO:1). The various forms of TNF-α mutants are characterized in having at least one amino acid mutation affecting the interaction of TNF-α with one or both of its two receptors: TNFR1 and TNFR2. This mutation may involve substitution, deletion, truncation or modification of the wild-type amino acid residue normally located at that position. Unless otherwise indicated, a TNF-α mutant may be referred to herein as a TNF-α mutant peptide sequence, TNF-α mutant polypeptide, TNF-α mutant protein or TNF-α mutant analog. Designation of various forms of TNF-α is herein made with respect to the sequence shown in SEQ ID NO:1. Various designations may be used herein to indicate the same mutation. For example, a mutation from serine at position 147 to alanine can be indicated as 147A, A147, A147, S147A, or Ser147Ala. The numbering of the positions of mutated amino acid residues is according to the wild-type human TNF-α sequence (SEQ ID NO:1). Without being bound by the theory, it is contemplated that single mutations A33T, N34A, N34H, Y87A, Y87F, S95A, A145G, A145V, S147A, and S147G each can reduce binding affinity between TNF-α and one or both of its receptors TNF-α receptor 1 (TNFR1) and TNF-α receptor 2 (TNFR2). In some embodiments, the wild-type TNF-α can be mutated to modulate self-interaction affinity between TNF-α polypeptides during oligomerization. In specific embodiments, wild type TNF-α can be mutated to include one or more cysteine residues, such that multimerization between TNF-α or TNF-α containing immunoconjugate molecules can be stabilized through the formation of the covalent disulfide bond between the added cysteine residues. Exemplary mutations that can facilitate multimerization of TNF-α or TNF-α contain immunoconjugate molecules include P08C, L55C, Q102C, E104C, S95C, G148C, I97C, Y115C, H73C, and P113C. In specific embodiments, a mutant TNF-α contains one or more mutations selected from P08C, L55C, Q102C, E104C, S95C, G148C, I97C, Y115C, H73C, and P113C. In specific embodiments, a mutant TNF-α contains double mutations selected from P08C, L55C, Q102C, E104C, S95C, G148C, I97C, Y115C, H73C, and P113C. In yet specific embodiments, a mutant TNF-α contains double mutations of (i) P08C and L55C, (ii) Q102C and E104C, (iii) S95C and G148C, (iv) I97C and Y115C, (v) H73C and P113C, or (vi) any combination of (i) to (v). Additional mutant TNF-α polypeptides that can be used in connection with the present disclosure include those described in, for example, Loetscher et al., Journal of Biological Chemistry, 268(35): 26350-26357, which is hereby incorporated by reference in its entirety.

As used herein, a “wild-type” form of TNF-α is a form of TNF-α that is otherwise the same as the mutant TNF-α polypeptide except that the wild-type form has a wild-type amino acid at each amino acid position of the mutant TNF-α polypeptide. For example, if the TNF-α mutant is the full-length TNF-α (i.e., TNF-α not fused or conjugated to any other molecule), the wild-type form of this mutant is full-length native TNF-α. If the TNF-α mutant is a fusion between TNF-α and another polypeptide encoded downstream of TNF-α (e.g., an antibody chain) the wild-type form of this TNF-α mutant is TNF-α with a wild-type amino acid sequence fused to the same downstream polypeptide. Furthermore, if the TNF-α mutant is a truncated form of TNF-α (the mutated or modified sequence within the non-truncated portion of TNF-α) then the wild-type form of this TNF-α mutant is a similarly truncated TNF-α that has a wild-type sequence. For the purpose of comparing TNF-α receptor binding affinity or biological activity of various forms of TNF-α mutants to the corresponding wild-type form of TNF-α, the term wild-type encompasses forms of TNF-α comprising one or more amino acid mutation that does not affect TNF-α receptor binding compared to the naturally occurring, native TNF-α. In certain embodiments according to the invention the wild-type TNF-α polypeptide to which the mutant TNF-α polypeptide is compared comprises the amino acid sequence of SEQ ID NO:1.

As used herein, a “corresponding site” in a polypeptide with respect to a reference polypeptide sequence can be determined by aligning and comparing the sequences. After aligning the sequences and introducing gaps, if necessary, to achieve the maximum percent sequence identity, those pairs of sites in the two sequences that are considered aligned with one another are corresponding sites. Alignment for purposes of determining corresponding sites in two amino acid sequences can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

The term “TNFR1” or “TNF-α receptor 1” as used herein, refers to any native TNFR1 from any vertebrate source, including mammals such as primates (e.g., humans) and rodents (e.g., mice and rats), unless otherwise indicated. The term encompasses “full-length”, unprocessed TNFR1 as well as any form of TNFR1 that results from processing in the cell. The term also encompasses naturally occurring variants of TNFR1, e.g., splice variants or allelic variants. In certain embodiments TNFR1 is human TNFR1. The amino acid sequence of an exemplary human TNFR1 is shown below:

(SEQ ID NO: 2)
GLSTVPDLLLPLVLLELLVGIYPSGVIGLVPHLGDREKRDSVCPQGKYI
HPQNNSICCTKCHKGTYLYNDCPGPGQDTDCRECESGSFTASENHLRHC
LSCSKCRKEMGQVEISSCTVDRDTVCGCRKNQYRHYWSENLFQCFNCSL
CLNGTVHLSCQEKQNTVCTCHAGFFLRENECVSCSNCKKSLECTKLCLP
QIENVKGTEDSGTTVLLPLVIFFGLCLLSLLFIGLMYRYQRWKSKLYSI
VCGKSTPEKEGELEGTTTKPLAPNPSFSPTPGFTPTLGFSPVPSSTFTS
SSTYTPGDCPNFAAPRREVAPPYQGADPILATALASDPIPNPLQKWEDS
AHKPQSLDTDDPATLYAVVENVPPLRWKEFVRRLGLSDHEIDRLELQNG
RCLREAQYSMLATWRRRTPRREATLELLGRVLRDMDLLGCLEDIEEALC
GPAALPPAPSLLR.

The term “TNFR2” or “TNF-α receptor 2” as used herein, refers to any native TNFR2 from any vertebrate source, including mammals such as primates (e.g., humans) and rodents (e.g., mice and rats), unless otherwise indicated. The term encompasses “full-length”, unprocessed TNFR2 as well as any form of TNFR2 that results from processing in the cell. The term also encompasses naturally occurring variants of TNFR2, e.g., splice variants or allelic variants. In certain embodiments TNFR2 is human TNFR2. The amino acid sequence of an exemplary human TNFR2 is shown below:

(SEQ ID NO: 3)
APVAVWAALAVGLELWAAAHALPAQVAFTPYAPEPGSTCRLREYYDQTA
QMCCSKCSPGQHAKVFCTKTSDTVCDSCEDSTYTQLWNWVPECLSCGSR
CSSDQVETQACTREQNRICTCRPGWYCALSKQEGCRLCAPLRKCRPGFG
VARPGTETSDVVCKPCAPGTFSNTTSSTDICRPHQICNVVAIPGNASMD
AVCTSTSPTRSMAPGAVHLPQPVSTRSQHTQPTPEPSTAPSTSFLLPMG
PSPPAEGSTGDFALPVGLIVGVTALGLLIIGVVNCVIMTQVKKKPLCLQ
REAKVPHLPADKARGTQGPEQQHLLITAPSSSSSSLESSASALDRRAPT
RNQPQAPGVEASGAGEARASTGSSDSSPGGHGTQVNVTCIVNVCSSSDH
SSQCSSQASSTMGDTDSSPSESPKDEQVPFSKEECAFRSQLETPETLLG
STEEKPLPLGVPDAGMKPS.

The term “tumor associated antigen” or “TAA”, as used herein, refers to an antigen expressed by a cancer cell or in the stroma of a solid tumor. The TAA can be a protein, nucleic acid, lipid or other antigen. In certain embodiments, the TAA can be a cell-surface expressed TAA. In the context of a solid tumor, the TAA can be expressed in the stroma of a solid tumor mass. The term “stroma” as used herein refers to components in a solid tumor mass other than a cancer cell. For example, the stroma can include fibroblasts, epithelial cells, other blood vessel components or extracellular matrix components. As used herein, the term “stroma” does not include components of the immune system, such as immune cells (e.g., B-cells, T-cells, dendritic cells, macrophages, natural killer cells, and the like). Various TAAs are known in the art. Identifying TAA can be performed using methods known in the art, such as disclosed in Zhang et al., Methods Mol. Biol., 520:1-10 (2009); the content of which is enclosed herein by reference.

The term “fibroblast activation protein” or “FAP” as used herein, refers to any native FAP from any vertebrate source, including mammals such as primates (e.g., humans) and rodents (e.g., mice and rats), unless otherwise indicated. The term encompasses unprocessed FAP as well as any form of FAP that results from processing in the cell. The term also encompasses naturally occurring variants of FAP, e.g., splice variants or allelic variants. The amino acid sequence of an exemplary human FAP is shown below

(SEQ ID NO: 4)
MKTWVKIVFGVATSAVLALLVMCIVLRPSRVHNSEENTMRALTLKDILN
GTFSYKTFFPNWISGQEYLHQSADNNIVLYNIETGQSYTILSNRTMKSV
NASNYGLSPDRQFVYLESDYSKLWRYSYTATYYIYDLSNGEFVRGNELP
RPIQYLCWSPVGSKLAYVYQNNIYLKQRPGDPPFQITFNGRENKIFNGI
PDWVYEEEMLATKYALWWSPNGKFLAYAEFNDTDIPVIAYSYYGDEQYP
RTINIPYPKAGAKNPVVRIFIIDTTYPAYVGPQEVPVPAMIASSDYYFS
WLTWVTDERVCLQWLKRVQNVSVLSICDFREDWQTWDCPKTQEHIEESR
TGWAGGFFVSTPVFSYDAISYYKIFSDKDGYKHIHYIKDTVENAIQITS
GKWEAINIFRVTQDSLFYSSNEFEEYPGRRNIYRISIGSYPPSKKCVTC
HLRKERCQYYTASFSDYAKYYALVCYGPGIPISTLHDGRTDQEIKILEE
NKELENALKNIQLPKEEIKKLEVDEITLWYKMILPPQFDRSKKYPLLIQ
VYGGPCSQSVRSVFAVNWISYLASKEGMVIALVDGRGTAFQGDKLLYAV
YRKLGVYEVEDQITAVRKFIEMGFIDEKRIAIWGWSYGGYVSSLALASG
TGLFKCGIAVAPVSSWEYYASVYTERFMGLPTKDDNLEHYKNSTVMARA
EYFRNVDYLLIHGTADDNVHFQNSAQIAKALVNAQVDFQAMWYSDQNHG
LSGLSTNHLYTHMTHFLKQCFSLSD.

The term “Tumor-associated calcium signal transducer 2,” or “Trop-2” as used herein, refers to any native Trop-2 from any vertebrate source, including mammals such as primates (e.g., humans) and rodents (e.g., mice and rats), unless otherwise indicated. The term encompasses unprocessed Trop-2 as well as any form of Trop-2 that results from processing in the cell. The term also encompasses naturally occurring variants of Trop-2, e.g., splice variants or allelic variants. The amino acid sequence of an exemplary human Trop-2 is shown below

(SEQ ID NO: 5)
ARGPGLAPPPLRLPLLLLVLAAVTGHTAAQDNCTCPTNKMTVCSPDGPG
GRCQCRALGSGMAVDCSTLTSKCLLLKARMSAPKNARTLVRPSEHALVD
NDGLYDPDCDPEGRFKARQCNQTSVCWCVNSVGVRRTDKGDLSLRCDEL
VRTHHILIDLRHRPTAGAFNHSDLDAELRRLFRERYRLHPKFVAAVHYE
QPTIQIELRQNTSQKAAGDVDIGDAAYYFERDIKGESLFQGRGGLDLRV
RGEPLQVERTLIYYLDEIPPKFSMKRLTAGLIAVIVVVVVALVAGMAVL
VITNRRKSGKYKKVEIKELGELRKEPSL.

The term “tumor microenvironment” refers to any and all elements of the neoplasia milieu that creates a structural and/or functional environment for the neoplastic process to survive, expand, and/or spread. As a non-limiting example, a tumor microenvironment is constituted by the cells, molecules, fibroblasts, extracellular matrix and/or blood vessels that surround and/or feed one or more neoplastic cells, such as a solid tumor. In certain embodiments, the neoplastic disease is a solid tumor. Exemplary cells or tissue within the tumor microenvironment include, but are not limited to, tumor vasculature, tumor infiltrating lymphocytes, fibroblast reticular cells, endothelial progenitor cells (EPC), cancer-associated fibroblasts, pericytes, other stromal cells, components of the extracellular matrix (ECM), dendritic cells, antigen presenting cells, T-cells, regulatory T-cells, macrophages, neutrophils, and other immune cells located proximal to a tumor. Exemplary cellular functions affecting the tumor microenvironment include, but are not limited to, production of cytokines and/or chemokines, response to cytokines, antigen processing and presentation of peptide antigen, regulation of leukocyte chemotaxis and migration, regulation of gene expression, complement activation, regulation of signaling pathways, cell-mediated cytotoxicity, cell-mediated immunity, humoral immune responses, and innate immune responses, etc.

The term “antibody,” “immunoglobulin,” or “Ig” is used interchangeably herein, and is used in the broadest sense and specifically encompasses, for example, individual monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments of antibodies, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptidic chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, such as a TNF-α polypeptide, a TNF-α fragment, or a TNF-α epitope. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments such as TNF-α-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments such as TNF-α-binding fragments) include single-chain Fvs (scFv) (e.g., including monospecific, bispecific, etc.), Fab fragments (e.g., including monospecific, bispecific, etc.), F(ab′) fragments, F(ab)₂ fragments, F(ab′)₂ fragments, disulfide-linked Fvs (dsFv), Fd fragments, Fv fragments, diabody, triabody, tetrabody, minibody, and single domain antibody (VHH or nanobody). In particular, antibodies provided herein include immunoglobulin molecules and immunologically active portions of immunoglobulin molecules, for example, antigen-binding domains or molecules that contain an antigen-binding site that binds to an TNF-α antigen (e.g., one or more CDRs of an anti-TNF-α antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule.

The term “monoclonal antibody” as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell, wherein the antibody binds to only one epitope as determined, for example, by ELISA or other antigen-binding or competitive binding assay known in the art. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., 1991, Nature 352:624-28 and Marks et al., 1991, J. Mol. Biol. 222:581-97, for example. Other methods for the preparation of clonal cell lines and of monoclonal antibodies expressed thereby are well known in the art. See, e.g., Short Protocols in Molecular Biology (Ausubel et al. eds., 5th ed. 2002). Exemplary methods of producing monoclonal antibodies are provided in the Examples herein.

“Polyclonal antibodies” as used herein refer to an antibody population generated in an immunogenic response to a protein having many epitopes and thus includes a variety of different antibodies directed to the same or different epitopes within the protein. Methods for producing polyclonal antibodies are known in the art (See, e.g., Short Protocols in Molecular Biology (Ausubel et al. eds., 5th ed. 2002)).

An “antigen” is a predetermined antigen to which an antibody can selectively bind. A target antigen may be a polypeptide, carbohydrate, nucleic acid, lipid, hapten, or other naturally occurring or synthetic compound. In some embodiments, the target antigen is a polypeptide.

The terms “antigen-binding fragment,” “antigen-binding domain,” “antigen-binding region,” and similar terms refer to that portion of an antibody, which comprises the amino acid residues that interact with an antigen and confer on the binding agent its specificity and affinity for the antigen (e.g., the CDRs).

A “bispecific antibody” as used herein refers to an antibody or antigen binding fragment thereof that is capable of binding with two different target antigens. A “two-in-one antibody” as used herein refers to a bispecific antibody that is capable of binding with two different target antigens via a single antigen binding domain. In some embodiments, the target antigens compete with one another for binding with the single antigen binding domain of the two-in-one antibody, such that the two-in-one antibody, upon binding with one target antigen, dissociates from the other target antigen.

An “epitope” is the site on the surface of an antigen molecule to which a single antibody molecule binds, such as a localized region on the surface of an antigen, such as a TNF-α polypeptide or a TNF-α polypeptide fragment, that is capable of being bound to one or more antigen binding regions of an antibody, and that has antigenic or immunogenic activity in an animal, such as a mammal (e.g., a human), that is capable of eliciting an immune response. An epitope having immunogenic activity is a portion of a polypeptide that elicits an antibody response in an animal. An epitope having antigenic activity is a portion of a polypeptide to which an antibody binds as determined by any method well known in the art, including, for example, by an immunoassay. Antigenic epitopes need not necessarily be immunogenic. Epitopes often consist of chemically active surface groupings of molecules such as amino acids or sugar side chains and have specific three dimensional structural characteristics as well as specific charge characteristics. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure. Induced epitopes are formed when the three dimensional structure of the protein is in an altered conformation, such as following activation or binding of another protein or ligand. Generally, an antigen has several or many different epitopes and may react with many different antibodies. In certain embodiments, an antigen (e.g., FAP) can have more than one epitopes that are recognized and bound by different anti-FAP antibodies. In certain embodiments, different anti-FAP antibodies compete with one another for binding with the same epitope of FAP.

An antibody binds “an epitope,” “essentially the same epitope,” or “the same epitope” as a reference antibody, when the two antibodies recognize identical, overlapping, or adjacent epitopes in a three-dimensional space. The most widely used and rapid methods for determining whether two antibodies bind to identical, overlapping, or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent, or enzyme labels.

“Epitope mapping” is the process of identifying the binding sites, or epitopes, of antibodies on their target antigens. “Epitope binning” is the process of grouping antibodies based on the epitopes they recognize. More particularly, epitope binning comprises methods and systems for discriminating the epitope recognition properties of different antibodies, using competition assays combined with computational processes for clustering antibodies based on their epitope recognition properties and identifying antibodies having distinct binding specificities.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as TNF-α, is the affinity of the antibody or functional fragment for that epitope. The ratio of dissociation rate (k_{off}) to association rate (kₒₙ) of an antibody to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent TNF-α, come in contact with antibodies containing multiple binding sites, the interaction of antibody with antigen at one site will increase the probability of a reaction at a second site. The strength of such multiple interactions between a multivalent antibody and antigen is called the avidity. The avidity of an antibody can be a better measure of its binding capacity than is the affinity of its individual binding sites. For example, high avidity can compensate for low affinity as is sometimes found for pentameric IgM antibodies, which can have a lower affinity than IgG, but the high avidity of IgM, resulting from its multivalence, enables it to bind antigen effectively.

The terms “antibodies that specifically bind to an antigen,” “antibodies that specifically bind to an epitope” and analogous terms are also used interchangeably herein and refer to antibodies that specifically bind to the antigen, or fragment, or epitope of the antigen. An antibody that specifically binds to an antigen can be identified, for example, by immunoassays, Biacore©, or other techniques known to those of skill in the art. An antibody binds specifically to an antigen when it binds to the antigen with higher affinity than to any cross-reactive antigen as determined using experimental techniques, such as radioimmunoassays (RIA) and enzyme linked immunosorbent assays (ELISAs). Typically, a specific or selective reaction will be at least twice background signal or noise and may be more than 10 times background. See, e.g., Fundamental Immunology 332-36 (Paul ed., 2d ed. 1989) for a discussion regarding antibody specificity. An antibody which “binds an antigen of interest” (e.g., a target antigen such as TNF-α) is one that binds the antigen with sufficient affinity such that the antibody is useful as a therapeutic agent in targeting a cell or tissue expressing the antigen, and does not significantly cross-react with other proteins. In such embodiments, the extent of binding of the antibody to a “non-target” protein will be less than about 10% of the binding of the antibody to its particular target protein, for example, as determined by fluorescence activated cell sorting (FACS) analysis or RIA. With regard to the binding of an antibody to a target molecule, the term “specific binding,” “specifically binds to,” or “is specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding,” “specifically binds to,” or “is specific for” a particular polypeptide or an epitope on a particular polypeptide target as used herein refers to binding where a molecule binds to a particular polypeptide or epitope on a particular polypeptide without substantially binding to any other polypeptide or polypeptide epitope. In certain embodiments, an antibody that binds to an antigen of the present disclosure has a dissociation constant (K_{D}) of less than or equal to 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM.

The term “compete” when used in the context of antibodies (e.g., antibodies and binding proteins that bind to a cell surface antigen and compete for the same epitope or binding site on a target) means competition as determined by an assay in which the antibody (or binding fragment) thereof under study prevents or inhibits the specific binding of a reference molecule (e.g., a reference ligand or reference antigen-binding protein, such as a reference antibody) to a common antigen (e.g., FAP or a fragment thereof). Numerous types of competitive binding assays can be used to determine if a test antibody competes with a reference antibody for binding to an antigen (e.g., human FAP). Examples of assays that can be employed include solid phase direct or indirect RIA, solid phase direct or indirect enzyme immunoassay (EIA), sandwich competition assay (see, e.g., Stahli et al., 1983, Methods in Enzymology 9:242-53), solid phase direct biotin-avidin EIA (see, e.g., Kirkland et al., 1986, J. Immunol. 137:3614-19), solid phase direct labeled assay, solid phase direct labeled sandwich assay (see, e.g., Harlow and Lane, Antibodies, A Laboratory Manual (1988)), solid phase direct label RIA using I-125 label (see, e.g., Morel et al., 1988, Mol. Immunol. 25:7-15), and direct labeled RIA (Moldenhauer et al., 1990, Scand. J. Immunol. 32:77-82). Typically, such an assay involves the use of a purified antigen (e.g., TNF-α) bound to a solid surface, or cells bearing either of an unlabelled test antigen-binding protein (e.g., test anti-TNF-α antibody) or a labeled reference antigen-binding protein (e.g., reference anti-TNF-α antibody). Competitive inhibition may be measured by determining the amount of label bound to the solid surface or cells in the presence of the test antigen-binding protein. Usually the test antigen-binding protein is present in excess. Antibodies identified by competition assay (competing antibodies) include antibodies binding to the same epitope as the reference antibody and/or antibodies binding to an adjacent epitope sufficiently proximal to the epitope bound by the reference for antibodies steric hindrance to occur. Additional details regarding methods for determining competitive binding are described herein. Usually, when a competing antibody protein is present in excess, it will inhibit specific binding of a reference antibody to a common antigen by at least 30%, for example 40%, 45%, 50%, 55%, 60%, 65%, 70%, or 75%. In some instance, binding is inhibited by at least 80%, 85%, 90%, 95%, 96%, 97%, 98%, 99%, or more.

The term “heavy chain” when used in reference to an antibody refers to a polypeptidic chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4. A heavy chain can be a human heavy chain.

The term “light chain” when used in reference to an antibody refers to a polypeptidic chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains. Light chain amino acid sequences are well known in the art. A light chain can be a human light chain.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a p sheet configuration, connected by three hypervariable regions, which form loops connecting, and in some cases form part of, the R sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering as in Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

A “CDR” refers to one of three hypervariable regions (H1, H2 or H3) within the non-framework region of the immunoglobulin (Ig or antibody) VH 3-sheet framework, or one of three hypervariable regions (L1, L2 or L3) within the non-framework region of the antibody VL 3-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., 1997, J. Biol. Chem. 252:6609-16; Kabat, 1978, Adv. Prot. Chem. 32:1-75). CDR region sequences also have been defined structurally by Chothia as those residues that are not part of the conserved β-sheet framework, and thus are able to adapt different conformations (Chothia and Lesk, 1987, J. Mol. Biol. 196:901-17). Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art.

The term “hypervariable region,” “HVR,” or “HV,” when used herein refers to the regions of an antibody variable region that are hypervariable in sequence and/or form structurally defined loops. Generally, antibodies comprise six hypervariable regions, three in the VH (H1, H2, H3) and three in the VL (L1, L2, L3). A number of hypervariable region delineations are in use and are encompassed herein. The Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dubel eds., 2d ed. 2010)). The “contact” hypervariable regions are based on an analysis of the available complex crystal structures. The residues from each of these hypervariable regions or CDRs are noted below.

Recently, a universal numbering system has been developed and widely adopted, ImMunoGeneTics (IMGT) Information System® (Lafranc et al., 2003, Dev. Comp. Immunol. 27(1):55-77). IMGT is an integrated information system specializing in immunoglobulins (IG), T cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Pluckthun, 2001, J. Mol. Biol. 309: 657-70. Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). In some embodiments, the CDRs are as defined by the IMGT numbering system. In other embodiments, the CDRs are as defined by the Kabat numbering system. In certain embodiments, the CDRs are as defined by the AbM numbering system. In other embodiments, the CDRs are as defined by the Chothia system. In yet other embodiments, the CDRs are as defined by the Contact numbering system.

| | IMGT | Kabat | AbM | Chothia | Contact |
|---|---|---|---|---|---|
| V_{H} CDR1 | 27-38 | 31-35 | 26-35 | 26-32 | 30-35 |
| V_{H} CDR2 | 56-65 | 50-65 | 50-58 | 53-55 | 47-58 |
| V_{H} CDR3 | 105-117 | 95-102 | 95-102 | 96-101 | 93-101 |
| V_{L} CDR1 | 27-38 | 24-34 | 24-34 | 26-32 | 30-36 |
| V_{L} CDR2 | 56-65 | 50-56 | 50-56 | 50-52 | 46-55 |
| V_{L} CDR3 | 105-117 | 89-97 | 89-97 | 91-96 | 89-96 |

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH. As used herein, the terms “HVR” and “CDR” are used interchangeably.

The term “constant region” or “constant domain” refers to a carboxyl terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

A “functional Fc region” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); antibody-dependent cellular phagocytosis (ADCP); cytokine secretion, downregulation of cell surface receptors (e.g., B cell receptor), and B cell activation, etc. Such effector functions generally require the Fc region to be combined with a binding region or binding domain (e.g., an antibody variable region or domain) and can be assessed using various assays as disclosed.

An “activating Fc receptor” is an Fc receptor that following engagement by an Fc region of an antibody elicits signaling events that stimulate the receptor-bearing cell to perform effector functions. Exemplary activating Fc receptors include FcγRIIIα (CD16α), FcγRI (CD64), FcγRIIα (CD32), and FcαRI (CD89).

A “native sequence Fc region” comprises an amino acid sequence identical to the amino acid sequence of an Fc region found in nature, and not manipulated, modified, and/or changed (e.g., isolated, purified, selected, including or combining with other sequences such as variable region sequences) by a human. Native sequence human IgG1 Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof. For example, a native human IgG1 Fc region amino acid sequence is provided below:

(SEQ ID NO: 6)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPGK.

A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith. For example, a variant.

A “modification” of an amino acid residue/position refers to a change of a primary amino acid sequence as compared to a starting amino acid sequence, wherein the change results from a sequence alteration involving said amino acid residue/position. For example, typical modifications include substitution of the residue with another amino acid (e.g., a conservative or non-conservative substitution), insertion of one or more (e.g., generally fewer than 5, 4, or 3) amino acids adjacent to said residue/position, and/or deletion of said residue/position.

A “modification promoting heterodimerization” is a manipulation of the peptide backbone or the post-translational modifications of a polypeptide, e.g., an immunoglobulin heavy chain, that reduces or prevents the association of the polypeptide with an identical polypeptide to form a homodimer. A modification promoting heterodimerization as used herein particularly includes separate modifications made to each of two polypeptides desired to form a dimer, wherein the modifications are complementary to each other so as to promote association of the two polypeptides. For example, a modification promoting heterodimerization may alter the structure or charge of one or both of the polypeptides desired to form a dimer so as to make their association sterically or electrostatically favorable, respectively. Heterodimerization occurs between two non-identical polypeptides, such as two immunoglobulin heavy chains wherein further immunoconjugate components fused to each of the heavy chains (e.g., TNF-α polypeptide) are not the same. In the immunoconjugates of the present disclosure, the modification promoting heterodimerization is in the heavy chain(s), specifically in the Fc domain, of an immunoglobulin molecule. In some embodiments the modification promoting heterodimerization comprises an amino acid mutation, specifically an amino acid substitution. In a particular embodiment, the modification promoting heterodimerization comprises a separate amino acid mutation, specifically an amino acid substitution, in each of the two immunoglobulin heavy chains.

The term “Fc domain” herein is used to define the C-terminal portion of an immunoglobulin composed of the Fc regions of both heavy chains of the immunoglobulin. Each heavy chain Fc region in an Fc domain is herein referred to as a subunit of the Fc domain. The two subunits of a Fc domain can be both native sequence Fc regions, or both variant Fc regions, or one native sequence Fc region and one variant Fc region. In certain embodiments, the Fc domain comprises a modification promoting hetero-dimerization of two non-identical immunoglobulin heavy chains. The site of most extensive protein-protein interaction between the two polypeptidic chains of a human IgG Fc domain is in the CH3 domain of the Fc regions. Thus, in one embodiment, said modification is in the CH3 domain of the Fc regions. In a specific embodiment said modification is a knob-into-hole modification, comprising a knob modification in one of the Fc subunits, referred to as “Fc-Knob,” and a hole modification in the other one of the Fc subunits, referred to as “Fc-hole.” The knob-into-hole technology is described e.g., in U.S. Pat. Nos. 5,731,168; 7,695,936; Ridgway et al., Prat Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). The protuberance and cavity can be made by altering the nucleic acid encoding the polypeptides, e.g., by site-specific mutagenesis, or by peptide synthesis. In a specific embodiment a knob modification comprises the amino acid substitution T366W in one of the two Fc subunits, and the hole modification comprises the amino acid substitutions T366S, L368A and Y407V in the other one of the two Fc subunits. In a further specific embodiment, the Fc subunit comprising the knob modification additionally comprises the amino acid substitution S354C, and the immunoglobulin heavy chain comprising the hole modification additionally comprises the amino acid substitution Y349C. Introduction of these two cysteine residues results in formation of a disulfide bridge between the two heavy chains, further stabilizing the dimer (Carter, J. Immunol Methods 248, 7-15 (2001)).

The term “variant” when used in relation to a peptide or polypeptide, to an antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a TNF-α variant may result from one or more (such as, for example, about 1 to about 25, about 1 to about 20, about 1 to about 15, about 1 to about 10, or about 1 to about 5) changes to an amino acid sequence of a native TNF-α. Also by way of example, a variant of an anti-TNF-α antibody may result from one or more (such as, for example, about 1 to about 25, about 1 to about 20, about 1 to about 15, about 1 to about 10, or about 1 to about 5) changes to an amino acid sequence of a native or previously unmodified anti-TNF-α antibody. Variants may be naturally occurring, such as allelic or splice variants, or may be artificially constructed. Polypeptide variants may be prepared from the corresponding nucleic acid molecules encoding the variants. In specific embodiments, the TNF-α variant or anti-TNF-α antibody variant at least retains TNF-α or anti-TNF-α antibody functional activity, respectively. In specific embodiments, an anti-TNF-α antibody variant is a bispecific antibody that binds to both FAP and TNF-α. In specific embodiments, an anti-TNF-α antibody variant is a bispecific antibody that binds to both Trop-2 and TNF-α. In certain embodiments, the variant is encoded by a single nucleotide polymorphism (SNP) variant of a nucleic acid molecule that encodes TNF-α or anti-TNF-α antibody VH or VL regions or subregions, such as one or more CDRs.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

“Antibody fragments” comprise a portion of an intact antibody, such as the antigen-binding or variable region of the intact antibody. Examples of antibody fragments include, without limitation, Fab, Fab′, F(ab′)₂, and Fv fragments; diabodies and di-diabodies (see, e.g., Holliger et al., 1993, Proc. Natl. Acad. Sci. 90:6444-48; Lu et al., 2005, J. Biol. Chem. 280:19665-72; Hudson et al., 2003, Nat. Med. 9:129-34; WO 93/11161; and U.S. Pat. Nos. 5,837,242 and 6,492,123); single-chain antibody molecules (see, e.g., U.S. Pat. Nos. 4,946,778; 5,260,203; 5,482,858; and 5,476,786); dual variable domain antibodies (see, e.g., U.S. Pat. No. 7,612,181); single domain antibodies (sdAbs) (see, e.g., Woolven et al., 1999, Immunogenetics 50: 98-101; and Streltsov et al., 2004, Proc Natl Acad Sci USA. 101:12444-49); and multispecific antibodies formed from antibody fragments.

A “functional fragment,” “binding fragment,” or “antigen-binding fragment” of a therapeutic antibody will exhibit at least one if not some or all of the biological functions attributed to the intact antibody, the function comprising at least binding to the target antigen (e.g., an TNF-α binding fragment or fragment that binds to TNF-α).

As used herein, the term “immunoconjugate” refers to a polypeptide molecule that includes at least one cytokine moiety and at least one antigen binding moiety. In certain embodiments, the immunoconjugate comprises at least one cytokine moiety (e.g., TNF-α), and at least two antigen binding moieties (e.g., a masking moiety and an anchoring moiety as described herein). Particularly, in certain embodiments, immunoconjugates according to the present disclosure comprise one cytokine moiety and two antigen binding moieties joined by one or more linker sequences. In certain embodiments, immunoconjugates according to the present disclosure comprises one cytokine moiety and two antigen binding moieties joined by an Fc domain of immunoglobulin. In various embodiments of the present disclosure, the antigen binding moiety can be joined to the cytokine moiety by a variety of interactions and in a variety of configurations as described herein.

The term “fusion,” “fuse” or other grammatical variants thereof when used in relation to a peptide or polypeptide, or to an antibody refers to the joining of a peptide or polypeptide, or fragment, variant, and/or derivative thereof, with a heterologous peptide or polypeptide.

An “affinity matured” antibody is one with one or more alterations (e.g., amino acid sequence variations, including changes, additions, and/or deletions) in one or more HVRs thereof which result in an improvement in the affinity of the antibody for antigen, compared to a parent antibody which does not possess those alteration(s). Affinity matured antibodies can have nanomolar or even picomolar affinities for the target antigen. Affinity matured antibodies are produced by procedures known in the art. For review, see Hudson and Souriau, 2003, Nature Medicine 9:129-34; Hoogenboom, 2005, Nature Biotechnol. 23:1105-16; Quiroz and Sinclair, 2010, Revista Ingeneria Biomedia 4:39-51.

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen et al., 1999, J. Mol Biol 293:865-81). The K_{D} or K_{D} value may also be measured by using surface plasmon resonance assays by Biacore®, using, for example, a Biacore® TM-2000 or a Biacore® TM-3000, or by biolayer interferometry using, for example, a Octet® QK384 or Gator™ system. An “on-rate” or “rate of association” or “association rate” or “kₒₙ” may also be determined with the same surface plasmon resonance or biolayer interferometry techniques described above using, for example, a Biacore® TM-2000 or a Biacore® TM-3000, or a Octet® QK384, or Gator™ system.

The term “inhibition” or “inhibit,” when used herein, refers to partial (such as, 1%, 2%, 5%, 10%, 20%, 25%, 50%, 75%, 90%, 95%, 99%) or complete (i.e., 100%) inhibition.

“Fc receptor” or “FcR” describes a receptor that binds to the Fc region of an antibody. An exemplary FcR is a native sequence human FcR. Moreover, an exemplary FcR is one that binds an IgG antibody (e.g., a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof (see, e.g., Daeron, 1997, Annu. Rev. Immunol. 15:203-34). Various FcRs are known (see, e.g., Ravetch and Kinet, 1991, Annu. Rev. Immunol. 9:457-92; Capel et al., 1994, Immunomethods 4:25-34; and de Haas et al., 1995, J. Lab. Clin. Med. 126:330-41). Other FcRs, including those to be identified in the future, are encompassed by the term “FcR” herein. The term also includes the neonatal receptor, FcRn, which is responsible for the transfer of maternal IgGs to the fetus (see, e.g., Guyer et al., 1976, J. Immunol. 117:587-93; and Kim et al., 1994, Eu. J. Immunol. 24:2429-34). Antibody variants with improved or diminished binding to FcRs have been described (see, e.g., WO 2000/42072; U.S. Pat. Nos. 7,183,387; 7,332,581; and 7,335,742; Shields et al. 2001, J. Biol. Chem. 9(2):6591-604).

The term “vector” refers to a substance that is used to carry or include a nucleic acid sequence, including for example, a nucleic acid sequence encoding an antibody or a cytokine polypeptide as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., an anti-FAP antibody as described herein), and it is further understood that expression levels can be optimized to obtain sufficient expression using methods well known in the art.

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces an antibody of the present disclosure may include a parent hybridoma cell, as well as bacterial and eukaryotic host cells into which nucleic acids encoding the antibodies have been introduced. Suitable host cells are disclosed below.

Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

The term “encoding nucleic acid” or grammatical equivalents thereof as it is used in reference to nucleic acid molecule refers to a nucleic acid molecule in its native state or when manipulated by methods well known to those skilled in the art that can be transcribed to produce mRNA, which is then translated into a polypeptide and/or a fragment thereof. The antisense strand is the complement of such a nucleic acid molecule, and the encoding sequence can be deduced therefrom.

The term “recombinant antibody” refers to an antibody that is prepared, expressed, created, or isolated by recombinant means. Recombinant antibodies can be antibodies expressed using a recombinant expression vector transfected into a host cell, antibodies isolated from a recombinant, combinatorial antibody library, antibodies isolated from an animal (e.g., a mouse or cow) that is transgenic and/or transchromosomal for human immunoglobulin genes (see, e.g., Taylor et al., 1992, Nucl. Acids Res. 20:6287-95), or antibodies prepared, expressed, created, or isolated by any other means that involves splicing of immunoglobulin gene sequences to other DNA sequences. Such recombinant antibodies can have variable and constant regions, including those derived from human germline immunoglobulin sequences (See Kabat et al., supra). In certain embodiments, however, such recombinant antibodies may be subjected to in vitro mutagenesis (or, when an animal transgenic for human Ig sequences is used, in vivo somatic mutagenesis), thus the amino acid sequences of the VH and VL regions of the recombinant antibodies are sequences that, while derived from and related to human germline VH and VL sequences, may not naturally exist within the human antibody germline repertoire in vivo.

The term “composition” is intended to encompass a product containing the specified ingredients (e.g., an immunoconjugate molecule provided herein) in, optionally, the specified amounts.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. The term “carrier” can also refer to a diluent, adjuvant (e.g., Freund's adjuvant (complete or incomplete)), excipient, or vehicle. Such carriers, including pharmaceutical carriers, can be sterile liquids, such as water and oils, including those of petroleum, animal, vegetable, or synthetic origin, such as peanut oil, soybean oil, mineral oil, sesame oil, and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard carriers such as pharmaceutical grades of mannitol, lactose, starch, magnesium stearate, sodium saccharine, cellulose, magnesium carbonate, etc. Examples of suitable pharmaceutical carriers are described in Remington and Gennaro, Remington's Pharmaceutical Sciences (18th ed. 1990). Compositions, including pharmaceutical compounds, may contain an antibody, for example, in isolated or purified form, together with a suitable amount of carriers.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

The term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.), and polyols (e.g., mannitol, sorbitol, etc.). See, also, Remington and Gennaro, Remington's Pharmaceutical Sciences (18th ed. 1990), which is hereby incorporated by reference in its entirety.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human.

“Administer” or “administration” refers to the act of injecting or otherwise physically delivering a substance as it exists outside the body (e.g., an immunoconjugate molecule as described herein) into a patient, such as by mucosal, intradermal, intravenous, intramuscular delivery, and/or any other method of physical delivery described herein or known in the art.

The term “effective amount” as used herein refers to the amount of an antibody or pharmaceutical composition provided herein which is sufficient to result in the desired outcome.

The terms “about” and “approximately” mean within 20%, within 15%, within 10%, within 9%, within 8%, within 7%, within 6%, within 5%, within 4%, within 3%, within 2%, within 1%, or less of a given value or range.

“Substantially all” refers to at least about 60%, at least about 65%, at least about 70%, at least about 75%, at least about 80%, at least about 85%, at least about 90%, at least about 95%, at least about 98%, at least about 99%, or about 100%.

The phrase “substantially similar” or “substantially the same” denotes a sufficiently high degree of similarity between two numeric values (e.g., one associated with an antibody of the present disclosure and the other associated with a reference antibody) such that one of skill in the art would consider the difference between the two values to be of little or no biological and/or statistical significance within the context of the biological characteristic measured by the values (e.g., K_{D} values). For example, the difference between the two values may be less than about 50%, less than about 40%, less than about 30%, less than about 20%, less than about 10%, or less than about 5%, as a function of the value for the reference antibody.

The phrase “substantially increased,” “substantially reduced,” or “substantially different,” as used herein, denotes a sufficiently high degree of difference between two numeric values (e.g., one associated with an antibody of the present disclosure and the other associated with a reference antibody) such that one of skill in the art would consider the difference between the two values to be of statistical significance within the context of the biological characteristic measured by the values. For example, the difference between said two values can be greater than about 10%, greater than about 20%, greater than about 30%, greater than about 40%, or greater than about 50%, as a function of the value for the reference antibody.

### 5.3 Compositions and Methods of Making the Same

In one aspect of the present disclosure, provided herein are cytokine-containing immunoconjugate molecules. In some embodiments, the immunoconjugate molecules are fusion proteins comprising a cytokine moiety and a non-cytokine portion operably linked to one another. According to the present disclosure, the cytokine-containing immunoconjugate molecules are capable of delivery and activation of cellular activities of the cytokine at particular tissue or cellular location in a subject. For example, in some embodiments, the cytokine activity is reduced or blocked when the immunoconjugate molecules are present in an environment lacking an activation signal for the cytokine. In some embodiments, the cytokine activity is activated or enhanced when the immunoconjugate molecule are present in an environment containing or enriched of the activation signal for the cytokine. For example, in some embodiments, the immunoconjugate molecules are configured for tissue-specific distribution upon administration to a subject. In particular embodiments, the immunoconjugate molecules are capable of being enriched in certain tissue or cellular environment providing the activation signal for the cytokine, thereby activating the cytokine activity specifically in such tissue or cellular environment.

In specific embodiments, the activation signal for the cytokine is the presence of a signal molecule in the target tissue or cellular environment where the cytokine activity is activated. In some embodiments, the signal molecule is enriched in the target tissue or cellular environment, while present at other non-target tissue or cellular environment at a lower amount or concentration. In some embodiments, the activation signal for the cytokine is the presence of a signal molecule in the target tissue or cellular environment at a concentration above a threshold. In some embodiments, the signal molecule is capable of interacting with the immunoconjugate molecule, thereby activates the cytokine activity. In some embodiments, the signal molecule is a peptide molecule.

In specific embodiments, the immunoconjugate molecules are configured for the targeted delivery and activation of the cytokine activity in cancerous tissues, such as a tumor. In those embodiments, the signal molecule for activating the cytokine can be an antigen that is expressed or enriched in the cancerous tissue, such as in the tumor microenvironment. In specific embodiments, the activation signal for the cytokine is an antigen expressed on the tumor cells. In other embodiments, the activation signal for the cytokine is an antigen expressed on the cells in the tumor microenvironment, such as tumor stromal cells. In specific embodiments, the activation signal for the cytokine is a tumor associated antigen.

In some embodiments, the non-cytokine portion of the immunoconjugate molecule comprises a masking moiety capable of binding with the cytokine moiety, and upon the binding, the masking moiety reduces or blocks the cytokine activity. In some embodiments, the immunoconjugate molecule comprises an antibody or antigen binding fragment thereof that is fused to a cytokine polypeptide, and the antibody or antigen binding fragment thereof is capable of binding with the cytokine polypeptide and reduces or blocks the cytokine activity.

In some embodiments, the intramolecular binding between the cytokine moiety and the masking moiety of an immunoconjugate molecule is reversible. Accordingly, in some embodiments, the immunoconjugate molecules can switch between cytokine active and inactive states, through the reversible binding and disassociation between the cytokine moiety and the masking moiety.

In some embodiments, the masking moiety is a bispecific two-in-one antibody or a binding fragment thereof, which is capable of binding to the cytokine moiety and a second target antigen that is different from the cytokine. In specific embodiments, when the immunoconjugate molecule is in an environment where the second target antigen is absent, the masking moiety comprising the two-in-one antibody or antigen binding fragment thereof binds with the cytokine moiety of the immunoconjugate molecule, thereby inhibiting the cytokine activity. In specific embodiments, when the immunoconjugate molecule is in an environment where the second target antigen is present at an amount or concentration below a certain threshold, the masking moiety comprising the two-in-one antibody or antigen binding fragment thereof binds with the cytokine moiety of the immunoconjugate molecule, thereby inhibiting the cytokine activity. In various embodiments, the environment is a cellular environment or a tissue-specific environment. In particular embodiments, the environment is a cancerous tissue or a tumor microenvironment. In particular embodiments, the second target antigen is an antigen expressed by the cancer cells. In other embodiments, the second target antigen is an antigen expressed by the cells in the tumor microenvironment, such as tumor stromal cells. In some embodiments, the second target antigen is a tumor associated antigen.

In some embodiments, the masking moiety is a bispecific two-in-one antibody or a binding fragment thereof, which is capable of binding to the cytokine moiety and a second target antigen that is different from the cytokine. In specific embodiments, when the immunoconjugate molecule is in an environment where the second target antigen is present, the masking moiety comprising the two-in-one antibody or antigen binding fragment thereof binds with the second antigen and disassociates from the cytokine moiety of the immunoconjugate molecule, thereby activating the cytokine activity. In specific embodiments, when the immunoconjugate molecule is in an environment where the second target antigen is present at an amount or concentration above a certain threshold, the masking moiety comprising the two-in-one antibody or antigen binding fragment thereof binds with the second antigen and disassociates from the cytokine moiety of the immunoconjugate molecule, thereby activating the cytokine activity. In various embodiments, the environment is a cellular environment or a tissue-specific environment. In particular embodiments, the environment is a cancerous tissue or a tumor microenvironment. In particular embodiments, the second target antigen is an antigen expressed by the tumor cells. In other embodiments, the second target antigen is an antigen expressed by the cells in the tumor microenvironment, such as tumor stromal cells. In some embodiments, the second target antigen is a tumor associated antigen.

In some embodiments, the masking moiety is a bispecific two-in-one antibody or a binding fragment thereof, which is capable of binding to the cytokine moiety and a second target antigen that is different from the cytokine. In specific embodiments, when the immunoconjugate molecule is in an environment where the second target antigen is present, the masking moiety comprising the two-in-one antibody or antigen binding fragment thereof binds with the second antigen and disassociates from the cytokine moiety of the immunoconjugate molecule, thereby activating the cytokine activity. In specific embodiments, when the immunoconjugate molecule is in an environment where the second target antigen is present at an amount or concentration above a certain threshold, the masking moiety comprising the two-in-one antibody or antigen binding fragment thereof binds with the second antigen and disassociates from the cytokine moiety of the immunoconjugate molecule, thereby activating the cytokine activity. In some embodiments, the second target antigen is expressed by a cell that also expresses a receptor for the cytokine forming part of the immunoconjugate molecule. In alternative embodiments, the second target antigen is expressed by a cell that does not itself express a receptor for the cytokine forming part of the immunoconjugate molecule and is nearby another cell expressing a receptor for the cytokine.

In specific embodiments, the immunoconjugate molecules of the present disclosure comprises a cytokine moiety and a non-cytokine portion, where the cytokine moiety comprises an TNF-α polypeptide, and the non-cytokine portion comprises a bispecific two-in-one antibody capable of binding to both the TNF-α polypeptide in the immunoconjugate molecule and a second target antigen that is not TNF-α. In particular embodiments, the second target antigen is an antigen expressed by the tumor cells. In other embodiments, the second target antigen is an antigen expressed by the cells in the tumor microenvironment, such as tumor stromal cells. In some embodiments, the second target antigen is a tumor associated antigen. In specific embodiments, the second target antigen is fibroblast activation protein (FAP). In specific embodiments, the second target antigen is Tumor-associated calcium signal transducer 2 (Trop-2). In yet specific embodiments, the TNF-α polypeptide is wild-type TNF-α polypeptide. In other embodiments, the TNF-α polypeptide is a mutant TNF-α polypeptide. In some embodiments, the TNF-α polypeptide is a human TNF-α polypeptide. In some embodiments, the TNF-α polypeptide is a monkey TNF-α polypeptide. In some embodiments, the TNF-α polypeptide is a mouse TNF-α polypeptide. In some embodiments, the TNF-α polypeptide is a mutant TNF-α polypeptide as described herein. In some embodiments, the TNF-α polypeptide is a mutant TNF-α polypeptide as described herein. In specific embodiments, the mutant TNF-α polypeptide is human TNF-α having one or more point mutation(s) selected from A33T, N34A, N34H, Y87A, Y87F, S95A, A145G, A145V, S147A, and S147G. In specific embodiments, the mutant TNF-α polypeptide is human TNF-α having one or more point mutation(s) selected from P08C, L55C, Q102C, E104C, S95C, G148C, I97C, Y115C, H73C, and P113C. In specific embodiments, a mutant TNF-α is human TNF-α containing (i) double mutations of P08C and L55C; (ii) double mutations of Q102C and E104C, (iii) double mutations of S95C and G148C, (iv) double mutations of I97C and Y115C; (v) double mutations of H73C and P113C; or (vi) any combinations of (i) to (v). In specific embodiments, the mutant TNF-α polypeptide is a non-human TNF-α having one or more point mutation(s) at corresponding sites of N34, Y87, S95, and S147 of human TNF-α sequence as provided herein. In specific embodiments, the mutant TNF-α polypeptide is a non-human TNF-α having one or more point mutation(s) corresponding to A33T, N34A, N34H, Y87A, Y87F, S95A, A145G, A145V, S147A, and S147G of human TNF-α sequence as provided herein. In specific embodiments, the mutant TNF-α polypeptide is a non-human TNF-α having one or more point mutation(s) at corresponding sites of P08C, L55C, Q102C, E104C, S95C, G148C, I97C, Y115C, H73C, and P113C of human TNF-α sequence as provided herein. In specific embodiments, the mutant TNF-α polypeptide is a non-human TNF-α having one or more point mutation(s) corresponding to P08C, L55C, Q102C, E104C, S95C, G148C, I97C, Y115C, H73C, and P113C of human TNF-α sequence as provided herein. In specific embodiments, the mutant TNF-α polypeptide is a non-human TNF-α having one or more point mutation(s) at corresponding sites of (i) double mutations of P08C and L55C; (ii) double mutations of Q102C and E104C, (iii) double mutations of S95C and G148C, (iv) double mutations of I97C and Y115C; (v) double mutations of H73C and P113C; or (vi) any combinations of (i) to (v) of human TNF-α sequence as provided herein. In specific embodiments, the mutant TNF-α polypeptide is a non-human TNF-α having one or more point mutation(s) corresponding to (i) double mutations of P08C and L55C; (ii) double mutations of Q102C and E104C, (iii) double mutations of S95C and G148C, (iv) double mutations of I97C and Y115C; (v) double mutations of H73C and P113C; or (vi) any combinations of (i) to (v) of human TNF-α sequence as provided herein. Additional mutant TNF-α polypeptides that can be used in connection with the present disclosure can be found in Loetscher et al., Journal of Biological Chemistry, 268(35): 26350-26357, which is hereby incorporated by reference in its entirety.

In some embodiments, the non-cytokine portion of the immunoconjugate molecule comprises an anchoring moiety configured to tether the immunoconjugate molecule to a target location of delivery. Hence, in some embodiments, immunoconjugate molecules of the present disclosure having the anchoring moiety can achieve tissue-specific distribution after being administered to a subject, such as after systemic administration to a subject. In some embodiments, the anchoring moiety of the immunoconjugate molecule is capable of specific binding to a target molecule that is present in the target location of delivery. In some embodiments, the anchoring moiety of the immunoconjugate molecule comprises an antibody or antigen binding fragment thereof capable of binding to an antigen present in the target location of delivery, thereby tethering the immunoconjugate molecule to the target location of delivery.

In some embodiments, the target location of delivery is a cellular environment, or a tissue-specific environment. In some embodiments, the target location of delivery also contains an activation signal for the cytokine of the immunoconjugate molecule, such that the cytokine activity can be activated in the target location.

In particular embodiments, the target location of delivery is a cancerous tissue or a tumor microenvironment. In some embodiments, the target location of delivery is a particular type of tissue or population of cells in a subject. In some embodiments, the anchoring moiety of the immunoconjugate molecule comprises an antibody or antigen binding fragment thereof that bind to an antigen expressed on a target cell. In some embodiments, the target cell also expresses a receptor for the cytokine forming part of the immunoconjugate molecule. In alternative embodiments, the target cell does not itself express a receptor for the cytokine forming part of the immunoconjugate molecule, and is nearby another cell expressing a receptor for the cytokine.

In particular embodiments, the target location of delivery is a cancerous tissue or a tumor microenvironment. In some embodiments, the target location of delivery is a particular type of tissue or population of cells in a subject. In some embodiments, the anchoring moiety of the immunoconjugate molecule comprises an antibody or antigen binding fragment thereof that bind to an antigen expressed on cancer cells. Accordingly, in those embodiments, the immunoconjugate molecule, upon administration to a subject having cancer, can bind to a population of cancer cells in the subject. In some embodiments, the anchoring moiety of the immunoconjugate molecule comprises an antibody or antigen binding fragment thereof that bind to an antigen present in the tumor microenvironment, such as an antigen expressed on surface of a tumor cells or antigen secreted by cells in the tumor microenvironment, such as tumor stromal cells. Accordingly, in those embodiments, the immunoconjugate molecule, upon administration to a subject having a solid tumor, can enrich in the tumor microenvironment in the subject.

In some embodiments, the immunoconjugate molecules of the present disclosure comprises a cytokine moiety, a masking moiety and an anchoring moiety that are operably connected with one another. In specific embodiments, the masking moiety is a bispecific two-in-one antibody or antigen binding fragment thereof capable of binding to both the cytokine moiety and a second target antigen that is not the cytokine. In specific embodiments, the anchoring moiety is an antibody or antigen binding fragment thereof capable of binding to a third target antigen, such as an antigen present in a target location of delivery for the immunoconjugate molecule. In some embodiments, the target location of delivery also contains the second target antigen in a sufficient amount to compete with the cytokine for binding with the masking moiety, resulting in disassociation of the masking moiety from the cytokine and activation of cytokine activity at the target location of delivery.

In some embodiments, the immunoconjugate molecules, upon administration to a subject, can achieve tissue-specific distribution and enrich in a target tissue or cellular environment in the subject that contains sufficient amount of the third antigen. In specific embodiments, the target tissue or cellular environment also contains the second target antigen in a sufficient amount to compete with the cytokine for binding with the masking moiety, resulting in disassociation of the masking moiety from the cytokine and activation of cytokine activity in the target tissue or cellular environment.

In specific embodiments, the second and the third target antigens respectively recognized by the masking moiety and the anchoring moiety of the immunoconjugate are the same antigen. In alternative embodiments, the second and the third target antigens respectively recognized by the masking moiety and the anchoring moiety of the immunoconjugate are different antigens.

In specific embodiments, the second and the third target antigens respectively recognized by the masking moiety and the anchoring moiety of the immunoconjugate are the same antigen, and such antigen is expressed by a cell that also expresses a receptor for the cytokine forming part of the immunoconjugate molecule. In alternative embodiments, the second and the third target antigens respectively recognized by the masking moiety and the anchoring moiety of the immunoconjugate are the same antigen, and such antigen is expressed by a cell that does not itself expresses a receptor for the cytokine forming part of the immunoconjugate molecule and is nearby another cell expressing the receptor for the cytokine.

In specific embodiments, the second and the third target antigens respectively recognized by the masking moiety and the anchoring moiety of the immunoconjugate are different antigens, and the second target antigen is expressed by a cell that also expresses a receptor for the cytokine forming part of the immunoconjugate molecule. In specific embodiments, the cell can also express the third target antigen. In alternative embodiments, the cell does not itself express the third target antigen and is nearby another cell expressing the third target antigen.

In specific embodiments, the second and the third target antigens respectively recognized by the masking moiety and the anchoring moiety of the immunoconjugate are different antigens, and the third target antigen is expressed by a cell that also expresses a receptor for the cytokine forming part of the immunoconjugate molecule. In specific embodiments, the cell can also express the second target antigen. In alternative embodiments, the cell does not itself express the second target antigen and is nearby another cell expressing the second target antigen.

In specific embodiments, the second and the third target antigens respectively recognized by the masking moiety and the anchoring moiety of the immunoconjugate are different antigens, and the second target antigen is expressed by a cell that does not itself expresses a receptor for the cytokine forming part of the immunoconjugate molecule and is nearby another cell expressing the receptor for the cytokine. In specific embodiments, the cell expressing the second target antigen can also express the third target antigen. In specific embodiments, the cell expressing the receptor for the cytokine can also express the third target antigen. In specific embodiments, the third target antigen is express a cell nearby the cell expressing the second target antigen and/or the cell expressing the receptor for the cytokine.

In specific embodiments, the second and the third target antigens respectively recognized by the masking moiety and the anchoring moiety of the immunoconjugate are different antigens, and the third target antigen is expressed by a cell that does not itself expresses a receptor for the cytokine forming part of the immunoconjugate molecule and is nearby another cell expressing the receptor for the cytokine. In specific embodiments, the cell expressing the third target antigen can also express the second target antigen. In specific embodiments, the cell expressing the receptor for the cytokine can also express the second target antigen. In specific embodiments, the second target antigen is express a cell nearby the cell expressing the third target antigen and/or the cell expressing the receptor for the cytokine.

In specific embodiments, the cytokine moiety comprises a TNF-α polypeptide, and the non-cytokine portion of the immunoconjugate molecule comprises a masking moiety comprising a bispecific two-in-one antibody capable of binding to both the TNF-α polypeptide in the immunoconjugate molecule and a second target antigen that is not TNF-α. In particular embodiments, the second target antigen is an antigen expressed by the tumor cells. In other embodiments, the second target antigen is an antigen expressed by the cells in the tumor microenvironment, such as tumor stromal cells. In some embodiments, the second target antigen is a tumor associated antigen. In specific embodiments, the second target antigen is fibroblast activation protein (FAP). In specific embodiments, the second target antigen is tumor-associated calcium signal transducer 2 (Trop-2). In specific embodiments, the non-cytokine portion of the immunoconjugate molecule further comprises an anchoring moiety comprising an antibody or antigen binding fragment capable of binding to a third target antigen that is not TNF-α. In particular embodiments, the third target antigen is an antigen expressed by the tumor cells. In some embodiments, the third target antigen is an antigen expressed by the cells in the tumor microenvironment, such as tumor stromal cells. In some embodiments, the third target antigen is a tumor associated antigen. In specific embodiments, the third target antigen is fibroblast activation protein (FAP). In specific embodiments, the third target antigen is tumor-associated calcium signal transducer 2 (Trop-2). In yet specific embodiments, the TNF-α polypeptide is wild-type TNF-α polypeptide. In other embodiments, the TNF-α polypeptide is a mutant TNF-α polypeptide. In some embodiments, the TNF-α polypeptide is a human TNF-α polypeptide. In some embodiments, the TNF-α polypeptide is a monkey TNF-α polypeptide. In some embodiments, the TNF-α polypeptide is a mouse TNF-α polypeptide. In some embodiments, the TNF-α polypeptide is a mutant TNF-α polypeptide as described herein. In specific embodiments, the mutant TNF-α polypeptide is human TNF-α having one or more point mutation(s) selected from A33T, N34A, N34H, Y87A, Y87F, S95A, A145G, A145V, S147A, and S147G. In specific embodiments, the mutant TNF-α polypeptide is human TNF-α having one or more point mutation(s) selected from P08C, L55C, Q102C, E104C, S95C, G148C, I97C, Y115C, H73C, and P113C. In specific embodiments, a mutant TNF-α is human TNF-α containing (i) double mutations of P08C and L55C; (ii) double mutations of Q102C and E104C, (iii) double mutations of S95C and G148C, (iv) double mutations of I97C and Y115C; (v) double mutations of H73C and P113C; or (vi) any combinations of (i) to (v). In specific embodiments, the mutant TNF-α polypeptide is a non-human TNF-α having one or more point mutation(s) at corresponding sites of N34, Y87, S95, and S147 of human TNF-α sequence as provided herein. In specific embodiments, the mutant TNF-α polypeptide is a non-human TNF-α having one or more point mutation(s) corresponding to A33T, N34A, N34H, Y87A, Y87F, S95A, A145G, A145V, S147A, and S147G of human TNF-α sequence as provided herein. In specific embodiments, the mutant TNF-α polypeptide is a non-human TNF-α having one or more point mutation(s) at corresponding sites of P08C, L55C, Q102C, E104C, S95C, G148C, I97C, Y115C, H73C, and P113C of human TNF-α sequence as provided herein. In specific embodiments, the mutant TNF-α polypeptide is a non-human TNF-α having one or more point mutation(s) corresponding to P08C, L55C, Q102C, E104C, S95C, G148C, I97C, Y115C, H73C, and P113C of human TNF-α sequence as provided herein. In specific embodiments, the mutant TNF-α polypeptide is a non-human TNF-α having one or more point mutation(s) at corresponding sites of (i) double mutations of P08C and L55C; (ii) double mutations of Q102C and E104C, (iii) double mutations of S95C and G148C, (iv) double mutations of I97C and Y115C; (v) double mutations of H73C and P113C; or (vi) any combinations of (i) to (v) of human TNF-α sequence as provided herein. In specific embodiments, the mutant TNF-α polypeptide is a non-human TNF-α having one or more point mutation(s) corresponding to (i) double mutations of P08C and L55C; (ii) double mutations of Q102C and E104C, (iii) double mutations of S95C and G148C, (iv) double mutations of I97C and Y115C; (v) double mutations of H73C and P113C; or (vi) any combinations of (i) to (v) of human TNF-α sequence as provided herein. Additional mutant TNF-α polypeptides that can be used in connection with the present disclosure can be found in Loetscher et al., Journal of Biological Chemistry, 268(35): 26350-26357, which is hereby incorporated by reference in its entirety.

In some embodiments, the present immunoconjugate molecule comprises an anchoring moiety, a masking moiety and a cytokine moiety that are operably linked to one another via peptidic linkers. In some embodiments, the peptidic linker has at least 5 amino acid residues. In some embodiments, the peptidic linker has at least 7 amino acid residues. In some embodiments, the peptidic linker has at least 10 amino acid residues. In some embodiments, the peptidic linker has at least 15 amino acid residues. In some embodiments, the peptidic linker has at least 20 amino acid residues. In some embodiments, the peptidic linker has at least 30 amino acid residues. In some embodiments, the peptidic linker has at least 40 amino acid residues. In some embodiments, the peptidic linker has at least 50 amino acid residues. In some embodiments, the peptidic linker has at least 60 amino acid residues. In some embodiments, the peptidic linker has at least 70 amino acid residues. In some embodiments, the peptidic linker has at least 80 amino acid residues. In some embodiments, the peptidic linker has at least 90 amino acid residues. In some embodiments, the peptidic linker has at least 100 amino acid residues. In some embodiments, the peptidic linker has at least 110 amino acid residues. In some embodiments, the peptidic linker has at least 120 amino acid residues. In some embodiments, the peptidic linker has at least 130 amino acid residues. In some embodiments, the peptidic linker has at least 140 amino acid residues. In some embodiments, the peptidic linker has at least 150 amino acid residues. In some embodiments, the peptidic linker has at least 160 amino acid residues. In some embodiments, the peptidic linker has at least 170 amino acid residues. In some embodiments, the peptidic linker has at least 180 amino acid residues. In some embodiments, the peptidic linker has at least 190 amino acid residues. In some embodiments, the peptidic linker has at least 200 amino acid residues. In some embodiments, the peptidic linker has at least 250 amino acid residues. In some embodiments, the peptidic linker has at least 300 amino acid residues. In exemplary embodiments, the peptidic linker comprises an amino acid fragment GGGGS (i.e., G4S). In exemplary embodiments, the peptidic linker comprises multiple tandem copies of the G4S fragments of varied length, such as 10, 15, 20, 25, 30, 35, 40, 45, 50, 55, 60, 65, 70, 75, 80, 85, 90, 95, 100, 110, 120, 130, 140, 150, 160, 170, 180, 190, 200, 150, or 300 amino acids.

In specific embodiments, the peptidic linker connecting the masking moiety and cytokine can have the same length as the peptidic linker connecting the masking moiety and the anchoring moiety. In alternative embodiments, the peptidic linker connecting the masking moiety and cytokine can have a different length from the peptidic linker connecting the masking moiety and the anchoring moiety. In specific embodiments, the peptidic linker connecting the masking moiety and cytokine can be at least 5, 7, 10, 15, 20, 30, 40, 50, 60, 70, 80, 90, 100, 110, 120, 130, 140, 150, 160, 170, 180, 190, 200, 250, or 300 amino acids. In specific embodiments, the peptidic linker connecting the masking moiety and anchoring moiety can be at least 5, 7, 10, 15, 20, 30, 40, 50, 60, 70, 80, 90, 100, 110, 120, 130, 140, 150, 160, 170, 180, 190, 200, 250, or 300 amino acids.

FIGS. 1B to 1L are schematic illustrations of complexes containing multimerized antibody-cytokine immunoconjugates having different molecular configurations according to the present disclosure. The multimerization between the immunoconjugate molecules can be via covalent (e.g., disulfide bond) or non-covalent interactions. Although FIGS. 1B to 1L illustrate the configurations in the timer form, based on these illustrations, those of ordinary skill in the art could also envision immunoconjugates in the monomer, dimer, or higher-order multimer (e.g., greater than 3) forms, which are also within the scope of the present disclosure.

In some embodiments, the present immunoconjugate comprises an anti-cytokine/anti-TAA two-in-one scFv antibody fused to a cytokine. In various embodiments, the immunoconjugate monomer can assume any one of the four different configurations: (i) cytokine-scFv(VH-VL), (ii) cytokine-scFv(VL-VH), (iii) scFv(VH-VL)-cytokine, and (iv) scFv(VL-VH)-cytokine, where “-” denotes a linker or direct fusion between different moieties, and the different moieties in a peptidic chain are listed according to the N to C orientation in the peptidic chain. In some embodiments, the monomer immunoconjugate molecule can multimerize into a complex, such as a trimer. In specific embodiments, multimerization is through interaction between the cytokine domains of two or more immunoconjugate molecules. In some embodiments, immunoconjugate molecules forming the multimer can each assume a configuration selected from (i) to (iv) of this paragraph, which configurations can be the same (homo-multimer complex) as, or different (hetero-multimer complex) from, one another. In specific embodiments, the cytokine is wild-type or mutant TNF-α. In specific embodiments, the multimerization is through interaction between the extensive trimer interfaces of the TNF-α polypeptides contained in the immunoconjugate molecules. In specific embodiments, the TAA is Trop-2 or FAP.

In some embodiments, the present immunoconjugate comprises an anti-cytokine/anti-TAA two-in-one Fab antibody fused to a cytokine. In various embodiments, the immunoconjugate monomer can assume any one of the four possible configurations: (i) cytokine-VH-CH1×VL-CL, (ii) cytokine-VL-CL×VH-CH1, (iii) VH-CH1-cytokine×VL-CL, and (iv) VL-CL-cytokine×VH-CH1, where “-” denotes a linker or direct fusion between different moieties, and the different moieties in a peptidic chain are listed according to the N to C orientation in the peptidic chain. In some embodiments, the monomer immunoconjugate molecule can multimerize into a complex, such as a trimer. In specific embodiments, multimerization is through interaction between the cytokine domains of two or more immunoconjugate molecules. In some embodiments, immunoconjugate molecules forming the multimer can each assume a configuration selected from (i) to (iv) of this paragraph, which configurations can be the same (homo-multimer complex) as, or different (hetero-multimer complex) from, one another. In specific embodiments, the cytokine is wild-type or mutant TNF-α. In specific embodiments, the multimerization is through interaction between the extensive trimer interfaces of the TNF-α polypeptides contained in the immunoconjugate molecules. In specific embodiments, the TAA is Trop-2 or FAP.

In some embodiments, the present immunoconjugate comprises an anti-cytokine/anti-TAA two-in-one scFab antibody fused to a cytokine. In various embodiments, the immunoconjugate monomer can assume any one of the four possible configurations: (i) cytokine-scFab(VH-CH1-VL-CL), (ii) cytokine-scFab(VL-CL-VH-CH1), (iii) scFab(VH-CH1-VL-CL)-cytokine, and (iv) scFab(VL-CL-VH-CH1)-cytokine, where “-” denotes a linker or direct fusion between different moieties, and the different moieties in a peptidic chain are listed according to the N to C orientation in the peptidic chain. In some embodiments, the monomer immunoconjugate molecule can multimerize into a complex, such as a trimer. In specific embodiments, multimerization is through interaction between the cytokine domains of two or more immunoconjugate molecules. In some embodiments, immunoconjugate molecules forming the multimer can each assume a configuration selected from (i) to (iv) of this paragraph, which configurations can be the same (homo-multimer complex) as, or different (hetero-multimer complex) from, one another. In specific embodiments, the cytokine is wild-type or mutant TNF-α. In specific embodiments, the multimerization is through interaction between the extensive trimer interfaces of the TNF-α polypeptides contained in the immunoconjugate molecules. In specific embodiments, the TAA is Trop-2 or FAP.

In some embodiments, the present immunoconjugate comprises (a) an anti-cytokine/anti-TAA two-in-one scFv antibody, (b) a cytokine polypeptide, and (c) an anti-TAA scFv antibody. In various embodiments, the immunoconjugate monomer can assume any one of the eight possible configurations: (i) cytokine-scFv(VH-VL)-scFv(VH-VL), (ii) cytokine-scFv(VL-VH)-scFv(VH-VL), (iii) cytokine-scFv(VH-VL)-scFv(VL-VH), (iv) cytokine-scFv(VL-VH)-scFv(VL-VH), (v) scFv(VH-VL)-scFv(VH-VL)-cytokine, (vi) scFv(VL-VH)-scFv(VH-VL)-cytokine, (vii) scFv(VH-VL)-scFv(VL-VH)-cytokine, and (viii) scFv(VL-VH)-scFv(VL-VH)-cytokine, where “-” denotes a linker or direct fusion between different moieties, and the different moieties in a peptidic chain are listed according to the N to C orientation in the peptidic chain. In some embodiments, the monomer immunoconjugate molecule can multimerize into a complex, such as a trimer. In specific embodiments, multimerization is through interaction between the cytokine domains of two or more immunoconjugate molecules. In some embodiments, immunoconjugate molecules forming the multimer can each assume a configuration selected from (i) to (viii) of this paragraph, which configurations can be the same (homo-multimer complex) as, or different (hetero-multimer complex) from, one another. In specific embodiments, the cytokine is wild-type or mutant TNF-α. In specific embodiments, the multimerization is through interaction between the extensive trimer interfaces of the TNF-α polypeptides contained in the immunoconjugate molecules. In specific embodiments, the TAA is Trop-2 or FAP.

In some embodiments, the present immunoconjugate comprises (a) an anti-cytokine/anti-TAA two-in-one scFv antibody, (b) a cytokine polypeptide, and (c) an anti-TAA VHH antibody. In various embodiments, the immunoconjugate monomer can assume any one of the four possible configurations: (i) cytokine-scFv(VH-VL)-VHH, (ii) cytokine-scFv(VL-VH)-VHH, (iii) scFv(VH-VL)-VHH-cytokine, and (iv) scFv(VL-VH)-VHH-cytokine, where “-” denotes a linker or direct fusion between different moieties, and the different moieties in a peptidic chain are listed according to the N to C orientation in the peptidic chain. In some embodiments, the monomer immunoconjugate molecule can multimerize into a complex, such as a trimer. In specific embodiments, multimerization is through interaction between the cytokine domains of two or more immunoconjugate molecules. In some embodiments, immunoconjugate molecules forming the multimer can each assume a configuration selected from (i) to (iv) of this paragraph, which configurations can be the same (homo-multimer complex) as, or different (hetero-multimer complex) from, one another. In specific embodiments, the cytokine is wild-type or mutant TNF-α. In specific embodiments, the multimerization is through interaction between the extensive trimer interfaces of the TNF-α polypeptides contained in the immunoconjugate molecules. In specific embodiments, the TAA is Trop-2 or FAP.

In some embodiments, the present immunoconjugate comprises (a) an anti-cytokine/anti-TAA two-in-one Fab antibody, (b) a cytokine polypeptide, and (c) an anti-TAA scFv antibody. In various embodiments, the immunoconjugate monomer can assume any one of the eight possible configurations: (i) cytokine-VH-CH1×scFv(VH-VL)-VL-CL, (ii) cytokine-VL-CL×scFv(VH-VL)-VH-CH1, (iii) cytokine-VH-CH1×scFv(VL-VH)-VL-CL, (iv) cytokine-VL-CL×scFv(VL-VH)-VH-CH1, (v) VH-CH1-cytokine×scFv(VH-VL)-VL-CL, (vi) VL-CL-cytokine×scFv(VH-VL)-VH-CH1, (vii) VH-CH1-cytokine×scFv(VL-VH)-VL-CL, and (viii) VL-CL-cytokine×scFv(VL-VH)—VH-CH1, where “-” denotes a linker or direct fusion between different moieties, and the different moieties in a peptidic chain are listed according to the N to C orientation in the peptidic chain. In some embodiments, the monomer immunoconjugate molecule can multimerize into a complex, such as a trimer. In specific embodiments, multimerization is through interaction between the cytokine domains of two or more immunoconjugate molecules. In some embodiments, immunoconjugate molecules forming the multimer can each assume a configuration selected from (i) to (viii) of this paragraph, which configurations can be the same (homo-multimer complex) as, or different (hetero-multimer complex) from, one another. In specific embodiments, the cytokine is wild-type or mutant TNF-α. In specific embodiments, the multimerization is through interaction between the extensive trimer interfaces of the TNF-α polypeptides contained in the immunoconjugate molecules. In specific embodiments, the TAA is Trop-2 or FAP.

In some embodiments, the present immunoconjugate comprises (a) an anti-cytokine/anti-TAA two-in-one Fab antibody, (b) a cytokine polypeptide, and (c) an anti-TAA VHH antibody. In various embodiments, the immunoconjugate monomer can assume any one of the four possible configurations: (i) cytokine-VH-CH1×VHH-VL-CL, (ii) cytokine-VL-CL×VHH-VH-CH1, (iii) VH-CH1-cytokine×VHH-VL-CL, and (iv) VL-CL-cytokine×VHH-VH-CH1, where “-” denotes a linker or direct fusion between different moieties, and the different moieties in a peptidic chain are listed according to the N to C orientation in the peptidic chain. In some embodiments, the monomer immunoconjugate molecule can multimerize into a complex, such as a trimer. In specific embodiments, multimerization is through interaction between the cytokine domains of two or more immunoconjugate molecules. In some embodiments, immunoconjugate molecules forming the multimer can each assume a configuration selected from (i) to (iv) of this paragraph, which configurations can be the same (homo-multimer complex) as, or different (hetero-multimer complex) from, one another. In specific embodiments, the cytokine is wild-type or mutant TNF-α. In specific embodiments, the multimerization is through interaction between the extensive trimer interfaces of the TNF-α polypeptides contained in the immunoconjugate molecules. In specific embodiments, the TAA is Trop-2 or FAP.

In some embodiments, the present immunoconjugate comprises (a) an anti-cytokine/anti-TAA two-in-one Fab antibody, (b) a cytokine polypeptide, and (c) an anti-TAA scFv antibody. In various embodiments, the immunoconjugate monomer can assume any one of the eight possible configurations: (i) cytokine-VH-CH1×VL-CL-scFv(VH-VL), (ii) cytokine-VL-CL×VH-CH1-scFv(VH-VL), (iii) cytokine-VH-CH1×VL-CL-scFv(VL-VH), (iv) cytokine-VL-CL×VH-CH1-scFv(VL-VH), (v) VH-CH1-cytokine×VL-CL-scFv(VH-VL), (vi) VL-CL-cytokine×VH-CH1-scFv(VH-VL), (vii) VH-CH1-cytokine×VL-CL-scFv(VL-VH), and (viii) VL-CL-cytokine×VH-CH1-scFv(VL-VH), where “-” denotes a linker or direct fusion between different moieties, and the different moieties in a peptidic chain are listed according to the N to C orientation in the peptidic chain. In some embodiments, the monomer immunoconjugate molecule can multimerize into a complex, such as a trimer. In specific embodiments, multimerization is through interaction between the cytokine domains of two or more immunoconjugate molecules. In some embodiments, immunoconjugate molecules forming the multimer can each assume a configuration selected from (i) to (viii) of this paragraph, which configurations can be the same (homo-multimer complex) as, or different (hetero-multimer complex) from, one another. In specific embodiments, the cytokine is wild-type or mutant TNF-α. In specific embodiments, the multimerization is through interaction between the extensive trimer interfaces of the TNF-α polypeptides contained in the immunoconjugate molecules. In specific embodiments, the TAA is Trop-2 or FAP.

In some embodiments, the present immunoconjugate comprises (a) an anti-cytokine/anti-TAA two-in-one Fab antibody, (b) a cytokine polypeptide, and (c) an anti-TAA scFv antibody. In various embodiments, the immunoconjugate monomer can assume any one of the eight possible configurations: (i) cytokine-VH-CH1-scFv(VH-VL)×VL-CL, (ii) cytokine-VL-CL-scFv(VH-VL)×VH-CH1, (iii) cytokine-VH-CH1-scFv(VL-VH)×VL-CL, (iv) cytokine-VL-CL-scFv(VL-VH)×VH-CH1, (v) VH-CH1-scFv(VH-VL)-cytokine×VL-CL, (vi) VL-CL-scFv(VH-VL)-cytokine×VH-CH1, (vii) VH-CH1-scFv(VL-VH)-cytokine×VL-CL, and (viii) VL-CL-scFv(VL-VH)-cytokine×VH-CH1, where “-” denotes a linker or direct fusion between different moieties, and the different moieties in a peptidic chain are listed according to the N to C orientation in the peptidic chain. In some embodiments, the monomer immunoconjugate molecule can multimerize into a complex, such as a trimer. In specific embodiments, multimerization is through interaction between the cytokine domains of two or more immunoconjugate molecules. In some embodiments, immunoconjugate molecules forming the multimer can each assume a configuration selected from (i) to (viii) of this paragraph, which configurations can be the same (homo-multimer complex) as, or different (hetero-multimer complex) from, one another. In specific embodiments, the cytokine is wild-type or mutant TNF-α. In specific embodiments, the multimerization is through interaction between the extensive trimer interfaces of the TNF-α polypeptides contained in the immunoconjugate molecules. In specific embodiments, the TAA is Trop-2 or FAP.

In some embodiments, the present immunoconjugate comprises (a) an anti-cytokine/anti-TAA two-in-one Fab antibody, (b) a cytokine polypeptide, and (c) an anti-TAA VHH antibody. In various embodiments, the immunoconjugate monomer can assume any one of the four possible configurations: (i) cytokine-VH-CH1-VHH×VL-CL, (ii) cytokine-VL-CL-VHH×VH-CH1, (iii) VH-CH1-VHH-cytokine×VL-CL, and (iv) VL-CL-VHH-cytokine×VH-CH1, where “-” denotes a linker or direct fusion between different moieties, and the different moieties in a peptidic chain are listed according to the N to C orientation in the peptidic chain. In some embodiments, the monomer immunoconjugate molecule can multimerize into a complex, such as a trimer. In specific embodiments, multimerization is through interaction between the cytokine domains of two or more immunoconjugate molecules. In some embodiments, immunoconjugate molecules forming the multimer can each assume a configuration selected from (i) to (iv) of this paragraph, which configurations can be the same (homo-multimer complex) as, or different (hetero-multimer complex) from, one another. In specific embodiments, the cytokine is wild-type or mutant TNF-α. In specific embodiments, the multimerization is through interaction between the extensive trimer interfaces of the TNF-α polypeptides contained in the immunoconjugate molecules. In specific embodiments, the TAA is Trop-2 or FAP.

In some embodiments, the present immunoconjugate comprises (a) an anti-cytokine/anti-TAA two-in-one Fab antibody, (b) a cytokine polypeptide, and (c) an anti-TAA VHH antibody. In various embodiments, the immunoconjugate monomer can assume any one of the four possible configurations: (i) cytokine-VH-CH1×VL-CL-VHH, (i) cytokine-VL-CL×VH-CH1-VHH, (i) VH-CH1-cytokine×VL-CL-VHH, and (i) VL-CL-cytokine×VH-CH1-VHH, where “-” denotes a linker or direct fusion between different moieties, and the different moieties in a peptidic chain are listed according to the N to C orientation in the peptidic chain. In some embodiments, the monomer immunoconjugate molecule can multimerize into a complex, such as a trimer. In specific embodiments, multimerization is through interaction between the cytokine domains of two or more immunoconjugate molecules. In some embodiments, immunoconjugate molecules forming the multimer can each assume a configuration selected from (i) to (iv) of this paragraph, which configurations can be the same (homo-multimer complex) as, or different (hetero-multimer complex) from, one another. In specific embodiments, the cytokine is wild-type or mutant TNF-α. In specific embodiments, the multimerization is through interaction between the extensive trimer interfaces of the TNF-α polypeptides contained in the immunoconjugate molecules. In specific embodiments, the TAA is Trop-2 or FAP.

In some embodiments, the present immunoconjugate molecule comprises an anchoring moiety, a masking moiety and a cytokine moiety that are operably linked to one another via a conjugating moiety. In some embodiments, the conjugating moiety comprises an immunoglobulin Fc domain composed of the Fc regions of both heavy chains of the immunoglobulin (each a subunit of the Fc domain). In some embodiments, the Fc domain is the Fc domain of an IgG molecule (e.g., IgG1, IgG2, IgG3, or IgG4).

In some embodiments, the two subunits of the Fc domain can be both native sequence Fc regions. In some embodiments, the two subunits of the Fc domain can be both variant Fc regions. In some embodiments, the two subunits of the Fc domain can be one native sequence Fc region and one variant Fc region. In certain embodiments, the Fc domain comprises a modification promoting hetero-dimerization of two non-identical immunoglobulin heavy chains. The site of most extensive protein-protein interaction between the two polypeptidic chains of a human IgG Fc domain is in the CH3 domain of the Fc regions. Thus, in one embodiment, said modification is in the CH3 domain of the Fc regions. In a specific embodiment said modification is a knob-into-hole modification, comprising a knob modification in one of the Fc subunits and a hole modification in the other one of the Fc subunits. The knob-into-hole technology is described e.g., in U.S. Pat. Nos. 5,731,168; 7,695,936; Ridgway et al., Prat Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). The protuberance and cavity can be made by altering the nucleic acid encoding the polypeptides, e.g., by site-specific mutagenesis, or by peptide synthesis. In a specific embodiment, a knob modification comprises the amino acid substitution T366W in one of the two Fc subunits, and the hole modification comprises the amino acid substitutions T366S, L368A and Y407V in the other one of the two Fc subunits. In a further specific embodiment, the Fc subunit comprising the knob modification additionally comprises the amino acid substitution S354C, and the immunoglobulin heavy chain comprising the hole modification additionally comprises the amino acid substitution Y349C. Introduction of these two cysteine residues results in formation of a disulfide bridge between the two heavy chains, further stabilizing the dimer (Carter, J. Immunol Methods 248, 7-15 (2001)).

In an alternative embodiment a modification promoting heterodimerization of two non-identical polypeptidic chains comprises a modification mediating electrostatic steering effects, e.g., as described in PCT publication WO 2009/089004. Generally, this method involves replacement of one or more amino acid residues at the interface of the two polypeptidic chains by charged amino acid residues so that homodimer formation becomes electro statically unfavorable but heterodimerization electrostatically favorable.

Without being bound by the theory, it is contemplated that an Fc domain confers to the immunoconjugate molecule favorable pharmacokinetic properties, including a long serum half-life which contributes to good accumulation in the target tissue and a favorable tissue-blood distribution ratio. At the same time an Fc domain may lead to undesirable targeting of the immunoconjugate molecules to cells expressing Fc receptors rather than to the target antigen-bearing cells. Moreover, the co-activation of Fc receptor signaling pathways may lead to cytokine release which, in combination with the cytokine polypeptide in the immunoconjugate molecule and the long half-life of the immunoconjugate, results in excessive activation of cytokine receptors and severe side effects upon systemic administration.

In certain embodiments, the modification to the Fc region of the antibody results in the decrease or elimination of an effector function of the antibody. In certain embodiments, the effector function is ADCC, ADCP, and/or CDC. In some embodiments, the effector function is ADCC. In other embodiments, the effector function is ADCP. In other embodiments, the effector function is CDC. In one embodiment, the effector function is ADCC and ADCP. In one embodiment, the effector function is ADCC and CDC. In one embodiment, the effector function is ADCP and CDC. In one embodiment, the effector function is ADCC, ADCP and CDC. This may be achieved by introducing one or more amino acid substitutions in an Fc region of the antibody. For example, substitutions into human IgG1 using IgG2 residues at positions 233-236 and IgG4 residues at positions 327, 330, and 331 were shown to greatly reduce ADCC and CDC (see, e.g., Armour et al., 1999, Eur. J. Immunol. 29(8):2613-24; and Shields et al., 2001, J. Biol. Chem. 276(9): 6591-604). Other Fc variants are provided elsewhere herein.

To increase the serum half-life of the antibody, one may incorporate a salvage receptor binding epitope into the antibody (especially an antibody fragment), for example, as described in U.S. Pat. No. 5,739,277. Term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

Accordingly, in some embodiments, the Fc domain forming part of the immunoconjugate molecule according to the present disclosure is engineered to have reduced binding affinity to an Fc receptor. In one such embodiment the Fc domain comprises one or more amino acid mutation that reduces the binding affinity of the Fc domain to an Fc receptor. In one such embodiment, the one or more such amino acid mutations are present in one of the two Fc subunits of the Fc domain. In another such embodiment, the one or more such amino acid mutations are present in both of the two Fc subunits of the Fc domain. In various embodiments, such amino acid mutations reduce the binding affinity of the immunoconjugate to the Fc receptor by at least 2-fold, at least 5-fold, or at least 10-fold.

In some embodiments where there is more than one amino acid mutation that reduces the binding affinity of the Fc domain composed of the present immunoconjugate molecule to the Fc receptor, the combination of these amino acid mutations can reduce the binding affinity of the Fc domain to the Fc receptor by at least 10-fold, at least 20-fold, or even at least 50-fold. In one embodiment the immunoconjugate comprising an engineered immunoglobulin molecule exhibits less than 20%, particularly less than 10%, more particularly less than 5% of the binding affinity to an Fc receptor as compared to an immunoconjugate comprising a non-engineered immunoglobulin molecule.

In some embodiments, the Fc receptor is an activating Fc receptor. In a specific embodiment the Fc receptor is an Fcγ receptor. More specifically, in some embodiments, the Fc receptor is an FcγRIIIα, FcγRI or FcγRIIα receptor. In some embodiments, binding of the Fc domain to each of these exemplary receptors is reduced. In some embodiments, binding affinity of the Fc domain to a complement component is reduced. Specifically in some embodiments, binding affinity of the Fc domain to C1q is reduced. In one embodiment, binding affinity to neonatal Fc receptor (FcRn) is not reduced. Substantially similar binding to FcRn, i.e., preservation of the binding affinity of the Fc domain to said receptor, is achieved when the immunoconjugate comprising said Fc domain exhibits greater than about 70% of the binding affinity of a non-engineered form of the immunoconjugate molecule comprising said non-engineered form of the Fc to FcRn. Immunoglobulins, or immunoconjugates comprising said immunoglobulins, may exhibit greater than about 80% and even greater than about 90% of such affinity.

In some embodiments, the Fc domain forming part of the present immunoconjugate molecule is not a native sequence Fc domain and has at least one amino acid mutation in one of its Fc subunits. In some embodiments, the Fc domain forming part of the present immunoconjugate molecule is not a native sequence Fc domain and has at least one amino acid mutation in both of its Fc subunits. In some embodiments, the amino acid mutations in both Fc subunits of an Fc domain are the same mutations. In some embodiments, the amino acid mutations in the two Fc subunits of an Fc domain are different mutations. In some embodiments, the amino acid mutation is selected from amino acid substitution, amino acid deletion and amino acid insertion. In particular embodiments, one or both of the Fc subunits in the Fc domain of the immunoconjugate molecule comprise one or more amino acid mutations at any one or more amino acid positions 228, 233, 234, 235, 236, 265, 297, 329, 330, and 331 of the Fc subunit, where the number of the residues in the Fc subunit is that of the EU index as in Kabat. In particular embodiments, such one or more amino acid substitutions comprise S228P. In particular embodiments, such one or more amino acid substitutions comprise E233P. In particular embodiments, such one or more amino acid substitutions comprise L234V or L234A. In particular embodiments, such one or more amino acid substitutions comprise L235A or L235E. In particular embodiments, such one or more amino acid deletion comprises ΔG236. In particular embodiments, such one or more amino acid substitutions comprise D265G. In particular embodiments, such one or more amino acid substitutions comprise N297A or N297D. In particular embodiments, such one or more amino acid substitutions comprise P329E, P329A or P329G, particularly P329E. In particular embodiments, such one or more amino acid substitutions comprise A330S. In particular embodiments, such one or more amino acid substitutions comprise P331S.

In particular embodiments, the Fc domain comprises amino acid mutations at positions E233, L234, L235, G236, A330, and P331. In specific embodiments, both of the two Fc subunits comprises amino acid mutations at positions E233, L234, L235, G236, A330, and P331. In particular embodiments, the Fc domain comprises amino acid mutations of E233P, L234V, L235A, ΔG236, A330S, and P331S. In specific embodiments, both of the two Fc subunits comprises amino acid mutations of E233P, L234V, L235A, ΔG236, P329S, A330S, and P331S.

In particular embodiments, the Fc domain comprises amino acid mutations at positions L234, L235, A330, and P331. In specific embodiments, both of the two Fc subunits comprise amino acid mutations at positions L234, L235, A330, and P331. In particular embodiments, the Fc domain comprises amino acid mutations of L234A, L235A, A330S, and P331S. In specific embodiments, both of the two Fc subunits comprise amino acid mutations of L234A, L235A, A330S, and P331S.

In particular embodiments, the Fc domain comprises amino acid mutations at positions E233, L234, L235, G236, P329, A330, and P331. In specific embodiments, both of the two Fc subunits comprise amino acid mutations at positions E233, L234, L235, G236, P329, A330, and P331. In particular embodiments, the Fc domain comprises amino acid mutations of E233P, L234V, L235A, ΔG236, P329E, A330S, and P331S. In specific embodiments, both of the two Fc subunits comprise amino acid mutations of E233P, L234V, L235A, ΔG236, P329E, A330S, and P331S.

In particular embodiments, the Fc domain comprises amino acid mutations at positions L234, L235, P329, A330, and P331. In specific embodiments, both of the two Fc subunits comprise amino acid mutations at positions L234, L235, P329, A330, and P331. In particular embodiments, the Fc domain comprises amino acid mutations of L234A, L235A, P329E, A330S, and P331S. In specific embodiments, both of the two Fc subunits comprise amino acid mutations of L234A, L235A, P329E, A330S, and P331S.

In particular embodiments, the Fc domain comprises amino acid mutations at positions E233, L234, L235, G236, and P329. In specific embodiments, both of the two Fc subunits comprise amino acid mutations at positions E233, L234, L235, G236, and P329. In particular embodiments, the Fc domain comprises amino acid mutations of E233P, L234V, L235A, ΔG236, and P329E. In specific embodiments, both of the two Fc subunits comprise amino acid mutations of E233P, L234V, L235A, ΔG236, and P329E.

In particular embodiments, the Fc domain comprises amino acid mutations at positions L234, L235, P329. In specific embodiments, both of the two Fc subunits comprise amino acid mutations at positions L234, L235, P329. In particular embodiments, the Fc domain comprises amino acid mutations of L234A, L235A, and P329E. In specific embodiments, both of the two Fc subunits comprise amino acid mutations of L234A, L235A, and P329E.

In particular embodiments, the Fc domain comprises amino acid mutations at positions E233, L234, L235, G236, D265, A330, and P331. In specific embodiments, both of the two Fc subunits comprise amino acid mutations at positions E233, L234, L235, G236, D265, A330, and P331. In particular embodiments, the Fc domain comprises amino acid mutations of E233P, L234V, L235A, ΔG236, D265G, A330S, and P331S. In specific embodiments, both of the two Fc subunits comprise amino acid mutations of E233P, L234V, L235A, ΔG236, D265G, A330S, and P331S. In these embodiments, the Fc domain has reduced binding affinity to the Fcγ receptor.

In particular embodiments, the Fc domain comprises amino acid mutations at positions L234, L235, D265, A330, and P331. In specific embodiments, both of the two Fc subunits comprise amino acid mutations at positions L234, L235, D265, A330, and P331. In particular embodiments, the Fc domain comprises amino acid mutations of L234A, L235A, D265G, A330S, and P331S. In specific embodiments, both of the two Fc subunits comprise amino acid mutations of L234A, L235A, D265G, A330S, and P331S.

In particular embodiments, the Fc domain comprises amino acid mutations at positions E233, L234, L235, G236, D265, P329, A330, and P331. In specific embodiments, both of the two Fc subunits comprise amino acid mutations at positions E233, L234, L235, G236, D265, P329, A330, and P331. In particular embodiments, the Fc domain comprises amino acid mutations of E233P, L234V, L235A, ΔG236, D265G, P329E, A330S, and P331S. In specific embodiments, both of the two Fc subunits comprise amino acid mutations of E233P, L234V, L235A, ΔG236, D265G, P329E, A330S, and P331S.

In particular embodiments, the Fc domain comprises amino acid mutations at positions L234, L235, D265, P329, A330, and P331. In specific embodiments, both of the two Fc subunits comprise amino acid mutations at positions L234, L235, D265, P329, A330, and P331. In particular embodiments, the Fc domain comprises amino acid mutations of L234A, L235A, D265G, P329E, A330S, and P331S. In specific embodiments, both of the two Fc subunits comprise amino acid mutations of L234A, L235A, D265G, P329E, A330S, and P331S.

In particular embodiments, the Fc domain comprises amino acid mutations at positions E233, L234, L235, G236, D265, and P329. In specific embodiments, both of the two Fc subunits comprise amino acid mutations at positions E233, L234, L235, G236, D265, and P329. In particular embodiments, the Fc domain comprises amino acid mutations of E233P, L234V, L235A, ΔG236, D265G, and P329E. In specific embodiments, both of the two Fc subunits comprise amino acid mutations of E233P, L234V, L235A, ΔG236, D265G, and P329E.

In particular embodiments, the Fc domain comprises amino acid mutations at positions L234, L235, D265, and P329. In specific embodiments, both of the two Fc subunits comprise amino acid mutations at positions L234, L235, D265, and P329. In particular embodiments, the Fc domain comprises amino acid mutations of L234A, L235A, D265G, and P329E. In specific embodiments, both of the two Fc subunits comprise amino acid mutations of L234A, L235A, D265G, and P329E.

In particular embodiments, the Fc domain comprises amino acid mutations at positions L234, L235, and P329. In specific embodiments, both of the two Fc subunits comprise amino acid mutations at positions L234, L235, and P329. In particular embodiments, the Fc domain comprises amino acid mutations of L234A, L235A, and P329G. In specific embodiments, both of the two Fc subunits comprise amino acid mutations of L234A, L235A, and P329G.

According to the present disclosure, the present immunoconjugate molecule comprises an anchoring moiety, a masking moiety and a cytokine moiety that are operably linked to one another via a conjugating moiety. In specific embodiments, the cytokine moiety comprises a cytokine polypeptide. In specific embodiments, the masking moiety comprises a bispecific two-in-one antibody or antigen binding fragment capable of binding to the cytokine polypeptide and a second target antigen. In specific embodiments, the anchoring moiety comprises an antibody or antigen binding fragment thereof capable of binding to a third target antigen. In specific embodiments, the conjugating moiety comprises an immunoglobulin Fc domain composed of two Fc regions of immunoglobulin heavy chains (each Fc region is referred to as a subunit of the Fc domain or “Fc subunit”). In some embodiments, the Fc domain comprises a modification promoting hetero-dimerization of the two Fc subunits. In specific embodiments, said modification is a knob-into-hole modification, comprising a knob modification in one of the Fc subunits (Fc-knob) and a hole modification in the other one of the Fc subunits (Fc-hole).

According to the present disclosure, in these embodiments, the cytokine moiety, the masking moiety, and the anchoring moiety of the immunoconjugate molecule can be operably linked to one another via the conjugating moiety in a variety of different configurations.

FIGS. 2B to 2U are schematic illustrations of antibody-cytokine immunoconjugates of different molecular configurations according to the present disclosure. In one exemplary embodiment, the cytokine moiety comprises a cytokine polypeptide that is fused to the C-terminus of one Fc subunit. In one exemplary embodiment, the masking moiety comprises an antibody or antigen binding fragment thereof that is fused to the C-terminus of one Fc subunit. In one exemplary embodiment, the cytokine moiety comprises a cytokine polypeptide that is fused to the C-terminus of one subunit of the Fc domain, and the masking moiety comprises an antibody or antigen binding fragment thereof that is fused to the C-terminus of the other Fc subunit. In some embodiments, the masking moiety is fused to the C-terminus of the Fc subunit. In some embodiments, the Fc domain comprises a modification promoting hetero-dimerization of the two Fc subunits. In specific embodiments, said modification is a knob-into-hole modification, comprising a knob modification in one of the Fc subunits (Fc-knob subunit) and a hole modification in the other one of the Fc subunits (Fc-hole subunit).

In one exemplary embodiment, the cytokine moiety comprises a cytokine polypeptide that is fused to the C-terminus of one Fc subunit. In one exemplary embodiment, the masking moiety comprises a bispecific two-in-one antibody or antigen binding fragment thereof that is fused to the C-terminus of one Fc subunit. In one exemplary embodiment, the anchoring moiety comprises an antibody or antigen binding fragment thereof that is fused to the N-terminus of one Fc subunit. In one exemplary embodiment, the cytokine moiety comprises a cytokine polypeptide that is fused to the C-terminus of one subunit of the Fc domain, and the masking moiety comprises a bispecific two-in-one antibody or antigen binding fragment thereof that is fused to the C-terminus of the other Fc subunit. In one exemplary embodiment, the cytokine moiety comprises a cytokine polypeptide that is fused to the C-terminus of one subunit of the Fc domain, the masking moiety comprises a bispecific two-in-one antibody or antigen binding fragment thereof that is fused to the C-terminus of the other Fc subunit, and the anchoring moiety comprises an antibody or antigen binding fragment thereof that is fused to the N-terminus of one subunit of the Fc domain. In specific embodiments, the anchoring moiety and the cytokine moiety are fused to the N- and C-terminus of the same Fc subunit, respectively. In specific embodiment, the masking moiety and the cytokine moiety are fused to the N- and C-terminus of the same Fc subunit, respectively. In some embodiments, the Fc domain comprises a modification promoting hetero-dimerization of the two Fc subunits. In specific embodiments, said modification is a knob-into-hole modification, comprising a knob modification in one of the Fc subunits (Fc-knob subunit) and a hole modification in the other one of the Fc subunits (Fc-hole subunit).

In one exemplary embodiment, the masking moiety comprises a bispecific two-in-one antibody or antigen binding fragment thereof that is fused to the N-terminus of one Fc subunit. In one exemplary embodiment, the cytokine moiety comprises a cytokine polypeptide that is fused to the masking moiety. In one exemplary embodiment, the masking moiety comprises a bispecific two-in-one antibody or antigen binding fragment thereof that is fused to the N-terminus of one Fc subunit, and the cytokine moiety comprises a cytokine polypeptide that is fused to the masking moiety. In one exemplary embodiment, the anchoring moiety comprises an antibody or antigen binding fragment thereof that is fused to the N-terminus of one Fc subunit. In one exemplary embodiment, the cytokine moiety comprises a cytokine polypeptide that is fused to the anchoring moiety. In one exemplary embodiment, the masking moiety comprises a bispecific two-in-one antibody or antigen binding fragment thereof that is fused to the N-terminus of one Fc subunit, the anchoring moiety comprises an antibody or antigen binding fragment thereof that is fused to the N-terminus of the other Fc subunit, and the cytokine moiety comprises a cytokine polypeptide that is fused to the masking moiety. In one exemplary embodiment, the masking moiety comprises a bispecific two-in-one antibody or antigen binding fragment thereof that is fused to the N-terminus of one Fc subunit, the anchoring moiety comprises an antibody or antigen binding fragment thereof that is fused to the N-terminus of the other Fc subunit, and the cytokine moiety comprises a cytokine polypeptide that is fused to the anchoring moiety. In some embodiments, the Fc domain comprises a modification promoting hetero-dimerization of the two Fc subunits. In specific embodiments, said modification is a knob-into-hole modification, comprising a knob modification in one of the Fc subunits (Fc-knob subunit) and a hole modification in the other one of the Fc subunits (Fc-hole subunit).

In one exemplary embodiment, the masking moiety comprises a bispecific two-in-one antibody or an antigen binding fragment thereof that is fused to the C-terminus of one Fc subunit. In one exemplary embodiment, the cytokine moiety comprises a cytokine polypeptide that is fused to the masking moiety. In one exemplary embodiment, the masking moiety comprises a bispecific two-in-one antibody or an antigen binding fragment thereof that is fused to the C-terminus of one Fc subunit, and the cytokine moiety comprises a cytokine polypeptide that is fused to the masking moiety. In one exemplary embodiment, the anchoring moiety comprising an antibody or antigen binding fragment thereof that is fused to the N terminus of one Fc subunit. In one exemplary embodiment, the masking moiety comprises a bispecific two-in-one antibody or an antigen binding fragment thereof that is fused to the C-terminus of one Fc subunit, the anchoring moiety comprises an antibody or antigen binding fragment thereof that is fused to the N-terminus of the other Fc subunit, and the cytokine moiety comprises a cytokine polypeptide fused to the masking moiety. In specific embodiments, the masking moiety and the anchoring moiety bind to the same Fc subunit. In specific embodiments, the masking moiety and the anchoring moiety bind to different Fc subunits. In some embodiments, the Fc domain comprises a modification promoting hetero-dimerization of the two Fc subunits. In specific embodiments, said modification is a knob-into-hole modification, comprising a knob modification in one of the Fc subunits (Fc-knob subunit) and a hole modification in the other one of the Fc subunits (Fc-hole subunit).

In one exemplary embodiment, the masking moiety comprises a bispecific two-in-one antibody or an antigen binding fragment thereof that is fused to the C-terminus of one Fc subunit. In one exemplary embodiment, the cytokine moiety comprises a cytokine polypeptide that is fused to the C-terminus of one Fc subunit. In one exemplary embodiment, the anchoring moiety comprises an antibody or antigen binding fragment thereof that is fused to the masking moiety. In some embodiments, the Fc domain comprises a modification promoting hetero-dimerization of the two Fc subunits. In specific embodiments, said modification is a knob-into-hole modification, comprising a knob modification in one of the Fc subunits (Fc-knob subunit) and a hole modification in the other one of the Fc subunits (Fc-hole subunit).

In one exemplary embodiment, the masking moiety comprises a bispecific two-in-one antibody or an antigen binding fragment thereof that is fused to the N-terminus of one Fc subunit. In one exemplary embodiment, the cytokine moiety comprises a cytokine polypeptide that is fused to the N-terminus of one Fc subunit. In one exemplary embodiment, the anchoring moiety comprises an antibody or antigen binding fragment thereof that is fused to the masking moiety. In some embodiments, the Fc domain comprises a modification promoting hetero-dimerization of the two Fc subunits. In specific embodiments, said modification is a knob-into-hole modification, comprising a knob modification in one of the Fc subunits (Fc-knob subunit) and a hole modification in the other one of the Fc subunits (Fc-hole subunit).

According to the present disclosure, in any of the embodiments described herein, the different moieties of the immunoconjugate molecule can be connected with a peptidic linker sequence. In some embodiments, the peptidic linker has at least 5 amino acid residues. In some embodiments, the peptidic linker has at least 7 amino acid residues. In some embodiments, the peptidic linker has at least 10 amino acid residues. In some embodiments, the peptidic linker has at least 15 amino acid residues. In some embodiments, the peptidic linker has at least 20 amino acid residues.

According to the present disclosure, in any of the embodiments described herein, non-limiting examples of an antibody forming part of the immunoconjugate molecule can be synthetic antibodies, recombinantly produced antibodies, camelized antibodies, intrabodies, anti-idiotypic (anti-Id) antibodies. In some embodiments, an antibody forming part of the immunoconjugate molecule is a monoclonal antibody. In any of the embodiments described herein, an antigen binding fragment forming part of the immunoconjugate molecule can be functional fragments of an antibody that retains some or all of the binding activity of the antibody from which the fragment was derived. Non-limiting examples of functional fragments (e.g., antigen-binding fragments such as TNF-α-binding fragments) include single-chain Fvs (scFv) (e.g., including monospecific, bispecific, etc.), Fab fragments (e.g., including monospecific, bispecific, etc.), F(ab′) fragments, F(ab)₂ fragments, F(ab′)₂ fragments, disulfide-linked Fvs (dsFv), Fd fragments, Fv fragments, diabody, triabody, tetrabody, minibody, and single domain antibody (VHH or nanobody). In specific embodiments, the immunoconjugate molecule can have any of the configurations as shown in FIGS. 1 and 2.

For example, in specific embodiments, the bispecific two-in-one antibody in the masking moiety of the present immunoconjugate molecule is a Fab fragment. For example, in specific embodiments, the bispecific two-in-one antibody in the masking moiety of the present immunoconjugate molecule is a ScFv fragment. For example, in specific embodiments, the bispecific two-in-one antibody in the masking moiety of the present immunoconjugate molecule is a single domain (VHH) antibody.

For example, in specific embodiments, the antibody in the anchoring moiety of the present immunoconjugate molecule is a Fab fragment. For example, in specific embodiments, the antibody in the anchoring moiety of the present immunoconjugate molecule is a ScFv fragment. For example, in specific embodiments, the antibody in the anchoring moiety of the present immunoconjugate molecule is a single domain (VHH) antibody.

For example, in specific embodiments, the bispecific two-in-one antibody in the masking moiety of the present immunoconjugate molecule is a Fab fragment, and the antibody in the anchoring moiety of the immunoconjugate molecule is also a Fab fragment. For example, in specific embodiments, the bispecific two-in-one antibody in the masking moiety of the present immunoconjugate molecule is a Fab fragment, and the antibody in the anchoring moiety of the immunoconjugate molecule is a ScFv fragment. For example, in specific embodiments, the bispecific two-in-one antibody in the masking moiety of the present immunoconjugate molecule is a Fab fragment, and the antibody in the anchoring moiety of the immunoconjugate molecule is a single domain (VHH) fragment.

For example, in specific embodiments, the bispecific two-in-one antibody in the masking moiety of the present immunoconjugate molecule is a ScFv fragment, and the antibody in the anchoring moiety of the immunoconjugate molecule is a Fab fragment. For example, in specific embodiments, the bispecific two-in-one antibody in the masking moiety of the present immunoconjugate molecule is a ScFv fragment, and the antibody in the anchoring moiety of the immunoconjugate molecule is also ScFv fragment. For example, in specific embodiments, the bispecific two-in-one antibody in the masking moiety of the present immunoconjugate molecule is a ScFv fragment, and the antibody in the anchoring moiety of the immunoconjugate molecule is a single domain (VHH) fragment.

For example, in specific embodiments, the bispecific two-in-one antibody in the masking moiety of the present immunoconjugate molecule is a single domain (VHH) antibody, and the antibody in the anchoring moiety of the immunoconjugate molecule is a Fab fragment. For example, in specific embodiments, the bispecific two-in-one antibody in the masking moiety of the present immunoconjugate molecule is a single domain (VHH) antibody, and the antibody in the anchoring moiety of the immunoconjugate molecule is ScFv fragment. For example, in specific embodiments, the bispecific two-in-one antibody in the masking moiety of the present immunoconjugate molecule is a single domain (VHH) antibody, and the antibody in the anchoring moiety of the immunoconjugate molecule is also a single domain (VHH) fragment.

### 5.3.1 Polyclonal Antibodies

The antibodies forming part of the immunoconjugate molecules of the present disclosure may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a polypeptide or a fusion protein thereof (e.g., TNF-α polypeptide or FAP polypeptide). It may be useful to conjugate the immunizing agent to a protein known to be immunogenic in the mammal being immunized or to immunize the mammal with the protein and one or more adjuvants. Examples of such immunogenic proteins include, but are not limited to, keyhole limpet hemocyanin, serum albumin, bovine thyroglobulin, and soybean trypsin inhibitor. Examples of adjuvants which may be employed include Ribi, CpG, Poly 1C, Freund's complete adjuvant, and MPL-TDM adjuvant (monophosphoryl Lipid A, synthetic trehalose dicorynomycolate). The immunization protocol may be selected by one skilled in the art without undue experimentation. The mammal can then be bled, and the serum assayed for antibody titer. If desired, the mammal can be boosted until the antibody titer increases or plateaus. Additionally or alternatively, lymphocytes may be obtained from the immunized animal for fusion and preparation of monoclonal antibodies from hybridoma as described below.

### 5.3.2 Monoclonal Antibodies

The antibodies forming part of the immunoconjugate molecules of the present disclosure may alternatively be monoclonal antibodies. Monoclonal antibodies may be made using the hybridoma method first described by Kohler et al., 1975, Nature 256:495-97, or may be made by recombinant DNA methods (see, e.g., U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice 59-103 (1986)).

The hybridoma cells thus prepared are seeded and grown in a suitable culture medium which, in certain embodiments, contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which prevent the growth of HGPRT-deficient cells.

Exemplary fusion partner myeloma cells are those that fuse efficiently, support stable high-level production of antibody by the selected antibody-producing cells, and are sensitive to a selective medium that selects against the unfused parental cells. Exemplary myeloma cell lines are murine myeloma lines, such as SP-2 and derivatives, for example, X63-Ag8-653 cells available from the American Type Culture Collection (Manassas, VA), and those derived from MOPC-21 and MPC-11 mouse tumors available from the Salk Institute Cell Distribution Center (San Diego, CA). Human myeloma and mouse-human heteromyeloma cell lines also have been described for the production of human monoclonal antibodies (Kozbor, 1984, Immunol. 133:3001-05; and Brodeur et al., Monoclonal Antibody Production Techniques and Applications 51-63 (1987)).

Culture medium in which hybridoma cells are growing is assayed for production of monoclonal antibodies directed against the antigen. The binding specificity of monoclonal antibodies produced by hybridoma cells is determined by immunoprecipitation or by an in vitro binding assay, such as RIA or ELISA. The binding affinity of the monoclonal antibody can, for example, be determined by the Scatchard analysis described in Munson et al., 1980, Anal. Biochem. 107:220-39.

Once hybridoma cells that produce antibodies of the desired specificity, affinity, and/or activity are identified, the clones may be subcloned by limiting dilution procedures and grown by standard methods (Goding, supra). Suitable culture media for this purpose include, for example, DMEM or RPMI-1640 medium. In addition, the hybridoma cells may be grown in vivo as ascites tumors in an animal, for example, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells can serve as a source of such DNA. Once isolated, the DNA may be placed into expression vectors, which are then transfected into host cells, such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., 1993, Curr. Opinion in Immunol. 5:256-62 and Plückthun, 1992, Immunol. Revs. 130:151-88.

In some embodiments, an antibody that binds an epitope comprises an amino acid sequence of a VH domain and/or an amino acid sequence of a VL domain encoded by a nucleotide sequence that hybridizes to (1) the complement of a nucleotide sequence encoding any one of the VH and/or VL domain described herein under stringent conditions (e.g., hybridization to filter-bound DNA in 6× sodium chloride/sodium citrate (SSC) at about 45° C. followed by one or more washes in 0.2×SSC/0.1% SDS at about 50-65° C.), under highly stringent conditions (e.g., hybridization to filter-bound nucleic acid in 6×SSC at about 45° C. followed by one or more washes in 0.1×SSC/0.2% SDS at about 68° C.), or under other stringent hybridization conditions which are known to those of skill in the art. See, e.g., Current Protocols in Molecular Biology Vol. I, 6.3.1-6.3.6 and 2.10.3 (Ausubel et al. eds., 1989).

In a further embodiment, monoclonal antibodies or antibody fragments can be isolated from antibody phage libraries generated using the techniques described in, for example, Antibody Phage Display: Methods and Protocols (O'Brien and Aitken eds., 2002). In principle, synthetic antibody clones are selected by screening phage libraries containing phages that display various fragments of antibody variable region (Fv) fused to phage coat protein. Such phage libraries are screened against the desired antigen. Clones expressing Fv fragments capable of binding to the desired antigen are adsorbed to the antigen and thus separated from the non-binding clones in the library. The binding clones are then eluted from the antigen and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., 1994, Ann. Rev. Immunol. 12:433-55.

Repertoires of VH and VL genes can be separately cloned by PCR and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., 1993, EMBO J 12:725-34. Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, 1992, J. Mol. Biol. 227:381-88.

Screening of the libraries can be accomplished by various techniques known in the art. For example, an antigen (e.g., a TNF-α polypeptide, fragment, or epitope) can be used to coat the wells of adsorption plates, expressed on host cells affixed to adsorption plates or used in cell sorting, conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., 1990, Proteins 8:309-14 and WO 92/09690, and by use of a low coating density of antigen as described in Marks et al., 1992, Biotechnol. 10:779-83.

Antibodies that form part of the immunoconjugate molecules described herein can be obtained by designing a suitable antigen screening procedure to select for the phage clone of interest followed by construction of a full-length antibody clone using VH and/or VL sequences (e.g., the Fv sequences), or various CDR sequences from VH and VL sequences, from the phage clone of interest and suitable constant region (e.g., Fc) sequences described in Kabat et al., supra.

In another embodiment, antibodies that form part of the immunoconjugate molecules is generated by using methods as described in Bowers et al., 2011, Proc Natl Acad Sci USA. 108:20455-60, e.g., the SHM-XHL™ platform (AnaptysBio, San Diego, CA). Briefly, in this approach, a fully human library of IgGs is constructed in a mammalian cell line (e.g., HEK293) as a starting library. Mammalian cells displaying immunoglobulin that binds to a target peptide or epitope are selected (e.g., by FACS sorting), then activation-induced cytidine deaminase (AID)-triggered somatic hypermutation is reproduced in vitro to expand diversity of the initially selected pool of antibodies. After several rounds of affinity maturation by coupling mammalian cell surface display with in vitro somatic hypermutation, high affinity, high specificity antibodies are generated. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

### 5.3.2.1 Antibody Fragments

The present disclosure provides antibodies and antibody fragments that form parts of an immunoconjugate molecule. In certain circumstances there are advantages of using antibody fragments, rather than whole antibodies. The smaller size of the fragments allows for rapid clearance, and may lead to improved access to cells, tissues, or organs. For a review of certain antibody fragments, see Hudson et al., 2003, Nature Med. 9:129-34.

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. Fab, Fv, and scFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., 1992, Bio/Technology 10:163-67). According to another approach, F(ab′)₂ fragments can be isolated directly from recombinant host cell culture. Fab and F(ab′)₂ fragment with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of an scFv (See, e.g., Borrebaeck ed., supra). The antibody fragment may also be a “linear antibody,” for example, as described in the references cited above. Such linear antibodies may be monospecific or multi-specific, such as bispecific.

Smaller antibody-derived binding structures are the separate variable domains (V domains) also termed single variable domain antibodies (sdAbs). Certain types of organisms, the camelids and cartilaginous fish, possess high affinity single V-like domains mounted on an Fc equivalent domain structure as part of their immune system. (Woolven et al., 1999, Immunogenetics 50: 98-101; and Streltsov et al., 2004, Proc Natl Acad Sci USA. 101:12444-49). The V-like domains (called VhH in camelids and V-NAR in sharks) typically display long surface loops, which allow penetration of cavities of target antigens. They also stabilize isolated VH domains by masking hydrophobic surface patches.

These VhH and V-NAR domains have been used to engineer sdAbs. Human V domain variants have been designed using selection from phage libraries and other approaches that have resulted in stable, high binding VL- and VH-derived domains.

Antibodies provided herein include, but are not limited to, immunoglobulin molecules and immunologically active portions of immunoglobulin molecules, for example, molecules that contain an antigen binding site that bind to an epitope (e.g., TNF-α epitope or FAP epitope). The immunoglobulin molecules provided herein can be of any class (e.g., IgG, IgE, IgM, IgD, and IgA) or any subclass (e.g., IgG1, IgG2, IgG3, IgG4, IgA1, and IgA2) of immunoglobulin molecule.

Variants and derivatives of antibodies include antibody functional fragments that retain the ability to bind to an epitope (e.g., TNF-α epitope or FAP epitope). Exemplary functional fragments include Fab fragments (e.g., an antibody fragment that contains the antigen-binding domain and comprises a light chain and part of a heavy chain bridged by a disulfide bond); scFab (e.g., an antibody fragment that contains the antigen-binding domain and comprises a light chain and part of a heavy chain linked by a peptidic linker); Fab′ (e.g., an antibody fragment containing a single antigen-binding domain comprising an Fab and an additional portion of the heavy chain through the hinge region); F(ab′)₂ (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen binding domains, each of which may be directed to a different epitope); a single chain comprising a variable region, also known as, scFv (e.g., the variable, antigen-binding determinative region of a single light and heavy chain of an antibody linked together by a chain of 10-25 amino acids); a disulfide-linked Fv, or dsFv (e.g., the variable, antigen-binding determinative region of a single light and heavy chain of an antibody linked together by a disulfide bond); a camelized VH (e.g., the variable, antigen-binding determinative region of a single heavy chain of an antibody in which some amino acids at the VH interface are those found in the heavy chain of naturally occurring camel antibodies); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); and a triabody (e.g., a trimerized scFv, formed in a manner similar to a diabody, but in which three antigen-binding domains are created in a single complex; the three antigen-binding domains may be directed towards the same or different epitopes).

### 5.3.2.2 Humanized Antibodies

In some embodiments, antibodies forming part of an immunoconjugate molecule provided herein can be humanized antibodies that bind, including human and/or cynomolgus antigen (such as human TNF-α or human FAP). Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, V_{L}6 subgroup I (V_{L}6I) and V_{H} subgroup III (V_{H}III). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., 2002, J. Immunol. 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In FR shuffling, whole FRs are combined with the non-human CDRs instead of creating combinatorial libraries of selected residue variants (see, e.g., Dall'Acqua et al., 2005, Methods 36:43-60). The libraries may be screened for binding in a two-step process, first humanizing V_{L}, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder et al., 2007, Mol. Immunol. 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

### 5.3.2.3 Human Antibodies

Human antibodies can be constructed by combining Fv clone variable domain sequence(s) selected from human-derived phage display libraries with known human constant domain sequences(s). Alternatively, human monoclonal antibodies of the present disclosure can be made by the hybridoma method. Human myeloma and mouse-human heteromyeloma cell lines for the production of human monoclonal antibodies have been described, for example, by Kozbor, 1984, J. Immunol. 133:3001-05; Brodeur et al., Monoclonal Antibody Production Techniques and Applications 51-63 (1987); and Boerner et al., 1991, J. Immunol. 147:86-95.

It is also possible to produce transgenic animals (e.g., mice) that are capable, upon immunization, of producing a full repertoire of human antibodies in the absence of endogenous immunoglobulin production. Transgenic mice that express human antibody repertoires have been used to generate high-affinity human sequence monoclonal antibodies against a wide variety of potential drug targets (see, e.g., Jakobovits, A., 1995, Curr. Opin. Biotechnol. 6(5):561-66; Bruggemann and Taussing, 1997, Curr. Opin. Biotechnol. 8(4):455-58; U.S. Pat. Nos. 6,075,181 and 6,150,584; and Lonberg et al., 2005, Nature Biotechnol. 23:1117-25).

Alternatively, the human antibody may be prepared via immortalization of human B lymphocytes producing an antibody directed against a target antigen (e.g., such B lymphocytes may be recovered from an individual or may have been immunized in vitro) (see, e.g., Cole et al., Monoclonal Antibodies and Cancer Therapy (1985); Boerner et al., 1991, J. Immunol. 147(1):86-95; and U.S. Pat. No. 5,750,373).

Gene shuffling can also be used to derive human antibodies from non-human, for example, rodent, antibodies, where the human antibody has similar affinities and specificities to the starting non-human antibody. According to this method, which is also called “epitope imprinting” or “guided selection,” either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone (e.g., the epitope guides (imprints) the choice of the human chain partner). When the process is repeated in order to replace the remaining non-human chain, a human antibody is obtained (see, e.g., PCT WO 93/06213; and Osbourn et al., 2005, Methods 36:61-68). Unlike traditional humanization of non-human antibodies by CDR grafting, this technique provides completely human antibodies, which have no FR or CDR residues of non-human origin. Examples of guided selection to humanize mouse antibodies towards cell surface antigens include the folate-binding protein present on ovarian cancer cells (see, e.g., Figini et al., 1998, Cancer Res. 58:991-96) and CD147, which is highly expressed on hepatocellular carcinoma (see, e.g., Bao et al., 2005, Cancer Biol. Ther. 4:1374-80).

A potential disadvantage of the guided selection approach is that shuffling of one antibody chain while keeping the other constant could result in epitope drift. In order to maintain the epitope recognized by the non-human antibody, CDR retention can be applied (see, e.g., Klimka et al., 2000, Br. J. Cancer. 83:252-60; and Beiboer et al., 2000, J. Mol. Biol. 296:833-49). In this method, the non-human VH CDR3 is commonly retained, as this CDR may be at the center of the antigen-binding site and may be the most important region of the antibody for antigen recognition. In some instances, however, VH CDR3 and VL CDR3, as well as VH CDR2, VL CDR2, and VL CDR1 of the non-human antibody may be retained.

### 5.3.3 Antibody Variants

In some embodiments, amino acid sequence modification(s) of the antibodies that form part of the immunoconjugate molecules described herein are contemplated. For example, it may be desirable to improve the binding affinity and/or other biological properties of the antibody, including but not limited to specificity, thermostability, expression level, effector functions, glycosylation, reduced immunogenicity, or solubility. Thus, in addition to the specific antibodies provided herein, it is contemplated that antibody variants can be prepared. For example, antibody variants can be prepared by introducing appropriate nucleotide changes into the encoding DNA, and/or by synthesis of the desired antibody or polypeptide. Those skilled in the art who appreciate that amino acid changes may alter post-translational processes of the antibody, such as changing the number or position of glycosylation sites or altering the membrane anchoring characteristics.

In some embodiments, antibodies provided herein are chemically modified, for example, by the covalent attachment of any type of molecule to the antibody. The antibody derivatives may include antibodies that have been chemically modified, for example, by increase or decrease of glycosylation, acetylation, pegylation, phosphorylation, amidation, derivatization by known protecting/blocking groups, chemical cleavage, proteolytic cleavage, linkage to a cellular ligand or other protein, etc. Additionally, the antibody may contain one or more non-classical amino acids.

Variations may be a substitution, deletion, or insertion of one or more codons encoding the antibody or polypeptide that results in a change in the amino acid sequence as compared with the native sequence antibody or polypeptide. Amino acid substitutions can be the result of replacing one amino acid with another amino acid having similar structural and/or chemical properties, such as the replacement of a leucine with a serine, e.g., conservative amino acid replacements. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the full-length or mature native sequence.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for antibody-directed enzyme prodrug therapy) or a polypeptide which increases the serum half-life of the antibody.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Alternatively, conservative (e.g., within an amino acid group with similar properties and/or side chains) substitutions may be made, so as to maintain or not significantly change the properties. Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H).

Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions entail exchanging a member of one of these classes for another class. Such substituted residues also may be introduced into the conservative substitution sites or, into the remaining (non-conserved) sites. Accordingly, in one embodiment, an antibody or fragment thereof that binds to an epitope comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a murine monoclonal antibody provided herein. The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al., 1982, Nucl. Acids Res. 10:6487-500), cassette mutagenesis (see, e.g., Wells et al., 1985, Gene 34:315-23), or other known techniques can be performed on the cloned DNA to produce the antibody variant DNA.

Any cysteine residue not involved in maintaining the proper conformation of the antibody also may be substituted, for example, with another amino acid, such as alanine or serine, to improve the oxidative stability of the molecule and to prevent aberrant crosslinking. Conversely, cysteine bond(s) may be added to the antibody to improve its stability (e.g., where the antibody is an antibody fragment such as an Fv fragment).

In some embodiments, an antibody or antigen binding fragment thereof forming part of the immunoconjugate molecule of the present disclosure is a “de-immunized” antibody. A “de-immunized” antibody is an antibody derived from a humanized or chimeric antibody, which has one or more alterations in its amino acid sequence resulting in a reduction of immunogenicity of the antibody, compared to the respective original non-de-immunized antibody. One of the procedures for generating such antibody mutants involves the identification and removal of T cell epitopes of the antibody molecule. In a first step, the immunogenicity of the antibody molecule can be determined by several methods, for example, by in vitro determination of T cell epitopes or in silico prediction of such epitopes, as known in the art. Once the critical residues for T cell epitope function have been identified, mutations can be made to remove immunogenicity and retain antibody activity. For review, see, for example, Jones et al., 2009, Methods in Molecular Biology 525:405-23.

### 5.3.3.1 In Vitro Affinity Maturation

In some embodiments, antibody variants having an improved property such as affinity, stability, or expression level as compared to a parent antibody may be prepared by in vitro affinity maturation. Like the natural prototype, in vitro affinity maturation is based on the principles of mutation and selection. Libraries of antibodies are displayed as Fab, scFv, or V domain fragments either on the surface of an organism (e.g., phage, bacteria, yeast, or mammalian cell) or in association (e.g., covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Affinity matured antibodies can have nanomolar or even picomolar affinities for the target antigen.

Phage display is a widespread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and used to infect bacteria to produce phage for further rounds of selection. For review, see, for example, Hoogenboom, 2002, Methods. Mol. Biol. 178:1-37; and Bradbury and Marks, 2004, J. Immunol. Methods 290:29-49.

In a yeast display system (see, e.g., Boder et al., 1997, Nat. Biotech. 15:553-57; and Chao et al., 2006, Nat. Protocols 1:755-68), the antibody may be displayed as single-chain variable fusions (scFv) in which the heavy and light chains are connected by a flexible linker. The scFv is fused to the adhesion subunit of the yeast agglutinin protein Aga2p, which attaches to the yeast cell wall through disulfide bonds to Aga1p. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling of yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the scFv. Expression has been shown to correlate with the stability of the displayed protein, and thus antibodies can be selected for improved stability as well as affinity (see, e.g., Shusta et al., 1999, J. Mol. Biol. 292:949-56). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods; however, a recent approach uses the yeast cells' mating system to create combinatorial diversity estimated to be 10¹⁴ in size (see, e.g., U.S. Pat. Publication 2003/0186374; and Blaise et al., 2004, Gene 342:211-18).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al., 2006, Nucleic Acids Res. 34:e127). In mRNA display, a covalent bond between antibody and mRNA is established using puromycin as an adaptor molecule (Wilson et al., 2001, Proc. Natl. Acad. Sci. USA 98:3750-55).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step.

In a mammalian cell display system (see, e.g., Bowers et al., 2011, Proc Natl Acad Sci USA. 108:20455-60), a fully human library of IgGs is constructed based on germline sequence V-gene segments joined to prerecombined D(J) regions. Full-length V regions for heavy chain and light chain are assembled with human heavy chain and light chain constant regions and transfected into a mammalian cell line (e.g., HEK293). The transfected library is expanded and subjected to several rounds of negative selection against streptavidin (SA)-coupled magnetic beads, followed by a round of positive selection against SA-coupled magnetic beads coated with biotinylated target protein, peptide fragment, or epitope. Positively selected cells are expanded, and then sorted by rounds of FACS to isolate single cell clones displaying antibodies that specifically bind to the target protein, peptide fragment, or epitope. Heavy and light chain pairs from these single cell clones are retransfected with AID for further maturation. Several rounds of mammalian cell display, coupled with AID-triggered somatic hypermutation, generate high specificity, high affinity antibodies.

Diversity may also be introduced into the CDRs or the whole V genes of the antibody libraries in a targeted manner or via random introduction. The former approach includes sequentially targeting all the CDRs of an antibody via a high or low level of mutagenesis or targeting isolated hot spots of somatic hypermutations (see, e.g., Ho et al., 2005, J. Biol. Chem. 280:607-17) or residues suspected of affecting affinity on experimental basis or structural reasons. In a specific embodiment, somatic hypermutation is performed by AID-triggered somatic hypermutation, e.g., using the SHM-XEL™ platform (AnaptysBio, San Diego, CA). Random mutations can be introduced throughout the whole V gene using E. coli mutator strains, error-prone replication with DNA polymerases (see, e.g., Hawkins et al., 1992, J. Mol. Biol. 226:889-96), or RNA replicases. Diversity may also be introduced by replacement of regions that are naturally diverse via DNA shuffling or similar techniques (see, e.g., Lu et al., 2003, J. Biol. Chem. 278:43496-507; U.S. Pat. Nos. 5,565,332 and 6,989,250). Alternative techniques target hypervariable loops extending into framework-region residues (see, e.g., Bond et al., 2005, J. Mol. Biol. 348:699-709) employ loop deletions and insertions in CDRs or use hybridization-based diversification (see, e.g., U.S. Pat. Publication No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

Screening of the libraries can be accomplished by various techniques known in the art. For example, a target antigen (such as TNF-α or FAP polypeptide can be immobilized onto solid supports, columns, pins, or cellulose/poly(vinylidene fluoride) membranes/other filters, expressed on host cells affixed to adsorption plates or used in cell sorting, or conjugated to biotin for capture with streptavidin-coated beads or used in any other method for panning display libraries.

For review of in vitro affinity maturation methods, see, e.g., Hoogenboom, 2005, Nature Biotechnology 23:1105-16; Quiroz and Sinclair, 2010, Revista Ingeneria Biomedia 4:39-51; and references therein.

### 5.3.3.2 Modifications of Antibodies

Covalent modifications of antibodies forming part of the immunoconjugate molecule of the present disclosure are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the antibody included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337.

An antibody forming part of the immunoconjugate molecule of the present disclosure may also be modified to form chimeric molecules comprising an antibody fused to another, heterologous polypeptide or amino acid sequence, for example, a cytokine polypeptide (see, e.g., Terpe, 2003, Appl. Microbiol. Biotechnol. 60:523-33) or the Fc region of an IgG molecule (see, e.g., Aruffo, Antibody Fusion Proteins 221-42 (Chamow and Ashkenazi eds., 1999)).

Also provided herein are fusion proteins comprising an antibody provided herein that binds to a target antigen and a heterologous polypeptide. In some embodiments, the antibody is useful to deliver and/or immobilize the heterologous polypeptide to which the antibody is fused to cells having cell surface-expressed target antigen.

Also provided herein are panels of antibodies that bind to a target antigen (e.g., TNF-α or FAP). In specific embodiments, the panels of antibodies have different association rates, different dissociation rates, different affinities for the target antigen, and/or different specificities for a target antigen. In some embodiments, the panels comprise or consist of about 10, about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96-well or 384-well plates, for assays such as ELISAs.

### 5.3.4 Preparation of Antibodies and Immunoconjugate Molecules

Antibodies and other peptide components (e.g., cytokine polypeptide) forming part of the present immunoconjugate molecules may be produced by culturing cells transformed or transfected with a vector containing the encoding nucleic acids. Polynucleotide sequences encoding polypeptide components of the antibody of the present disclosure can be obtained using standard recombinant techniques. Desired polynucleotide sequences may be isolated and sequenced from antibody producing cells such as hybridomas cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in host cells. Many vectors that are available and known in the art can be used for the purpose of the present disclosure. Selection of an appropriate vector will depend mainly on the size of the nucleic acids to be inserted into the vector and the particular host cell to be transformed with the vector. Host cells suitable for expressing antibodies of the present disclosure include prokaryotes such as Archaebacteria and Eubacteria, including Gram-negative or Gram-positive organisms, eukaryotic microbes such as filamentous fungi or yeast, invertebrate cells such as insect or plant cells, and vertebrate cells such as mammalian host cell lines. Host cells are transformed with the above-described expression vectors and cultured in conventional nutrient media modified as appropriate for inducing promoters, selecting transformants, or amplifying the genes encoding the desired sequences. Antibodies produced by the host cells are purified using standard protein purification methods as known in the art.

Methods for antibody production including vector construction, expression, and purification are further described in Plückthun et al., Antibody Engineering: Producing antibodies in Escherichia coli: From PCR to fermentation 203-52 (McCafferty et al. eds., 1996); Kwong and Rader, E. coli Expression and Purification of Fab Antibody Fragments, in Current Protocols in Protein Science (2009); Tachibana and Takekoshi, Production of Antibody Fab Fragments in Escherischia coli, in Antibody Expression and Production (Al-Rubeai ed., 2011); and Therapeutic Monoclonal Antibodies: From Bench to Clinic (An ed., 2009).

It is, of course, contemplated that alternative methods, which are well known in the art, may be employed to prepare antibodies. For instance, the appropriate amino acid sequence, or portions thereof, may be produced by direct peptide synthesis using solid-phase techniques (see, e.g., Stewart et al., Solid-Phase Peptide Synthesis (1969); and Merrifield, 1963, J. Am. Chem. Soc. 85:2149-54). In vitro protein synthesis may be performed using manual techniques or by automation. Various portions of the antibody may be chemically synthesized separately and combined using chemical or enzymatic methods to produce the desired antibody. Alternatively, antibodies may be purified from cells or bodily fluids, such as milk, of a transgenic animal engineered to express the antibody, as disclosed, for example, in U.S. Pat. Nos. 5,545,807 and 5,827,690.

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of antibodies as provided herein, including, for example, antibodies with higher affinities and lower dissociation rates (see, e.g., U.S. Pat. Nos. 5,605,793; 5,811,238; 5,830,721; 5,834,252; and U.S. Pat. No. 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-13). Antibodies, or the encoded antibodies, may be altered by being subjected to random mutagenesis by error-prone PCR, random nucleotide insertion, or other methods prior to recombination. A polynucleotide encoding an antibody provided herein may be recombined with one or more components, motifs, sections, parts, domains, fragments, etc. of one or more heterologous molecules.

Methods for fusing or conjugating moieties (including polypeptides) to antibodies are known (see, e.g., Arnon et al., Monoclonal Antibodies for Immunotargeting of Drugs in Cancer Therapy, in Monoclonal Antibodies and Cancer Therapy 243-56 (Reisfeld et al. eds., 1985); Hellstrom et al., Antibodies for Drug Delivery, in Controlled Drug Delivery 623-53 (Robinson et al. eds., 2d ed. 1987); Thorpe, Antibody Carriers of Cytotoxic Agents in Cancer Therapy: A Review, in Monoclonal Antibodies: Biological and Clinical Applications 475-506 (Pinchera et al. eds., 1985); Analysis, Results, and Future Prospective of the Therapeutic Use of Radiolabeled Antibody in Cancer Therapy, in Monoclonal Antibodies for Cancer Detection and Therapy 303-16 (Baldwin et al. eds., 1985); Thorpe et al., 1982, Immunol. Rev. 62:119-58; U.S. Pat. Nos. 5,336,603; 5,622,929; 5,359,046; 5,349,053; 5,447,851; 5,723,125; 5,783,181; 5,908,626; 5,844,095; and 5,112,946; EP 307,434; EP 367,166; EP 394,827; PCT publications WO 91/06570, WO 96/04388, WO 96/22024, WO 97/34631, and WO 99/04813; Ashkenazi et al., 1991, Proc. Natl. Acad. Sci. USA, 88: 10535-39; Traunecker et al., 1988, Nature, 331:84-86; Zheng et al., 1995, J. Immunol. 154:5590-600; and Vil et al., 1992, Proc. Natl. Acad. Sci. USA 89:11337-41).

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of antibodies as provided herein, including, for example, antibodies with higher affinities and lower dissociation rates (see, e.g., U.S. Pat. Nos. 5,605,793; 5,811,238; 5,830,721; 5,834,252; and U.S. Pat. No. 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-13). Antibodies, or the encoded antibodies, may be altered by being subjected to random mutagenesis by error-prone PCR, random nucleotide insertion, or other methods prior to recombination. A polynucleotide encoding an antibody provided herein may be recombined with one or more components, motifs, sections, parts, domains, fragments, etc. of one or more heterologous molecules.

Conjugates of the antibody and agent may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that conjugates of antibodies and agents may be prepared using any suitable methods as disclosed in the art (see, e.g., Bioconjugate Techniques (Hermanson ed., 2d ed. 2008)).

Conventional conjugation strategies for antibodies and agents have been based on random conjugation chemistries involving the F-amino group of Lys residues or the thiol group of Cys residues, which results in heterogenous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen binding (see, e.g., Junutula et al., 2008, J. Immunol. Meth. 332: 41-52; and Junutula et al., 2008, Nature Biotechnol. 26:925-32). In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., 2008, Proc. Natl. Acad. Sci. USA 105:12451-56; and Hofer et al., 2009, Biochemistry 48(50):12047-57).

### 5.4 Methods of Using the Immunoconjugate Molecules and Compositions

As would be appreciated from the present disclosure, the immunoconjugate molecules according to the present disclosure can be used for delivering a cytokine and/or activating a cytokine activity at a target site of interest in a subject. Without being bound by the theory, it is also contemplated that when systemic exposure to certain cytokine activity can result in toxic side-effect in a subject, the immunoconjugate molecules of the present disclosure can be used for reducing toxicity or other side-effects of the cytokine by preventing the activation of the cytokine-mediated effect in locations other than the target site in the subject.

Accordingly, in one aspect, provided herein is a method for site-specific delivery of a cytokine molecule in a subject, the method comprising incorporating the cytokine into an immunoconjugate molecule according to the present disclosure, and delivering the immunoconjugate molecule to the subject. Particularly, in certain embodiments, the immunoconjugate molecule comprises the cytokine and an anchoring moiety capable of binding to a target antigen present at the target site in the subject, such that when the immunoconjugate molecule arrives at the target site, the anchoring moiety binds to the target antigen, thereby immobilizing the immunoconjugate molecule at the target site. In some embodiments, the method results in a higher concentration of the administered immunoconjugate molecule at the target site in the subject as compared to a non-target site.

In a related aspect, provided herein is also a method for site-specific activation of a cytokine activity in a subject, the method comprising incorporating the cytokine into an immunoconjugate molecule according to the present disclosure, and delivering the immunoconjugate molecule to the subject. Particularly, in certain embodiments, the immunoconjugate molecule comprises the cytokine and a masking moiety that binds to and inhibits the cytokine activity via intramolecular interaction. Particularly, the masking moiety is also capable of binding to a target antigen present at the target site, such that when the immunoconjugate molecule arrives at the target site, the masking moiety binds to the target antigen and disassociates from the cytokine, thereby activating the cytokine activity at the target site. In some embodiments, the method results in a higher cytokine activity at the target site in the subject as compared to a non-target site.

In specific embodiments, the immunoconjugate molecule used in the present methods comprises both a masking moiety and an anchoring moiety. In various embodiments, the target antigen recognized by the masking moiety and the anchoring moiety of the immunoconjugate molecule can be the same antigen or different antigens. In specific embodiments, the immunoconjugate molecule used in the present methods further comprises a conjugating moiety that operably connecting one or more of the cytokine moiety, masking moiety and anchoring moiety. In specific embodiments, the immunoconjugate molecule used in the present methods can be any of the immunoconjugate molecules as described in Section 5.3.

In some embodiments, the present methods result in reduced cytokine toxicity to the subject as compared a method that administered an equivalent amount of the cytokine in an unconjugated form. Accordingly, in a related aspect, provided herein is also a method for reducing a side-effect associated with the administration of an unconjugated form of the cytokine to a subject. In particular embodiments, the method comprises administering an immunoconjugate molecule comprising the cytokine to the subject in place of the administration of an unconjugated form of the cytokine. In particular embodiments, the subject is under an ongoing cytokine treatment comprising the administration of the cytokine in an unconjugated form, and the method comprises discontinuing the ongoing cytokine treatment and administering to the subject an immunoconjugate molecule comprising an equivalent amount of the cytokine. In particular embodiments, the side effect is toxicity of the cytokine. In particular embodiments, the side effect is measured by the change in body weight of the subject treated with the cytokine. In particular embodiments, the side effect is measured by the change in life-span of the subject treated with the cytokine. In particular embodiments, the side effect is measured by the change of the level of an immune response in the subject treated with the cytokine. In particular embodiments, the side effects are measured by the change in the level of an inflammatory reaction in the subject treated with the cytokine.

In some embodiments, the cytokine is TNF-α, and the cytokine-mediated effect according to the present methods includes activation of apoptosis of tumor cells in a subject. A non-limiting example of activation of apoptosis of tumor cells is tumor necrosis. Accordingly, in certain embodiments, provided herein is also a method for promoting tumor necrosis at a target site in a subject by administering an TNF-α containing immunoconjugate molecule according to the present disclosure.

Exemplary target sites for delivering and/or activating the cytokine activity according to the present methods include but are not limited to a cellular environment, such as a particular type of tissue, a particular organ, a particular population of cells. In some embodiments, the target site of the present methods can be distinguished from a non-target site based on the expression of the target antigen recognized by the immunoconjugate molecule used in the method. Particularly, in some embodiments, the target antigen is present at the target site but is not present in the non-target site. In some embodiments, the target antigen is produced by cells that are present at the target site but are not present at a non-target site. In some embodiments, the target antigen is present at the target site at a higher concentration or in a greater amount as compared to the target antigen at the non-target site. In particular embodiments, the target antigen is present at the target site (but not a non-target site) in a sufficient amount that enables the anchoring moiety of immunoconjugate molecule to immobilize the immunoconjugate molecule at the target site through the binding to the target antigen. In particular embodiments, the target antigen is present at the target site (but not a non-target site) in a sufficient amount that enables the masking moiety of immunoconjugate molecule to disassociate from the cytokine through the binding to the target antigen. In specific embodiments, the target site of the present methods contains a population of cancer cells. In specific embodiments, the target site for the present methods is a tumor microenvironment of a solid tumor. In specific embodiments, the target antigen recognized by the immunoconjugate molecule used in the methods is an antigen expressed by cancer cells, such as a tumor associated antigen (TAA). In other embodiments, the target antigen recognized by the immunoconjugate molecule used in the methods is an antigen expressed by non-cancer cells in a tumor microenvironment, such as stromal cells.

In particular embodiments of the present methods, the cytokine is TNF-α. In particular embodiments, the target antigen is fibroblast activation protein (FAP). Hence, in particular embodiments, the immunoconjugate molecule used in the present methods comprises a two-in-one antibody capable of binding to both TNF-α and FAP. In particular embodiments of the present methods, the cytokine is TNF-α. In particular embodiments, the target antigen is Tumor-associated calcium signal transducer 2 (Trop-2). Hence, in particular embodiments, the immunoconjugate molecule used in the present methods comprises a two-in-one antibody capable of binding to both TNF-α and Trop-2.

In one aspect, provided herein is a method for inhibiting growth or induce apoptosis of a cancer cell comprising contacting the cancer cell with an effective amount of the immunoconjugate molecule comprising an TNF-α polypeptide as described herein.

In one aspect, provided herein is a method of treating cancer in a subject in need thereof, comprising administering to the subject an effective amount of the immunoconjugate molecule comprising an TNF-α polypeptide as described herein. In some embodiments, the method further comprises co-administration of a second therapy. In some embodiments, the second therapy comprises a chemotherapeutic agent. In some embodiments, the second therapy comprises Actinomycin D.

In some embodiments of each or any of the above- or below-mentioned embodiments, the present immunoconjugate molecules are used for treating solid tumor cancer. In other embodiments, the present immunoconjugate molecules are used for treating blood cancer.

In some embodiments of each or any of the above- or below-mentioned embodiments, the disease or disorder is a disease of abnormal cell growth and/or dysregulated apoptosis. Examples of such diseases include, but are not limited to, cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In some embodiments of each or any of the above- or below-mentioned embodiments, the disease or disorder is selected from the group consisting of bladder cancer, brain cancer, breast cancer, bone marrow cancer, cervical cancer, chronic lymphocytic leukemia, acute lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small-cell lung cancer, prostate cancer, small-cell lung cancer and spleen cancer.

In some embodiments of each or any of the above- or below-mentioned embodiments, the disease or disorder is a hematological cancer, such as leukemia, lymphoma, or myeloma. In some embodiments of each or any of the above- or below-mentioned embodiments, the cancer is selected from a group consisting of Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CML), and chronic monocytic leukemia. In a specific embodiment, the disease or disorder is myelodysplastic syndromes (MDS). In another specific embodiment, the disease or disorder is acute myeloid leukemia (AML). In another specific embodiment, the disease or disorder is chronic lymphocytic leukemia (CLL). In yet another specific embodiment, the disease or disorder is multiple myeloma (MM).

In other embodiments, the disease or disorder is a solid tumor cancer. In some embodiments of each or any of the above- or below-mentioned embodiments, the solid tumor cancer is selected from a group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, a liver cancer and a lung cancer.

In some embodiments of each or any of the above- or below-mentioned embodiments, the cancer is an adrenal cancer. In some embodiments of each or any of the above- or below-mentioned embodiments, the cancer is an anal cancer. In some embodiments of each or any of the above- or below-mentioned embodiments, the cancer is an appendix cancer. In some embodiments of each or any of the above- or below-mentioned embodiments, the cancer is a bile duct cancer. In some embodiments of each or any of the above- or below-mentioned embodiments, the cancer is a bladder cancer. In some embodiments of each or any of the above- or below-mentioned embodiments, the cancer is a bone cancer. In some embodiments of each or any of the above- or below-mentioned embodiments, the cancer is a brain cancer. In some embodiments of each or any of the above- or below-mentioned embodiments, the cancer is a breast cancer. In some embodiments of each or any of the above- or below-mentioned embodiments, the cancer is a cervical cancer. In some embodiments of each or any of the above- or below-mentioned embodiments, the cancer is a colorectal cancer. In some embodiments of each or any of the above- or below-mentioned embodiments, the cancer is an esophageal cancer. In some embodiments of each or any of the above- or below-mentioned embodiments, the cancer is a gallbladder cancer. In some embodiments of each or any of the above- or below-mentioned embodiments, the cancer is a gestational trophoblastic. In some embodiments of each or any of the above- or below-mentioned embodiments, the cancer is a head and neck cancer. In some embodiments of each or any of the above- or below-mentioned embodiments, the cancer is a Hodgkin lymphoma. In some embodiments of each or any of the above- or below-mentioned embodiments, the cancer is an intestinal cancer. In some embodiments of each or any of the above- or below-mentioned embodiments, the cancer is a kidney cancer. In some embodiments of each or any of the above- or below-mentioned embodiments, the cancer is a leukemia. In some embodiments of each or any of the above- or below-mentioned embodiments, the cancer is a liver cancer. In some embodiments of each or any of the above- or below-mentioned embodiments, the cancer is a lung cancer. In some embodiments of each or any of the above- or below-mentioned embodiments, the cancer is a melanoma. In some embodiments of each or any of the above- or below-mentioned embodiments, the cancer is a mesothelioma. In some embodiments of each or any of the above- or below-mentioned embodiments, the cancer is a multiple myeloma (MM). In some embodiments of each or any of the above- or below-mentioned embodiments, the cancer is a neuroendocrine tumor. In some embodiments of each or any of the above- or below-mentioned embodiments, the cancer is a non-Hodgkin lymphoma. In some embodiments of each or any of the above- or below-mentioned embodiments, the cancer is an oral cancer. In some embodiments of each or any of the above- or below-mentioned embodiments, the cancer is an ovarian cancer. In some embodiments of each or any of the above- or below-mentioned embodiments, the cancer is a pancreatic cancer. In some embodiments of each or any of the above- or below-mentioned embodiments, the cancer is a prostate cancer. In some embodiments of each or any of the above- or below-mentioned embodiments, the cancer is a sinus cancer. In some embodiments of each or any of the above- or below-mentioned embodiments, the cancer is a skin cancer. In some embodiments of each or any of the above- or below-mentioned embodiments, the cancer is a soft tissue sarcoma spinal cancer. In some embodiments of each or any of the above- or below-mentioned embodiments, the cancer is a stomach cancer. In some embodiments of each or any of the above- or below-mentioned embodiments, the cancer is a testicular cancer. In some embodiments of each or any of the above- or below-mentioned embodiments, the cancer is a throat cancer. In some embodiments of each or any of the above- or below-mentioned embodiments, the cancer is a thyroid cancer. In some embodiments of each or any of the above- or below-mentioned embodiments, the cancer is a uterine cancer endometrial cancer. In some embodiments of each or any of the above- or below-mentioned embodiments, the cancer is a vaginal cancer. In some embodiments of each or any of the above- or below-mentioned embodiments, the cancer is a vulvar cancer.

In some embodiments of each or any of the above- or below-mentioned embodiments, the adrenal cancer is an adrenocortical carcinoma (ACC), adrenal cortex cancer, pheochromocytoma, or neuroblastoma. In some embodiments of each or any of the above- or below-mentioned embodiments, the anal cancer is a squamous cell carcinoma, cloacogenic carcinoma, adenocarcinoma, basal cell carcinoma, or melanoma. In some embodiments of each or any of the above- or below-mentioned embodiments, the appendix cancer is a neuroendocrine tumor (NET), mucinous adenocarcinoma, goblet cell carcinoid, intestinal-type adenocarcinoma, or signet-ring cell adenocarcinoma. In some embodiments of each or any of the above- or below-mentioned embodiments, the bile duct cancer is an extrahepatic bile duct cancer, adenocarcinomas, hilar bile duct cancer, perihilar bile duct cancer, distal bile duct cancer, or intrahepatic bile duct cancer. In some embodiments of each or any of the above- or below-mentioned embodiments, the bladder cancer is transitional cell carcinoma (TCC), papillary carcinoma, flat carcinoma, squamous cell carcinoma, adenocarcinoma, small-cell carcinoma, or sarcoma. In some embodiments of each or any of the above- or below-mentioned embodiments, the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer. In some embodiments of each or any of the above- or below-mentioned embodiments, the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma. In some embodiments of each or any of the above- or below-mentioned embodiments, the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer. In some embodiments of each or any of the above- or below-mentioned embodiments, the cervical cancer is a squamous cell carcinoma, or adenocarcinoma. In some embodiments of each or any of the above- or below-mentioned embodiments, the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma. In some embodiments of each or any of the above- or below-mentioned embodiments, the esophageal cancer is an adenocarcinoma or squamous cell carcinoma. In some embodiments of each or any of the above- or below-mentioned embodiments, the gall bladder cancer is an adenocarcinoma, papillary adenocarcinoma, adenosquamous carcinoma, squamous cell carcinoma, small cell carcinoma, or sarcoma. In some embodiments of each or any of the above- or below-mentioned embodiments, the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT). In some embodiments of each or any of the above- or below-mentioned embodiments, the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer. In some embodiments of each or any of the above- or below-mentioned embodiments, the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL). In some embodiments of each or any of the above- or below-mentioned embodiments, the intestinal cancer is a small intestine cancer, small bowel cancer, adenocarcinoma, sarcoma, gastrointestinal stromal tumors, carcinoid tumors, or lymphoma. In some embodiments of each or any of the above- or below-mentioned embodiments, the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma. In some embodiments of each or any of the above- or below-mentioned embodiments, the leukemia is an acute lymphocytic leukemia (ALL), acute myeloid leukemia (AML), chronic lymphocytic leukemia (CLL), chronic myeloid leukemia (CML), hairy cell leukemia (HCL), or a myelodysplastic syndrome (MDS). In a specific embodiment, the leukemia is AML. In some embodiments of each or any of the above- or below-mentioned embodiments, the liver cancer is a hepatocellular carcinoma (HCC), fibrolamellar HCC, cholangiocarcinoma, angiosarcoma, or liver metastasis. In some embodiments of each or any of the above- or below-mentioned embodiments, the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor. In some embodiments of each or any of the above- or below-mentioned embodiments, the melanoma is a superficial spreading melanoma, nodular melanoma, acral-lentiginous melanoma, lentigo maligna melanoma, amelanotic melanoma, desmoplastic melanoma, ocular melanoma, or metastatic melanoma. In some embodiments of each or any of the above- or below-mentioned embodiments, the mesothelioma is a pleural mesothelioma, peritoneal mesothelioma, pericardial mesothelioma, or testicular mesothelioma. In some embodiments of each or any of the above- or below-mentioned embodiments, the multiple myeloma is an active myeloma or smoldering myeloma. In some embodiments of each or any of the above- or below-mentioned embodiments, the neuroendocrine tumor is a gastrointestinal neuroendocrine tumor, pancreatic neuroendocrine tumor, or lung neuroendocrine tumor. In some embodiments of each or any of the above- or below-mentioned embodiments, the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma. In some embodiments of each or any of the above- or below-mentioned embodiments, the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer. In some embodiments of each or any of the above- or below-mentioned embodiments, the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst. In some embodiments of each or any of the above- or below-mentioned embodiments, the pancreatic cancer is a pancreatic exocrine gland cancer, pancreatic endocrine gland cancer, or pancreatic adenocarcinoma, islet cell tumor, or neuroendocrine tumor. In some embodiments of each or any of the above- or below-mentioned embodiments, the prostate cancer is a prostate adenocarcinoma, prostate sarcoma, transitional cell carcinoma, small cell carcinoma, or neuroendocrine tumor. In some embodiments of each or any of the above- or below-mentioned embodiments, the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer. In some embodiments of each or any of the above- or below-mentioned embodiments, the skin cancer is a basal cell carcinoma, squamous cell carcinoma, melanoma, Merkel cell carcinoma, Kaposi sarcoma (KS), actinic keratosis, skin lymphoma, or keratoacanthoma. In some embodiments of each or any of the above- or below-mentioned embodiments, the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma. In some embodiments of each or any of the above- or below-mentioned embodiments, the spinal cancer is a spinal metastatic tumor. In some embodiments of each or any of the above- or below-mentioned embodiments, the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid. In some embodiments of each or any of the above- or below-mentioned embodiments, the testicular cancer is a seminoma, non-seminoma, embryonal carcinoma, yolk sac carcinoma, choriocarcinoma, teratoma, gonadal stromal tumor, leydig cell tumor, or sertoli cell tumor. In some embodiments of each or any of the above- or below-mentioned embodiments, the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer. In some embodiments of each or any of the above- or below-mentioned embodiments, the thyroid cancer is a papillary carcinoma, follicular carcinoma, Hürthle cell carcinoma, medullary thyroid carcinoma, or anaplastic carcinoma. In some embodiments of each or any of the above- or below-mentioned embodiments, the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma. In some embodiments of each or any of the above- or below-mentioned embodiments, the vaginal cancer is a squamous cell carcinoma, adenocarcinoma, melanoma, or sarcoma. In some embodiments of each or any of the above- or below-mentioned embodiments, the vulvar cancer is a squamous cell carcinoma or adenocarcinoma.

### 5.5 Pharmaceutical Compositions

In one aspect, the present disclosure further provides pharmaceutical compositions comprising at least one immunoconjugate molecule of the present disclosure. In some embodiments, a pharmaceutical composition comprises 1) the immunoconjugate molecule, and 2) a pharmaceutically acceptable carrier.

Pharmaceutical compositions comprising an antibody or antibody-containing immunoconjugate molecule are prepared for storage by mixing the antibody or the immunoconjugate molecule having the desired degree of purity with optional physiologically acceptable carriers, excipients, or stabilizers (see, e.g., Remington, Remington's Pharmaceutical Sciences (18th ed. 1980)) in the form of aqueous solutions or lyophilized or other dried forms.

The immunoconjugate molecule of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

An immunoconjugate molecule provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with an antibody or antibody-containing molecules such as the immunoconjugate molecule as described herein, including, but not limited to, encapsulation in liposomes, microparticles, microcapsules, recombinant cells capable of expressing the antibody, receptor-mediated endocytosis (see, e.g., Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., an antibody that binds to PD-1 as described herein) or a composition of the invention (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more antibodies that bind to PD-1 as described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

### 5.6 Kits

Also provided herein are kits comprising an immunoconjugate molecule as provided herein, or a composition (e.g., a pharmaceutical composition) thereof, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain antibodies provided herein, or cells that contain nucleic acids encoding the antibodies provided herein. The cells in the kit can be maintained under appropriate storage conditions until ready to use.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

All applications, publications, patents and other references, GenBank citations and ATCC citations cited herein are incorporated by reference in their entirety. In case of conflict, the specification, including definitions, will control.

As used herein, the singular forms “a,” “and,” and “the” include plural referents unless the context clearly indicates otherwise. Thus, for example, reference to “a peptide sequence” includes a plurality of such sequences and so forth.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges include combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### 6. EXAMPLES

The examples in this section (i.e., Section 6) are offered by way of illustration, and not by way of limitation.

### 6.1 Example 1: General Methods

### 6.1.1 Cell Lines and Culturing Conditions

If not indicated differently, all cell culture media and supplements were obtained from Gibco by Thermofisher. The HEK 293T cell line was purchased from (Fenghui ShengWu, China) and was maintained in DMEM supplemented with 10% fetal bovine serum (FBS), 1% L-glutamine (L-glu), 1% Na Pyruvate, 1% penicillin and streptomycin (P/S). The HEK blue TNF-α reporter cell line was purchased from InvivoGen, USA and was maintained in DMEM supplemented with 10% heat-inactivated fetal bovein serum (FBS), 1% L-glutamine (L-glu), 1% Na Pyruvate, 1% penicillin and streptomycin (P/S) with 100 μg/mL Normacin (InvivoGen). The murine L929 cell line was purchased from Procell, China and was maintained in RPMI supplemented with 10% FBS, 1% P/S.

### 6.1.2 Generation of a Stable FAP-Expressing Cell Line

Adherent HEK 293T cells stably expressing FAP were generated as described below. An FAP-expressing vector with a hygromycin resistance gene was purchased from Sino Biologics. The plasmid was transfected into HEK 293T cells using Lipofectamine 2000 (Thermofisher) system. 24 hrs after the transfection, 150 μg/mL hygromycin was added into the cell culture, and fresh media was changed when necessary in the following two weeks. The surviving cells were expanded, and the expression of FAP was confirmed using flow cytometer (CYTOFLEX, Beckman Coulter) labeled with anti-FAP mAB (Thermofisher) and Goat anti-mouse Alexa 488 (Thermofisher). FAP-expressing clones were sorted from the pool using BD FACSAria III sorter. A clone with high-level expression designated as HEK 293T-FAP-E5 was selected and used in the assays as described below, and its receptor density was calibrated around 3×10⁶/cell using the Quantum Alexa Fluor 488 MESF kit (Bangslab, USA).

### 6.1.3 Affinity Determination by Biolayer Interferometry

Binding kinetics of antibody-antigen interactions were determined by biolayer interferometry using the Gator BLI system. Particularly, biotinylated antigen was immobilized on a sensor coated with streptavidin to a response level of ˜0.5-1.0 nm Candidate antibodies were constructed in the form of a monovalent Fab-Fc fusion protein containing a Knob-in-Hole modification in the Fc portion, and were subjected to serial two-fold dilutions that resulted in final concentrations in the range of 100 nM to 3.1 nM. The antibody sample was applied to the sensor and incubated for up to 240 seconds to allow antibody-antigen association, which was followed by incubating the sensor in PBST-BSA for up to 420 seconds to allow disassociation. Binding constants (kₒₙ, k_{off} and K_{D}) were determined after referencing by subtracting responses from sensors without immobilized antigen. Data were fit globally with Gator software using a 1:1 binding interaction while fitting the responses to an unlinked Rₘₐₓ. All protein samples were diluted in PBST-BSA. Typical protocol for K_{D} determination:

| Step | Time | Solution |
|---|---|---|
| Baseline | 120 seconds | PBST-BSA |
| Antigen | 180 seconds | Biotinylated |
| Immobilization | | Antigen (10-100 nM) |
| Baseline | 120 seconds | PBST-BSA |
| Association | 240 seconds | Fab-Fc(Knob-in-Hole),_various |
| | | concentrations |
| Dissociation | 240-420 seconds | PBST-BSA |

### 6.2 Example 2: Generation of Antibodies

### 6.2.1 Generation of Anti-FAP Antibodies

The parental FAP mAbs were initially generated by phage display methods using human Fibroblast Activation Protein (FAP) antigen. Fabs were isolated from phage antibody libraries constructed by Kunkel mutagenesis where codon-based sequence diversification of the CDRs was introduced by phosphoramidite trinucleotide-based primers. Three to four rounds phage panning were performed with antigen immobilized on Streptavidin beads. Initial characterization of a phage pool by monoclonal phage ELISA identified 82 subclones producing anti-human FAP antibodies with K_{D} ˜1-100 nM to soluble FAP antigen (data not shown), and were designated as IgG-1 through IgG-82, respectively. Antibodies were sub-cloned into the IgG1 and Fab-Fc format for further studies.

HEK 293T and HEK 293T-FAP-E5 cells were used for cell binding to confirm selected antibodies were able to bind to the epitope on cell surface, and to exclude antibodies with non-specific binding to cells. HEK 293T-FAP-E5 is a single clone expressing ˜1×10⁶ FAP/cell. It was generated by transient transfection on parental HEK 293T cells, sorted by FACS, selected by hygromycin resistance, and its receptor density was quantified by Quantum MESF 488 (Bangs Laboratories, USA) following the manufacture-provided procedure. Both live and fixed cells were used for cell binding. For cell fixation, HEK 293T and HEK 293T-FAP-E5 cells were detached, washed twice with PBS, fixed with 4% paraformaldehyde, and stored in PBS-1% BSA.

Antibody binding to cells was assayed by flow cytometry with Cytoflex (Beckman Coulter). In brief, 1 μg/mL purified antibody was incubated with 5×10⁴ cells in the volume of 100 uL for 30 minutes. Cells were centrifuged and washed once with PBS-1% BSA. 1 μg/mL secondary antibody goat anti-human Alexa488 (A-11013, Thermofisher) was incubated with washed cells in the volume of 100 μL for 30 minutes. Cells were centrifuged and washed once with PBS-1% BSA. The labeled cells were resuspended in 300 μL and loaded on to Cytoflex using the FTIC settings. The pair of primary polyclonal FAP antibody (PA5-95481, Thermofisher) and secondary goat anti-rabbit Alexa488 (A-11008, Thermofisher) were used as positive control. The pair of antibodies including an isotype antibody DP47GS and secondary antibody goat anti-human Alexa488 (A-11013, Thermofisher) were used as negative controls.

A panel of 13 clones (872-2, 872-5, 872-10, 872-11, 872-19, 872-26, 872-39, 872-44, 872-58, 872-59, 872-67, 872-70 and 872-75) were selected and subjected to the epitope binning study.

### 6.2.2 Epitope Binning

To determine whether the selected anti-FAP antibodies share non-overlapping epitopes, epitope binning studies were performed on the Gator™ Biolayer interferometry (BLI) system. Antigen (biotinylated FAP) was immobilized to a response level of ˜0.5-1 nm. After establishing a baseline, sensors were subjected to saturating levels (>1 μM) of anti-FAP IgG antibodies. After a short dissociation period of about 60 seconds, the sensor was incubated in a solution containing a second antibody and the response was monitored. Antibody pairs were considered to have different epitopes if the binding event of the second antibody resulted in a response >50% of the first antibody binding event. Antibody pairs where the second antibody event did not result in any further increase in the signal after the first antibody binding event were considered to share overlapping epitopes. Table 1 below summarizes the results of the epitope binning study.

TABLE 1

| | 2^{nd} Binding Ab | 1^{st} Binding Ab | | | |
|---|---|---|---|---|---|
| | Antibody | IgG 5 | IgG 59 | IgG 67 | IgG 70 |
| 1 | 872-2 | Same | Same | Ambiguous | Ambiguous |
| 2 | 872-5 | Same | Same | Different | Different |
| 3 | 872-10 | Same | Same | Same | Same |
| 4 | 872-11 | Same | Same | Different | Same |
| 5 | 872-19 | Same | Same | Different | Same |
| 6 | 872-26 | Same | Different | Different | Different |
| 7 | 872-39 | Same | Same | Different | Different |
| 8 | 872-44 | Same | Same | Same | Same |
| 9 | 872-58 | Same | Same | Same | Different |
| 10 | 872-59 | Same | Same | Different | Different |
| 11 | 872-67 | Same | Different | Same | Different |
| 12 | 872-70 | Different | Different | Different | Same |
| 13 | 872-75 | Same | Same | Same | Same |

### 6.2.3 Cell-Based FAP Binding Assay

Antibodies with confirmed binding by biolayer interferometry were subsequently screened for the ability to bind to HEK-293 cells expressing human FAP. Three anti-FAP IgG antibodies (produced by Clones IgG 5, IgG 59, and IgG 70, respectively, and designated as antibodies 872-5, 872-59, and 872-70, respectively) that bind to two non-overlapping epitopes of FAP at nM level of dissociation constants (Table 2) were selected.

TABLE 2

| Antibody | kₒₙ (M⁻¹s⁻¹) | k_{off} (s⁻¹) | K_{D} (nM) |
|---|---|---|---|
| 872-5 | 4.2 × 10⁵ | 2.8 × 10⁻³ | 6.6 |
| 872-59 | 3.6 × 10⁵ | 5.6 × 10⁻³ | 15.5 |
| 872-70 | 1.1 × 10⁵ | Slow | <1 |

### 6.2.4 Generation of Anti-TNF-α/Anti-TAA Bispecific Antibodies

Phage displaying libraries were constructed using a commercial anti-TNF-α antibody as the starting antibody. Particularly, using Kunkel mutagenesis, each codon encoding an amino acid residue in the 6 complementarity-determining regions (CDR) of a starting antibody was replaced with the degenerate codon NNK, one position at a time. Saturation mutagenesis of each mutated residue within a CDR were pooled for subsequent library preparation and phage panning. DNA of the constructed libraries was electroporated into SS320 cells pre-infected with M13K07 following published procedures. Phage was prepared for panning similarly to selections of naïve libraries, with several modifications. 1 pmol of biotinylated antigen was used for phage panning. During the course of the selection, 1 μM of soluble competitor was added to well E of the KingFisher plate. This allowed for “off-rate” selection where soluble antigen can compete for binding to phage once it had dissociated from the antigen-coated beads due to the vast molar excess of soluble competitor. Additionally, a parallel selection was performed with biotinylated anti-CH1 antibody to monitor expression bias within the panning experiment. Three rounds of phage panning were performed and the resulting outputs were prepared for next-generation sequencing. The resulting sequence data were analyzed for amino acid distribution preferences within the CDRs.

Secondary libraries were constructed to introduce amino acid diversity into CDR positions found to be amenable to mutation by saturation mutagenesis-NGS sequencing analysis. Phage libraries were constructed in a similar manner to naïve antibody libraries using Kunkel mutagenesis with synthesized primers coupled with electroporation into E. coli strain SS320 pre-infected with M13K07 helper phage.

### 6.2.5 Phage Panning for Bispecific Antibody Isolation

Constructed antibody libraries were subjected to four rounds of phage panning. Particularly, 500 μL of solution containing the starting phage library was diluted to an A₂₆₈=1 (˜1×10¹² colony forming units/mL) in PBST-BSA (Phosphate buffered saline supplemented with 0.2% Tween 20, 2% Bovine Serum Albumin). The phage library was pre-cleared by incubation with 20 μL M280 Streptavidin Dynabeads for one hour. After pre-clearance, the phage library was incubated with 20 μL Dynabeads coated with 50 pmoles biotinylated FAP (Sino Biological) or biotinylated-Trop2 (Acro Biosciences). Samples were incubated at room temperature for ˜1 hour with gentle mixing. Beads were then sedimented with a magnetic stand to remove unbound phage. Samples were washed three times with 500 μL PBST-BSA and then incubated with 200 μL 0.1 M glycine (pH 2.7) for 15 minutes to elute the phage from the beads. The supernatant of the elution was then separated from the beads, neutralized with 40 μL 1 M HEPES, pH 7.2. The elution and beads were added to 5 mL mid log-phase XL1-blue cells and allowed to incubate at room temperature for 30 minutes. The infected cells were then sub-cultured by addition of 25 mL 2×YT supplemented with Ampicillin (50 μg/mL) and M13K07 helper phage (˜10¹⁰ pfu/mL). Cell cultures were allowed to grow for ˜16 hours at 37° C. with vigorous shaking.

Rounds 2-4 of phage panning were performed similarly. Particularly, after culturing infected cells overnight, the cells were centrifuged to pellet and removed. The resulting supernatant was precipitated by adding 1/5 volume PEG/NaCl solution and incubated for 30 minutes on ice. After centrifugation at 10,000×g for 15 minutes, the supernatant was removed and the phage pellet was resuspended in 200 μL PBS. The resuspended phage was centrifuged at 14,000×g for 5 minutes to remove insoluble materials. The phage was then transferred to a fresh Eppendorf® tube and precipitated a second time by adding 40 μL PEG/NaCl solution. The samples were placed on ice for ˜15 minutes before spinning at 10,000×g for 10 minutes. The supernatant was removed and phage was resuspended in 200 μL PBS. Samples were again centrifuged at 14,000×g for 5 minutes to remove insoluble materials. Phage was quantitated and prepared for pre-clearance with streptavidin beads. Here, 250 μL of phage A₂₆₈=0.2-0.5 (round 2 uses A₂₆₈=0.5, rounds 3 and 4 use A₂₆₈=0.2) in PBST-BSA was incubated with 10 μL M280 Streptavidin Dynabeads for 30 minutes. After pre-clearance, the phage was added to Well C of a 200 uL KingFisher™ plate for bead manipulation. The following was added to the KingFisher™ plate for phage panning:

| Well | Solution | Volume |
|---|---|---|
| Well A | Streptavidin beads + Antigen | 100 μL |
| | (variable concentration) | |
| Well B | 10 μM Biotin solution in PBS | 100 μL |
| Well C | Pre-cleared phage solution | 100 μL |
| Well D | PBST-BSA | 100 μL |
| Well E | PBST-BSA | 100 μL |
| Well F | PBST-BSA | 100 μL |
| Well G | PBST-BSA | 100 μL |
| Well H | 0.1M Glycine pH 2.7 | 100 μL |

The KingFisher™ protocol used for phage panning was the following:

| Well | Solution | Time |
|---|---|---|
| Well A | 15 minutes incubation with fast mixing | 15 min. |
| Well B | Transfer to well B with fast mixing | 5 min. |
| Well C | Transfer to well C with fast mixing | 15 min. |
| Well D | Transfer to well D with fast mixing | 5 min. |
| Well E | Transfer to well E with fast mixing | 1 min. |
| Well F | Transfer to well F with fast mixing | 1 min. |
| Well G | Transfer to well G with fast mixing | 1 min. |
| Well H | 15 minutes with fast mixing | 15 min. |
| Well G | Release beads | N/A |

After the KingFisher™ phage panning protocol, the phage was neutralized with 20 μL 1 M HEPES. 50 μL of the phage elution was added to 500 μL mid log phase XL1 for 30 minutes for infection. The infected cells were then sub-cultured in 2.5 mL 2×YT supplemented with Ampicillin (50 μg/mL) and M13K07 helper phage (˜10¹⁰ pfu/mL). Cell cultures were grown overnight at 37° C. with vigorous shaking to amplify phage. Additionally, phage was quantitated by plating serial dilutions of the infection to monitor the number of colony forming units in the elution of each round.

Phage panning was continued through four rounds. The concentration of antigen used during each round was in the range of 100 nM to 10 nM with lower concentrations used during later rounds. Phage panning experiments were monitored for increases in phage titer in successive rounds of panning.

### 6.2.6 Monoclonal Phage ELISA

To identify individual clones from the phage panning studies with the desired binding properties (e.g., antibodies capable of binding to both FAP and TNF-α), monoclonal phage ELISA study was performed. Phage from the elution was used to infect XL1-blue cells. After 30 minutes, the cells were plated on LB-Ampicillin plates so that individual colonies could be isolated and picked. After incubation overnight at 37° C., the colonies on the plate were picked and placed in a 96-deep well block and incubated with 400 μL 2×YT supplemented with Ampicillin (50 μg/mL) and M13K07 helper phage (˜10¹⁰ pfu/mL). Plates were incubated overnight at 37° C. with vigorous shaking. After ˜14 hours of incubation, the plates were centrifuged (4000×g for 10 minutes) to pellet cells. The resulting phage-containing supernatant was diluted 10-fold in PBST-BSA. 50 μL of the diluted phage-containing supernatant was incubated in three separate wells of a Maxisorp™ ELISA plate. Well #1 contained 2.5 pmoles of immobilized, TNF-α Well #2 contained 2.5 pmoles of immobilized FAP or Trop2, and Well #3 was coated with BSA. Phage was incubated for 30 minutes before washing three times with PBST. The plates were then incubated with 50 μL 0.2 μg/mL anti-M13-HRP antibody (Sino Biological,) for 30 minutes. ELISA plates were again washed three times in PBST. Horseradish Peroxidase activity was detected with 1-Step™ Ultra TMB-ELISA TMB substrate (ThermoFisher). ELISA plates were allowed to develop for approximately five minutes and reactions were quenched with 1 M M Phosphoric acid. Reactions were quantitated by measuring absorbance at 410 nm. Samples with significant signal (>3 times about background) were sent for sequence analysis.

Through the screening of the phage libraries, multiple variants of the parent antibody were identified and designated as TN01 to TN13 (Trop2/TNF-α two-in-one antibodies), respectively, and FN01 to FN17 (FAP/TNF-α two-in-one antibodies), respectively. These variants were able to bind to the TNF-α polypeptide and a variety of TNF-α mutants.

### 6.2.7 Generation of Antibody Variants

In order to examine possible effects of the molecular configuration of the immunoconjugate on the activity of the molecule, Fab and scFv variants of parent antibodies were generated.

Particularly, Fab and scFv variants of the antibodies were recombinantly produced by combining the binding sequences of the parental antibodies. Two-in-one Fab-Fc variants generated in this study were subjected to biolayer interferometry analysis described in Section 6.1.3 above to measure binding kinetics of the two-in-one Fab-Fc variants to both TNF-α and tumor associated antigen. In total twelve two-in-one Fab-Fc variants were tested: six two-in-one Fab-Fc variants capable of binding to both TNF-α and FAP (FIGS. 3A to 3F); and six two-in-one Fab-Fc variants capable of binding to both TNF-α and Trop2 (FIGS. 3G to 3L).

The original results are shown in FIGS. 3A through 3L and are further summarized in Table 3 and Table 4 below.

TABLE 3
Binding Kinetics of Antibody Variants to FAP and TNF-α.

| | | TNF-α | | | FAP | | |
|---|---|---|---|---|---|---|---|
| Antibody | Type | K_{D} (nM) | kₒₙ | k_{off} | K_{D} (nM) | kₒₙ | k_{off} |
| FN06 | Fab-Fc | 21.8 | 1.14E+05 | 2.47E−03 | 16.0 | 5.18E+04 | 8.29E−04 |
| FN11 | Fab-Fc | 35.4 | 1.11E+05 | 3.92E−04 | 13.6 | 1.12E+05 | 1.53E−03 |
| FN12 | Fab-Fc | 27.2 | 1.10E+05 | 2.98E−03 | 19.9 | 6.43E+04 | 1.28E−03 |
| FN15 | Fab-Fc | 20.5 | 1.48E+05 | 3.04E−03 | 2.19 | 1.43 + 05 | 3.14E−04 |
| FN16 | Fab-Fc | 63.4 | 5.61E+04 | 3.56E−03 | 27.4 | 7.58E+04 | 2.08E−03 |
| FN17 | Fab-Fc | 16.0 | 1.15E+05 | 1.84E−03 | 11.6 | 1.92E+05 | 2.22E−03 |

TABLE 4
Binding Kinetics of Antibody Variants to Trop-2 and TNF-α.

| | | TNF-α | | | Trop2 | | |
|---|---|---|---|---|---|---|---|
| Antibody | Type | K_{D} (nM) | Kₒₙ | K_{off} | K_{D} (nM) | kₒₙ | k_{off} |
| TN01 | Fab-Fc | 16.5 | 1.30E+05 | 2.15E−03 | 101 | 2.74E+04 | 3.38E−03 |
| TN03 | Fab-Fc | 12.4 | 1.11E+05 | 1.38E−03 | Biphasic | Uncertain | Uncertain |
| TN04 | Fab-Fc | 1.22 | 1.10E+05 | 1.34E−03 | Biphasic | Uncertain | Uncertain |
| TN10 | Fab-Fc | 17.7 | 1.56E+05 | 2.76E−03 | 2620 | N.D. | N.D. |
| TN11 | Fab-Fc | 43.1 | 8.95E+04 | 3.86E−03 | 2160 | 3.94E+04 | 2.39E−02 |
| TN13 | Fab-Fc | 21.2 | 1.45E+04 | 3.07E−04 | >1000 | N.D. | N.D. |

As shown in FIGS. 3A through 3L, as well as Table 3 and Table 4, the binding affinities of Fab-Fc variants to FAP were generally higher than those of Fab-Fc variants to Trop2. In the meantime, the binding affinities of all Fab-Fc variants to TNF-α were between 10 nM to 90 nM. It is worth noting that FN15 has high binding affinity to both FAP and TNF-α. Particularly, the K_{D} value was 25.7 nM for the interaction between FN15 with TNF-α and was 13.1 nM for the interaction between FN15 with FAP. The high binding affinities may explain FN15's favorable behavior in cell-based assays described in Sections 6.6.4 through 6.6.7 below.

### 6.3 Example 3: Generation of Recombinant Antibody-Cytokine Immunoconjugate

Antibody-cytokine immunoconjugates having different molecular configurations as illustrated in FIGS. 1B, 1C, 1E, and 1G were recombinantly generated and screened for the ability of shielding and de-shielding. Particularly, DNA sequences of immunoconjugates were codon optimized and cloned into pcDNA3.4 vector (Thermofisher) with a signal peptide as secreted proteins. Each peptidic chain was cloned into an independent vector. Proteins were expressed in Expi293F expression system (Thermofisher), and proteins were purified with either Protein-L (Genescript) or anti-FLAG (Genescript) affinity resin. In brief, plasmids of individual chain were combined at equal mass ratio and transfected to Expi293F cells using ExpiFectamine. The cells were fed ˜18 hours after transfection and the supernatant were harvested within 5-7 days after expression by centrifugation at 4000 rpm for 5 minutes. After Protein-L or anti-FLAG resin was incubated with supernatant and washed, the protein L resin were eluted by 0.1M glycine pH 4.0, neutralized with 1/5 volume of 1 M Tris pH 8.0, and dialyzed in PBS pH7.4. The anti-FLAG resin was eluted by in 1 mg/mL DYKDDDK peptide in PBS7.4.

### 6.4 Example 4: Cell-Based TNF-α Signaling Assays

### 6.4.1 TNF-α Signaling Assay in HEK Blue TNF-α Reporter Cell Line

The HEK Blue TNF-α reporter cell line (InvivoGen) used herein was engineered with high affinity human TNFR1 receptor on the cell surface. The receptor's dose-dependent response to TNF-α induces NF-κB activation and is correlated with the level of secreted embryonic alkaline phosphatase (SEAP) in the supernatant of the cell culture. The level of SEAP, and thus TNF-α activity, was measured using an enzymatic assay with the QUANTI-Blue Solution. The EC₅₀ concentration was calculated using least squares analysis (TREND analysis from Excel).

Particularly, to assay TNF-α activity, 40,000 HEK Blue TNF-α cells were cultured in flat bottom 96-well plates, and unconjugated TNF-α polypeptide or TNF-α containing immunoconjugates were added to the cell culture at the indicated gradient of concentrations. After 20-hour incubation, 20 μL supernatant of the cell culture was added into 180 μL QUANTI-Blue Solution (InVivoGen) and the reaction was incubated at 37° C. for 1-3 hrs. The absorbance at 635 nm (A₆₃₅) was determined using a TECAN plate reader, which reflected the SEAP level and thus TNF-α activity.

To assay influence of tumor associated antigen present on the target cells (through cis-presentation) on the activation of TNF-α activity of immunoconjugates, 40,000 HEK Blue TNF-α cells or 40,000 HEK Blue TNF-α-FAP cells where were stably transfected to co-express FAP and human TNFR1 receptor, were cultured in flat bottom 96-well plates. Soluble TNF-α containing immunoconjugates were added to the cell culture at the indicated gradient of concentrations. After 20-hour incubation, 20 μL supernatant of the cell culture was added into 180 μL QUANTI-Blue Solution (InVivoGen) and the reaction was incubated at 37° C. for 1-3 hrs. The absorbance at 635 nm (A₆₃₅) was determined using a TECAN plate reader, which reflected the SEAP level and thus TNF-α activity.

To assay influence of tumor associated antigen present on cells nearby the target cells on the activation of TNF-α activity of immunoconjugates, 20,000 HEK Blue TNF-α cells were co-cultured with either 20,000 HEK293T cells or 20,000 HEK293T cells expressing FAP on the surface (HEK 293T-FAP-E5 cells) into flat bottom 96-well plates. TNF-α containing immunoconjugates were added to the cell culture at the indicated gradient of concentrations. After 20-hour incubation, 20 μL of supernatant was added into 180 μL QUANTI-Blue buffer (InVivoGen) and the reaction was incubated at 37° C. for 1-3 hrs. The absorbance at 635 nm (A₆₃₅) was determined using a TECAN plate reader, which reflected the SEAP level thus TNF-α activity.

The results are plotted in FIGS. 22A and 22B. As shown, the immunoconjugate did not exhibit measurable TNF-α activity in the absence of HEK blue cells that expressed FAP, while TNF-α activity of the immunoconjugate was induced in the presence of FAP-expressing cells.

### 6.4.2 TNF-α Signaling Assay in Murine L929 Cell Line

The murine L929 cell line expresses TNFR1 on the cell surface. The receptor's dose-dependent response to TNF-α induces NF-κB activation and cell death, which was then measured using Alamar blue assay (Thermofisher). The EC₅₀ concentration was calculated using least squares analysis (TREND analysis from Excel).

Particularly, to assay TNF-α activity, 20,000 murine L929 cells were cultured in flat bottom 96-well plates, and unconjugated TNF-α polypeptide or TNF-α containing immunoconjugates were added to the cell culture at the indicated gradient of concentrations. 1 μg/mL Actinomycin D (Sigma Aldrich) was added in all wells. After 20-hour incubation, Alamar blue solution (10% final concentration in PBS) was added to each well. After two hours, the fluorescence was monitored using an excitation wavelength of 560 nm and an emission wavelength of 590 nm. The intensity of the fluorescence reflected the amount of live cells, which is reversely related to apoptotic activity by TNF-α.

### 6.5 Example 5: Biophysical Properties

### 6.5.1 Accelerated Stability

Size exclusion chromatography was performed on an Agilent 1200 HPLC system with a TSKgel G3000SW (05789, TOSH Bioscience) column. A flow rate of 0.35 mL/mL with PBS as running buffer was used, and retention time for each sample was assigned based on the major peak. As shown in FIG. 24A, TNF-α-containing immunoconjugate protein remained stable after storage at 40° C. for four weeks, or 5 rounds of freeze-thaw cycles.

### 6.5.2 In Vivo Half-Life

The pharmacokinetics of interested molecules were measured in health C57BL/6 mice. Mice were injected with desired amounts of molecules (4 μg or 25p g) in a volume of 150 μL in the tail vein using a slow push. At various time points, small blood samples (20-100 μL) were taken by retro-orbital bleeding and collected in tubes coated with heparin to prevent clotting. After centrifugation to remove the cells, the plasma was assayed by ELISA with human FAP as capture reagent (prepared in house) and Goat anti-human kappa light chain secondary antibody, biotin (A18857, Invitrogen) as detection antibody. Results were normalized to the initial concentration in the serum of each mouse taken immediately after injection. As shown in FIG. 24B, the half-life of the control molecule (TNF-α polypeptide), was 0.44 hour. The immunoconjugate molecule tested had the half-life extended to about 44 hours. The half-life was analyzed and listed in the table.

### 6.5.3 In Vivo Toxicity

Female C57BL/6J mice were administered TNF-α containing immunoconjugates through tail vein injection on Day 0 and administration was repeated two days (Day 2). Samples included unconjugated TNF-α polypeptide (4 μg) and an TNF-α-containing immunoconjugate (200 μg) and were compared to a PBS control. Toxicity of the immunoconjugates was monitored by measurement of body weight and mouse survival daily. As shown in FIG. 24C, administration of PBS and 200 μg immunoconjugate did not induce significant body weight change in mice, while administration of 4 μg TNF-α significantly reduced body weight immediately following the first administration. These data demonstrate that TNF-α immunoconjugate molecules described herein have significantly reduced toxicity compared to unconjugated TNF-α polypeptide.

### 6.6 Example 6: Inhibition and Restoration of TNF-α Activity of Immunoconjugates

### 6.6.1 Inhibition of TNF-α Activity Via Intramolecular Interaction in TNF-α Containing Immunoconjugates

Fab and scFv variants of anti-TNF-α monoclonal antibodies were recombinantly produced by combining the binding sequences of the parental antibodies as described above. TNF-α containing immunoconjugates were then constructed by fusing wild-type or mutant TNF-α polypeptide to anti-TNF-α antibody or Fab or scFv variant thereof via subcloning followed by protein expression and purification using methods described above or know in the art.

FIG. 4A shows an exemplary embodiment of a homotrimer complex containing three immunoconjugate molecules containing a TNF-α polypeptide fused to an anti-TNF-α scFv. Immunoconjugates having the configuration in FIG. 4A were subjected to cell-based TNF-α signaling assays described in Sections 6.4.1 and 6.4.2 above. The results are shown in FIGS. 4B and 4C, respectively.

Particularly, two different immunoconjugates having the configuration in FIG. 4A were tested. Each of the two immunoconjugates contained a different anti-TNF-α scFv, designated as adascFv and cerfvhvl, respectively. A commercially available wild-type TNF-α polypeptide in the non-conjugated form was also included in the assay as a negative control (Sino Biological; square). Moreover, an unconjugated wild-type TNF-α polypeptide with a FLAG-tag at the C-terminus was included as an additional negative control in the TNF-α signaling assay described in Section 6.4.2 above.

As shown in FIG. 4B, the unconjugated TNF-α polypeptide (square) elicited strong responses to TNF-α in HEK reporter cells in a dose-dependent manner. In contrast, TNF-α activities were significantly inhibited in the two tested immunoconjugates having the configuration in FIG. 4A (solid circle and up triangle), as reflected in the significantly increased EC₅₀ values.

As shown in FIG. 4C, two versions of wild-type TNF-α polypeptide in the non-conjugated form (square and circle) both elicited strong responses to TNF-α in murine L929 cells in a dose-dependent manner. In contrast, TNF-α activities was significantly inhibited in the two tested immunoconjugates having the configuration in FIG. 4A (up triangle and diamond).

TNF-α activities of immunoconjugates having the configuration in FIG. 4A can be more than 10⁴-fold weaker than that of the wild-type TNF-α, indicating that TNF-α activities of such immunoconjugates are inhibited by anti-TNF-α scFvs in these immunoconjugates.

### 6.6.2 TNF-α Activity is Further Diminished in Mutated TNF-α

In order to further reduce TNF-α activity in immunoconjugate molecules in the shielded configuration, point mutations were introduced into the wild-type TNF-α. Immunoconjugates containing mutant TNF-α were then constructed by fusing mutant TNF-α to anti-TNF-α scFvs via subcloning followed by protein expression and purification. Methods involved herein are known in the art. A schematic illustration of a trimer of the above-described immunoconjugates containing the mutant TNF-α is shown in FIG. 5A.

Immunoconjugates having the configuration in FIG. 5A were subjected to TNF-α signaling assays described in Sections 6.4.1 and 6.4.2 above. The results are shown in the top and bottom panels of FIGS. 5B to 5D, respectively.

Particularly, three different immunoconjugates having the configuration in FIG. 5A were tested. Each of the three immunoconjugates contained a different point mutation of TNF-α: S147A in FIG. 5B, N34H in FIG. 5C, and S95A in FIG. 5D. Three forms of negative control were included in each assay: the unconjugated wild-type TNF-α polypeptide (Sino Biological, Beijing, China), the unconjugated wild-type TNF-α polypeptide fused to a FLAG-tag via a peptidic linker, and the unconjugated mutant TNF-α polypeptide fused to a FLAG tag via a peptidic linker.

As shown in the top panels of FIGS. 5B to 5D, the unconjugated wild-type TNF-α polypeptide (solid square) and the unconjugated wild-type TNF-α polypeptide with a FLAG-tag at the C-terminus (empty square) both elicited strong responses to TNF-α in HEK reporter cells in a dose-dependent manner. The unconjugated mutant TNF-α polypeptide with a FLAG-tag at the C-terminus (solid circle) also elicited similar strong responses to TNF-α. In contrast, TNF-α activities were significantly inhibited in all three immunoconjugate molecules containing mutant TNF-α having the configuration in FIG. 5A (open circle).

As shown in the bottom panels of FIGS. 5B to 5D, the unconjugated wild-type TNF-α polypeptide (solid square) and the unconjugated wild-type TNF-α polypeptide with a FLAG-tag at the C-terminus (empty square) both elicited strong responses to TNF-α in murine L929 cells in a dose-dependent manner. The unconjugated mutant TNF-α polypeptide with a FLAG-tag at the C-terminus (solid circle) also elicited similar strong responses to TNF-α. In contrast, TNF-α activities were significantly inhibited in all three immunoconjugate molecules having mutant TNF-α having the configuration in FIG. 5A (open circle).

TNF-α activities of immunoconjugates having the configuration in FIG. 5A can be up to 107-fold weaker than that of the corresponding unconjugated TNF-α. These results demonstrate that mutant TNF-α polypeptide can have further diminished activity under the shielded condition but is capable of mediating a cellular effect in the unconjugated form.

### 6.6.3 Antigen-Dependent Partial Restoration of TNF-α Activity in Immunoconjugates Containing Anchoring Moieties

Anti-FAP scFvs were used as the anchoring moieties of immunoconjugates in this exemplary embodiment. Such anti-FAP scFvs were operably linked to the immunoconjugates containing mutant TNF-α as described in Section 6.6.2 above via subcloning followed by protein expression and purification. Furthermore, anti-TNF-α scFvs were replaced with anti-TNF-α Fabs as the masking moieties to inhibit the cellular effect of TNF-α. Methods involved herein are as described above or known in the art. A schematic illustration of a trimer of the above-described immunoconjugates containing the mutant TNF-α, the masking moiety (anti-TNF-α Fab), and the anchoring moiety (anti-FAP scFv) is shown in FIG. 6A.

Immunoconjugates having the configuration in FIG. 6A were subjected to the TNF-α signaling assay described in Section 6.4.1 above. The results are shown in FIGS. 6B and 6C.

Particularly, four different immunoconjugates having the configuration in FIG. 6A were tested in the absence or presence of FAP expressed on the cell surface of HEK Blue TNF-α reporter cells. Each of the four immunoconjugates contained a S147G or Y87F point mutation of TNF-α and a 50 amino acids or 70 amino acids linker linking the anchoring moiety to the immunoconjugate. The unconjugated wild-type TNF-α polypeptide (Sino Biological, Beijing, China) was included as a negative control.

As shown in FIGS. 6B and 6C, the unconjugated wild-type TNF-α polypeptide elicited strong responses to TNF-α in HEK reporter cells in a dose-dependent manner in the absence (solid circle) and presence (open circle) of FAP expression. In the absence of FAP expression, TNF-α activities remained significantly inhibited in the tested immunoconjugates (solid triangle). In contrast, in the presence of FAP expression, TNF-α activities of the tested immunoconjugates were partially restored (open triangle). The EC₅₀ concentration of the four tested immunoconjugates, as well as the unconjugated wild-type TNF-α polypeptide are shown in Table 5 below.

TABLE 5
EC₅₀ Concentration of Tested Immunoconjugates or Unconjugated Wild-type TNF-α.

| | | EC₅₀ (pM) | |
|---|---|---|---|
| | | Without FAP | With FAP |
| Protein ID | Protein Description | Expression | Expression |
| 15 | scFv70-sc50-Cert(VL-CL) × TNF-α (S147G)- | | 353.60 |
| | Cert(VH-CH1) | | |
| 16 | scFv70-sc50-Cert(VL-CL) × TNF-α (Y87F)- | | 589.00 |
| | Cert(VH-CH1) | | |
| 17 | scFv70-sc70-Cert(VL-CL) x TNF-α (S147G)- | | 297.70 |
| | Cert(VH-CH1) | | |
| 18 | scFv70-sc70-Cert(VL-CL) × TNF-α (Y87F)- | | 428.30 |
| | Cert(VH-CH1) | | |
| TNF-α | Unconjugated wild-type TNF-α | 0.47 | 0.21 |

“scFv70” = single chain variable fragment derived from anti-FAP antibody 872-70; “Cert” = Fab derived from a commercially available anti-TNF-α antibody; “sc15” “sc30”, “sc50”, “sc70”, “sc80”, or “sc105” = repeats of G4S fragment at the specified length (for example, sc15 consists of three GGGGS fragments of 15 amino acids length as GGGGS GGGGS GGGGS (SEQ ID NO: 7)); “(VL-CL)” = antibody light chain portion of a Fab; “(VH-CH1)” = antibody heavy chain portion of a Fab; “x” separating different peptidic chains forming part of a immunoconjugate molecule; “-” linker or connection (i.e. direct fusion) between different moieties; moieties in a peptidic chain are listed according to the N to C orientation in the peptidic chain; “/” TNF-α activity not detectable.

TNF-α activities of immunoconjugates having the configuration in FIG. 6A can be restored by 10³ fold with the cis-presentation of FAP on the surface of HEK Blue TNF-α reporter cells. Such results indicate that anchoring the immunoconjugates to the target cells (e.g., cells expressing TNF-α receptors) can partially restore the inhibited TNF-α activities in immunoconjugates containing mutant TNF-α in an antigen-dependent manner.

### 6.6.4 Inhibition and Restoration of TNF-α Activity in Immunoconjugates Containing Two-In-One Masking Moieties

In order to demonstrate shielding and de-shielding of the TNF-α activity through intramolecular interaction and disassociation between the cytokine moiety and the masking moiety of the present immunoconjugates, a bispecific two-in-one antibody capable of (a) binding to TNF-α and inhibiting the cellular effect of TNF-α, and (b) binding to FAP were created following the procedures described above. The two-in-one antibody was then fused to mutant TNF-α polypeptide and anti-FAP scFv via peptidic linkers to generate an immunoconjugate. Methods for the molecular cloning, protein expression and purification involved herein are as described above or known in the art. A schematic illustration of a trimer of the above-described immunoconjugates containing the mutant TNF-α, the anchoring moiety, and the two-in-one masking moiety is shown in FIG. 7A.

Immunoconjugates having the configuration in FIG. 7A were subjected to the TNF-α signaling assay described in Section 6.4.1 above. The results are shown in FIGS. 7B through 7G. 004261 Particularly, six different immunoconjugates having the configuration as shown in FIG. 7A were tested in the absence or presence of FAP expressed on the cell surface of HEK Blue TNF-α reporter cells. Each of the six immunoconjugates contained a Y87F or S147G point mutation of TNF-α˜, a different two-in-one masking moiety, and a different anchoring moiety. The unconjugated wild-type TNF-α˜ polypeptide (Sino Biological, Beijing, China) was included as a negative control.

As shown in FIGS. 7B through 7G, the unconjugated wild-type TNF-αL polypeptide elicited strong responses to TNF-αL in HEK reporter cells in a dose-dependent manner in the absence (solid circle) and presence (open circle) of FAP expression. In the absence of FAP-expressing cells, TNF-αL activities remained significantly inhibited in the tested immunoconjugates (solid triangle). In contrast, in the presence of FAP-expressing cells, TNF-αL activities of the tested immunoconjugates were partially restored (open triangle). The EC₅₀ concentration of the six tested immunoconjugates, as well as the unconjugated wild-type TNF-α˜ polypeptide, are shown in Table 6 below.

TABLE 6
EC₅₀ Concentration of Tested Immunoconjugates or Unconjugated Wild-type TNF-α.

| | | EC50 | |
|---|---|---|---|
| | | With FAP- | Without FAP- |
| Protein ID | Protein Description | Expression | Expression |
| T001 | TNFα(S147G)-VH(FN06)-CH1 x | 0.2 pM | 510 pM |
| | scFV70-sc50-VL(FN06)-CL | | |
| T002 | TNFα(Y87F)-VH(FN06)-CH1 x | 1.2 pM | >100 nM |
| | scFV70-sc50-VL(FN06)-CL | | |
| T003 | TNFα(S147G)-VH(FN15)-CH1 x | 1.4 pM | 11.2 nM |
| | scFV70-sc50-VL(FN15)-CL | | |
| T004 | TNFα(Y87F)-VH(FN15)-CH1 x | 7.5 pM | >100 nM |
| | scFV70-sc50-VL(FN15)-CL | | |
| T005 | TNFα(Y87F)-VH(FN06)-CH1 x | 29.5 pM | >100 nM |
| | scFV5-sc50-VL(FN06)-CL | | |
| T006 | TNFα(Y87F)-VH(FN06)-CH1 x | 16.2 pM | >100 nM |
| | scFV59-sc50-VL(FN06)-CL | | |
| TNF-α | Unconjugated wild-type TNF-a | 0.6 pM | 0.3 pM |

“scFv70” = single chain variable fragment derived from anti-FAP antibody 872-70; “scFV59” = single chain variable fragment derived from anti-FAP antibody 872-59; “sc15” “sc30”, “sc50”, “sc70”, “sc80”, or “sc105” = repeats of G4S fragment at the specified length (for example, sc15 consists of three GGGGS fragments of 15 amino acids length as GGGGS GGGGS GGGGS (SEQ ID NO: 7)); “x” separating different peptidic chains forming part of a immunoconjugate molecule; “-” linker or connection (i.e. direct fusion) between different moieties; moieties in a peptidic chain are listed according to the N to C orientation in the peptidic chain.

The above results demonstrate that TNF-α˜ activities in the present immunoconjugates can be significantly inhibited via intramolecular interaction between the TNF-α˜ and the masking moiety. Further, TNF-αL activities can be restored when the target cells (e.g., cells expressing TNF-αL receptors) express the antigen recognized by the anchoring moiety and two-in-one masking moiety in the immunoconjugates.

### 6.6.5 Trans-Presentation of FAP Allows Bystander Restoration of WTNF-α Activity in Immunoconjugates Containing Two-In-One Masking Moieties

TNF-α(activities of the same six immunoconjugates described in Section 6.6.4 above, as well as that of the unconjugated wild-type TNF-α˜ polypeptide (Sino Biological, Beijing, China), were tested in the absence or presence of FAP-expressing HEK293 cells co-cultured with the HEK Blue TNF-α˜ reporter cells. The TNF-α˜ signaling assay used herein is described in Section 6.4.1 above. The results are shown in FIGS. 8A through 8F.

As shown in FIGS. 8A through 8F, the unconjugated wild-type TNF-αL polypeptide elicited strong responses to TNF-αL in HEK reporter cells in a dose-dependent manner in the absence (solid circle) and presence (open circle) of FAP-expressing cells. In the absence of FAP-expressing cells, TNF-αL activities remained significantly inhibited in the tested immunoconjugates (solid triangle). In contrast, in the presence of FAP-expressing cells, TNF-αL activities of the tested immunoconjugates were partially restored (open triangle). The EC₅₀ concentration of the six tested immunoconjugates, as well as the unconjugated wild-type TNF-αL polypeptide, are shown in Table 7 below.

TABLE 7
EC₅₀ Concentration of Tested Immunoconjugates or Unconjugated Wild-type TNF-α.

| | | EC50 | |
|---|---|---|---|
| Protein | | With FAP- | Without FAP- |
| ID | Protein Description | Expressing Cells | Expressing Cells |
| T001 | TNFα(S147G)-VH(FN06)-CH1 x | 30.7 pM | 495 pM |
| | scFV70-sc50-VL(FN06)-CL | | |
| T002 | TNFα(Y87F)-VH(FN06)-CH1 x | 510 pM | >100 nM |
| | scFV70-sc50-VL(FN06)-CL | | |
| T003 | TNFα(S147G)-VH(FN15)-CH1 x | 13.6 pM | 9.4 nM |
| | scFV70-sc50-VL(FN15)-CL | | |
| T004 | TNFα(Y87F)-VH(FN15)-CH1 x | 323 pM | >100 nM |
| | scFV70-sc50-VL(FN15)-CL | | |
| T005 | TNFα(Y87F)-VH(FN06)-CH1 x | 411 pM | >100 nM |
| | scFV5-sc50-VL(FN06)-CL | | |
| T006 | TNFα(Y87F)-VH(FN06)-CH1 x | 446 pM | >100 nM |
| | scFV59-sc50-VL(FN06)-CL | | |
| TNF-a | Unconjugated wild-type TNF-α | 1.9 pM | 1.5 pM |

“scFv70” = single chain variable fragment derived from anti-FAP antibody 872-70; “scFV59” = single chain variable fragment derived from anti-FAP antibody 872-59; “sc15” “sc30”, “sc50”, “sc70”, “sc80”, or “sc105” = repeats of G4S fragment at the specified length (for example, sc15 consists of three GGGGS fragments of 15 amino acids length as GGGGS GGGGS GGGGS (SEQ ID NO: 7)); “x” separating different peptidic chains forming part of a immunoconjugate molecule; “-” linker or connection (i.e., direct fusion) between different moieties; moieties in a peptidic chain are listed according to the N to C orientation in the peptidic chain.

The above results demonstrate that TNF-α activities in the present immunoconjugates can be significantly inhibited via intramolecular interaction between the TNF-α and the masking moiety. Further, TNF-α activities can be restored cells nearby the target cells (e.g., cells expressing TNF-α receptors) express the antigen recognized by the anchoring moiety and two-in-one masking moiety in the immunoconjugate.

### 6.6.6 TNF-α Activity in Immunoconjugates Containing Two-In-One Masking Moieties can be Partially Restored in the Absence of Anchoring Moieties

The anchoring moiety in the immunoconjugates described in Section 6.6.4 above was removed. Moreover, the two-in-one Fab capable of binding both TNF-α and FAP was replaced with a two-in-one scFv with the same binding capacity. A schematic illustration of a trimer of the above-described immunoconjugates containing the mutated TNF-α and the two-in-one masking moiety is shown in FIG. 9A.

Immunoconjugates having the configuration in FIG. 9A were subjected to the TNF-α signaling assay described in Section 6.4.1 above. The results are shown in FIG. 9B.

Particularly, two different immunoconjugates having the configuration in FIG. 9A were tested in the absence or presence of FAP-expressing HEK293 cells co-cultured with the HEK Blue TNF-α reporter cells. In both tested immunoconjugates, the mutant TNF-α polypeptide had a Y87F point mutation. Two two-in-one scFv antibodies designated as FN06 and FN15 were used as the masking moiety in the two tested immunoconjugates, respectively.

As shown in FIG. 9B, in the absence of FAP-expressing cells, TNF-α activities remained significantly inhibited in the tested immunoconjugates (circle and up triangle). In contrast, in the presence of FAP-expressing cells, TNF-α activities of the tested immunoconjugates were partially restored (down triangle and diamond). The EC₅₀ concentration of the two tested immunoconjugates are shown in Table 8 below.

TABLE 8
EC₅₀ Concentration of Tested Immunoconjugates.

| | | EC50 | |
|---|---|---|---|
| | | With FAP- | Without FAP- |
| | | Expressing | Expressing |
| Protein ID | Protein Description | Cells | Cells |
| 37 | TNF-α (Y87F)-SC₅₀- | 92 pM | 3.4 nM |
| | scFv(FN06) | | |
| 39 | TNF-α (Y87F)-SC₅₀- | 29 pM | >100 nM |
| | scFv(FN15) | | |

The above results demonstrate that the inhibited TNF-α activities in immunoconjugates containing mutated TNF-α and two-in-one masking moieties can be partially restored even in the absence of anchoring moieties. This is probably achieved due to the de-shielding from TNF-α by the masking moiety upon the masking moiety's binding to FAP, thus activating TNF-α activity.

6.6.7 Regulating TNF-α Activity Via Adjusting the Length of Linkers within TNF-α Containing Immunoconjugates

In order to examine the impact of the length of linkers within TNF-α containing immunoconjugates on TNF-α activity, immunoconjugates having the configuration in FIG. 7A were constructed with different lengths of the linker between mutant TNF-α polypeptide and the two-in-one masking moiety (50, 70, or 160 amino acids long) or the linker between the anchoring moiety and the two-in-one masking moiety (70 or 160 amino acids long). Methods for the molecular cloning, protein expression and purification involved herein are as described above or known in the art. Such immunoconjugates were subjected to the TNF-α signaling assay described in Section 6.4.1 above. The results are shown in FIGS. 10A through 10D.

Particularly, the immunoconjugates tested in FIG. 10A contained a S147G point mutation in the TNF-α polypeptide and FN06 as the two-in-one masking moiety; the immunoconjugates tested in FIG. 10B contained a S147G point mutation in the TNF-α polypeptide and FN15 as the two-in-one masking moiety; the immunoconjugates tested in FIG. 10C contained a Y87F point mutation in the TNF-α polypeptide and FN06 as the two-in-one masking moiety; the immunoconjugates tested in FIG. 10D contained a Y87F point mutation in the TNF-α polypeptide and FN15 as the two-in-one masking moiety; the anchoring moiety of all tested immunoconjugates is scFv70. In addition, the commercially available wild-type TNF-α polypeptide in the non-conjugated form was also included in the assay as a control (Sino Biological; solid square).

As shown in FIGS. 10A through 10D, the unconjugated wild-type TNF-α polypeptide elicited strong responses to TNF-α in HEK reporter cells in a dose-dependent manner in the absence of FAP expression (solid square). In the absence of FAP-expressing cells, TNF-α activities remained significantly inhibited in the tested immunoconjugates (solid circle and triangles); in the presence of FAP-expressing cells, TNF-α activities of the tested immunoconjugates were at least partially restored (open circle and triangles). Moreover, although there is no meaningful correlation between TNF-α activity and the length of the linker connecting the anchoring moiety and the masking moiety, there is a modest (˜10-fold) difference in TNF-α activity between the shortest and the longest length of the linker connecting TNF-αL and the masking moiety. The EC₅₀ concentration of the tested immunoconjugates, as well as the unconjugated wild-type TNF-αL polypeptide, are shown in Table 9 below.

TABLE 9
EC₅₀ Concentration of Tested Immunoconjugates.

| | | EC50 | | |
|---|---|---|---|---|
| Protein | | Without FAP- | With FAP- | |
| ID | Protein Description | Expression | Expression | Ratio |
| TNF-α | Unconjugated wild-type TNF-α | 0.43 pM | | |
| T014 | TNFα(S147G)-sc50-VH(FN06)-CH1 x | 75.12 pM | 0.38 pM | 199 |
| | scFV70-sc70-VL(FN06)-CL | | | |
| T018 | TNFα(S147G)-sc70-VH(FN06)-CH1 x | 191.00 pM | 0.49 pM | 386 |
| | scFV70-sc70-VL(FN06)-CL | | | |
| T022 | TNFα(S147G)-sc80X2-VH(FN06)-CH1 x | 84.27 pM | 0.31 pM | 275 |
| | scFV70-sc70-VL(FN06)-CL | | | |
| T026 | TNFα(S147G)-sd50-VH(FN06)-CH1 x | 478.27 pM | 0.61 pM | 784 |
| | scFV70-sc80X2-VL(FN06)-CL | | | |
| T028 | TNFα(S147G)-sd70-VH(FN06)-CH1 x | 152.56 pM | 0.31 pM | 488 |
| | scFV70-sc80X2-VL(FN06)-CL | | | |
| T030 | TNFα(S147G)-sc80X2-VH(FN06)-CH1 x | 41.53 PM | 0.19 pM | 217 |
| | scFV70-sc80X2-VL(FN06)-CL | | | |
| T016 | TNFα(S147G)-sc50-VH(FN15)-CH1 x | 549.78 pM | 0.38 pM | 1430 |
| | scFV70-sc70-VL(FN15)-CL | | | |
| T020 | TNFα(S147G)-sc70-VH(FN15)-CH1 x | 326.92 pM | 0.42 pM | 783 |
| | scFV70-sc70-VL(FN15)-CL | | | |
| T024 | TNFα(S147G)-sc80X2-VH(FN15)-CH1 x | 69.89 pM | 0.23 pM | 309 |
| | scFV70-sc70-VL(FN15)-CL | | | |
| T015 | TNFα(Y87F)-sc50-VH(FN06)-CH1 x | >10 nM | 2.16 pM | |
| | scFV70-sc70-VL(FN06)-CL | | | |
| T019 | TNFα(Y87F)-sc70-VH(FN06)-CH1 x | >10 nM | 1.24 pM | |
| | scFV70-sc70-VL(FN06)-CL | | | |
| T023 | TNFα(Y87F)-sc80X2-VH(FN06)-CH1 x | >10 nM | 0.53 pM | |
| | scFV70-sc70-VL(FN06)-CL | | | |
| T027 | TNFα(Y87F)-sc50-VH(FN06)-CH1 x | >10 nM | 2.72 pM | |
| | scFV70-sc80X2-VL(FN06)-CL | | | |
| T029 | TNFα(Y87F)-sc70-VH(FN06)-CH1 x | >10 nM | 2.33 pM | |
| | scFV70-sc80X2-VL(FN06)-CL | | | |
| T031 | TNFα(Y87F)-sc80X2-VH(FN06)-CH1 x | >10 nM | 0.59 pM | |
| | scFV70-sc80X2-VL(FN06)-CL | | | |
| T017 | TNFα(Y87F)-sc50-VH(FN15)-CH1 x | >10 nM | 1.67 pM | |
| | scFV70-sc70-VL(FN15)-CL | | | |
| T021 | TNFα(Y87F)-sc70-VH(FN15)-CH1 x | >10 nM | 0.74 pM | |
| | scFV70-sc70-VL(FN15)-CL | | | |
| T025 | TNFα(Y87F)-sc80X2-VH(FN15)-CH1 x | >10 nM | 0.77 pM | |
| | scFV70-sc70-VL(FN15)-CL | | | |

“scFv70” = single chain variable fragment derived from anti-FAP antibody 872-70; “scFV59” = single chain variable fragment derived from anti-FAP antibody 872-59; “sc15” “sc30”, “sc50”, “sc70”, “sc80”, or “sc105” = repeats of G4S fragment at the specified length (for example, sc15 consists of three GGGGS fragments of 15 amino acids length as GGGGS GGGGS GGGGS (SEQ ID NO: 7)); “sc80X2” = two repeats of sc80 linker fused tandemly; “x” separating different peptidic chains forming part of a immunoconjugate molecule; “-” linker or connection (i.e., direct fusion) between different moieties; moieties in a peptidic chain are listed according to the N to C orientation in the peptidic chain.

The above results demonstrate that the linker length between TNF-α and the masking moiety modestly affects the ability of the masking moiety to inhibit the activity of TNF-α. A shorter linker tends to inhibit the activity of TNF-α more significantly.

### 6.7 Example 7: Construction of Covalent Disulfide-Linked TNF-α Variants

Wild-type TNF-α is a non-covalent trimer, and the equilibrium dissociation constant K_{D} of oligomerization is ˜0.2 nM. In order to retain the trimeric oligomerization state of TNF-α at low concentrations, covalent disulfide-linked TNF-α variants were created.

Particularly, eleven different double cysteine mutants as shown in Table 10 below, as well as a FLAG-tag at the C-terminus, were introduced into wild-type TNF-α. The mutant TNF-α variants were expressed in Expi293F cells and were purified using FLAG-tag purification. The size of the purified protein was further confirmed via SDS-PAGE (see e.g., FIG. 24A left). Methods for the molecular cloning, protein expression and purification, and SDS-PAGE involved herein are as described above or known in the art.

TABLE 10
Description and Yield of Covalent
Disulfide-linked TNF-α Variants

| ID | Protein Description | Yield (μg/ml) |
|---|---|---|
| 1 | TNFα(I155C L157C)-G4S.3- | 3.96 |
| | Flag-signal peptide H3 | |
| 2 | TNFα(P08C L55C)-G4S.3- | 30.48 |
| | Flag-signal peptide H3 | |
| 3 | TNFα(V123C I155C)-G4S.3- | 2.80 |
| | Flag-signal peptide H3 | |
| 4 | TNFα(F124C N34C)-G4S.3- | 2.56 |
| | Flag-signal peptide H3 | |
| 5 | TNFα(A96C P117C)-G4S.3- | 7.89 |
| | Flag-signal peptide H3 | |
| 6 | TNFα(P117C K98C)-G4S.3- | 4.86 |
| | Flag-signal peptide H3 | |
| 7 | TNFα(I97C Y115C)-G4S.3- | 18.36 |
| | Flag-signal peptide H3 | |
| 8 | TNFα(S99C W114C)-G4S.3- | 2.93 |
| | Flag-signal peptide H3 | |
| 9 | TNFα(Q102C E104C)-G4S.3- | 33.86 |
| | Flag-signal peptide H3 | |
| 10 | TNFα(H73C P113C)-G4S.3- | 16.39 |
| | Flag-signal peptide H3 | |
| 11 | TNFα(S95C G148C)-G4S.3- | 9.68 |
| | Flag-signal peptide H3 | |

As shown in the top panel of FIG. 11A, five (Protein IDS: 2, 7, 9, 10, and 11) of the eleven purified protein samples have protein bands at the approximate size of a TNF-α trimer (51-kDa). As shown in the bottom panel of FIG. 11A, after disulfide bonds were reduced by dithiothreitol (DTT) treatment, the same five purified protein samples (Protein ID NOs: 2, 7, 9, 10, and 11) appeared to have protein bands at the approximate size of a TNF-α monomer (17-kDa).

The five disulfide-linked TNF-α variants (Protein ID NOs: 2, 7, 9, 10, and 11) were subjected to cell-based TNF-α signaling assay described in Sections 6.4.1. In addition, the commercially available wild-type TNF-α was also included in the assay as a control (Sino Biological). As shown in FIG. 11B, three disulfide-linked TNF-α variants (Protein ID NOs: 2, 9, and 11) maintained or exceeded the potency of wild-type TNF-α (solid circle). The mutant disulfide pairs in those three variants were: P08C-L55C, Q102C-E104C, and S95C-G148C. The EC₅₀ concentration of the five covalent disulfide-linked TNF-α variants, as well as the wild-type TNF-α, are shown in Table 11 below.

TABLE 11
EC50 Concentration of Tested Covalent
Disulfide-linked TNF-α Variants

| ID | Protein Description | EC50 |
|---|---|---|
| s-TNFα | Sino TNFα | 0.97 |
| 2 | TNFα(P08C L55C)-G4S.3- | 0.40 |
| | Flag-signal peptide H3 | |
| 7 | TNFα(I97C Y115C)-G4S.3- | 33.03 |
| | Flag-signal peptide H3 | |
| 9 | TNFα(Q102C E104C)-G4S.3- | 0.77 |
| | Flag-signal peptide H3 | |
| 10 | TNFα(H73C P113C)-G4S.3- | 301.16 |
| | Flag-signal peptide H3 | |
| 11 | TNFα(S95C G148C)-G4S.3- | 1.39 |
| | Flag-signal peptide H3 | |

The above results demonstrate that covalent disulfide-linked TNF-α variants are capable of maintaining, or even exceeding, TNF-α activities of wild-type non-covalent TNF-α.

### 6.8 Example 8: In Vitro Tumor Cell Killing

The activity of TNFα immunoconjugate molecules were assessed for their ability to enable enhanced killing of tumor cell line WEHI-164 in the presence or absence of a co-cultured WEHI-164 cell line ectopically expressing FAP (WEHI-164 Clone M2C2). Cells added to the 96 well plate kept constant at 20,000 cells per well while varying the ratio of cells containing FAP (20,000 WEHI-164, 10,000 WEHI-164+10,000 WEHI-164 M2C2 or 20,000 WEHI-164 M2C2). The cells were allowed to proliferate for 2 days in RPMI-1640 Medium supplemented with 10% FBS at 37° C., 5% CO₂ before proteins were added at concentrations ranging from 0-100 nM in the presence or absence of Actinomycin D (concentrations ranging from 0-0.6 μg/mL). Cells were allowed to proliferate for an additional two days. Cell viability was determined with the alamarBlue assay (Invitrogen, Waltham, Massachusetts) monitoring fluorescence (540 nm excitation, 590 nm emission). Data were plotted as relative fluorescence (y-axis, relative fluorescence units) vs. immunoconjugate concentration (x-axis, in picomolar). The signal of the y-axis is proportional to the number of viable cells in the assay. Molecules screened with this assay are summarized in the Table below, and different configurations of the screened molecule are shown in FIG. 12. Screening conditions and results are plotted in FIGS. 13 to 21.

| Molecule ID | Protein Description | Configuration |
|---|---|---|
| F-TNF-α | Unconjugated wild-type TNF-α | |
| T017 | TNFa(Y87F)-SC50-VH(FN15)-CH1 × | 6 |
| | scFV70-linker(sc70)-VL(FN15)-CL | |
| T038 | TNFa(S95A)-SC50-VH(FN15)-CH1 × | 2 |
| | VH(FN15)-CL | |
| T059 | TNFa(N34H)-SC50-VH(FN15)-CH1 × | 6 |
| | scFV70-linker(sc70)-VL(FN15)-CL | |
| T061 | TNFa(S95A)-SC50-VH(FN15)-CH1 × | 6 |
| | scFV70-linker(sc70)-VL(FN15)-CL | |
| T062 | TNFa(A145G)-SC50-VH(FN15)-CH1 × | 6 |
| | scFV70-linker(sc70)-VL(FN15)-CL | |
| T064 | TNFa(S147A)-SC50-VH(FN15)-CH1 × | 6 |
| | scFV70-linker(sc70)-VL(FN15)-CL | |
| T067 | TNFa(N34H)-SC50-VH(FN15)-CH1 × | 6 |
| | SibrotuzumabSCFV-linker(sc70)-VL(FN15)-CL | |
| T074 | TNFa(N34H)-SC50-VH(FN15)-CH1 × | 12 |
| | VHH E03-SC70-VHH E03-SC70-VL(FN15)-CL | |
| T075 | TNFa(N34H)-SC50-VH(FN15)-CH1 × | 12 |
| | VHH E04-SC70-VHH E04-SC70-VL(FN15)-CL | |
| T076 | TNFa(N34H)-SC50-VH(FN15)-CH1 × | 12 |
| | VHH EO6-SC70-VHH E05-SC70-VL(FN15)-CL | |
| T077 | TNFa(A33T N34H)-SC50-VH(FN15)-CH1 × | 6 |
| | SibrotuzumabSCFV-linker(sc70)-VL(FN15)-CL | |
| T078 | TNFa(N34H A145G)-SC50-VH(FN15)-CH1 × | 6 |
| | SibrotuzumabSCFV-linker(sc70)-VL(FN15)-CL | |
| T079 | TNFa(N34H A145V)-SC50-VH(FN15)-CH1 × | 6 |
| | SibrotuzumabSCFV-linker(sc70)-VL(FN15)-CL | |
| T080 | TNFa(A33T A145G)-SC50-VH(FN15)-CH1 × | 6 |
| | SibrotuzumabSCFV-linker(sc70)-VL(FN15)-CL | |
| T081 | TNFa(A33T A145V)-SC50-VH(FN15)-CH1 × | 6 |
| | SibrotuzumabSCFV-linker(sc70)-VL(FN15)-CL | |
| T082 | TNFa(S95A)-SC50-VH(FN15)-CH1 × | 6 |
| | SibrotuzumabSCFV-linker(sc70)-VL(FN15)-CL | |
| T083 | TNFa(S147A)-SC50-VH(FN15)-CH1 × | 6 |
| | SibrotuzumabSCFV-linker(sc70)-VL(FN15)-CL | |
| T084 | TNFa(S147A)-SC50-VH(FN15)-CH1 × | 12 |
| | VHH E04-SC70-VHH E04-SC70-VL(FN15)-CL | |
| T085 | TNFa(S95A)-SC50-VH(FN15)-CH1 × | 12 |
| | VHH E06-SC70-VHH E05-SC70-VL(FN15)-CL | |
| T086 | TNFa(S147A)-SC50-VH(FN15)-CH1 × | 12 |
| | VHH E06-SC70-VHH E05-SC70-VL(FN15)-CL | |
| T087 | TNFa(S95A)-SC50-VH(FN15)-CH1 × | 12 |
| | VHH E04-SC70-VHH E04-SC70-VL(FN15)-CL | |
| T098 | TNFa(S95A)-10X(G4S)-VH(FN15)-CH1 × | 6 |
| | SibrotuzumabSCFV-14X(G4S)-VL(FN15)-CL | |
| T099 | TNFa(S95A)-SC50-VH(FN15)-CH1 × | 12 |
| | VHH E04-SC70-VHH E04-SC70-VL(FN15)-CL | |
| T100 | TNFa(S95A)-SC50-VH(FN15)-CH1 × | 12 |
| | VHH E04-SC70-VHH E04-SC70-VL(FN15)-CL | |
| T101 | TNFa(A33T A145G)-SC50-VH(FN15)-CH1 × | 12 |
| | VHH E04-SC70-VHH E04-SC70-VL(FN15)-CL | |
| T102 | TNFa(S95A)-SC50-VH(FN15)-CH1 × | 7 |
| | VHH E21-14X(G4S)-VL(FN15)-CL | |
| T103 | TNFa(S95A)-SC50-VH(FN15)-CH1 × | 7 |
| | VHH E22-14X(G4S)-VL(FN15)-CL | |
| T104 | TNFa(S95A)-SC50-VH(FN15)-CH1 × | 7 |
| | VHH E23-14X(G4S)-VL(FN15)-CL | |
| T105 | TNFa(S95A)-SC50-VH(FN15)-CH1 × | 7 |
| | VHH E24-14X(G4S)-VL(FN15)-CL | |
| T106 | TNFa(S95A)-SC50-VH(FN15)-CH1 × | 7 |
| | VHH E25-14X(G4S)-VL(FN15)-CL | |
| T107 | TNFa(S95A)-SC50-VH(FN15)-CH1 × | 7 |
| | VHH E26-14X(G4S)-VL(FN15)-CL | |
| T108 | TNFa(S95A)-SC50-VH(FN15)-CH1 × | 7 |
| | VHH E27-14X(G4S)-VL(FN15)-CL | |
| T109 | TNFa(S95A)-SC50-VH(FN15)-CH1 × | 7 |
| | VHH E28-14X(G4S)-VL(FN15)-CL | |
| T110 | TNFa(S95A)-SC50-VH(FN15)-CH1 × | 6 |
| | SibrotuzumabSCFV-linker(sc70)- | |
| | VL(FN15-L1 Rev1)-CL | |
| T111 | TNFa(S95A)-SC50-VH(FN15)-CH1 × | 2 |
| | P102-FN15-L1 Rev1 | |

Impact of FAP binding capability was assessed with TNF-α containing immunoconjugate molecules having different configurations as shown in FIG. 7A (full immunoconjugate), FIG. 17C (immunoconjugate lacking FAP binding activity in the masking moiety) and FIG. 17E (immunoconjugate lacking FAP-binding anchoring moiety). The results are plotted in FIGS. 17A, 17B and 17D, respectively. As shown, cell killing activity comparable to soluble TNF-α was observed in immunoconjugate molecule having a masking moiety with intact FAP binding capability in addition to FAP-binding anchoring moiety.

The assays reported in FIG. 18A to 18L show screening of various TNF-α containing immunoconjugate molecules constructed with different anchoring moieties and/or two-in-one masking moiety derived from FAP/TNF-α two-in-one antibody FN15 using the WEHI-164 co-culture assay. FIG. 18B shows the activity of immunoconjugate T082 in the presence of 1 μg/mL Actinomycin D where cell killing was critically dependent on the surface expression of FAP. Here, the potency as determined by the EC₅₀ was ˜10⁵-fold different dependent upon the expression of FAP on the cell surface. By contrast, FIG. 18A shows that there was both a significant shift in the potency of cell killing (˜100-fold) as well as lower overall cell killing reflected by the higher residual signal after killing compared to the TNF-α control. The immunoconjugate in FIG. 18A contains mutations that eliminate FAP binding in the two-in-one antibody demonstrating that the anchor alone is insufficient to impart potent and complete cell killing. The difference in these data demonstrate that potent cell killing occurred when both an anchoring moiety and a functional two-in-one masking moiety were present. FIG. 18C depicts cell killing activities for an immunoconjugate that differs from T082 in that it lacks a VHH anchor. This data demonstrate that the anchoring moiety endowed the immunoconjugate with highly potent cell killing activity in the presence of FAP-expressing cells. FIG. 18D show cell killing activity of an immunoconjugate lacking FAP binding capability in the two-in-one antibody in addition to lacking an anchor. Here, virtually no activity was observed demonstrating the importance of both the anchor and two-in-one functionality of the immunoconjugate. FIG. 18E-FIG. 18L show the activity of the immunoconjugate where the anchor is varied. Here, a variety of VHH moieties specific to FAP were introduced demonstrating a range of potencies.

Additionally, the assay reported in FIGS. 21A to 21E shows cell killing by T102 in the presence or absence of WEHI-164 cells expressing FAP, and in the presence or absence of Actinomycin D. FIGS. 22A to 22D show cell killing quantitated as normalized cell viability. As shown, the increasing amount of Actinomycin D from 0.2 μg/mL to 1 μg/mL can increase cells' apoptotic sensitivity to TNF-α for both WEHI-164 and WEHI-164-FAP. In the presence of 0.2 μg/mL Actinomycin D, T102 is able to kill ˜50% WEHI cells (open down triangle), likely the 50% WEHI-164-FAP cells which are responsive to T102. In presence of 1 μg/mL Actinomycin D, T102 is able to kill almost all cells comparable to unconjugated TNF-α (solid down triangle). Since the 50% WEHI-164 cells were killed as well and they are not responding to T102 directly, they have to be impacted by the trans-presentation of FAP from adjacent WEHI-164-FAP cells. It suggests the FAP activated TNF-α from T102 can be potent in both cis-presentation and trans-presentation.

### 6.9 Example 9: In Vitro 3D Methylcellulose Assay of Patient-Derived Xenograft Tumor

A tumor from the human sarcoma cell line SA3831 (CrownBio) was harvested from a tumor-bearing mouse. The tumor was minced into tiny pieces, digested with collagenase for 1 hour and filtered through a Falcon cell strainer. Red blood cells were removed by ACK lysing buffer (GIBCO, Cat. No: A1049201). The cell suspension was centrifuged at 1000 rpm for 5 minutes and washed with PBS. The cell suspension was centrifuged once more, the supernatant was removed and cells were resuspended in DMEM/F12+10% FBS+Pen/Strep. Cells were seeded in a 96 well plate (90 μL, ˜15,000/well) after dilution with 1% methylcellulose solution (final concentration 0.65% methylcellulose). Cells were incubated overnight in a humidified incubator at 37° C. with 5% CO₂. The following day, 10 μL of a 10× concentration immunoconjugate or chemical-immunoconjugate combination were added to the 96 well plate. The plates were allowed to incubate with compounds for two days with readings taken at 24 and 48 hours. Cell viability was monitored with CellTiter-Glo® Luminescent Cell Viability kit (Promega, Cat. No: G7572). The results are plotted in FIG. 23A, which shows that human soft tissue sarcoma cells (SA3831) express high levels of FAP ((˜1×10⁶ FAP/cell). FIG. 23B shows either 100 pM TNF-α or 10 nM TNF-α conjugate alone didn't affect SA3831 cells. In the presence of 0.1 μg/mL Actinomycin D, a TNF-α immunoconjugate molecule as described herein is synergistic with chemotherapeutic agent Actinomycin D on human primary tumor cells within therapeutically relevant concentrations. This demonstrates TNF-α immunoconjugates are capable of directly killing human tumor cells expressing FAP. Furthermore, the potency of the T098 immunoconjugate is comparable to unshielded TNF-α cytokine. Taken together, combination of chemotherapy and TNF-α immunoconjugate has direct clinical relevance for FAP-positive and TNF-α responsive human cancers.

### 6.10 Example 10: In Vivo Tumor Growth Inhibition (TGI) by TNF-α Containing Immunoconjugate

To establish a WEHI-164 tumor model, 5×10⁶ WEHI-164 mouse fibrosarcoma cells ectopically expressing FAP were implanted subcutaneously in the flank of female BALB/c mice. Tumors size was monitored by caliper (Tumor volume (mm³)=(length (mm)×width (mm)²)/2). Tumors were allowed to grow to ˜100 mm³ before beginning treatment. Dosing of PBS, unconjugated TNFα (4 μg) and T098 (25 μg) was performed on days 0, 2 and 4 via tail vein injection. Tumor size was measured every 2-3 days and body weight and survival were monitored daily. As shown in FIGS. 25A and 25B, T098 significantly inhibited tumor growth in the mice compared to PBS treated group, while inducing significantly reduced toxicity as compared to unconjugated TNF-α polypeptide as measured by body weight change following administration.

To establish a WEHI-164 tumor model in immune compromised mice, 5×10⁶ WEHI-164 mouse fibrosarcoma cells ectopically expressing FAP were implanted subcutaneously in the flank of female M-NSG mice (NOD-Prkd^{scid} Il2rg^{eml}/Smoc, NM-NSG-001, ModelOrg, Shanghai). Tumor size was monitored by caliper (Tumor volume (mm³)=(length (mm)×width (mm)²)/2). Tumors were allowed to grow to ˜100 mm³ before beginning treatment. Dosing of PBS, unconjugated TNFα (4 μg) and T098 (25 μg) was performed on days 0, 2 and 4 via tail vein injection. Tumor size was measured every 2-3 days and body weight and survival were monitored daily. As shown in FIG. 25C, neither TNF-α nor T098 inhibited tumor growth in the mice compared to PBS treated group, which is consistent with concept that TNF-α induced tumor regression is immune cell dependent.

### 6.11 Example 11: In Vivo Tumor Growth Inhibition (TGI) by TNF-α Containing Immunoconjugate in Combination with Actinomycin D

To further explore in vivo tumor inhibition effect of T102 in combination with Actinomycin D, 5×10⁶ WEHI-164 mouse fibrosarcoma cells ectopically expressing FAP were implanted subcutaneously in the flank of female BALB/c mice. Tumors size was monitored by caliper (Tumor volume (mm³)=(length (mm)×width (mm)²)/2). Tumors were allowed to grow to ˜100 mm³ before beginning treatment. Dosing of PBS, TNFα (4 μg) and T102 (100 μg) was performed on days 0, 2 and 4 while Actinomycin D (1 μg) was administered on days 0 and 4 via tail vein injection. Tumor size was measured every 2-3 days and body weight and survival were monitored daily.

As shown in FIG. 26A, mice treated with the combination of T102 and Actinomycin D exhibited complete tumor rejection with no observable increase in tumor size at the end of the observation period, while toxicity as measured by immediate body weight loss following administration was significantly reduced comparing to unconjugated TNF-α.

After the tumor size reaches ˜200 mm³ with WEHI-164-FAP cells in Balb/c mice, the PBS or 25 μg TNF-α containing immunoconjugate was administered intravenously through the tail vein. Tumor was harvest 48 hr after dosing and fixed with 4% paraformaldehyde, and applied for Hematoxylin and Eosin staining. FIG. 26B shows Hematoxylin and Eosin (H&E) staining 48 hours after first dosing of PBS (control) or 25 μg TNF-α-containing immunoconjugate as described herein, indicting the administered immunoconjugate molecule induced destruction and lymphocyte infiltration in tumor. These data demonstrated that while TNF-α is known to be able to induce tumor destruction and necrosis, the TNF-α immunoconjugate is able to cause tumor destruction in FAP-positive WEHI-164-FAP tumor.

Furthermore, 2×10⁶ WEHI-164 mouse fibrosarcoma cells that was not engineered to express hFAP were implanted subcutaneously in the flank of female Balb/c mice. Tumors size was monitored by caliper (Tumor volume (mm³)=(length (mm)×width (mm)²)/2). Tumors were allowed to grow to ˜100 mm³ before beginning treatment. Dosing of PBS, unconjugated TNFα (4 μg) and TNF-α containing immunoconjugate (25 μg) was performed on days 0, 2 and 4 via tail vein injection. Tumor size was measured every 2-3 days and body weight and survival were monitored daily. FIG. 26C shows the tumor inhibition efficacy in WEHI-164 WT model in Balb/c mice of an example of a TNF-α containing immunoconjugates molecule as described herein. While the TNF-α was still efficacious to inhibit WEHI-164 WT tumor, the TNF-α immunoconjugate was ineffective. This result demonstrates that tumor inhibition efficacy of this TNF-α containing immunoconjugates depends on the presence of FAP to unshield TNF-α activity.

What is claimed:

1. An immunoconjugate molecule comprising
(a) a cytokine moiety comprising a cytokine polypeptide having a cytokine activity;
(b) a masking moiety;
wherein the masking moiety comprises a bispecific antibody or antigen binding fragment thereof capable of binding to the cytokine polypeptide and a first target antigen;
wherein when binding to the cytokine polypeptide, the masking moiety reduces or inhibits the cytokine activity;
wherein when binding to the first target antigen, the masking moiety disassociates from the cytokine polypeptide, thereby activating the cytokine activity; and
wherein the cytokine polypeptide is wild-type or mutant TNF-α.

2. The immunoconjugate molecule of claim 1, wherein the masking moiety comprises an intact antibody, a Fab, a scFab, a Fab′, a F(ab′)₂, a Fv, a scFv, a dsFv, a diabody, a triabody, a tetrabody, or a VHH formed from antibody fragments.

3. The immunoconjugate molecule of claim 1 or 2, wherein the bispecific antibody is a two-in-one antibody.

4. The immunoconjugate molecule of any one of claims 1 to 3, wherein the first target antigen is not the cytokine polypeptide.

5. The immunoconjugate molecule of any one of claims 1 to 4, wherein the first target antigen is expressed on a first cell surface.

6. The immunoconjugate molecule of claim 5, wherein the first cell does not express a TNF-α receptor.

7. The immunoconjugate molecule of claim 5, wherein the first cell also expresses a TNF-α receptor.

8. The immunoconjugate molecule of any one of claims 5 to 7, wherein the first cell is a cancer cell or a cell in a tumor microenvironment.

9. The immunoconjugate molecule of any one of claims 1 to 4, wherein the first target antigen is soluble.

10. The immunoconjugate molecule of any one of claims 1 to 9, wherein the first target antigen is a tumor associated antigen.

11. The immunoconjugate molecule of any one of claims 1 to 9, wherein the first target antigen is fibroblast activation protein (FAP).

12. The immunoconjugate molecule of any one of claims 1 to 9, wherein the first target antigen is Tumor-associated calcium signal transducer 2 (TROP2).

13. The immunoconjugate molecule of any one of claims 1 to 12, wherein the TNF-α is wild-type or mutant human TNF-α.

14. The immunoconjugate molecule of claim 13, wherein the mutant human TNF-α comprises at least one point mutation selected from
(a) A33T, N34A, N34H, Y87A, Y87F, S95A, A145G, A145V, S147A, and S147G in SEQ ID NO:1;
(b) P08C, L55C, Q102C, E104C, S95C, G148C, I97C, Y115C, H73C, and P113C of SEQ ID NO:1; or
(c) (i) double mutations of P08C and L55C; (ii) double mutations of Q102C and E104C, (iii) double mutations of S95C and G148C, (iv) double mutations of I97C and Y115C; (v) double mutations of H73C and P113C; or (vi) any combinations of (i) to (v), of SEQ ID NO:1.

15. The immunoconjugate molecule of any one of claims 1 to 12, wherein the TNF-α is wild-type or mutant TNF-α from a non-human species.

16. The immunoconjugate molecule of claim 15, wherein the mutant TNF-α comprises at least one point mutation corresponding to at least one mutation selected from
(a) A33T, N34A, N34H, Y87A, Y87F, S95A, A145G, A145V, S147A, and S147G in SEQ ID NO:1;
(b) P08C, L55C, Q102C, E104C, S95C, G148C, I97C, Y115C, H73C, and P113C of SEQ ID NO:1; or
(c) (i) double mutations of P08C and L55C; (ii) double mutations of Q102C and E104C, (iii) double mutations of S95C and G148C, (iv) double mutations of I97C and Y115C; (v) double mutations of H73C and P113C; or (vi) any combinations of (i) to (v), of SEQ ID NO:1.

17. The immunoconjugate molecule of any one of claims 1 to 16, further comprising:
(c) an anchoring moiety comprising an antibody or antigen binding fragment thereof that specifically binds to a second target antigen.

18. The immunoconjugate molecule of claim 17, wherein the second target antigen is expressed on a second cell surface.

19. The immunoconjugate molecule of claim 17 or 18, wherein the second cell is a cancer cell or a cell in a tumor microenvironment.

20. The immunoconjugate molecule of any one of claims 17 to 19, wherein the second target antigen is soluble.

21. The immunoconjugate molecule of any one of claims 17 to 20, wherein the second target antigen is a tumor associated antigen.

22. The immunoconjugate molecule of any one of claims 17 to 21, wherein the first and second target antigens are the same.

23. The immunoconjugate molecule of claim 22, wherein the bispecific masking moiety and the anchoring moiety bind to the same epitope of the first or second target antigen.

24. The immunoconjugate molecule of claim 22, wherein the bispecific masking moiety and the anchoring moiety bind to different epitopes of the first or second target antigen.

25. The immunoconjugate molecule of any one of claims 17 to 24, wherein the second target antigen is fibroblast activation protein (FAP) or Tumor-associated calcium signal transducer 2 (TROP2).

26. The immunoconjugate molecule of any one of claims 17 to 21, wherein the first and second target antigens are different.

27. The immunoconjugate molecule of any one of claims 17 to 26, wherein the first and second cells are the same or different.

28. The immunoconjugate molecule of any one of claims 17 to 27, wherein the anchoring moiety comprises an intact antibody, a Fab, a scFab, a Fab′, a F(ab′)₂, a Fv, a scFv, a dsFv, a diabody, a triabody, a tetrabody, or a VHH formed from antibody fragments.

29. The immunoconjugate molecule of any one of claims 17 to 28, wherein the bispecific antibody or antigen binding fragment of the masking moiety is a Fab, scFv or VHH, and wherein the antibody or antigen binding fragment thereof of the anchoring moiety is a Fab, scFv or VHH.

30. The immunoconjugate molecule of any one of claims 17 to 28, wherein the bispecific antibody or antigen binding fragment of the masking moiety is a Fab, and wherein the antibody or antigen binding fragment thereof of the anchoring moiety is a scFv.

31. The immunoconjugate molecule of any one of claims 1 to 30, wherein the masking moiety is fused directly to the cytokine moiety.

32. The immunoconjugate molecule of any one of claims 1 to 30, wherein the masking moiety is fused to the cytokine moiety via a peptidic linker.

33. The immunoconjugate molecule of claim 31 or 32, wherein the masking moiety is fused to the C-terminus the TNF-α polypeptide.

34. The immunoconjugate molecule of claim 31 or 32, wherein the masking moiety is fused to the N-terminus the TNF-α polypeptide.

35. The immunoconjugate molecule of any one of claims 17 to 34, wherein the anchoring moiety is fused directly to the cytokine moiety or is fused directly to the masking moiety.

36. The immunoconjugate molecule of any one of claims 17 to 34, wherein the anchoring moiety is fused to the cytokine moiety or the masking moiety via a peptidic linker.

37. The immunoconjugate molecule of any one of claims 17 to 36, wherein the masking moiety comprises a Fab having a heavy chain subunit and a light chain subunit, wherein the N terminus of the heavy chain subunit is fused via a linker to the TNF-α polypeptide, and wherein the N terminus of light chain subunit is fused via a linker to the anchoring moiety.

38. The immunoconjugate molecule of claim 37, wherein the anchoring moiety is a scFv.

39. The immunoconjugate molecule of any one of claims 1 to 30, further comprising:
(d) a conjugating moiety, wherein the conjugating moiety operably connects two or more of the cytokine moiety, the masking moiety, and the anchoring moiety.

40. The immunoconjugate molecule of claim 39, wherein the conjugating moiety comprises an immunoglobulin Fc domain or a mutant thereof.

41. The immunoconjugate molecule of claim 40, wherein the Fc domain comprises a first subunit and a second subunit that are two non-identical polypeptidic chains; and
wherein the Fc domain comprises a first modification promoting hetero-dimerization of the two non-identical polypeptidic chains.

42. The immunoconjugate molecule of claim 41, wherein the first modification is a knob-into-hole modification comprising a knob modification in the first subunit and a hole modification in the second subunit.

43. The immunoconjugate molecule of any one of claims 40 to 42, wherein the Fc domain comprises a second modification, wherein the Fc domain has reduced binding affinity to an Fc receptor compared to a native Fc domain without said second modification.

44. The immunoconjugate molecule of claim 43, wherein the Fc domain has reduced binding affinity to a Fcγ receptor as compared to the native Fc domain without said second modification.

45. The immunoconjugate molecule of claim 44, wherein the Fcγ receptor is an FcγRIIIα, FcγRI or FcγRIIα receptor.

46. The immunoconjugate molecule of claim 43, wherein the Fc domain has reduced binding affinity to a complement component as compared to the native Fc domain without said second modification.

47. The immunoconjugate molecule of claim 46, wherein the complement component is C1q.

48. The immunoconjugate molecule of claim 43, wherein the Fc domain has reduced Fc effector function as compared to an Fc domain without said second modification.

49. The immunoconjugate molecule of claim 48, wherein the reduced Fc effector function is selected from complement dependent cytotoxicity (CDC), antibody-dependent cell-mediated cytotoxicity (ADCC), antibody-dependent cellular phagocytosis (ADCP), cytokine secretion, downregulation of cell surface receptors, and B cell activation.

50. The immunoconjugate molecule of any one of claims 43 to 49, wherein the second modification comprises one or more mutations selected from S228P, E233P, L234V, L234A, L235A, L235E, ΔG236, D265G, N297A, N297D, P329E, P329S, P329A, P329G, A330S, or P331S, wherein the numbering is that of the EU index as in Kabat.

51. The immunoconjugate molecule of any one of claims 43 to 49, wherein the second modification comprises one or more mutations selected from E233P, L234V, L234A, L235A, ΔG236, D265G, P327E, A328S, P329E, A330S, or P331S, wherein the numbering is that of the EU index as in Kabat.

52. The immunoconjugate molecule of any one of claims 40 to 51, wherein the cytokine moiety is connected to the C-terminus of one of the first and second subunits of the Fc domain, and the masking moiety is connected to the C-terminus of the other of the first and second subunits of the Fc domain.

53. The immunoconjugate molecule of claim 52, wherein the anchoring moiety is connected to the N-terminus of one of the first and second subunits of the Fc domain.

54. The immunoconjugate molecule of claim 53, wherein the anchoring moiety and the cytokine moiety are connected to the same subunit of the Fc domain.

55. The immunoconjugate molecule of claim 53, wherein the anchoring moiety and the masking moiety are connected to the same subunit of the Fc domain.

56. The immunoconjugate molecule of any one of claims 40 to 51, wherein the masking moiety is connected to the C-terminus of one of the first and second subunits of the Fc domain; and wherein the cytokine moiety is connected to the masking moiety.

57. The immunoconjugate molecule of claim 56, wherein the anchoring moiety is connected to the N-terminus of one of the first and second subunits of the Fc domain.

58. The immunoconjugate molecule of claim 57, wherein the anchoring moiety and the masking moiety are connected to the same subunit of the Fc domain; or wherein the anchoring moiety and the masking moiety are connected to different subunits of the Fc domain.

59. The immunoconjugate molecule of any one of claims 40 to 51, wherein the masking moiety is connected to the N-terminus of one of the first and second subunits of the Fc domain, and the cytokine moiety is connected to the masking moiety.

60. The immunoconjugate molecule of any one of claims 40 to 51, wherein the masking moiety is connected to the N-terminus of one of the first and second subunits of the Fc domain, and wherein the anchoring moiety is connected to the N-terminus of the other one of the first and second subunits of the Fc domain.

61. The immunoconjugate molecule of claim 60, wherein the cytokine moiety is connected to the masking moiety.

62. The immunoconjugate molecule of claim 60, wherein the cytokine moiety is connected to the anchoring moiety.

63. The immunoconjugate molecule of any one of claims 39 to 62, wherein the connection between two or more of the cytokine moiety, the masking moiety, the anchoring moiety and the conjugating moiety is via a peptidic linker.

64. A complex comprising a homotrimer of the immunoconjugate molecule of any one of claims 1 to 63, and wherein the trimerization is through the TNF-α polypeptide in the cytokine moiety of the immunoconjugate molecule.

65. A composition comprising the immunoconjugate molecule of any one of claims 1 to 63 or the complex of claim 64, and a pharmaceutical acceptable carrier.

66. A polynucleotide encoding the immunoconjugate molecule of any one of claims 1 to 63, or a fragment thereof.

67. The polynucleotide of claim 66, wherein the polynucleotide is operably linked to a promoter.

68. A vector comprising the polynucleotide of claim 66 or 67.

69. A cell comprising the polynucleotide of any one of claims 65 to 67.

70. A cell comprising the vector of claim 68.

71. An isolated cell producing the immunoconjugate molecule of any one of claims 1 to 63 or the complex of claim 64.

72. A kit comprising the immunoconjugate molecule of any one of claims 1 to 63 or the complex of claim 64.

73. A method of making an immunoconjugate molecule, comprising culturing the cell of any one of claims 69 to 71 to express the immunoconjugate molecule, wherein the immunoconjugate molecule is in a monomeric, dimeric, or trimeric form.

74. A method of making an immunoconjugate molecule, comprising expressing the polynucleotide of claim 66 or 67, wherein the immunoconjugate molecule is in a monomeric, dimeric, or trimeric form.

75. A method for activating a TNF-α mediated effect at a target site, the method comprising delivering to the target site an immunoconjugate molecule comprising the TNF-α polypeptide and a masking moiety;
wherein the masking moiety comprises a two-in-one antibody or antigen binding fragment thereof that binds to TNF-α through intramolecular interaction and inhibits the TNF-α mediated effect;
wherein the two-in-one antibody or antigen binding fragment is capable of binding to a first target antigen in the target site;
wherein when the immunoconjugate molecule is at the target site, the two-in-one antibody binds to the first target antigen and disassociate from TNF-α; and
wherein the TNF-α mediated effect is activated at the target site.

76. The method of claim 75, wherein the immunoconjugate molecule further comprises an anchoring moiety; wherein the anchoring moiety comprises an antibody or antigen binding fragment thereof capable of binding to a second target antigen in the target site.

77. The method of claim 76, wherein when the immunoconjugate molecule is at the target site, the antibody or antigen binding fragment of the anchoring moiety binds to the second target antigen; and wherein the immunoconjugate molecule is immobilized at the target site.

78. The method of any one of claims 75 to 77, wherein delivering the immunoconjugate molecule to the target site comprises administering the immunoconjugate molecule to a subject.

79. The method of claim 78, wherein the TNF-α mediated effect is at least about 10%, 20%, 30%, 40%, 45%, 50%, 55%, 60%, 65%, 70%, 75%, 80%, 85%, 90%, 95%, or 98% lower at a non-target site as compared to the TNF-α mediated effect at the target site after administering the immunoconjugate molecule to the subject.

80. The method of any one of claims 75 to 79, wherein the TNF-α mediated effect is cell apoptosis.

81. A method for enriching a TNF-α polypeptide at a target site, the method comprising delivering to the target site an immunoconjugate molecule comprising a TNF-α polypeptide and an anchoring moiety;
wherein the anchoring moiety comprises an antibody or antigen binding fragment thereof capable of binding to a second target antigen in the target site;
wherein when the immunoconjugate molecule is at the target site, the anchoring moiety binds to the second target antigen; and
wherein the TNF-α polypeptide is distributed at a higher concentration at the target site compared to a non-target site.

82. The method of claim 81, wherein delivering the immunoconjugate molecule to the target site comprises administering the immunoconjugate molecule to a subject.

83. The method of claim 82, wherein the concentration of the TNF-α polypeptide is at least about 10%, 20%, 30%, 40%, 45%, 50%, 55%, 60%6, 65%, 70%, 75%, 80%, 85%, 90%, 95%, or 98% lower at a non-target site as compared to the concentration of the TNF-α polypeptide at the target site after administering the immunoconjugate molecule to the subject.

84. The method of claim 78, 79, 82 or 83, wherein a toxicity or side-effect associated with TNF-α in the subject is reduced.

85. The method of claim 84, wherein cytokine toxicity or side-effect is reduced at least about 10%, 20%, 30%, 40%, 45%, 50%, 55%, 60%, 65%, 70%, 75%, 80%, 85%, 90%, 95%, or 98% as compared to administering to the subject an equivalent amount of TNF-α in an unconjugated form.

86. The method of claim 84 or 85, wherein the reduction in toxicity or side-effect is measured as the elongation of life span of the administered subject.

87. The method of claim 84 or 85, wherein the reduction in toxicity or side-effect is measured as reduction in loss of body weight of the administered subject.

88. The method of claim 84 or 85, wherein the reduction in toxicity or side-effect is measured as change in the level of an immune response in the administered subject.

89. The method of claim 84 or 85, wherein the reduction in toxicity or side-effect is measured as a change in an inflammatory response in the administered subject.

90. The method of claim 81, wherein the immunoconjugate molecule further comprises a masking moiety;
wherein the masking moiety comprises a two-in-one antibody or antigen binding fragment thereof that binds to the TNF-α polypeptide through intramolecular interaction and inhibits an TNF-α mediated effect;
wherein the two-in-one antibody or antigen binding fragment thereof is capable of binding to a first target antigen in the target site;
wherein when the immunoconjugate molecule is at the target site, the two-in-one antibody binds to the first target antigen and disassociate from the TNF-α polypeptide; and
wherein the TNF-α mediated effect is activated at the target site.

91. The method of claim 76, 77, or 90 wherein the first antigen and second antigen are the same antigen or different antigens.

92. The method of any one of claims 75 to 91, wherein the target site is a tumor microenvironment.

93. The method of any one of claims 75 to 91, wherein the target site is a cancerous cell.

94. The method of claim 92 or 93, wherein the first and/or second antigen is expressed on the surface of cancer cells.

95. The method of claim 92, wherein the first and/or second antigen is expressed by cells in the tumor microenvironment.

96. The method of claim 94 or 95, wherein the cell expressing the first and/or second antigen also expresses a TNF-α receptor.

97. The method of claim 94 or 95, wherein the cell expressing the first and/or second antigen does not express a TNF-α receptor.

98. The method of any one of claims 95 to 97, wherein the first and/or second antigen is fibroblast activation protein (FAP) or Tumor-associated calcium signal transducer 2 (Trop-2).

99. The method of any one of claims 75 to 98, wherein the immunoconjugate molecule is the immunoconjugate molecule of any one of claims 1 to 63.

100. The method of any one of claims 75 to 99, wherein the immunoconjugate molecule is in a monomeric, dimeric, or trimeric form.

101. A method for inhibiting growth or induce apoptosis of a cancer cell comprising contacting the cancer cell with an effective amount of the immunoconjugate molecule of any one of claims 1 to 63 or the complex of claim 64.

102. A method for treating cancer in a subject in need thereof, wherein the method comprising administering an effective amount of the immunoconjugate molecule of any one of claims 1 to 63 or the complex of claim 64.


SEQUENCE LISTING (7 sequences)

SEQ ID NO: 1 - protein, 157 aa, Homo sapiens [sequence omitted]

SEQ ID NO: 2 - protein, 454 aa, Homo sapiens
  sequence of an exemplary human TNFR1
GLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 3 - protein, 460 aa, Homo sapiens
  sequence of an exemplary human TNFR2
APVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 4 - protein, 760 aa, Homo sapiens
  sequence of an exemplary human FAP
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 5 - protein, 322 aa, Homo sapiens
  sequence of an exemplary human Trop-2
ARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 6 - protein, 227 aa, Homo sapiens
  a native human IgG1 Fc region amino acid sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 15 aa, Artificial Sequence
  REPEAT (1)..(5): repeats of G4S fragment at the specified length
GGGGSGGGGSGGGGS